(19) United States
(12) Patent Application Publication
(10) Pub. No.: US 2024/0374573 A1
(43) Pub. Date: Nov. 14, 2024

(54) IMIDAZOLE CONTAINING COMPOUNDS, DERIVATIVES THEREFORE, AND USES THEREOF

(71) Applicants: Lingyu Zhu, Pawcatuck, CT (US); AglaeaPharma Inc., Germantown, MD (US)

(72) Inventors: Lingyu Zhu, Pawcatuck, CT (US); Zhiqiang Cheng, Ellicott City, MD (US)

(21) Appl. No.: 18/644,976

(22) Filed: Apr. 24, 2024

Related U.S. Application Data
(60) Provisional application No. 63/498,049, filed on Apr. 25, 2023.
(60) Provisional application No. 63/515,229, filed on Jul. 24, 2023.
(60) Provisional application No. 63/550,274, filed on Feb. 6, 2024.
(60) Provisional application No. 63/550,228, filed on Feb. 6, 2024.
(60) Provisional application No. 63/557,039, filed on Feb. 23, 2024.
(60) Provisional application No. 63/571,160, filed on Mar. 28, 2024.

(51) Int. Cl. A61K 31/4174; A61K 31/4178; A61K 31/421; A61K 31/426; A61K 31/427; A61K 31/4409; A61K 31/4439; A61K 31/454; A61K 31/496; A61K 31/5377; A61K 31/664; A61P 25/02; A61P 25/04; A61P 35/00

(52) U.S. Cl. CPC A61K 31/4174; A61K 31/4178; A61K 31/421; A61K 31/426; A61K 31/427; A61K 31/4409; A61K 31/4439; A61K 31/454; A61K 31/496; A61K 31/5377; A61K 31/664; A61P 25/02; A61P 25/04; A61P 35/00

(57) ABSTRACT

The present disclosure relates to novel alpha2 adrenergic receptor (α2AR) agonists and uses thereof. In particular, the present disclosure relates to imidazole containing compounds, in particular, of formula (I-A), formula (I-B), formula (I-C), formula (I-D), or formula (II). These compounds can be useful as peripherally selective α2AR agonists for the treatment or prevention of disease thereof.

### CROSS-REFERENCE TO RELATED APPLICATION

This application claims priority to U.S. Patent Application No. 63/498,049 filed Apr. 25, 2023, U.S. Patent Application No. 63/515,229 filed Jul. 24, 2023, U.S. Patent Application No. 63/550,274 filed Feb. 6, 2024, U.S. Patent Application No. 63/550,228 filed Feb. 6, 2024, U.S. Patent Application No. 63/557,039 filed Feb. 23, 2024, and U.S. Patent Application No. 63/571,160 filed Mar. 28, 2024, the disclosures of which are incorporated herein by reference in their entireties.

### FIELD OF THE DISCLOSURE

The present disclosure describes novel alpha2 adrenergic receptor (α2AR) agonists and uses thereof. In particular, the present disclosure describes novel imidazole containing compounds and their derivatives. These compounds can be useful as α2AR agonists for the treatment or prevention of diseases thereof.

### BACKGROUND OF THE DISCLOSURE

The alpha2 adrenergic receptor (α2AR) family, as part of the G-protein-coupled receptors, plays a critical role for many central nervous system (CNS) biological functions. α2ARs are key in modulating neurotransmitter release, thus influencing a spectrum of central physiological processes. Agonists targeting these receptors, such as clonidine and dexmedetomidine, have been successfully used to treat several conditions predominantly within the CNS. Related applications include treating hypertension, sedation in intensive care, and for problems like attention-deficit/hyperactivity disorder (ADHD) and agitation associated with schizophrenia or bipolar disorder.

Clonidine was first developed to manage hypertension. Administered orally, clonidine diffuses into the CNS and activates the α2AR in the nucleus tractus solitarii (NTS), which in turn triggers a pathway inhibiting excitatory cardiovascular neurons. This cascade effectively reduces sympathetic outflow from the CNS, leading to a clinical decrease in arterial blood pressure.

Later, clonidine was found to induce sedation by acting through the activation of central pre- and postsynaptic α2AR in the locus coeruleus (LC), a nucleus in the medial dorsal pons, thereby inducing sedative effects. The later development and approval of dexmedetomidine for sedation, particularly in initially intubated and mechanically ventilated adult patients in intensive care settings, was attributed to its superior α2AR selectivity and pharmacokinetic properties better suited for sedation.

Beyond its antihypertensive and sedation effects, clonidine has been approved for epidural use under the trade name Duraclon, marking a significant advancement in the treatment of cancer pain. The analgesic mechanism is widely attributed to clonidine's diffusion into the spinal cord and activation of α2ARs in the dorsal horn, thereby attenuating pain transmission to higher CNS centers. This central action enables α2AR agonists to produce significant analgesic effects, making them an important method for managing pain.

However, the therapeutic application of α2AR agonists on analgesia comes with challenges, primarily due to the range of other biological adverse effects they can cause in CNS. Duraclon has been documented to induce centrally mediated sedation, hypotension, bradycardia, and depression of its applications, which persist throughout the analgesic treatment process. Such sedation effect significantly limits the dosages that can be administered safely. As a result, although α2AR agonists like clonidine and dexmedetomidine are considered important for pain treatment in both academic research and clinical settings, the sedation effect poses substantial hurdles to their widespread use in medical applications.

Therefore, it is desired to develop new classes of α2AR agonists that could provide substantial therapeutic benefits in pain management such as reduced sedation effect, thereby expanding the range of therapeutic alternatives to address the prevailing unmet medical needs.

### BRIEF SUMMARY OF THE DISCLOSURE

In one general aspect, the present disclosure relates to methods of treating or preventing a disease in a subject in need thereof, the method comprising administering to the subject a therapeutically effective amount of a peripherally selective alpha2 adrenergic receptor (α2AR) agonist.

In some embodiments, the peripherally selective α2AR agonist has a Kp,uu,brain is lower than 0.05, 0.02, or 0.01.

In some embodiments, the peripherally selective α2AR agonist activates at least one sub type of α2AR, particularly α2A AR, α2B AR, or α2C AR.

In some embodiments, the disease is chosen from pain, rosacea, spasticity, and aging.

In some embodiments, the peripherally selective α2AR agonist causes reduced biological effects mediated by CNS, such as sedation, hypotension, and bradycardia, than treating with a non-peripherally selective α2AR agonist.

In another general aspect, the present disclosure provides a peripherally selective α2AR agonist that comprises an α2AR activation moiety covalently linked to a peripheral distribution moiety, and its uses in the treatment of a disease.

In another general aspect, the present disclosure relates to methods of treating or preventing a disease in a subject in need thereof, the method comprising administering to the subject a therapeutically effective amount of a peripherally selective α2AR agonist, wherein the peripherally selective α2AR agonist comprises an α2AR activation moiety covalently linked to a peripheral distribution moiety.

In another general aspect, the present disclosure provides a pharmaceutical composition comprising: (a) a means for increasing the activation of α2AR of the peripheral nervous system of a subject, and (b) a pharmaceutically acceptable carrier.

In another general aspect, the present disclosure provides a pharmaceutical composition comprising: (a) a molecule comprising (i) a first means for activating α2AR, and (ii) a second means for increasing distribution of the molecule to the peripheral nervous system of a subject, wherein the first means is covalently linked to the second means, and (b) a pharmaceutically acceptable carrier.

In some embodiments, the peripherally selective α2AR agonist causes less sedation than treating with a non-peripherally selective α2AR agonist.

In another general aspect, the present disclosure relates to a compound of formula (I-A):

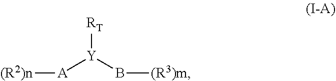

or a stereoisomer, tautomer, pharmaceutically acceptable salt or solvate thereof,
wherein,
Y is C(R¹), N, —O—C, —C—NH—, —CH₂—C(O)—, or —CH═N—;
   when Y is C(R¹), R¹ is chosen from H, D, and halogen;
   when Y is —O—C—, the oxygen atom is connected to A, and the carbon atom is connected to both R_{T} and B; and
   when Y is —C—NH—, the carbon atom is connected to both R_{T} and A, and the nitrogen atom is connected to B;
A is a ring chosen from phenyl, pyridinyl, thienyl, furyl, pyrrolyl, 4H-pyran, 4H-thiopyran, 1,2,3,4-tetrahydro-1-naphthyl, tetrahydrozoline, quinoxalinyl, pyrimidinyl, and 2,1,3-benzothiadiazol;
B is

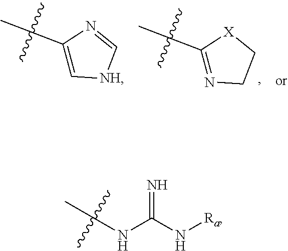

wherein X is NH, O, or S, and Ra is H and methyl;
n is 0, 1, 2, or 3;
each R² is independently chosen from H, D, halogen, alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl, OR⁴, —CN, N₃, NO₂, N(R⁴)₂, OR₄, SR⁴, C(O)R⁴, SO₂N(R⁴)₂, CH₂SR⁴, wherein the alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl is optionally substituted with one or more R⁵;
R⁴ is chosen from H, D, halogen, alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl, and the alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl is optionally substituted with one or more R⁵;
R⁵ is chosen from halogen, hydroxyl, —CN, —NO₂, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;
alternatively, when A is a phenyl ring and two R² are substituted at adjacent positions of the phenyl ring, the two R² groups, with the carbon atoms they are connected to, form a ring that is fused to ring A to form a bicyclic ring, such as quinolinyl, indolyl, benzothienyl, benzofuryl, benzofuranyl, benzodioxolyl, 2,3-dihydrobenzo[b][1,4]dioxin-6-yl, cinnolinyl, quinoxalinyl, or 1,2,4-benzotriazinyl;
m is 0, 1, 2, or 3;
each R³ is independently chosen from H, D, halogen, —OH, —SH, optionally substituted alkyl, optionally substituted heterocycle, and optionally substituted aryl;
alternatively, R³ is a group connected to the —NH of the imidazole ring, and R³ is of the formula of:

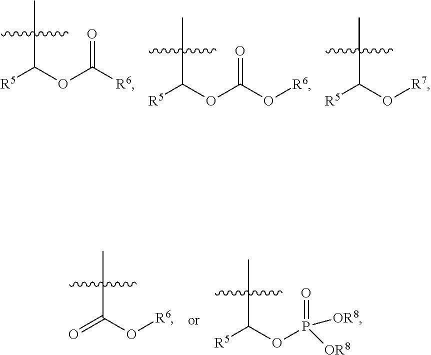

wherein:
R⁵ is hydrogen or alkyl;
R⁶ is hydrogen, alkyl, cycloalkyl, or alkenyl;
R⁷ is an amino acid residue; and
R⁸ is alkyl or cycloalkyl;
R_{T} is R_{L}—R_{P}, and R_{P} is optionally substituted with R_{C}, wherein:
R_{L} is a linker, wherein one end is connected to R_{P} and the other end is connected to Y;
R_{P} is a moiety that is connected to one end of R_{L}; and
R_{C} is a cap, which is a moiety that is connected to R_{P}.

In another general aspect, the present disclosure relates to a compound of formula (I-B):

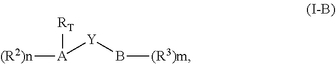

or a stereoisomer, tautomer, pharmaceutically acceptable salt or solvate thereof,
wherein,
Y is a bond, CH(R¹), NH, —O—CH—, —C—NH—, —CH₂—C(O)—, or —CH═N—;
   when Y is C(R¹), R¹ is chosen from H, D, and halogen;
   when Y is —O—C—, the oxygen atom is connected to A, and the carbon atom is connected to B;
   when Y is —C—NH—, the carbon atom is connected to A, and the nitrogen atom is connected to B; and
A, B, R², n, R³, m, and R_{T} are defined as above in formula (I-A).

In another general aspect, the present disclosure relates to a compound of formula (I-C):

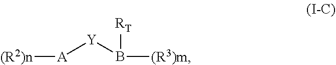

or a stereoisomer, tautomer, pharmaceutically acceptable salt or solvate thereof,
wherein,
Y is a bond, CH(R¹), NH, —O—CH—, —C—NH—, —CH₂—C(O)—, or —CH═N—;
   when Y is C(R¹), R¹ is chosen from H, D, and halogen;
   when Y is —O—C—, the oxygen atom is connected to A, and the carbon atom is connected to B;
   when Y is —C—NH—, the carbon atom is connected to A, and the nitrogen atom is connected to B; and
A, B, R², n, R³, m, and R_{T} are defined as above in formula (I-A).

In another general aspect, the present disclosure relates to a compound of formula (I-D):

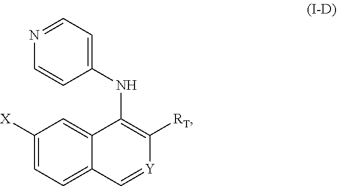

or a stereoisomer, tautomer, pharmaceutically acceptable salt or solvate thereof,
wherein,
X is CH, or N;
Y is chosen from H, D, and halogen;
R_{T} is defined as above in formula (I-A).

In another general aspect, the present disclosure relates to a compound of formula (II):

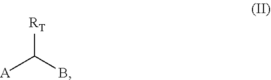

or a stereoisomer, tautomer, pharmaceutically acceptable salt or solvate thereof,
wherein,
A is one chosen from:

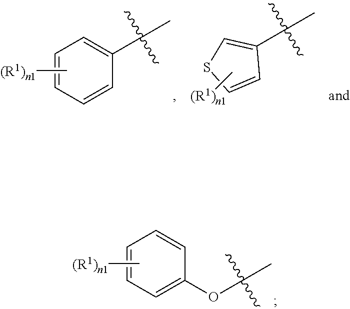

n1 is 1 or 2;
each R¹ is independently chosen from hydrogen, halogen, haloalkyl, hydroxyl, hydroxyalkyl, alkoxy, alkyl, and —COOH;
B is one chosen from:

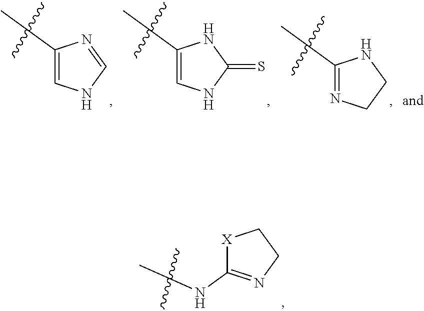

wherein X is S, O, or NH;
R_{T} is

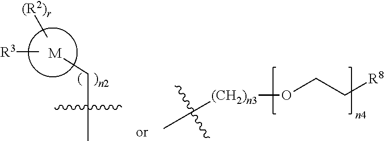

ring M is C₃₋₁₂ cycloalkyl, C₂₋₁₂ heterocyclyl, C₆₋₁₂ aryl, or C₁₋₁₂ heteroaryl, wherein the C₃₋₁₂ cycloalkyl or C₂₋₁₂ heterocyclyl is optionally fused with an aryl;
r is 1 or 2;
n2 is 0, 1, or 2;
each R² is independently chosen from hydrogen, halogen, hydroxyl, and alkoxy;
R³ is chosen from CN, hydroxy, alkoxy, —C(O)—C₀₋₁₂ alkylene-CN, —C₀₋₁₂ alkylene-C₂₋₁₂ heterocyclyl, —SO₂-alkyl, —C(O)—NR⁴R^{4′}, —SO₂—NR⁴R^{4′}, —C₀₋₁₂ alkylene-R^{3′}, —O—C₀₋₁₂ alkylene-COOH, —C₀₋₁₂ alkylene-N(R⁴)—C(O)—R⁵, —C₀₋₁₂ alkylene-N(R⁴)—SO₂—R⁵, —C₀₋₁₂ alkylene-C₁₋₁₂ heteroaryl, —C₀₋₁₂ alkylene-O—C₀₋₁₂ alkylene-N(R⁴)—SO₂—R⁵, —C₀₋₁₂ alkylene-P(═O)(R⁴) (R^{4′}),

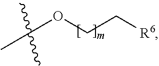

### —NH—R⁷, or

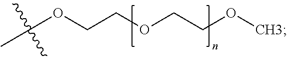

wherein one-CH₂— group in the —C₀₋₁₂ alkylene-R^{3′} is optionally replaced by oxygen atom or

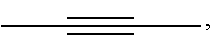

the —C₀₋₁₂ alkylene-R^{3′} is optionally substituted with one or more substitutes chosen from amino and alkylamino, and the C₂₋₁₂ heterocyclyl and C₁₋₁₂ heteroaryl are each optionally substituted with one or more R^{4a};
R^{3′} is chosen from —C(O)—NR⁴R^{4′}, —SO₂—NR⁴R^{4′}, —C₀₋₁₂ alkylene-COOH, —C₀₋₁₂ alkylene-N(R⁴)—C(O)—R⁵, —C₀₋₁₂ alkylene-N(R⁴)—SO₂—R⁵, C₀₋₁₂ alkylene-C₁₋₁₂ heteroaryl;
each R^{4a} is independently chosen from hydroxy, alkyl, oxo, ketone, and —C₂₋₁₂ heterocyclyl;
each of R⁴ and R^{4′} is independently hydrogen, alkyl, alkoxy, —SO₂—N(R⁶²) t, —C₀₋₁₂ alkylene-COOH, —C₀₋₁₂ alkylene-N(R^{6a})ₜ, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or hydroxyalkyl, wherein the hydroxyalkyl is optionally substituted with alkoxy; wherein each of the alkyl, C₃₋₁₂ Cycloalkyl, C₂₋₁₂ heterocyclyl, and C₁₋₁₂ heteroaryl is optionally substituted with one or more R^{4a};
or R⁴ and R^{4′}, together with the nitrogen atom that they are attached to, form a heterocycle comprising one or more heteroatoms chosen from O, N, and S;
alternatively, when one R² is adjacent to R³, the R² and R³, together with the atoms that they are attached to, form a ring optionally substituted with one or more R^{4a};
R⁵ is amino, alkylamino, C₁₋₁₂ haloalkyl, —C₀₋₁₂ alkylene-OR^{6a}, —C₀₋₁₂ alkylene-N(R^{6a}) t, —C₀₋₁₂ alkylene-SR^{6a}, —C₀₋₁₂ alkylene-CN, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₂₋₁₂ alkenyl, or alkyl optionally substituted with cyano, amido, trialkylammonium, or thiolate; wherein each of the C₃₋₁₂ cycloalkyl, C₂₋₁₂ heterocyclyl, and C₁₋₁₂ heteroaryl is optionally substituted with one or more R^{4a},
each R^{6a} is independently chosen from hydrogen, C₁₋₁₂ alkyl, C₁₋₁₂ alkoxy, —C₀₋₁₂ alkylene-C₃₋₁₂ cycloalkyl, —C₀₋₁₂ alkylene-C₂₋₁₂ heterocyclyl, —C₀₋₁₂ alkylene-C₆₋₁₂ aryl, and —C₀₋₁₂ alkylene-C₁₋₁₂ heteroaryl; wherein each of the alkyl, C₃₋₁₂ cycloalkyl, C₂₋₁₂ heterocyclyl, and C₁₋₁₂ heteroaryl, is optionally substituted with one or more R^{4a};
R⁶ is alkoxy, amino, sulfonamide, carbamide, or alkyl optionally substituted with cyano;
R⁷ is hydrogen, alkyl, —C₀₋₁₂ alkylene-COOH, optionally substituted C₃₋₁₂ Cycloalkyl, C₂₋₁₂ aryl, C₁₋₁₂ heteroaryl, —C₀₋₁₂ alkylene-N(R⁴)—SO₂—R⁵, —C₀₋₁₂ alkylene-P(═O)(R⁴) (R^{4′}), —C₀₋₁₂ alkylene-N(R⁴)—C(═S)—R⁵, —C(═S)—R⁵, or alkyl optionally substituted with cyano;
R⁸ is alkoxy, amino, alkylamino, amide, sulfonamide, or carbamide;
n3 is 0, 1, 2, 3, or 4;
n4 is 1, 2, 3, 4, 5, or 6;
t is 2 or 3;
m is 0, 1, 2, 3, 4, or 5; and
n is 0, 1, 2, 3, or 4.

In some embodiments, the compound of formula (II) is a compound of formula (II-A):

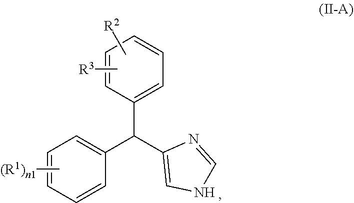

wherein R¹, R², R³, and n1 are defined as above in formula (II).

In some embodiments, the compound of formula (II) is a compound of formula (II-B):

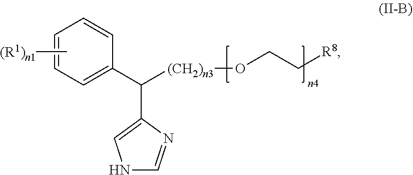

wherein, R¹, R⁸, n1, n3, and n4 are defined as above in formula (II).

In some embodiments, the compound of formula (II) is a compound of formula (II-C):

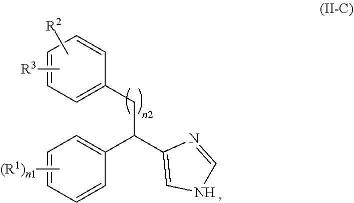

wherein,
n2 is 1 or 2; and
R¹, R², R³, and n1 are defined as above in formula (II).

In some embodiments, the compound of formula (II) is a compound of formula (II-D):

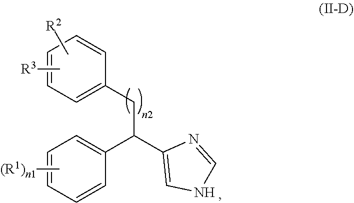

wherein,
n2 is 0 or 1;
R³ is chosen from —C(O)—NHR⁴, —SO₂—NHR⁴, —NH—C(O)—R⁵, and —NH—SO₂—R⁵, and —NH—R⁷;
R⁴ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein and each of the C₃₋₁₂ cycloalkyl, C₂₋₁₂ heterocyclyl, and C₁₋₁₂ heteroaryl is optionally substituted with one or more R^{4a};
R⁵ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein each of the C₃₋₁₂ cycloalkyl, C₂₋₁₂ heterocyclyl, and C₁₋₁₂ heteroaryl is optionally substituted with one or more R^{4a};
each R^{6a} is independently chosen from —C₀₋₁₂ alkylene-C₃₋₁₂ cycloalkyl, —C₀₋₁₂ alkylene-C₂₋₁₂ heterocyclyl, and —C₀₋₁₂ alkylene-C₁₋₁₂ heteroaryl; wherein each of the C₃₋₁₂ cycloalkyl, C₂₋₁₂ heterocyclyl, and C₁₋₁₂ heteroaryl is optionally substituted with one or more R^{4a};
R⁷ is C₁₋₁₂ heteroaryl, —C₀₋₁₂ alkylene-N(R⁴)—SO₂—R⁵, —C₀₋₁₂ alkylene-P(═O)(R⁴) (alkoxy), or —C₀₋₁₂ alkylene-N(R⁴)—C(═S)—R⁵; and
R¹, R², R^{4a}, and n1 and are defined as above in formula (II).

In some embodiments, the compound of formula (II) is a compound of formula (II-E):

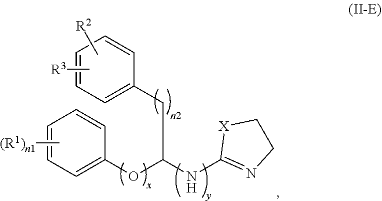

wherein
x is 0 or 1;
y is 0 or 1;
X is S, O, or NH; and
R¹, R², R³, n1, and n2 are defined as above in formula (II).

In some embodiments, the compound of formula (II) is a compound of formula (II-F):

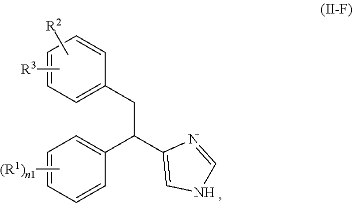

wherein,
R² is adjacent to R³, and R² and R³, together with the carbon atoms that they are attached to, form a heterocycle optionally substituted with one or more R^{4a}; and
R¹, R^{4a}, and n1 are defined as above in formula (II).

In some embodiments, the compound of formula (II) is a compound of formula (II-G):

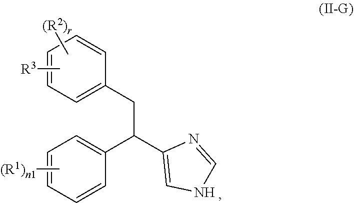

wherein,
each R² is independently chosen from hydroxyl and alkoxy;
R³ is chosen from hydroxy and alkoxy; and
R¹, n1 and r are defined as above in formula (II).

In some embodiments, the compound of formula (II) is a compound of formula (II-H):

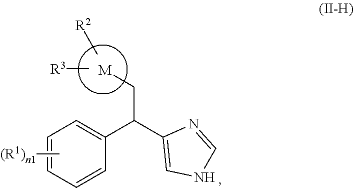

wherein,
M is C₆₋₁₂ aryl or C₁₋₁₂ heteroaryl;
R³ is chosen from —C₀₋₁₂ alkylene-COOH, —O—C₀₋₁₂ alkylene-COOH, —C₀₋₁₂ alkylene-P(O)(OH)₂, —C(O)—NH—SO₂—R⁵, —C(O)—NH—C₀₋₁₂ alkylene-COOH, —NH—C₀₋₁₂ alkylene-COOH, —SO₂—OH, and

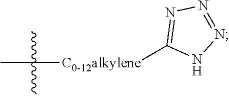

wherein the —C₀₋₁₂ alkylene-COOH is optionally substituted with one or more substitutes chosen from amino and alkylamino; and
R¹, R², and n1 are defined as above in formula (II).

In one general aspect, the present disclosure provides a means for activating «2 adrenergic receptor (α2AR).

In some embodiments, the means for activating α2AR is an α2AR agonist.

In another general aspect, the present disclosure provides a means for increasing distribution of the molecule to the peripheral nervous system of a subject.

In some embodiments, the R_{T} in formula I-A, I-B, I-C, or II is a means for increasing distribution of the molecule to the peripheral nervous system of a subject.

In another aspect, the present disclosure relates to a pharmaceutical composition comprising a compound as described herein or a stereoisomer, tautomer, pharmaceutically acceptable salt or solvate thereof, and a pharmaceutically acceptable carrier.

In another aspect, the present disclosure relates to the use of a compound as described herein or a stereoisomer, tautomer, pharmaceutically acceptable salt or solvate thereof, for treating or preventing a disease, including pain, glaucoma, pain, spasticity, nasal congestion, rosacea, rhinitis, anesthesia, presbyopia, acute kidney injury, insomnia, inflammatory disease, cancer, etc. in a subject in need thereof.

Other features and advantages of the present disclosure are apparent from additional descriptions provided herein, including different examples. The provided examples illustrate different components and methodology useful in practicing the present disclosure. Such examples do not limit the claimed disclosure. Based on the present disclosure, the skilled artisan can identify and employ other components and methodology useful for practicing the present disclosure.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1: PWT value of sham and SNI mouse model, vehicle and drug treatment groups at 1 hour after dosing. The drugs including A: 3 mg/mL pregabalin p.o.; B: 1 mg/mL morphine s.c.; C: 1 mg/mL 1-B p.o. and 10 mg/mL 1-B p.o.; D: 1 mg/mL 10-B p.o.; E: 1 mg/mL 44-B p.o. and 1 mg/mL 45-B p.o.; F: 1 mg/mL 46-B p.o. and 1 mg/mL 47-B p.o.; G: 2 mg/mL 121 p.o. and 2 mg/mL 136 p.o.; and H: 2 mg/mL 118 p.o. and 2 mg/mL 156 p.o.

FIG. 2: PWT value of sham and bone cancer pain mouse model, vehicle and drug treatment groups at 1 hour after dosing. The drugs including A: 3 mg/mL pregabalin p.o.; B: 1 mg/mL morphine s.c.; C: 1 mg/mL 44-B p.o.; and D: 20 mg/mL 1-B p.o. and: 20 mg/mL 44-B p.o.

FIG. 3: PWT value of sham and post-surgery pain mouse model, vehicle and drug treatment groups at 1 hour after dosing. The drugs including A: 10 mg/mL 1-B p.o.; B: 10 mg/mL 44-B p.o.; and C: 3 mg/mL morphine s.c.

FIG. 4: Body weight curve (A), tumor volume growth curve (B)), and tumor volume in day 17 (C) of mice in each group in subcutaneous colorectal cancer syngeneic model MC38, including group 1 (control group, 0 mg/kg, p.o., QD*Day0-17), group 2 (clonidine, 5 mg/kg, p.o., QD*Day0-3; 2 mg/kg, p.o., QD* Day4-17), group 3 (1-B HCl, 5 mg/kg, p.o., BID*Day0-17), and group 4 (1-B HCl, 10 mg/kg, p.o., BID*Day0-3; 5 mg/kg, p.o., QD* Day4-17). Data is expressed as “average±standard error”.

FIG. 5 Total distance travelled in 0-60 min of the test (A) for clonidine and compound 1-B HCl and the test (B) for clonidine, brimonidine tartrate and compound 44-B HCl. Data were expressed as Mean±SEM (n=6). ***p<0.001 compared with Vehicle group, one-way ANOVA followed by Dunnutt's multiple comparisons.

FIG. 6. Effects of clonidine and 44-B HCl compounds on rotarod test in C57BL/6 mice 30 min after administration (A). And its latency time at 30 min (B), 60 min (C), and 120 min (D).

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which the present disclosure pertains. Otherwise, certain terms used herein have the meanings as set forth in the specification.

As used herein and in the appended claims, the singular forms “a,” “an,” and “the” include plural reference (one or more) unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and conjuntive options. For instance, where two elements are conjoined by “and/or,” a first option refers to the applicability of the first element without the second. A second option refers to the applicability of the second element without the first. A third option refers to, conjunctively, the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes +10% of the recited value. For example, the recitation of “10-fold” includes 9-fold and 11-fold. As used herein, the use of a numerical range expressly includes all possible permutations and combinations of subranges, all individual numerical values within that range, including integers within such ranges and fractions of the values unless the context clearly indicates otherwise.

As used herein, “subject” means any animal, such as a mammal, particularly a human, to whom will be or has been treated by a method described herein. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc.

The phrase “pharmaceutically acceptable salt(s)” means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present disclosure can form pharmaceutically acceptable salts with various amino acids. Suitable base salts include, but are not limited to, aluminum, calcium, lithium, magnesium, potassium, sodium, zinc, bismuth, and diethanolamine salts. For a review on pharmaceutically acceptable salts see Berge et al., 66 J. Pharm. Sci. 1-19 (1977), incorporated herein by reference.

As used herein, the term “alkyl” means a saturated, monovalent, unbranched or branched hydrocarbon chain. An alkyl group can be unsubstituted or substituted with one or more suitable substituents. Examples of alkyl groups include, but are not limited to, methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When numbers appear in a subscript after the symbol “C”, the subscript defines with more specificity the number of carbon atoms which that particular alkyl can contain. For example, “C₁ to C₁₀ alkyl” or “C₁₋₁₀ alkyl” is intended to include alkyl groups having 1, 2, 3, 4, 5, 6, 7, 8, 9, and 10 carbon atoms. Additionally, for example, “C₁ to C₈ alkyl” or “C₁₋₈ alkyl” denotes an alkyl having 1, 2, 3, 4, 5, 6, 7, or 8 carbon atoms.

As used herein, the term “alkenyl” refers to an unbranched or branched hydrocarbon chain containing at least one carbon-carbon double bond. An alkenyl group can be unsubstituted or substituted with one or more suitable substituents. Examples of alkenyl groups include ethenyl, propenyl, butadienyl (including 1,2-butadienyl and 1,3-butadienyl). When numbers appear in a subscript after the symbol “C”, the subscript defines with more specificity the number of carbon atoms which that particular alkenyl can contain. For example, “C₂ to C₁₀ alkenyl” or “C₂₋₁₀ alkenyl” is intended to include alkenyl groups having 2, 3, 4, 5, 6, 7, 8, 9, and 10 carbon atoms. Additionally, for example, “C₂ to C₈ alkenyl” or “C₂₋₈ alkenyl” denotes an alkenyl having 2, 3, 4, 5, 6, 7, or 8 carbon atoms.

As used herein, the term “alkynyl” refers to an unbranched or branched hydrocarbon chain containing at least one carbon-carbon triple bond. An alkynyl group can be unsubstituted or substituted with one or more suitable substituents. The term “alkynyl” also includes those groups having one triple bond and one double bond. When numbers appear in a subscript after the symbol “C”, the subscript defines with more specificity the number of carbon atoms which that particular alkynyl can contain. For example, “C₂ to C₁₀ alkynyl” or “C₂₋₁₀ alkynyl” is intended to include alkynyl groups having 2, 3, 4, 5, 6, 7, 8, 9, and 10 carbon atoms. Additionally, for example, “C₂ to C₈ alkynyl” or “C₂₋₈ alkynyl” denotes an alkynyl having 2, 3, 4, 5, 6, 7, or 8 carbon atoms.

As used herein, the term “cycloalkyl” refers to any stable monocyclic or polycyclic saturated hydrocarbon ring system. A cycloalkyl group can be unsubstituted or substituted with one or more suitable substituents. A cycloalkyl group can have a specified number of carbon atoms. For example, “C₃ to C₆ cycloalkyl” or “C₃₋₆ cycloalkyl” includes cycloalkyl groups having 3, 4, 5, or 6 ring carbon atoms, i.e., cyclopropyl, cyclobutyl, cyclopentyl, and cyclohexyl. Polycyclic cycloalkyls include bridged, fused, and spiro ring structures in which all ring atoms are carbon atoms. A “spiro ring” is a polycyclic ring system in which two rings share one carbon atom, referred to as the “spiro atom,” which is typically a quaternary carbon atom. A “fused ring” is a polycyclic ring system in which two rings share two adjacent atoms, referred to as “bridgehead atoms,” i.e., the two rings share one covalent bond such that the bridgehead atoms are directly connected. A “bridged ring” is a polycyclic ring system in which two rings share three or more atoms separating the bridgehead atoms by a bridge containing at least one atom. Examples of cycloalkyl groups include, but are not limited to, cyclopropyl, cyclobutyl, cyclopentyl, cyclohexyl, cycloheptyl, cyclooctyl, etc.

The term “aryl” as used herein is a group that contains any carbon-based aromatic group including, but not limited to, phenyl, naphthyl, anthracenyl, phenanthranyl, and the like. Aryl moieties are well known and described, for example, in Lewis, R. J., ed., Hawley's Condensed Chemical Dictionary, 13th Edition, John Wiley & Sons, Inc., New York (1997). An aryl group can be substituted or unsubstituted with one or more suitable substituents. An aryl group can comprise a single ring structure (i.e., monocyclic) or multiple ring structures (i.e., polycyclic, e.g., bicyclic or tricyclic). For example, an aryl group can be a monocyclic aryl group, e.g., phenyl.

The term “heterocyclyl” includes stable monocyclic and polycyclic hydrocarbons that contain at least one heteroatom ring member, such as sulfur, oxygen, or nitrogen, wherein the ring structure is saturated or partially unsaturated, provided the ring system is not fully aromatic. A heterocyclyl group can be unsubstituted, or substituted with one or more suitable substituents at any one or more of the carbon atom(s) and/or nitrogen heteroatom(s) of the heterocyclyl. A heterocyclyl can comprise a single ring structure (i.e., monocyclic) or multiple ring structures (i.e., polycyclic, e.g., bicyclic). Polycyclic heterocyclyls include bridged, fused, and spiro ring structures in which at least one ring atom of at least one of the rings of the polycyclic ring system is a heteroatom, for instance oxygen, nitrogen, or sulfur, wherein bridged, fused, and spiro rings are as defined above. A heterocyclyl ring can be attached to the parent molecule at any suitable heteroatom (typically nitrogen) or carbon atom of the ring. The term “4- to 9-membered monocyclic or bicyclic heterocyclyl” includes any four, five, six, seven, eight, or nine membered monocyclic or bicyclic ring structure containing at least one heteroatom ring member selected from oxygen, nitrogen, and sulfur, or independently selected from oxygen and nitrogen, optionally containing one to three additional heteroatoms independently selected from oxygen, nitrogen, and sulfur, or independently selected from oxygen and nitrogen, wherein the ring structure is saturated or partially unsaturated, provided the ring structure is not fully aromatic.

In certain embodiments, the term “heterocyclyl” refers to 4-, 5-, 6-, or 7-membered monocyclic groups and 6-, 7-, 8-, or 9-membered bicyclic groups which have at least one heteroatom (O, S, or N) in at least one of the rings, wherein the heteroatom-containing ring(s) typically has 1, 2, or 3 heteroatoms, such as 1 or 2 heteroatoms, independently selected from O, S, and/or N, or independently selected from O and N. When numbers appear in a subscript after the symbol “C”, the subscript defines with more specificity the number of carbon atoms which that particular heterocycly can contain, in addition to the heteroatoms which that particular heterocycly can contain. For example, “C₁ to C₁₀ heterocycl” or “C₁₋₁₀ heterocycl” is intended to include heterocycl groups having 1, 2, 3, 4, 5, 6, 7, 8, 9, and 10 carbon atoms. Additionally, for example, “C₁ to C₈ heterocycly” or “C₁₋₈ heterocycly” denotes a heterocycl having 1, 2, 3, 4, 5, 6, 7, or 8 carbon atoms.

Examples of monocyclic heterocyclyl groups include, but are not limited to azetidinyl, oxctanyl, tetrahydrofuranyl, pyrrolidinyl, imidazolidinyl, pyrazolidinyl, oxazolidinyl, isoxazolidinyl, thiazolidinyl, isothiazolidinyl, dioxolanyl, dithiolanyl, piperidinyl, piperazinyl, dioxanyl, morpholinyl, azepanyl, oxepanyl, oxazepanyl (e.g., 1,4-oxazepanyl, 1,2-oxazepanyl) and the like. Examples of bicyclic heterocyclyl groups include, but are not limited to, 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, and 5-azaspiro[2.3]hexanyl and the like.

As used herein, the term “heteroaryl” includes stable monocyclic and polycyclic aromatic hydrocarbons that contain at least one heteroatom ring member such as sulfur, oxygen, or nitrogen. A heteroaryl group can be unsubstituted or substituted with one or more suitable substituents. A heteroaryl can comprise a single ring structure (i.e., monocyclic) or multiple ring structures (i.e., polycyclic, e.g., bicyclic or tricyclic). Each ring of a heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring, but the other fused ring or rings can be aromatic or non-aromatic. For example, for a bicyclic heteroaryl, the fused rings completing the bicyclic group can contain only carbon atoms and can be saturated, partially saturated, or unsaturated. A heteroaryl can be attached to the parent molecule at any available nitrogen or carbon atom of any ring of the heteroaryl group. In some embodiments, the term “heteroaryl” refers to 5- or 6-membered monocyclic groups and 9- or 10-membered bicyclic groups which have at least one heteroatom (O, S, or N) in at least one of the rings, wherein the heteroatom-containing ring typically has 1, 2, or 3 heteroatoms, such as 1 or 2 heteroatoms, selected from O, S, and/or N. A heteroaryl group can be unsubstituted, or substituted with one or more suitable substituents at any one or more of the carbon atom(s) and/or nitrogen heteroatom(s) of the heteroaryl. The nitrogen and sulfur heteroatom(s) of a heteroaryl can optionally be oxidized (i.e., N→O and S(O) r, wherein r is 0, 1 or 2).

When numbers appear in a subscript after the symbol “C”, the subscript defines with more specificity the number of carbon atoms which that particular heteroaryl can contain, in addition to the heteroatoms which that particular heteraryl can contain. For example, “C₁ to C₁₀ heteroaryl” or “C₁₋₁₀ heteroaryl” is intended to include heteroaryl groups having 1, 2, 3, 4, 5, 6, 7, 8, 9, and 10 carbon atoms. Additionally, for example, “C₁ to C₈ heteroaryl” or “C₁₋₈ heteroaryl” denotes a heteroaryl having 1, 2, 3, 4, 5, 6, 7, or 8 carbon atoms.

Exemplary monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl. Exemplary bicyclic heteroaryl groups include, but are not limited to, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridinyl, furopyridinyl, dihydroisoindolyl, and tetrahydroquinolinyl.

The term “alkoxy” as used herein refers to an —O-alkyl group, wherein alkyl is as defined above. An alkoxy group is attached to the parent molecule through a bond to an oxygen atom. An alkoxy group can have a specified number of carbon atoms. For example, “C₁ to C₁₀ alkoxy” or “C₁₋₁₀ alkoxy” is intended to include alkoxy groups having 1, 2, 3, 4, 5, 6, 7, 8, 9, and 10 carbon atoms. Additionally, for example, “C₁ to C₄ alkoxy” or “C₁₋₄ alkoxy” denotes an alkoxy having 1, 2, 3, or 4 carbon atoms. Examples of alkoxy include, but are not limited to, methoxy, ethoxy, propoxy (e.g., n-propoxy, isopropoxy), butoxy (e.g., n-butoxy, isobutoxy, tert-butoxy), pentyloxy (e.g., n-pentyloxy, isopentyloxy, neopentyloxy), etc. An alkoxy group can be unsubstituted or substituted with one or more suitable substituents. Similarly, “alkylthio” or “thioalkoxy” represents an alkyl group as defined above attached to the parent molecule through a bond to a sulfur atom, for example, —S-methyl, —S-ethyl, etc. Representative examples of alkylthio include, but are not limited to, —SCH₃, —SCH₂CH₃, etc.

As used herein, the term “halogen” means fluorine, chlorine, bromine, or iodine. Correspondingly, the term “halo” means fluoro, chloro, bromo, and iodo.

“Haloalkyl” is intended to include both branched and straight-chain saturated aliphatic hydrocarbon radicals substituted with one or more halogen atoms. “Fluorinated alkyl” or “fluoroalkyl” in particular refers to any alkyl group as defined above substituted with at least one fluoro atom, e.g., one to three fluoro atoms, such as one, two, or three fluoroatoms. Examples of haloalkyl include, but are not limited to, fluoromethyl, difluoromethyl, trifluoromethyl, chloromethyl, dichloromethyl, trichloromethyl, pentafluoroethyl, pentachloroethyl, 2,2,2-trifluoroethyl, heptafluoropropyl, and heptachloropropyl. Suitable examples of fluoroalkyl in particular include, but are not limited to, —CF3, —CHF2, —CH₂CF3, —CF2CF3, and the like.

The terms “hydroxy” and “hydroxyl” can be used interchangeably, and refer to —OH.

The term “carboxy” and “carboxyl” can be used interchangeably, and refers to —COOH.

The term “ester” refers to —COOR, wherein R is alkyl as defined above.

The term “cyano” refers to —CN.

The term “oxo” refers to a double bonded oxygen group, i.e., a substituent group of the formula ═O.

The term “keto” refers to —C(O)R, wherein R is alkyl as defined above.

As used herein, the term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

The term “aminoalkyl” as used herein is intended to include both branched and straight-chain saturated aliphatic hydrocarbon groups substituted with one or more amino groups. For example, “C₁₋₄ aminoalkyl” is intended to include alkyl groups having 1, 2, 3, or 4 carbon atoms substituted with one or more amino groups. Aminoalkyl groups are attached to the parent molecule through a bond to a carbon atom of the alkyl moiety of the aminoalkyl group. Representative examples of aminoalkyl groups include, but are not limited to, —CH₂NH₂, —CH₂CH₂NH₂, and —CH₂CH(NH₂)CH₃.

As used herein, “amido” refers to —C(O) N(R)₂, wherein each R is independently an alkyl group (including both branched and straight-chain alkyl groups) or a hydrogen atom. Examples of amido groups include, but are not limited to, —C(O)NH₂, —C(O)NHCH₃, and —C(O)N(CH₃)₂.

The terms “hydroxyl-substituted alkyl,” “hydroxylalkyl” and “hydroxyalkyl” are used interchangeably, and refer to a branched or straight-chain aliphatic hydrocarbon group substituted with one or more hydroxyl groups. Hydroxyalkyl groups are attached to the parent molecule through a bond to a carbon atom of the alkyl moiety of the hydroxyalkyl group. A hydroxyalkyl group can have a specified number of carbon atoms. For example, “C₁ to C₁₀ hydroxyalkyl” or “C₁₋₁₀ hydroxyalkyl” is intended to include hydroxyalkyl groups having 1, 2, 3, 4, 5, 6, 7, 8, 9, and 10 carbon atoms. Additionally, for example, “C₁ to C₄ hydroxylalkyl” or “C₁₋₄ hydroxyalkyl” denotes a hydroxyalkyl group having 1, 2, 3, or 4 carbon atoms. Examples of hydroxyalkyl include, but are not limited to, hydroxylmethyl (—CH₂OH), hydroxylethyl (—CH₂CH₂OH), etc.

As used herein, “amide” refers to —N(R′)C(O)R, wherein each R and R′ is independently chosen from hydrogen, alkyl, cycloalkyl, aryl, and heteroaryl. Examples of amide groups include, but are not limited to, —NHC(O)CH₃, —NHC(O)CH₂CH₃, and —N(CH₃)C(O)CH₃.

As used herein, “carbamide” refers to —N(R′)C(O)N(R)₂, wherein each R and R′ is independently chosen from hydrogen, alkyl, cycloalkyl, aryl, and heteroaryl. Examples of carbamide groups include, but are not limited to, —NHC(O)NH₂, —NHC(O)NHCH₃ (methyl carbamide), and —NHC(O)NH(Ph).

As used herein, “sulfonamide” refers to —N(R′)SO₂—R, wherein each R and R′ is independently chosen from hydrogen, alkyl, cycloalkyl, aryl, and heteroaryl. Examples of sulfonamide groups include, but are not limited to, —NHSO₂CH₃ (methyl sulfonamide), and —NH SO₂Ph.

In accordance with convention used in the art:

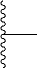

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

The term “substituted” as used herein with respect to any organic radical (e.g., alkyl, cycloalkyl, heteroaryl, aryl, heterocyclyl, etc.) means that at least one hydrogen atom is replaced with a non-hydrogen group, provided that all normal valencies are maintained and that the substitution results in a stable compound. When a particular group is “substituted,” that group can have one or more substituents, such as from one to five substituents, one to three substituents, or one to two substituents, independently selected from the list of substituents. The term “independently” when used in reference to substituents, means that when more than one of such substituents is possible, such substituents can be the same or different from each other. Examples of suitable substituents include, but are not limited to, alkyl, halo, haloalkyl, alkoxy, amido, hydroxy, hydroxyalkyl, amino, carboxyl, ester, oxo, cyano and the like.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

The terms “optional” or “optionally” mean that the event or circumstance described can, but need not, occur, and such a description includes the situation in which the event or circumstance does or does not occur. For example, “optionally substituted heterocyclyl” means that a substituent group can be, but need not be, present, and such a description includes the situation of the heterocyclyl group being substituted by a suitable substituent and the heterocyclyl group not being substituted by any substituent.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds, or otherwise indicated as having a particular configuration (e.g., R or S) around one or more atoms, contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or diastereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Certain examples contain chemical structures that comprise (R) or(S) terminology. When (R) or(S) is used in the name of a compound or in the chemical representation of the compound, it is intended to mean that the compound is a single isomer at that stereocenter, with established absolute configuration of either (R) or(S).

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diasteromeric salts with optically active acids or bases or by chiral chromatography. Pure stereoisomers can also be prepared synthetically from appropriate stereochemically pure starting materials, or by using stereoselective reactions.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the present disclosure.

Compounds described herein can exist in solvated and unsolvated forms. The term “solvate” means a physical association, e.g., by hydrogen bonding, of a compound described herein with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds described herein can form solvates with water (i.e., hydrates) or common organic solvents. Exemplary solvates include, but are not limited to, hydrates, ethanolates, methanolates, and isopropanolates. Methods of solvation are generally known in the art.

Also included within the scope of the present disclosure are all isotopes of atoms occurring in the compounds described herein, including intermediates and final products. Isotopes include those atoms having the same atomic number but different mass numbers. By way of general example and without limitation, isotopes of hydrogen include deuterium and tritium. Isotopes of carbon include 13C and 14C.

The present disclosure further includes isotopically-labeled compounds. An “isotopically-labeled” or “radio-labeled” compound is a compound of the present disclosure where one or more atoms are replaced or substituted by an atom having an atomic mass or mass number different from the atomic mass or mass number typically found in nature (i.e., naturally occurring). Isotopically-labeled compounds can generally be prepared by conventional techniques known to those skilled in the art or by processes analogous to those described herein, using an appropriate isotopically-labeled reagent in place of the non-labeled reagent otherwise employed.

As used herein, the name of a compound is intended to encompass all possible existing isomeric forms, including stereoisomers (e.g., enantiomers, diastereomers, racemate or racemic mixture, and any mixture thereof) of the compound.

In one general aspect, the present disclosure relates to methods of treating or preventing a disease in a subject in need thereof, the method comprising administering to the subject a therapeutically effective amount of a peripherally selective α2AR agonist.

In some embodiments, the peripherally selective α2AR agonist activates at least one sub type of α2AR, particularly α2A AR, α2B AR, or α2C AR.

In some embodiments, the disease is glaucoma, pain, spasticity, nasal congestion, rosacea, rhinitis, anesthesia, presbyopia, acute kidney injury, insomnia, inflammatory disease, cancer, etc.

In certain embodiments, the disease is chosen from pain, rosacea, spasticity, and aging.

In some embodiments, treating with the peripherally selective α2AR agonist causes less sedation than treating with a non-peripherally selective α2AR agonist, such as at similar or comparable dosage.

In some embodiments, the peripherally selective α2AR agonist comprises an α2AR activation moiety covalently linked to a peripheral distribution moiety.

In another general aspect, the present disclosure provides a peripherally selective α2AR agonist that comprises an α2AR activation moiety covalently linked to a peripheral distribution moiety, and its uses in the treatment or prevention of a disease.

In some embodiments, the disease is glaucoma, pain, spasticity, nasal congestion, rosacea, rhinitis, anesthesia, presbyopia, acute kidney injury, insomnia, inflammatory disease, cancer, etc.

In certain embodiments, the disease is pain.

Pain, as a complex and multidimensional sensory and emotional experience, poses a significant challenge to human health. It is not only an important symptom of physical diseases but also a key factor affecting the quality of life, causing great physical and mental distress to patients. Crucial components of pain are neuropathic pain and nociceptive pain. Neuropathic pain are caused by a lesion or disease of the somatosensory nervous system. Neuropathic pain can be divided into central neuropathic pain and peripheral neuropathic pain. Central neuropathic pain includes spinal cord injury, post-stroke pain, and MS pain, while peripheral neuropathic pain includes diabetic neuropathy, postherpetic neuralgia, HIV-associated pain, chemotherapy-induced peripheral neuropathy, and post-surgical neuropathic pain. Currently, first-line treatment drugs include Gabapentinoids, tricyclic antidepressants, and noradrenaline/serotonin uptake inhibitors. Although these drugs can relieve pain to some extent, the side effects of long-term use still cause a decrease in the quality of life of patients. Second-line treatment drugs, such as opiate receptor agonist, not only have side effects but also have a high addiction rate, which has caused many social impacts and cannot well address the demand for neuropathic pain drugs.

α2AR agonists, such as clonidine and dexmedetomidine, are considered an important method for treating pain in academic research and clinical applications. Scientists have found that the intraspinal administration of α2AR agonists can effectively relieve pain. However, the therapeutic benefits of α2AR agonists are not without limitations. Existing α2AR agonists are often associated with a range of biological reactions, including sedation, hypotension, bradycardia, drowsiness, dizziness, depression, bradycardia, orthostatic hypotension, constipation, nausea, gastric upset, dry mouth (xerostomia), dry nasal mucosa, impotence, fluid retention, edema, and pupil size. These other biological effects, especially sedation, set limits on the dosages that can be safely administered, thereby constraining the wide-scale utility of these drugs in long-term pain management. This not only affects the quality of life for patients but also restricts the applicability of these drugs for various types and levels of pain symptoms. These biological effects, especially sedation, seriously impact the application of α2AR agonists in the field of medical application.

Thus, there is a need to develop novel α2AR agonist compounds for treating pain with reduced sedation. Our work seeks to make significant contributions to the field of pain management by offering not just more effective but also safer long-term non-opioid alternatives. The compounds and methods described herein can be useful for addressing such unmet need.

As used herein, “an effective amount” means an amount of a composition or compound that elicits a biological or medicinal response in a tissue system or subject that is being sought by a researcher, veterinarian, medical doctor or other professional, which can include alleviation of the symptoms of the disease, disorder, or condition being treated. An effective amount can vary depending upon a variety of factors, such as the physical condition of the subject, age, weight, health, etc.; and the particular disease, disorder, or condition to be treated. An effective amount can readily be determined by one of ordinary skill in the art in view of the present disclosure.

According to particular embodiments, an effective amount refers to the amount of a composition or compound described herein which is sufficient to activate α2AR. In another particular embodiment, an effective amount refers to the amount of a composition or compound described herein which is sufficient to treat or prevent the disease or alleviate the symptoms associated with the disease.

In some embodiments, the pain is nociceptive pain, neuropathic pain such as peripheral neuropathic pain, or mixed pain.

In some embodiments, the neuropathic pain is cancer-associated pain, diabetic neuropathy, postherpetic neuralgia, trigeminal neuralgia, peripheral neuropathy, immune-mediated neuropathies, HIV-associated pain, post-stroke pain syndrome, phantom limb pain, chemotherapy-induced peripheral neuropathy, complex regional pain syndrome, and metabolic, endocrine, toxic neuropathies, chronic postsurgical pain, traumatic peripheral nerve injury, entrapment syndrome, heritable neuropathy, etc.

In some embodiments, the pain is post-surgery pain.

In some embodiments, the peripherally selective α2AR agonist comprises an α2AR activation moiety covalently linked to a peripheral distribution moiety.

In another general aspect, the present disclosure relates to methods of treating or preventing a disease in a subject in need thereof, the method comprising administering to the subject a therapeutically effective amount of a peripherally selective α2AR agonist, wherein the peripherally selective α2AR agonist comprises an α2AR activation moiety covalently linked to a peripheral distribution moiety.

In some embodiments, the disease is glaucoma, pain, spasticity, nasal congestion, rosacea, rhinitis, anesthesia, presbyopia, acute kidney injury, insomnia, inflammatory disease, cancer, etc. In some embodiments, the disease is glaucoma or cancer.

In some embodiments, the disease is pain.

In some embodiments, the pain is nociceptive pain, neuropathic pain such as peripheral neuropathic pain, or mixed pain.

In some embodiments, the neuropathic pain is cancer-associated pain, diabetic neuropathy, postherpetic neuralgia, trigeminal neuralgia, peripheral neuropathy, immune-mediated neuropathies, HIV-associated pain, post-stroke pain syndrome, phantom limb pain, chemotherapy-induced peripheral neuropathy, complex regional pain syndrome, and metabolic, endocrine, toxic neuropathies, chronic postsurgical pain, traumatic peripheral nerve injury, entrapment syndrome, heritable neuropathy, etc.

In some embodiments, the pain is post-surgery pain.

In some embodiments, treating with the peripherally selective α2AR agonist causes less side effects than treating with a non-peripherally selective α2AR agonist, such as at similar or comparable dosage.

In some embodiments, treating with the peripherally selective α2AR agonist causes no side effects.

The following embodiments apply to all general aspects described above.

In some embodiments, the side effect is sedation, decreasing heart rate, and decreasing blood pressure, particularly the side effect is sedation.

As used herein, the term “a non-peripherally selective α2AR agonist” refers to a compound can be readily distributed into the CNS after being administered into a subject, binds to and activates α2AR receptor in both the central nervous system (brain and spinal cord) and the peripheral nervous system. Examples of non-peripherally selective α2AR agonists include, but not limited to, dexmedetomidine, and clonidine.

Without binding to the theory, if an α2AR agonist binds to and activates α2AR in the central nervous system, it can produce the above mentioned side effects in patients, such as sedation, decreased heart rate, blood pressure, depression, bradycardia, orthostatic hypotension, constipation, nausea, gastric upset, dry mouth (xerostomia), dry nasal mucosa, impotence, fluid retention, edema, and pupil size.

As used herein, the term “a peripherally selective α2AR agonist” refers to a compound that primarily exerts its effects outside of the central nervous system (CNS), typically because it is impeded by the blood-CNS barrier. Blood-CNS barrier, the physical barrier between blood and the CNS, safeguards the CNS from both toxic and pathogenic agents in the blood. The blood-CNS barrier comprises the blood-brain barrier, the blood-spinal cord barrier, and the blood-CSF (cerebrospinal fluid) barrier. By being largely impeded from entering the CNS, a compound may act on the rest of the body with less or no side-effects related to their effects on the brain or spinal cord. Examples of peripherally selective α2AR agonists include, but not limited to, the compounds described herein, such as compounds of formula (I-A), (I-B), (I-C), (I-D), or (II), described herein.

The peripherally selective α2AR agonist primarily binds to or activates α2AR outside CNS, thus herby producing less or no foregoing side effects, compared to the non-peripherally selective α2AR agonists. The present invention satisfies an unmet need, and has developed a series of peripherally selective α2AR agonists.

In some embodiments, the peripherally selective α2AR agonist binds to α2AR with a Ki ranging from 250 nM to1000 nM, 50 nM to 250 nM, 10 nM to 50 nM, or less than 10 nM. In some other embodiments, the peripherally selective α2AR agonist activates α2AR with an EC50 ranging from 250 nM to 1000 nM, 50 nM to 250 nM, 10 nM to 50 nM, or less than 10 nM.

The non-peripherally selective α2AR agonist and the peripherally selective α2AR agonist can be differentiated in terms of blood-brain barrier (BBB) permeability. Drugs that specifically target the central nervous system (CNS) must first traverse the BBB. In contrast, peripherally selective drugs primarily exert their effects outside of CNS, largely because they are impeded by the blood-brain barrier (BBB). The blood-brain barrier (BBB) substantially limits the entry of these drugs into the central nervous system (CNS), leading to a predominance of the drug concentration outside the CNS compared to inside. Any methods known in the field can be used to measure a compound's BBB permeability. For example, one experimental measure of BBB permeability is Kp, which is the concentration of drug in the brain divided by concentration in the blood.

As used herein, “Kp”, or “B/P ratio”, refers to the ratio of the concentration of a compound in the brain and in the blood. Kp is often calculated as “log BB”, which refers to the logarithmic ratio of the concentration of a compound in the brain and in the blood. Kp is a common numeric value for describing permeability across the blood-brain barrier. In some embodiments, a compound is considered “peripherally selective” if, upon administration to a subject, its Kp is lower than 0.4, 0.2, 0.1, 0.05, 0.02, or 0.01.

Kp,uu,brain is another common numeric value for describing permeability across the blood-brain barrier. As used herein, “Kp,uu,brain” or “Kp,uu”, refers to the unbound brain-to-plasma partition coefficient. It represents the ability of a drug to cross the blood-brain barrier (BBB) after systemic administration. Kp,uu provides a more accurate measure of distribution equilibrium between unbound fractions in brain and plasma.

Any methods known in the field can be used to measure Kp, uu,brain. One example of Area Under the Curve (AUC) method, which calculates the AUC of the unbound drug concentration-time profile in both brain and plasma after a single dose. Another example is Steady-State Concentrations, which uses the steady-state unbound concentrations of the drug in brain interstitial fluid (C_u,brain,ss) and in plasma (C_u,plasma,ss).

In some embodiments, a compound is considered “peripherally selective” if, upon administration to a subject, its Kp, uu,brain is lower than 0.4, 0.2, 0.1, 0.05, 0.02, or 0.01. In some further embodiments, a compound is considered “peripherally selective” if, upon administration to a subject, its Kp,uu,brain is lower than 0.05, 0.02, or 0.01.

In some embodiments, the peripherally selective α2AR agonist comprises an α2AR activation moiety that is covalently linked to a peripheral distribution moiety.

In certain embodiments, the α2AR activation moiety is a non-peripherally selective α2AR agonist or another peripherally selective α2AR agonist.

In certain embodiments, the α2AR activation moiety is an α2AR agonist that is chosen from (R)-3-nitrobiphenyline, A-193080, ADX-415, AGN 192836, AGN-191103, AGN-197075, AGN-201781, AGN-241622, amitraz, Apraclonidine, AR-08, Bethanidine, Brimonidine, BRL-48962, Bromocriptine, Cirazoline, Clonidine, Detomidine, Detomidine carboxylic acid, Dexmedetomidine, Dipivefrin, DL-Methylephedrine, Droxidopa, Epinephrine, ergotamine, etilefrine, Etomidate, Fadolmidine, Guanabenz, Guanethidine, Guanfacine, Guanoxabenz, indanidine, Lofexidine, Medetomidine, mephentermine, Metamfetamine, metaraminol, methoxamine, Methyldopa, Methyldopate, Methyldopate hydrochloride, Methylnorepinephrine, mivazerol, Moxonidine, naphazoline, Norepinephrine, norfenefrine, octopamine, ODM-105, Oxymetazoline, Pergolide, phenylpropanolamine, Povafonidine, propylhexedrine, Pseudoephedrine, Racepinephrine, rezatomidine, rilmenidine, romifidine, synephrine, talipexole, tasipimidine, Tiamenidine, Tizanidine, Xylazine, Xylometazoline, and a functional derivative thereof.

As used herein, the functional derivative of an α2AR agonist refers to any compound that is derived from the α2AR agonist by a chemical reaction. Examples of the derivatives include, but not limited to, acid or base salts, prodrugs, compounds containing protected functional groups such as hydroxyl, amino, carboxyl and carbonyl groups.

In certain embodiments, the α2AR activation moiety is a non-peripherally selective α2AR agonist, such as dexmedetomidine, brimonidine, and clonidine.

In certain embodiments, the α2AR activation moiety is dexmedetomidine.

As used herein, the term “a peripheral distribution moiety” refers to a moiety that can increase or improve the peripheral selectivity of an α2AR agonist. In some embodiments, the peripheral selectivity is increased or improved so that the α2AR agonist is a peripherally selective α2AR agonist.

According to the embodiments of the present disclosure, the peripheral distribution moiety can be the following chemical fragments:
type A fragments; those that can increase the overall molecular polarity of the compound or reduce the overall lipophilicity of the compound;
type B fragments: those that can increase the overall molecular weight or the molecular size of the compound; and
type C fragments: those that comprises a substrate element of an efflux transporter.

In some embodiments, the peripheral distribution moiety is a type A fragment.

In certain embodiments, the type A fragment increases the total number of intermolecular hydrogen bond (H-bond) within the compound, such as H-bond donors and H-bond acceptors. In a preferred embodiment, the type A fragment is a H-bond donor.

In certain embodiments, the type A fragment increases the overall molecular polarity of the compound. For example, such type A fragments can comprise a polar functional group or a charged group. Examples of the polar functional group include, but not limited to, hydroxyl, amine, amide, sulfonamide, carboxyl, ether, imine, hydroxylamine, ester, aldehyde, ketone, nitro, phosphate, thioether, and sulfone groups. Examples of the charged group include, but not limited to, quaternary ammonium and organic acids such as carboxylic acids and sulfonic acids.

In certain embodiments, the type A fragment reduces the overall lipophilicity of the compound. Examples of such type A fragments include, but not limited to, alkyl or acyl that is added to a function group such as hydroxyl and amino.

In certain embodiments, the type A fragment is not tertiary amine or one that can help form an intramolecular H-bond.

In some embodiments, the peripheral distribution moiety is a type B fragment.

In certain embodiments, the type B fragment is a bulky group, which can increase the overall molecular weight and the molecular size of the compound. Examples of such type B fragments include, but not limited to, long alkyl chains, polyethylene glycol (PEG), large aromatic groups, and extra cyclic or heterocyclic groups.

In some embodiments, the peripheral distribution moiety is a type C fragment.

In certain embodiments, the type C fragment comprises a substrate element of an efflux transporter, wherein the efflux transporter is P-glycoprotein (P-gp) transporter, breast cancer resistance protein (BCRP) transporter, or multidrug resistance protein 2 (MRP2) transporter. As used herein, the term of “a substrate element of an efflux transporter” refers to a fragment that makes the compound to become a substrate of the efflux transporter. In other words, the term of “a substrate element of an efflux transporter” refers to a fragment of a substrate of the efflux transporter.

In certain embodiments, the type C fragment comprises a substrate element of P-gp.

Without binding to the theory, P-gp efflux is a significant limitation to BBB permeation. Any methods known in the filed can be used to determine whether a compound is a P-gp substrate. For example, the efflux ratio obtained from in vitro P-gp assay, MDCK-MDR1, can be used to identify the substrate of P-gp. A compound is considered as a P-gp substrate if its efflux ratio is greater than 2, 5, 8, 10, 50, or 100.

Alternatively, there are some rules for determining potential P-gp efflux substrates:
the total number of N atom and O atom (N+O)≥8;
molecular weight (MW)>400; and/or
acid with pKa>4.

In contrast, if a compound has N+O<4, MW<400, and/or is a base with pKa<8, then it is a non-substrate of P-gp.

Certain structural modifications can improve P-gp efflux, such as removing steric hindrance to the hydrogen bond donating atoms by attachment of a bulky group or by unmethylation the nitrogen atom, and improving hydrogen bonding potential by removal of an adjacent electron withdrawing group or by introducing the hydrogen bonding group such as amide.

In certain embodiments, the substrate element for P-gp contains one or more of the structural modifications described above.

In certain embodiments, the substrate element for P-gp is chosen from:

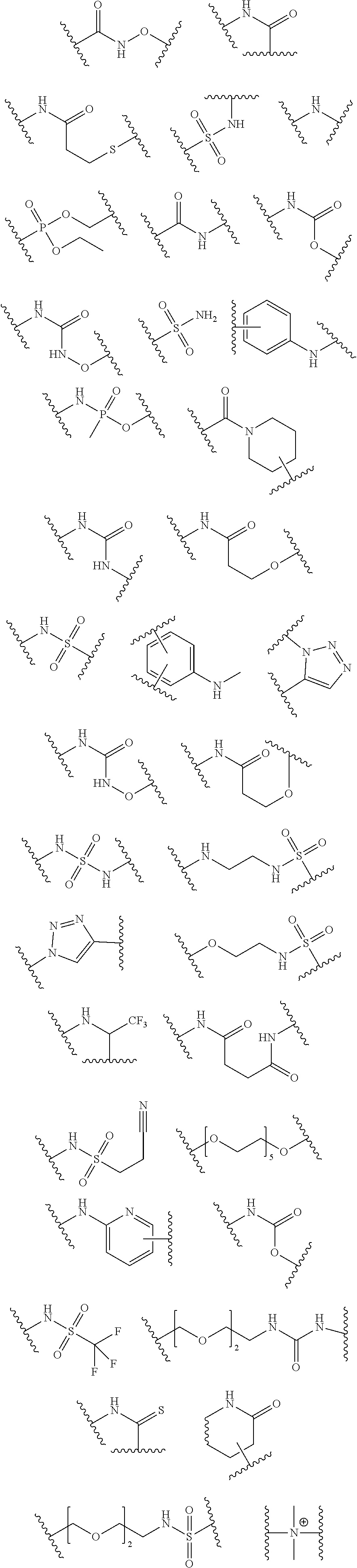

In certain embodiments, the type C fragment comprises a substrate element of BCPR transporter.

In certain embodiments, the type C fragment comprises a substrate element of MPR2 transporter.

In certain embodiments, the type C fragment does not comprise a substrate element of uptake transporter, such as LAT1, GLUT1, MCT1, CAT1, CNT2, OATP, PEPT1, PEPT2, and OCT.

In some embodiments, the peripheral distribution moiety reduces and/or minimizes brain exposure to a peripherally selective α2AR agonist.

In certain embodiments, the peripheral distribution moiety decreases passive transcellular BBB permeability by increasing topological polar surface area (TPSA), increasing molecule weight, increasing polarity, or adding hydrogen binding, especially hydrogen bond donor.

In certain embodiments, the peripheral distribution moiety introduces an acidic group to the peripherally selective α2AR agonist.

In certain embodiments, the peripheral distribution moiety comprises a substrate element for P-gp, wherein the substrate element for P-gp increases P-gp efflux by increasing lipophilicity, increasing hydrogen bond acceptors, removing steric hindrance around hydrogen bind acceptors, or removing electron-withdrawing group adjacent to hydrogen bond acceptor.

In certain embodiments, the peripheral distribution moiety makes a compound to become a dual substrate for both P-gp and BCRP.

### Compounds

In a general aspect, the present disclosure relates to a compound of formula (I-A):

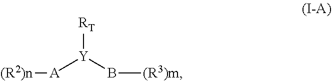

or a stereoisomer, tautomer, pharmaceutically acceptable salt or solvate thereof,
wherein,
Y is C(R¹), N, —O—C, —C—NH—, —CH₂—C(O)—, or —CH═N—;
   when Y is C(R¹), R′ is chosen from H, D, and halogen;
   when Y is —O—C—, the oxygen atom is connected to A, and the carbon atom is connected to both R_{T} and B; and
   when Y is —C—NH—, the carbon atom is connected to both R_{T} and A, and the nitrogen atom is connected to B;
A is a ring chosen from phenyl, pyridinyl, thienyl, furyl, pyrrolyl, 4H-pyran, 4H-thiopyran, 1,2,3,4-tetrahydro-1-naphthyl, tetrahydrozoline, quinoxalinyl, pyrimidinyl, and 2,1,3-benzothiadiazol;
B is

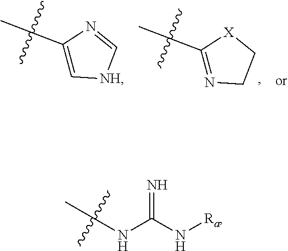

wherein X is NH, O, or S, and Ra is H and methyl;
n is 0, 1, 2, or 3;
each R₂ is independently chosen from H, D, halogen, alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl, OR⁴, —CN, N₃, NO₂, N(R⁴)₂, OR₄, SR⁴, C(O)R⁴, SO₂N(R⁴)₂, CH₂SR⁴, wherein the alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl is optionally substituted with one or more R⁵;
R⁴ is chosen from H, D, halogen, alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl, and the alkyl, alkenyl, alkynyl, alkoxyl, ester, cycloalkyl, cycloalkoxy, aryl, aryloxy, arylalkyl, heteroaryl, heteroarylalkyl, heterocyclyl, heterocyclolalkyl is optionally substituted with one or more R⁵;
R⁵ is chosen from halogen, hydroxyl, —CN, —NO₂, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;
alternatively, when A is a phenyl ring and two R² are substituted at adjacent positions of the phenyl ring, the two R² groups, with the carbon atoms they are connected to, form a ring that is fused to ring A to form a bicyclic ring, such as quinolinyl, indolyl, benzothienyl, benzofuryl, benzofuranyl, benzodioxolyl, 2,3-dihydrobenzo[b][1,4]dioxin-6-yl, cinnolinyl, quinoxalinyl, or 1,2,4-benzotriazinyl;
m is 0, 1, 2, or 3;
each R³ is independently chosen from H, D, halogen, —OH, —SH, optionally substituted alkyl, optionally substituted heterocycle, and optionally substituted aryl;
alternatively, R³ is a group connected to the —NH of the imidazole ring, and R³ is of the formula of:

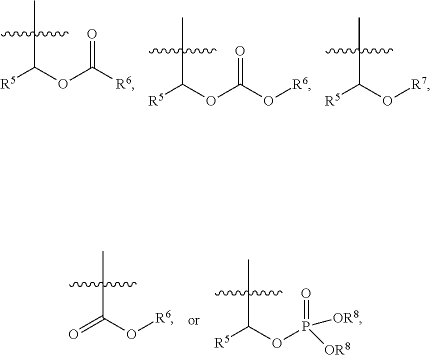

wherein:
R⁵ is hydrogen or alkyl;
R⁶ is hydrogen, alkyl, cycloalkyl, or alkenyl;
R⁷ is an amino acid residue; and
R⁸ is alkyl or cycloalkyl;

R_{T} is R_{L}—R_{P}, and R_{P} is optionally substituted with R_{C}, wherein:
R_{L} is a linker, wherein one end is connected to R_{P} and the other end is connected to Y;
R_{P} is a moiety that is connected to one end of R_{L}; and
R_{C} is a cap, which is a moiety that is connected to R_{P}.

As used herein, the term R_{L} is a moiety that covalently connects two functional groups or moieties within a single molecule. One end of R_{L} is connected to R_{P} and the other end of R_{L} is connected to Y. R_{L} can be any moiety that serves the linking function, such as the linkers used in proteolysis targeting chimeras (PROTACs) and non-cleavable linkers used in antibody-drug conjugates (ADCs). Examples of R_{L} include, but are not limited to, polyethylene glycol (PEG) and alkyl chains of varying lengths, glycols, alkynes, triazoles, saturated heterocycles such as piperazine and piperidine, thioethers, maleimidocaproyl linker.

In some embodiments, R_{L} is chosen from alkyl, polyethylene glycol, other glycol, cycloalkyl, heterocycle, aryl, and heteroaryl; wherein the cycloalky, heterocycle, aryl, or heteroaryl is optionaly substituted with at least one substituent chosen from halogen, hydroxyl, alkyl, haloalkyl, alkoxy and hydroxyalkyl.

In certain embodiments, R_{L} is one selected from the followings:

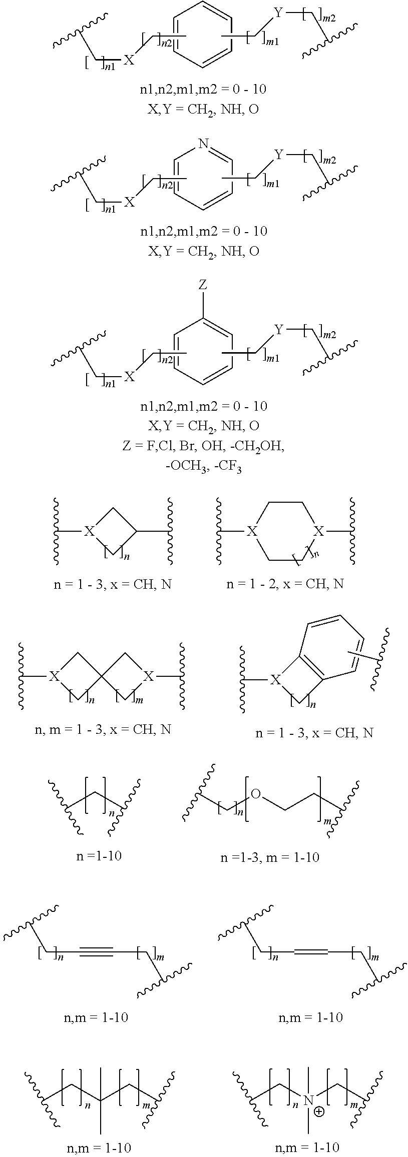

or a combination of two or more thereof. When R_{L} is a combination of two or more the above fragments, the fragments can be connected in any order.

According to the embodiments of the present disclosure, Rₚ can be the following chemical moieties:
those that can increase the overall molecular weight of the compound, such as bulky functional groups and additional molecular structures, including long alkyl chains, large aromatic groups, and extra cyclic structures like cyclohexane or cyclopentane rings;
those that can increase the overall molecular polarity of the compound, such as hydroxyl, amine, amide, sulfonamide, ether, imine, hydroxylamine, ester, aldehyde, ketone, nitro, phosphate, thioether; and
those that can bring charge to the compound, such as the functional groups that ionize at physiological pH, including carboxylic acid, quaternary ammonium and quaternary phosphonium.

In some embodiments, when R_{P} is not substituted with R_{c}, R_{P} is:

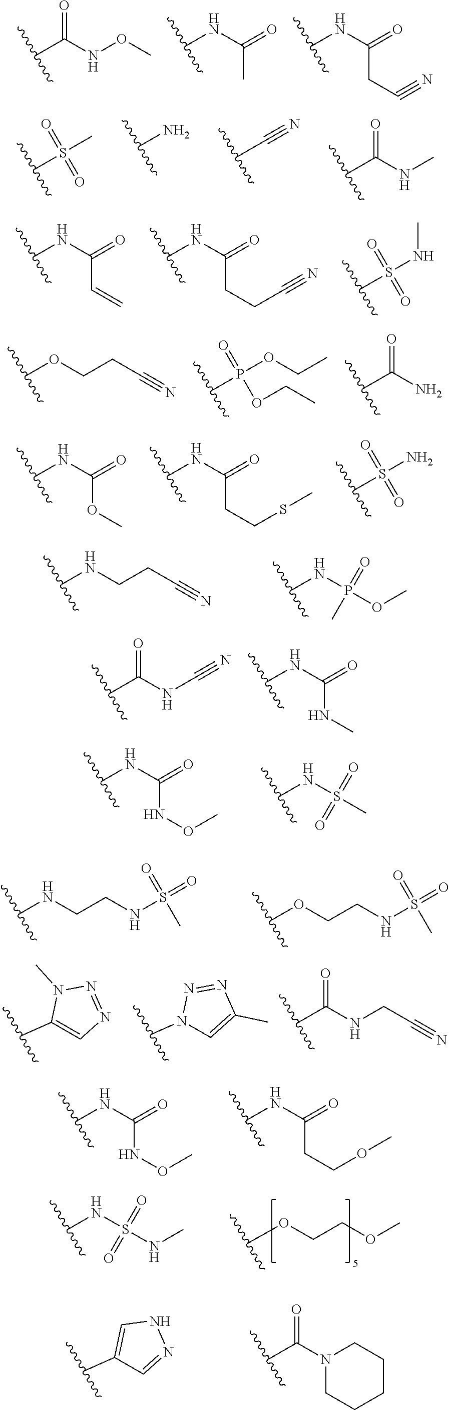

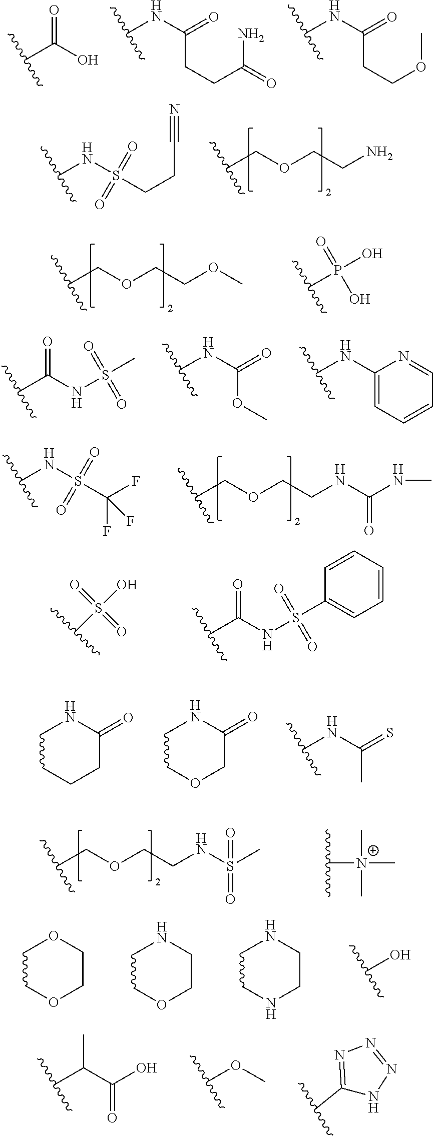

In some embodiments, when R_{P} is substituted with R_{C}. R_{P} is:

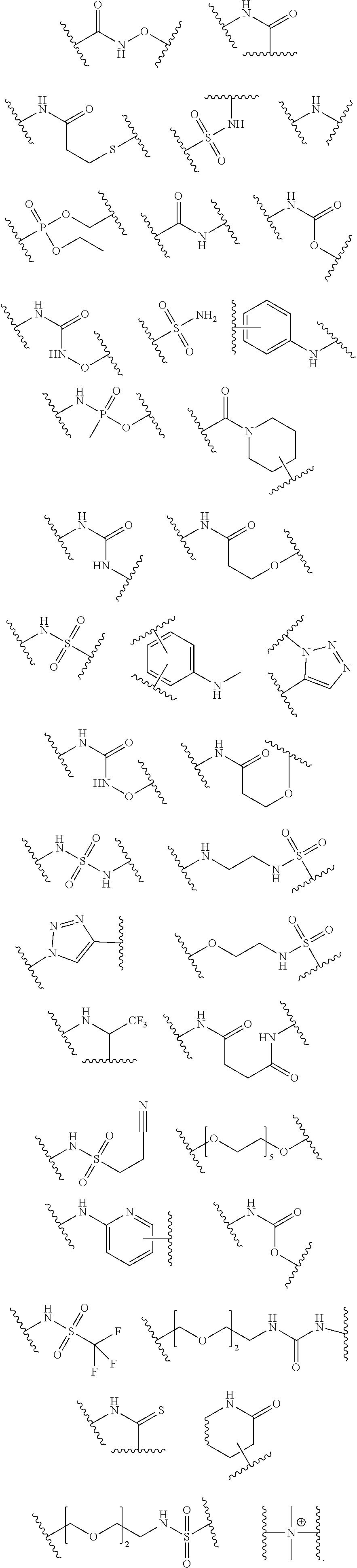

As used herein, the term R_{c} refers to a chemical moiety covalently attached to the end of R_{P}.

In some embodiments, R_{c} is chosen from —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —NH—C₀₋₁₂ alkylene-C₃₋₁₂ cycloalkyl, —NH—C₀₋₁₂ alkylene-C₂₋₁₂ heterocyclyl, —NH—C₀₋₁₂ alkylene-C₁₋₁₂ heteroaryl, —O—C₀₋₁₂ alkylene-C₃₋₁₂ cycloalkyl, —O—C₀₋₁₂ alkylene-C₂₋₁₂ heterocyclyl, —O—C₀₋₁₂ alkylene-C₁₋₁₂ heteroaryl, and alkyl substituted with trialkylammonium, wherein each of the C₃₋₁₂ cycloalkyl, C₂₋₁₂ heterocyclyl, and C₁₋₁₂ heteroaryl is optionally substituted with one or more substituents chosen from hydroxy, alkyl, oxo, and ketone.

In certain embodiments, R_{c} is:

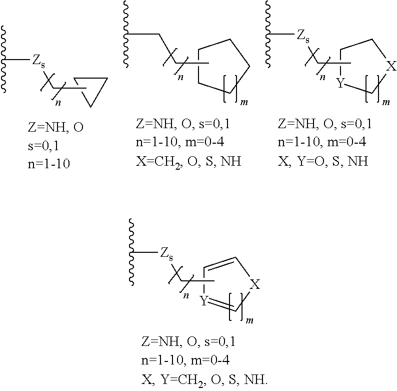

In certain embodiments, R_{C} is:

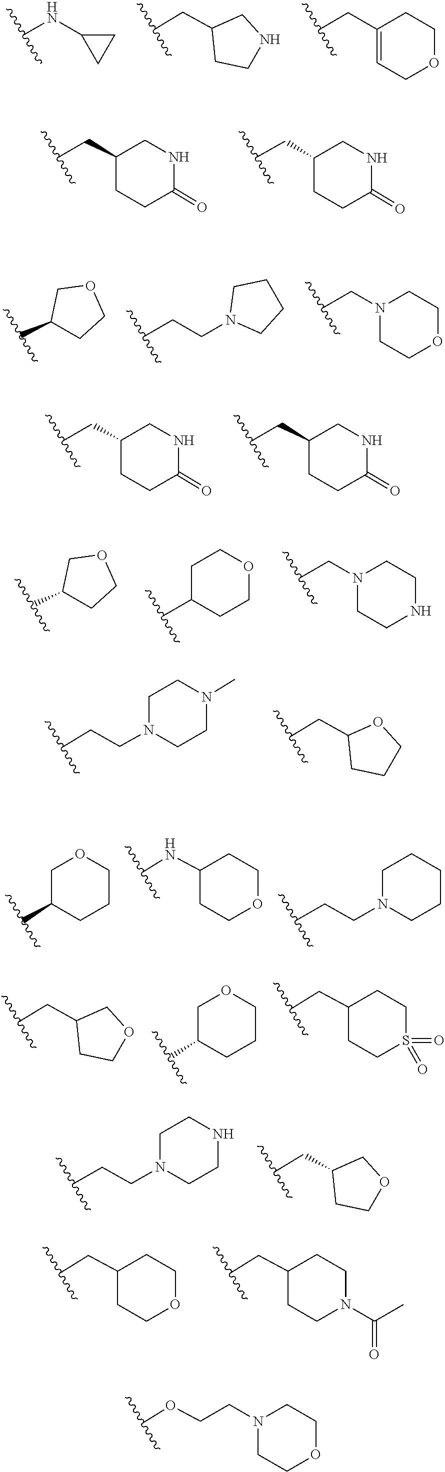

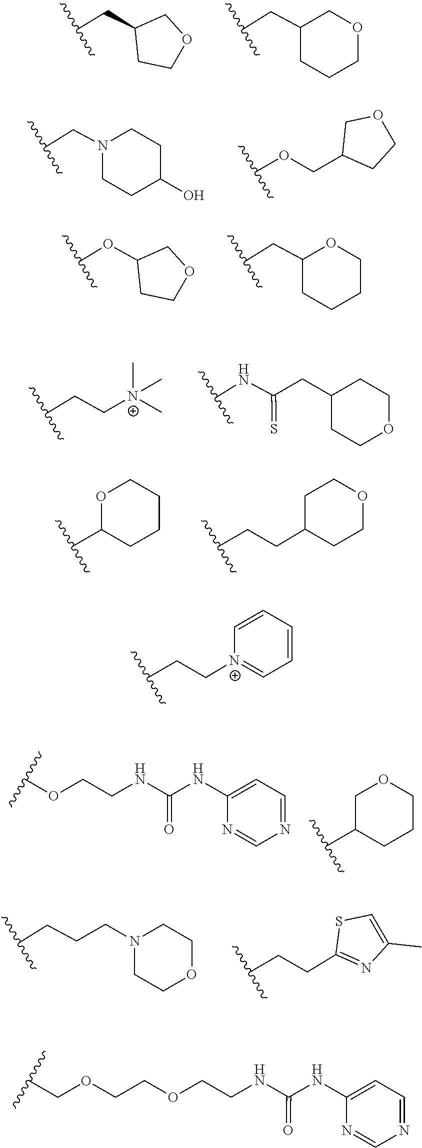

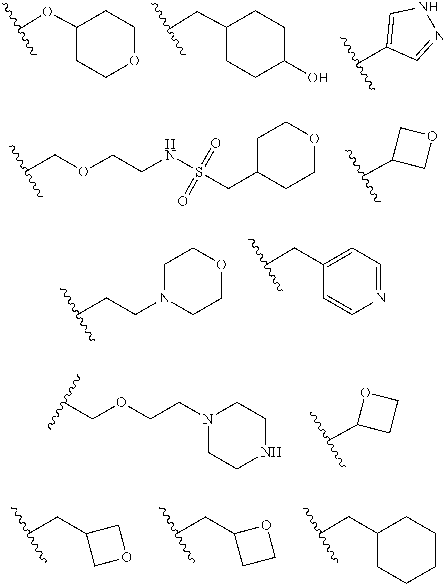

In another general aspect, the present disclosure relates to a compound of formula (I-B):

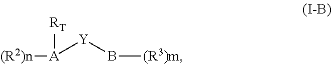

or a stereoisomer, tautomer, pharmaceutically acceptable salt or solvate thereof,
wherein,
Y is a bond, CH(R¹), NH, —O—CH—, —C—NH—, —CH₂—C(O)—, or —CH═N—;
   when Y is C(R¹), R¹ is chosen from H, D, and halogen;
   when Y is —O—C—, the oxygen atom is connected to A, and the carbon atom is connected to B;
   when Y is —C—NH—, the carbon atom is connected to A, and the nitrogen atom is connected to B; and
A, B, R², n, R³, m, and R_{T} are defined as above in formula (I-A).

In another general aspect, the present disclosure relates to a compound of formula (I-C):

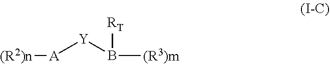

or a stereoisomer, tautomer, pharmaceutically acceptable salt or solvate thereof,
wherein,
Y is a bond, CH(R¹), NH, —O—CH—, —C—NH—, —CH₂—C(O)—, or —CH═N—;
   when Y is C(R¹), R¹ is chosen from H, D, and halogen;
   when Y is —O—C—, the oxygen atom is connected to A, and the carbon atom is connected to B;
   when Y is —C—NH—, the carbon atom is connected to A, and the nitrogen atom is connected to B; and
A, B, R², n, R³, m, and R_{T} are defined as above in formula (I-A).

In another general aspect, the present disclosure relates to a compound of formula (I-D):

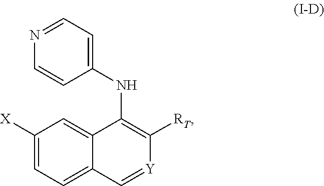

or a stereoisomer, tautomer, pharmaceutically acceptable salt or solvate thereof,
wherein,
X is CH, or N;
Y is chosen from H, D, and halogen; and
R_{T} is defined as above in formula (I-A).

In some embodiments, the compound of formula (I-A) has the formula (I-A-1):

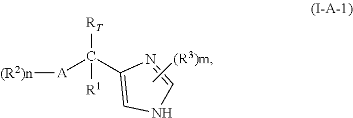

or a stereoisomer, tautomer, pharmaceutically acceptable salt or solvate thereof,
wherein,
R¹ is chosen from H, D, and halogen;
A is a ring chosen from phenyl, pyridinyl, thienyl, furyl, pyrrolyl, 4H-pyran, or 4H-thiopyran;
R², n, R³, m, and R_{T} are defined as above in formula (I-A).

In some embodiments, the compound of formula (I-A) has the formula (I-A-2):

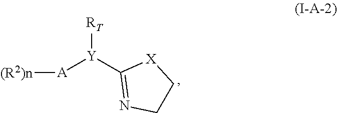

or a stereoisomer, tautomer, pharmaceutically acceptable salt or solvate thereof,
wherein,
A is a ring chosen from phenyl, 1,2,3,4-tetrahydro-1-naphthyl, quinoxalinyl, pyrimidinyl, and 2,1,3-benzothiadiazol;
Y is CH, N, —O—CH—, or —C—NH—;
   when Y is —O—C—, the oxygen atom is connected to A, and the carbon atom is connected to both R_{T} and

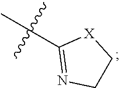

when Y is —C—NH—, the carbon atom is connected to both R_{T} and A, and the nitrogen atom is connected to

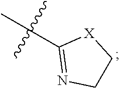

X is NH, O, or S; and
R², n, and R_{T} are defined as above in formula (I-A).

In certain embodiments, when the compound of formula (I-A) has the formula (I-A-1) or (I-A-2), R_{T} is

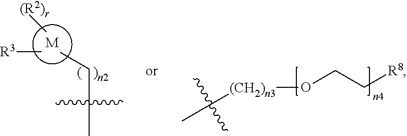

wherein:
ring M is C₃₋₁₂ Cycloalkyl, C₂₋₁₂ heterocyclyl, C₆₋₁₂ aryl, or C₁₋₁₂ heteroaryl, wherein the C₃₋₁₂ cycloalkyl or C₂₋₁₂ heterocyclyl is optionally fused with an aryl;
r is 1 or 2;
n2 is 0, 1, or 2;
each R₂ is independently chosen from hydrogen, halogen, hydroxyl, and alkoxy;
R³ is chosen from CN, hydroxy, alkoxy, —C(O)—C₀₋₁₂ alkylene-CN, —C₀₋₁₂ alkylene-C₂₋₁₂ heterocyclyl, —SO₂-alkyl, —C(O)—NR⁴R^{4′}, —SO₂—NR⁴R^{4′}, —C₀₋₁₂ alkylene-R^{3′}, —O—C₀₋₁₂ alkylene-COOH, —C₀₋₁₂ alkylene-N(R⁴)—C(O)—R⁵, —C₀₋₁₂ alkylene-N(R⁴)—SO₂—R⁵, —C₀₋₁₂ alkylene-C₁₋₁₂ heteroaryl, —C₀₋₁₂ alkylene-O—C₀₋₁₂ alkylene-N(R⁴)—SO₂—R⁵, —C₀₋₁₂ alkylene-P(═O)(R⁴) (R^{4′}),

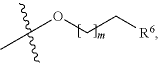

### —NH—R⁷, or

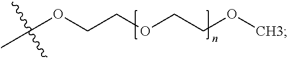

wherein one-CH₂— group in the —C₀₋₁₂ alkylene-R^{3′} is optionally replaced by oxygen atom or

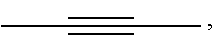

the —C₀₋₁₂ alkylene-R^{3′} is optionally substituted with one or more substitutes chosen from amino and alkylamino, and the C₂₋₁₂ heterocyclyl and C₁₋₁₂ heteroaryl are each optionally substituted with one or more R^{4a};
R^{3′} is chosen from —C(O)—NR⁴R^{4′}, —SO₂—NR⁴R^{4′}, —C₀₋₁₂ alkylene-COOH, —C₀₋₁₂ alkylene-N(R⁴)—C(O)—R⁵, —C₀₋₁₂ alkylene-N(R⁴)—SO₂-R⁵, C₀₋₁₂ alkylene-C₁₋₁₂ heteroaryl;
each R^{4a} is independently chosen from hydroxy, alkyl, oxo, ketone, and —C₂₋₁₂ heterocyclyl;
each of R⁴ and R^{4′} is independently hydrogen, alkyl, alkoxy, —SO₂—N(R⁶ª) t, —C₀₋₁₂ alkylene-COOH, —C₀₋₁₂ alkylene-N(R^{6a}) t, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or hydroxyalkyl, wherein the hydroxyalkyl is optionally substituted with alkoxy; wherein each of the alkyl, C₃₋₁₂ cycloalkyl, C₂₋₁₂ heterocyclyl, and C₁₋₁₂ heteroaryl is optionally substituted with one or more R^{4a};
or R⁴ and R^{4′}, together with the nitrogen atom that they are attached to, form a heterocycle comprising one or more heteroatoms chosen from O, N, and S;
alternatively, when one R² is adjacent to R³, the R² and R³, together with the atoms that they are attached to, form a ring optionally substituted with one or more R^{4a};
R⁵ is amino, alkylamino, C₁₋₁₂ haloalkyl, —C₀₋₁₂ alkylene-OR^{6a}, —C₀₋₁₂ alkylene-N(R⁶ª) t, —C₀₋₁₂ alkylene-SR^{6a}, —C₀₋₁₂ alkylene-CN, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₂₋₁₂ alkenyl, or alkyl optionally substituted with cyano, amido, trialkylammonium, or thiolate; wherein each of the C₃₋₁₂ cycloalkyl, C₂₋₁₂ heterocyclyl, and C₁₋₁₂ heteroaryl is optionally substituted with one or more R^{4a};
each R^{6a} is independently chosen from hydrogen, C₁₋₁₂ alkyl, C₁₋₁₂ alkoxy, —C₀₋₁₂ alkylene-C₃₋₁₂ cycloalkyl, —C₀₋₁₂ alkylene-C₂₋₁₂ heterocyclyl, —C₀₋₁₂ alkylene-C₆₋₁₂ aryl, and —C₀₋₁₂ alkylene-C₁₋₁₂ heteroaryl; wherein each of the alkyl, C₃₋₁₂ cycloalkyl, C₂₋₁₂ heterocyclyl, and C₁₋₁₂ heteroaryl, is optionally substituted with one or more R^{4a};
R⁶ is alkoxy, amino, sulfonamide, carbamide, or alkyl optionally substituted with cyano;
R⁷ is hydrogen, alkyl, —C₀₋₁₂ alkylene-COOH, optionally substituted C₃₋₁₂ cycloalkyl, C₂₋₁₂ aryl, C₁-12 heteroaryl, —C₀₋₁₂ alkylene-N(R⁴)—SO₂-R⁵, —C₀₋₁₂ alkylene-P(—O)(R⁴) (R^{4′}), —C₀₋₁₂ alkylene-N(R⁴)—C(═S)—R⁵, —C(═S)—R⁵, or alkyl optionally substituted with cyano;
R⁸ is alkoxy, amino, alkylamino, amide, sulfonamide, or carbamide;
n3 is 0, 1, 2, 3, or 4;
n4 is 1, 2, 3, 4, 5, or 6;
t is 2 or 3;
m is 0, 1, 2, 3, 4, or 5; and
n is 0, 1, 2, 3, or 4.

In another general aspect, the present disclosure relates to a compound of formula (II):

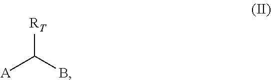

or a stereoisomer, tautomer, pharmaceutically acceptable salt or solvate thereof,
wherein,
A is one chosen from:

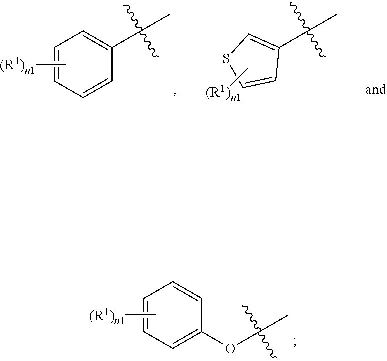

n1 is 1 or 2;
each R¹ is independently chosen from hydrogen, halogen, haloalkyl, hydroxyl, hydroxyalkyl, alkoxy, alkyl, and —COOH;
B is one chosen from:

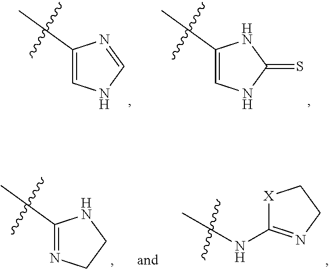

wherein X is S, O, or NH;
R_{T} is

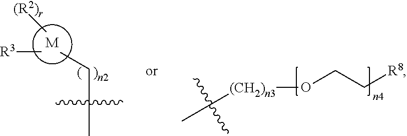

ring M is C₃₋₁₂ Cycloalkyl, C₂₋₁₂ heterocyclyl, C₆₋₁₂ aryl, or C₁₋₁₂ heteroaryl, wherein the C₃₋₁₂ cycloalkyl or C₂₋₁₂ heterocyclyl is optionally fused with an aryl;
r is 1 or 2;
n2 is 0, 1, or 2;
each R₂ is independently chosen from hydrogen, halogen, hydroxyl, and alkoxy;
R³ is chosen from CN, hydroxy, alkoxy, —C(O)—C₀₋₁₂ alkylene-CN, —C₀₋₁₂ alkylene-C₂₋₁₂ heterocyclyl, —SO₂-alkyl, —C(O)—NR⁴R^{4′}, —SO₂—NR⁴R^{4′}, —C₀₋₁₂ alkylene-R^{3′}, —O—C₀₋₁₂ alkylene-COOH, —C₀₋₁₂ alkylene-N(R⁴)—C(O)—R⁵, —C₀₋₁₂ alkylene-N(R⁴)—SO₂—R⁵, —C₀₋₁₂ alkylene-C₁₋₁₂ heteroaryl, —C₀₋₁₂ alkylene-O—C₀₋₁₂ alkylene-N(R⁴)—SO₂—R⁵, —C₀₋₁₂ alkylene-P(═O)(R⁴) (R^{4′}),

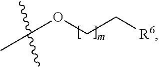

### —NH—R⁷, or

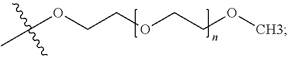

wherein one-CH₂— group in the —C₀₋₁₂ alkylene-R^{3′} is optionally replaced by oxygen atom or

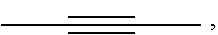

the —C₀₋₁₂ alkylene-R³ is optionally substituted with one or more substitutes chosen from amino and alkylamino, and the C₂₋₁₂ heterocyclyl and C₁₋₁₂ heteroaryl are each optionally substituted with one or more R^{4ª;}
R^{3′} is chosen from —C(O)—NR⁴R^{4′}, —SO₂—NR⁴R^{4′}, —C₀₋₁₂ alkylene-COOH, —C₀₋₁₂ alkylene-N(R⁴)—C(O)—R⁵, —C₀₋₁₂ alkylene-N(R⁴)—SO₂—R⁵, C₀₋₁₂ alkylene-C₁₋₁₂ heteroaryl;
each R^{4a} is independently chosen from hydroxy, alkyl, oxo, ketone, and —C₂₋₁₂ heterocyclyl;
each of R⁴ and R^{4′} is independently hydrogen, alkyl, alkoxy, —SO₂—N(R^{6a}) t, —C₀₋₁₂ alkylene-COOH, —C₀₋₁₂ alkylene-N(R^{6a})ₜ, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or hydroxyalkyl, wherein the hydroxyalkyl is optionally substituted with alkoxy; wherein each of the alkyl, C₃₋₁₂ cycloalkyl, C₂₋₁₂ heterocyclyl, and C₁₋₁₂ heteroaryl is optionally substituted with one or more R^{4a};
or R⁴ and R^{4′}, together with the nitrogen atom that they are attached to, form a heterocycle comprising one or more heteroatoms chosen from O, N, and S;
alternatively, when one R² is adjacent to R³, the R² and R³, together with the atoms that they are attached to, form a ring optionally substituted with one or more R^{4a};
R⁵ is amino, alkylamino, C₁₋₁₂ haloalkyl, —C₀₋₁₂ alkylene-OR^{6a}, —C₀₋₁₂ alkylene-N(R^{6a})ₜ, —C₀₋₁₂ alkylene-SR^{6a}, —C₀₋₁₂ alkylene-CN, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₂₋₁₂ alkenyl, or alkyl optionally substituted with cyano, amido, trialkylammonium, or thiolate; wherein each of the C₃₋₁₂ cycloalkyl, C₂₋₁₂ heterocyclyl, and C₁₋₁₂ heteroaryl is optionally substituted with one or more R^{4a};
each R^{6a} is independently chosen from hydrogen, C₁₋₁₂ alkyl, C₁₋₁₂ alkoxy, —C₀₋₁₂ alkylene-C₃₋₁₂ cycloalkyl, —C₀₋₁₂ alkylene-C₂₋₁₂ heterocyclyl, —C₀₋₁₂ alkylene-C₆₋₁₂ aryl, and —C₀₋₁₂ alkylene-C₁₋₁₂ heteroaryl; wherein each of the alkyl, C₃₋₁₂ cycloalkyl, C₂₋₁₂ heterocyclyl, and C₁₋₁₂ heteroaryl, is optionally substituted with one or more R^{4a},
R⁶ is alkoxy, amino, sulfonamide, carbamide, or alkyl optionally substituted with cyano;
R⁷ is hydrogen, alkyl, —C₀₋₁₂ alkylene-COOH, optionally substituted C₃₋₁₂ cycloalkyl, C₂₋₁₂ aryl, C₁₋₁₂ heteroaryl, —C₀₋₁₂ alkylene-N(R⁴)—SO₂—R⁵, —C₀₋₁₂ alkylene-P(═O)(R⁴) (R^{4′}), —C₀₋₁₂ alkylene-N(R⁴)—C(═S)—R⁵, —C(═S)—R⁵, or alkyl optionally substituted with cyano;
R⁸ is alkoxy, amino, alkylamino, amide, sulfonamide, or carbamide;
n3 is 0, 1, 2, 3, or 4;
n4 is 1, 2, 3, 4, 5, or 6;
t is 2 or 3;
m is 0, 1, 2, 3, 4, or 5; and
n is 0, 1, 2, 3, or 4.

In some embodiments, A is

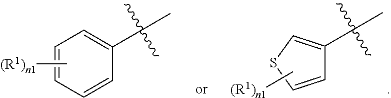

In some embodiments, A is

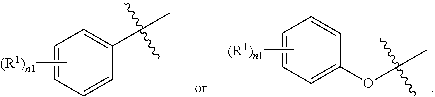

In some embodiments, R¹ is alkyl, such as methyl.

In some embodiments, R¹ is halogen, such as fluorine or chlorine.

In some embodiments, R¹ is alkoxy, such as —OMe.

In some embodiments, R¹ is hydroxyl or —COOH or —CH₂OH.

In some embodiments, R¹ is haloalkyl, such as trifluoromethyl or —CH₂CH₂F.

In some embodiment, B is

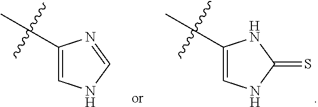 [SMILES: *c1c[nH]cn1 ; *c1c[nH]c(=S)[nH]1]

In some embodiment, B is

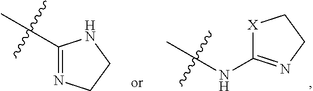

wherein X is S, O, or NH.

In some embodiments, R_{T} is

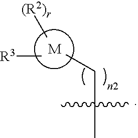

In some embodiments, R_{T} is

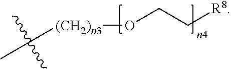

In some embodiments, ring M is C₆₋₁₂ aryl or C₁₋₁₂ heteroaryl.

In some embodiments, ring M is C₃₋₁₂ cycloalkyl or C₂₋₁₂ heterocyclyl, wherein the C₃₋₁₂ cycloalkyl or C₂₋₁₂ heterocyclyl is optionally fused with an aryl.

In some embodiments, ring M is phenyl, pyridinyl, pyrimidinyl, thiophenyl, cyclopentyl, or cyclohexyl.

In some embodiments, ring M is

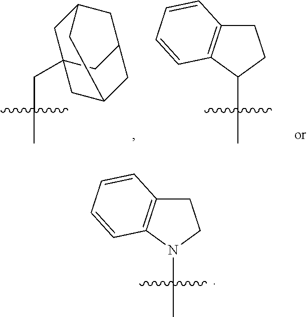

In some embodiments, R² is hydrogen.

In some embodiments, R² is hydroxyl.

In some embodiments, R² is halogen, such as fluorine or chlorine.

In some embodiments, the pharmaceutically acceptable salt of the compound of formula (I) is trifluoroacetate or hydrochloride.

In some embodiments, the compound of formula (II) is a compound of formula (II-A):

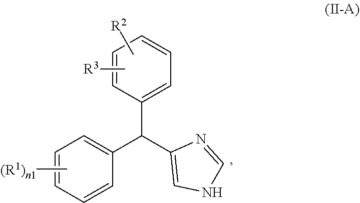

wherein R¹, R², R³, and n1 are defined as above in formula (II).

In some embodiments, R¹ is halogen, haloalkyl, hydroxyl, alkyl, or —COOH.

In certain embodiments, R¹ is methyl, ethyl, hydroxyl, fluorine, chlorine, trifluoromethyl, —CH₂CH₂F, or —COOH.

In some embodiments, n1 is 2.

In some embodiments, R² is hydrogen, hydroxyl, or halogen.

In certain embodiments, R² is fluorine or chlorine.

In some embodiments, R³ is —C(O)—NR⁴R^{4′} or —SO₂—NR⁴R^{4′}, wherein each of R⁴ and R^{4′} is independently hydrogen, alkyl, alkoxy, —C₀₋₁₂ alkylene-N(R⁶ª) t, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or hydroxyalkyl, and the hydroxyalkyl is optionally substituted with alkoxy; wherein each of the alkyl, C₃₋₁₂ cycloalkyl, C₂₋₁₂ heterocyclyl, and C₁₋₁₂ heteroaryl is optionally substituted with one or more R^{4a}, wherein R^{4a}, R^{6a}, and t are defined as above.

In certain embodiments, each of R⁴ and R^{4′} is independently hydrogen, alkyl, alkoxy, or hydroxyalkyl.

In certain embodiment, each of R⁴ and R^{4′} is independently

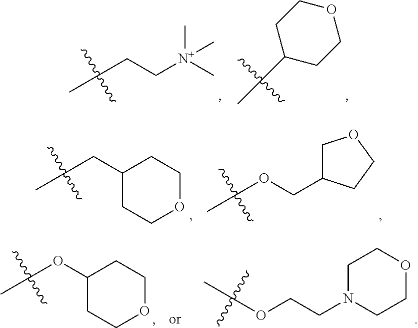

In certain embodiments, each of R⁴ and R^{4′} is independently hydroxyalkyl substituted with alkoxy, such as

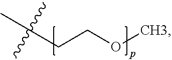

wherein p is 0, 1, 2, or 3, particularly p is 2.

In some embodiments, R³ is —C(O)—NR⁴R⁴ or —SO₂—NR⁴R^{4′}, wherein R⁴ and R^{4′}, together with the nitrogen atom that they are attached to, form a heterocycle comprising one or more heteroatoms chosen from O, N, and S, particularly, R⁴ and R^{4′}, together with the nitrogen atom that they are attached to, form a six-membered heterocycle.

In some embodiments, R³ is hydroxyl, —COOH, —CH(CH₃)—COOH, —CN,

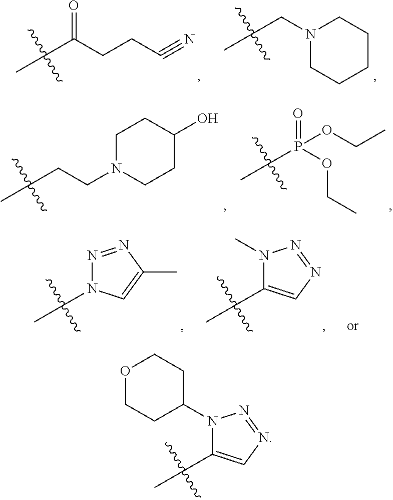

In some embodiments, R³ is C₀₋₁₂ alkylene-N(R⁴)—C(O)—R⁵, —C₀₋₁₂ alkylene-N(R⁴)—SO₂—R⁵, or —C₀₋₁₂ alkylene-O—C₀₋₁₂ alkylene-N(R⁴)—SO₂—R⁵, wherein R⁴ is hydrogen or alkyl, and R⁵ is amino, alkylamino, C₁₋₁₂ haloalkyl, —C₀₋₁₂ alkylene-OR^{6a}, —C₀₋₁₂ alkylene-N(R^{6a}) t, —C₀₋₁₂ alkylene-SR^{6a}, —C₀₋₁₂ alkylene-CN, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₂₋₁₂ alkenyl, or alkyl optionally substituted with cyano or amido; wherein each of the C₃₋₁₂ cycloalkyl, C₂₋₁₂ heterocyclyl, and C₁₋₁₂ heteroaryl is optionally substituted with one or more R^{4a}, wherein R^{4a}, R^{6a}, and t are defined as above.

In certain embodiments, R⁵ is amino, alkylamino, alkoxy, alkyl, or —C₂₋₁₂ alkenyl.

In certain embodiments, R⁵ is alkyl substituted with cyano, such as —CH₂CN.

In certain embodiments, R⁵ is alkyl substituted with amido, such as —CH₂CH₃CONH₂.

In certain embodiments, R⁵ is alkyl substituted with alkoxy, trialkylammonium, or thiolate.

In certain embodiments, when R⁵ is —C₀₋₁₂ alkylene-C₃₋₁₂ cycloalkyl, —C₀₋₁₂ alkylene-C₂₋₁₂ heterocyclyl, or —C₀₋₁₂ alkylene-C₁₋₁₂ heteroaryl, the C₃₋₁₂ cycloalkyl, C₂₋₁₂ heterocyclyl, and C₁₋₁₂ heteroaryl is chosen from

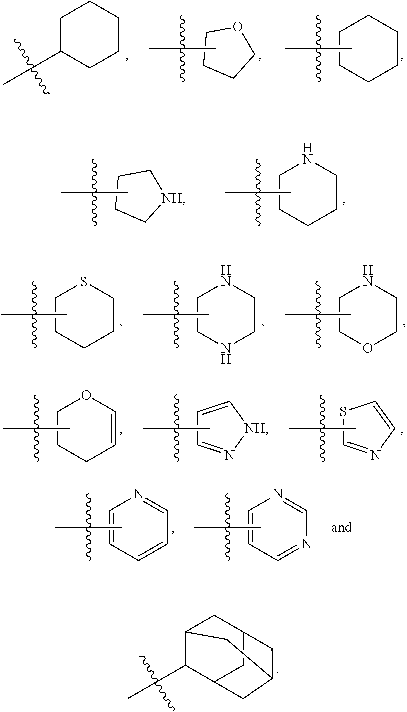

In certain embodiments, when each of the C₃₋₁₂ cycloalkyl, C₂₋₁₂ heterocyclyl, and C₁₋₁₂ heteroaryl is substituted with one or more R^{4a}, R^{4a} is hydroxyl, methyl, oxo, or —C(O)-Me.

In some embodiments, R³ is

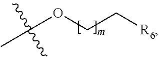

wherein m is 0, 1, 2, 3, 4, or 5, and R⁶ is sulfonamide, carbamide, or alkyl optionally substituted with cyano.

In certain embodiments, R⁶ is sulfonamide of formula —N(R′)SO₂—R, wherein each R and R′ is independently chosen from hydrogen and alkyl, particularly R⁶ is —NHSO₂CH₃.

In certain embodiments, R⁶ is carbamide of formula —N(R′)C(O)N(R)₂, wherein each R and R′ is independently chosen from hydrogen, alkyl, and heteroaryl, particularly R⁶ is

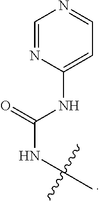 [SMILES: *NC(=O)Nc1ccncn1]

In certain embodiments, R⁶ is alkyl optionally substituted with cyano, such as C₁₋₄ alkyl optionally substituted with cyano, particularly C₁₋₄ alkyl substituted with cyano.

In certain embodiments, m is 1, 2, or 3, particularly 2.

In some embodiments, R³ is —NH—R⁷, wherein R⁷ is hydrogen, optionally substituted C₃₋₁₂ cycloalkyl, C₁₋₁₂ heteroaryl, —C₀₋₁₂ alkylene-N(R⁴)—SO₂—R⁵, —C₀₋₁₂ alkylene-P(═O)(R⁴) (R^{4′}), —C₀₋₁₂ alkylene-N(R⁴)—C(═S)—R⁵, —C(═S)—R⁵, or alkyl optionally substituted with cyano; wherein R⁴, R^{4′} and R⁵ are defined as above.

In certain embodiments, R⁷ is hydrogen.

In certain embodiments, R⁷ is

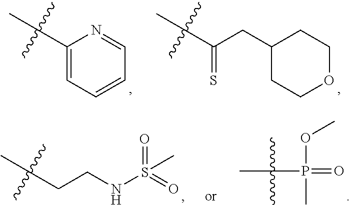

In certain embodiments, R⁷ is alkyl optionally substituted with cyano, such as C₁₋₄ alkyl optionally substituted with cyano, particularly C₁₋₄ alkyl substituted with cyano.

In certain embodiments, R³ is

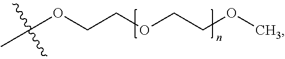

n is 3 or 4, particularly 4.

In some embodiments, the compound of formula (II) is a compound of formula (II-B):

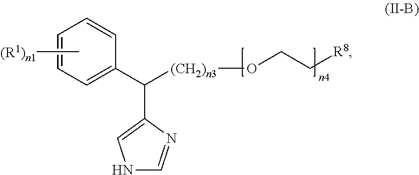

wherein R¹, R⁸, n1, n3, and n4 are defined as above in formula (II).

In some embodiments, R¹ is hydrogen or alkyl, such as alkyl, particularly methyl.

In some embodiments, n1 is 2.

In some embodiments, n3 is 0, 1 or 3.

In some embodiments, n4 is 2, 3, or 5.

In some embodiments, R⁸ is alkoxy, such as C₁₋₄ alkoxy, particularly methoxy or ethoxy.

In some embodiments, R⁸ is amino.

In some embodiments, R⁸ is alkylamino, such as C₁₋₄ alkylamino, particularly methylamino.

In some embodiments, R⁸ is amide of formula —N(R′)C(O)R, wherein each R and R′ is independently chosen from hydrogen, alkyl, cycloalkyl, aryl, and heteroaryl.

In some embodiments, R⁸ is amide of formula —N(R′)C(O)R, particularly R⁸ is —NHCOCH₃.

In some embodiments, R⁸ is sulfonamide of formula —N(R′)SO₂—R, wherein each R and R′ is independently chosen from hydrogen, alkyl, cycloalkyl, aryl, and heteroaryl.

In certain embodiments, R⁸ is sulfonamide of formula —N(R′)SO₂—R, wherein each R and R′ is independently chosen from hydrogen, —C₀₋₁₂ alkylene-C₂₋₁₂ heterocyclyl, and alkyl, particularly R³ is —NHSO₂CH₃.

In some embodiments, R⁸ is carbamide of formula —N(R′)C(O)N(R)₂, wherein each R and R′ is independently chosen from hydrogen, alkyl, cycloalkyl, aryl, and heteroaryl.

In certain embodiments, R⁸ is carbamide of formula —N(R′)C(O)N(R)₂, wherein each R and R′ is independently chosen from hydrogen, alkyl, and heteroaryl, particularly R³ is —NHC(O)NHCH₃ or

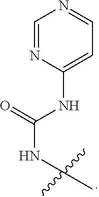 [SMILES: *NC(=O)Nc1ccncn1]

In some embodiments, when the compound is a compound of formula (II), R⁸ is —OCH₃, —NH₂, —NHCH₃, —NHC(O)CH₃,

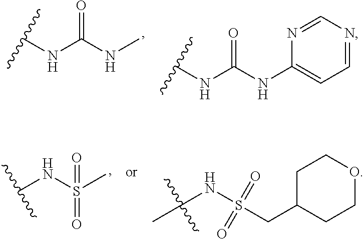

In some embodiments, the compound of formula (II) is a compound of formula (II-C):

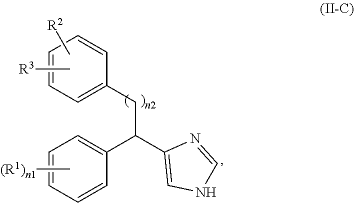

wherein,
n2 is 1 or 2; and
R¹, R², R³, and n1 are defined as above in formula (II).

In some embodiments, R¹ is alkyl, such as methyl.

In some embodiments, n1 is 2.

In some embodiments, n2 is 1.

In some embodiments, R² is hydrogen or halogen.

In certain embodiments, R² is fluorine.

In some embodiments, R³ is —C(O)—NR⁴R^{4′}, wherein each of R⁴ and R^{4′} is independently hydrogen, hydroxy, alkyl, alkoxy, —SO₂—NHCH₃, —SO₂—NH-Ph, —CH₂—COOH, —CH₂—CH₂—COOH, or

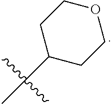 [SMILES: *C1CCOCC1]

In some embodiments, R³ is —SO₂—NR⁴R^{4′}, wherein each of R⁴ and R^{4′} is independently hydrogen, hydroxy, or —C₀₋₁₂ alkylene-C₂₋₁₂ heterocyclyl.

In some embodiments, R³ is —NH—C(O)—R⁵, —N(CH₃)—C(O)—R⁵ or —NH—SO₂—R⁵, wherein R⁵ is alkyl, —C₀₋₁₂ alkylene-alkoxy, —C₀₋₁₂ alkylene-C₃₋₁₂ cycloalkyl, —C₀₋₁₂ alkylene-NH—C₁₋₁₂ alkyl, —C₀₋₁₂ alkylene-NH—C₂₋₁₂ heterocyclyl, or —C₀₋₁₂ alkylene-C₂₋₁₂ heterocyclyl.

In certain embodiments, R⁵ is alkyl, such as methyl.

In certain embodiments, R⁵ is —C₀₋₁₂ alkylene-alkoxy, such as —CH₂—OCH₃.

In certain embodiments, R⁵ is —C₀₋₁₂ alkylene-C₃₋₁₂ cycloalkyl, such as

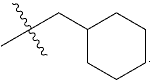 [SMILES: *CC1CCCCC1]

In certain embodiments, R⁵ is —C₀₋₁₂ alkylene-NH—C₁₋₁₂ alkyl, such as —NH—CH₃.

In certain embodiments, R⁵ is —C₀₋₁₂ alkylene-NH—C₂₋₁₂ heterocyclyl, such as

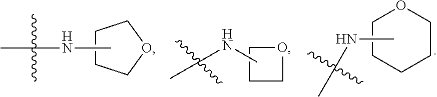

In certain embodiments, R⁵ is —C₀₋₁₂ alkylene-C₂₋₁₂ heterocyclyl, such as

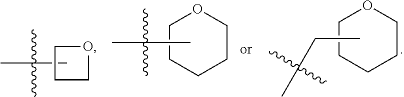

In some embodiments, R³ is —SO₂-alkyl, such as —SO₂—CH₃.

In some embodiments, R³ is —C₀₋₁₂ alkylene-COOH, such as —COOH, —CH₂—COOH, —C (Me)₂—COOH, —CH₂—CH₂—COOH.

In some embodiments, R³ is —C₀₋₁₂ alkylene-P(═O)(R⁴) (R^{4′}), such as

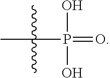 [SMILES: *P(=O)(O)O]

In some embodiments, R³ is —C₀₋₁₂ alkylene-C₁₋₁₂ heteroaryl, such as

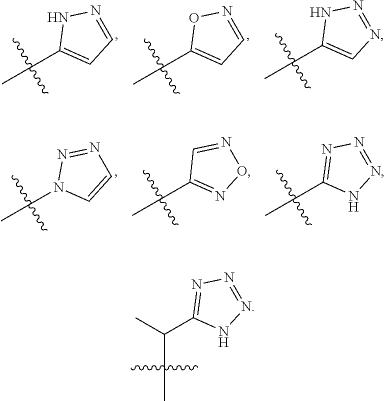

In some embodiments, R³ is —NH—R⁷, such as —NH—CH₃,

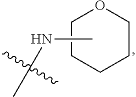

—NH—C(═S)—R⁵ such as

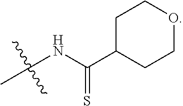 [SMILES: *NC(=S)C1CCOCC1]

In some embodiments, the compound of formula (II) is a compound of formula (II-D):

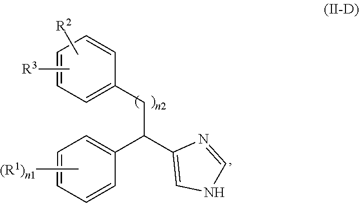

wherein,
n2 is 0 or 1;
R³ is chosen from —C(O)—NHR⁴, —SO₂—NHR⁴, —NH—C(O)—R⁵, and —NH—SO₂—R⁵, and —NH—R⁷;
R⁴ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein and each of the C₃₋₁₂ cycloalkyl, C₂₋₁₂ heterocyclyl, and C₁₋₁₂ heteroaryl is optionally substituted with one or more R^{4a},
R⁵ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein each of the C₃₋₁₂ Cycloalkyl, C₂₋₁₂ heterocyclyl, and C₁₋₁₂ heteroaryl is optionally substituted with one or more R^{4a},
each R^{6a} is independently chosen from —C₀₋₁₂ alkylene-C₃₋₁₂ cycloalkyl, —C₀₋₁₂ alkylene-C₂-12 heterocyclyl, and —C₀₋₁₂ alkylene-C₁₋₁₂ heteroaryl; wherein each of the C₃₋₁₂ cycloalkyl, C₂₋₁₂ heterocyclyl, and C₁₋₁₂ heteroaryl is optionally substituted with one or more R^{4a};
R⁷ is C₁₋₁₂ heteroaryl, —C₀₋₁₂ alkylene-N(R⁴)—SO₂—R⁵, —C₀₋₁₂ alkylene-P(═O)(R⁴) (alkoxy), or —C₀₋₁₂ alkylene-N(R⁴)—C(═S)—R⁵; and
R¹, R², R^{4a}, and n1 and are defined as above in formula (II).

In some embodiments, R¹ is alkyl, such as methyl.

In some embodiments, R¹ is alkoxy, such as —OMe.

In some embodiments, n1 is 1.

In some embodiments, n1 is 2.

In some embodiments, n2 is 0.

In some embodiments, n2 is 1.

In some embodiments, R² is hydrogen or halogen.

In some embodiments, R³ is —C(O)—NHR⁴ or —SO₂—NHR⁴, wherein R⁴ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein and each of the C₃₋₁₂ cycloalkyl, C₂₋₁₂ heterocyclyl, and C₁₋₁₂ heteroaryl is optionally substituted with one or more R^{4a}, wherein R^{4a} and R^{6a} are defined as above.

In certain embodiment, R⁴ is

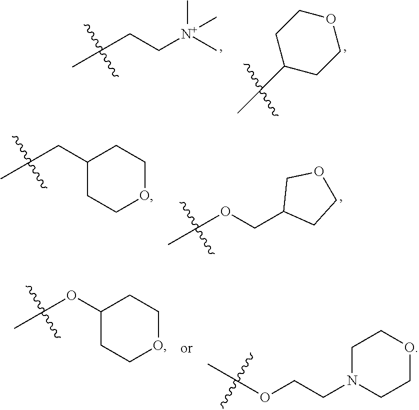

In some embodiments, R³ is —NH—C(O)—R⁵, or —NH—SO₂—R⁵, wherein R⁵ is —C₀₋₁₂ alkylene-NHR^{6a}, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or alkyl substituted with trialkylammonium; wherein each of the C₃₋₁₂ cycloalkyl, C₂₋₁₂ heterocyclyl, and C₁₋₁₂ heteroaryl is optionally substituted with one or more R^{4a}, wherein R^{4a} and R^{6a} are defined as above.

In certain embodiments, R⁵ is alkyl substituted with trialkylammonium.

In certain embodiments, when R⁵ is —C₀₋₁₂ alkylene-C₃₋₁₂ cycloalkyl, —C₀₋₁₂ alkylene-C₂₋₁₂ heterocyclyl, or —C₀₋₁₂ alkylene-C₁₋₁₂ heteroaryl, the C₃₋₁₂ cycloalkyl, C₂₋₁₂ heterocyclyl, and C₁₋₁₂ heteroaryl is chosen from

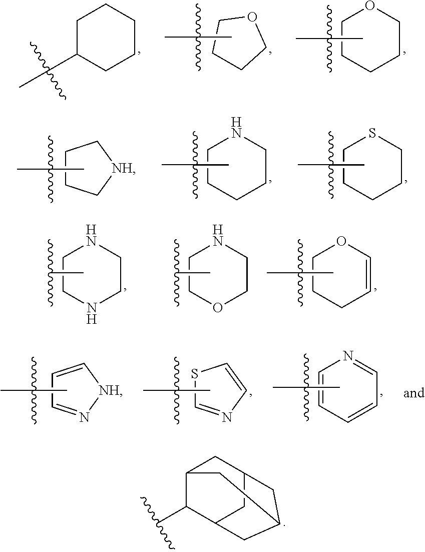

In certain embodiments, when each of the C₃₋₁₂ cycloalkyl, C₂₋₁₂ heterocyclyl, and C₁₋₁₂ heteroaryl is substituted with one or more R^{4a}, R^{4a} is hydroxyl, methyl, oxo, or —C(O)-Me.

In some embodiments, R³ is —NH—R⁷, wherein R⁷ is C₁₋₁₂ heteroaryl, —C₀₋₁₂ alkylene-N(R⁴)—SO₂—R⁵, —C₀₋₁₂ alkylene-P(═O)(R⁴) (alkoxy), or —C₀₋₁₂ alkylene-N(R⁴)—C(═S)—R⁵; wherein R⁴ and R⁵ are defined as above.

In certain embodiments, R⁷ is

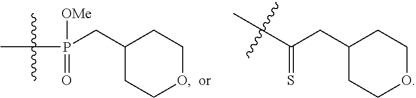

In some embodiments, the compound of formula (II) is a compound of formula (II-E):

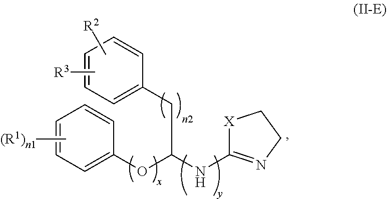

wherein
x is 0 or 1;
y is 0 or 1;
X is S, O, or NH; and
R¹, R², R³, n1, and n2 are defined as above in formula (II).

In some embodiments, x is 0, y is 1, and X is S, O, or NH.

In some embodiments, x is 0 or 1, y is 0, and X is NH.

In some embodiments, each R¹ is independently chosen from hydrogen, halogen, alkoxy, and alkyl.

In certain embodiments, R¹ is methyl, chlorine, or methoxy.

In some embodiments, n2 is 1.

In some embodiments, n2 is 2.

In some embodiments, R² is hydrogen.

In some embodiments, R³ is —C(O)—NR⁴R^{4′}, wherein each of R⁴ and R^{4′} is independently hydrogen or alkoxy.

In some embodiments, R³ is —SO₂—NR⁴R^{4′}, wherein each of R⁴ and R^{4′} is independently hydrogen or alkyl.

In some embodiments, R³ is —NH—C(O)—R⁵ or —NH—SO₂—R⁵, wherein R⁵ is alkyl or —C₀₋₁₂ alkylene-C₂₋₁₂ heterocyclyl.

In certain embodiments, R⁵ is alkyl, such as methyl.

In certain embodiments, R⁵ is —C₀₋₁₂ alkylene-C₂₋₁₂ heterocyclyl, such as

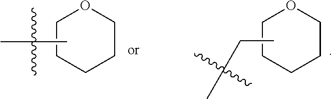

In some embodiments, the compound of formula (II) is a compound of formula (II-F):

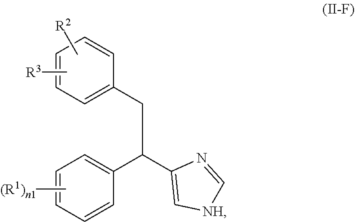

wherein,
R² is adjacent to R³, and R² and R³, together with the carbon atoms that they are attached to, form a heterocycle optionally substituted with one or more R^{4a}; and
R¹, R^{4a}, and n1 are defined as above in formula (II).

In some embodiments, R² and R³, together with the carbon atoms that they are attached to, form a 5- or 6-membered heterocycle optionally substituted with one or more R^{4a}.

In some embodiments, the compound of formula (II-F) is a compound of formula (II-F-1):

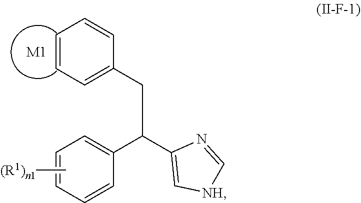

wherein M1 is a heterocycle optionally substituted with one or more R^{4a}.

In some embodiments, the compound of formula (II) is a compound of formula (II-G):

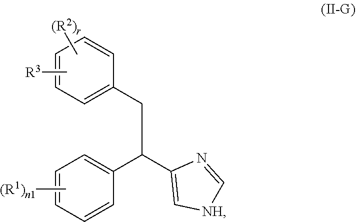

wherein,
each R² is independently chosen from hydroxyl and alkoxy;
R³ is chosen from hydroxy and alkoxy; and
R¹, n1 and r are defined as above in formula (II).

In some embodiments, one R² is adjacent to R³.

In some embodiments, each R₂ is independently chosen from hydroxyl and methoxy.

In some embodiments, R³ is chosen from hydroxyl and methoxy.

In some embodiments, the compound of formula (II-G) is a compound of formula (II-G-1) of (II-G-2):

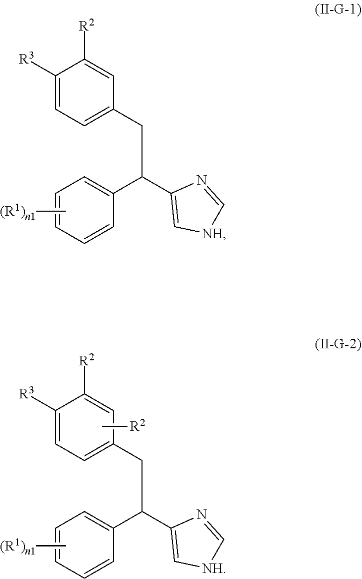

In some embodiments, the compound of formula (II) is a compound of formula (II-H):

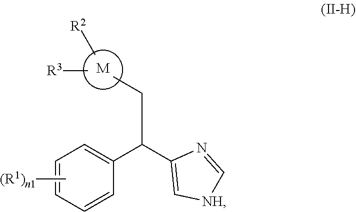

wherein,
M is C₆₋₁₂ aryl or C₁₋₁₂ heteroaryl;
R³ is chosen from —C₀₋₁₂ alkylene-COOH, —O—C₀₋₁₂ alkylene-COOH, —C₀₋₁₂ alkylene-P(O)(OH) 2, —C(O)—NH—SO₂—R⁵, —C(O)—NH—C₀₋₁₂ alkylene-COOH, —NH—C₀₋₁₂ alkylene-COOH, —SO₂—OH, and

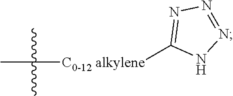

wherein the —C₀₋₁₂ alkylene-COOH is optionally substituted with one or more substitutes chosen from amino and alkylamino; and

R¹, R², and n1 are defined as above in formula (II).

In some embodiments, M is phenyl.

In some embodiments, M is pyridinyl.

Exemplary compounds of formula (I-A), (I-B), (I-C), (I-D), or (II) include, but are not limited to, the compounds described herein, and any tautomer, stereoisomer, pharmaceutically acceptable salt or solvate thereof.

In particular embodiments, provided is a compound selected from Compounds 1-251, 401-403, 501-509, 601, and 602, or a tautomer, stereoisomer, pharmaceutically acceptable salt or solvate thereof.

All possible combinations of the above-indicated embodiments of compounds of formula (I-A), (I-B), (I-C), (I-D), or (II) and their tautomers, stereoisomers, pharmaceutically acceptable salts and solvates are considered to be embraced within the scope of the present disclosure.

Exemplary compounds of formula (I-A), (I-B), (I-C), (I-D), or (II) include, but are not limited to, the following compounds, and any tautomer, stereoisomer, pharmaceutically acceptable salt or solvate thereof:

TABLE 1

| No. | Structure |
|---|---|
| 1 | 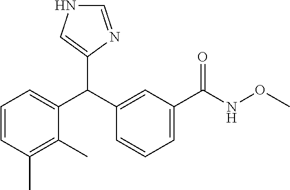 [SMILES: CONC(=O)c1cccc(C(c2c[nH]cn2)c2cccc(C)c2C)c1] |
| 2 | 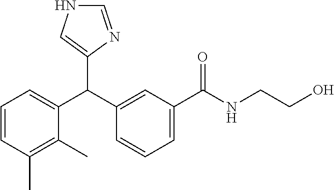 [SMILES: Cc1cccc(C(c2cccc(C(=O)NCCO)c2)c2c[nH]cn2)c1C] |
| 3 | 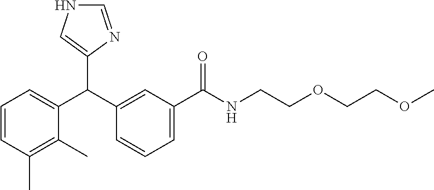 [SMILES: COCCOCCNC(=O)c1cccc(C(c2c[nH]cn2)c2cccc(C)c2C)c1] |
| 4 | 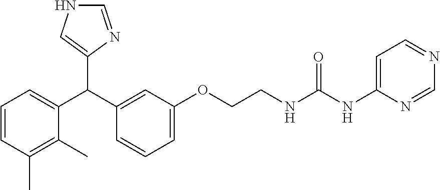 [SMILES: Cc1cccc(C(c2cccc(OCCNC(=O)Nc3ccncn3)c2)c2c[nH]cn2)c1C] |
| 5 | 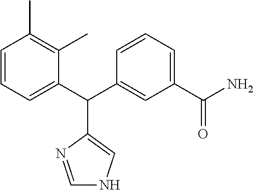 [SMILES: Cc1cccc(C(c2cccc(C(N)=O)c2)c2c[nH]cn2)c1C] |
| 6 | 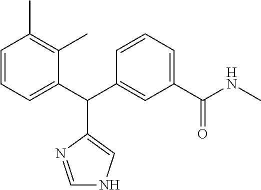 [SMILES: CNC(=O)c1cccc(C(c2c[nH]cn2)c2cccc(C)c2C)c1] |
| 7 | 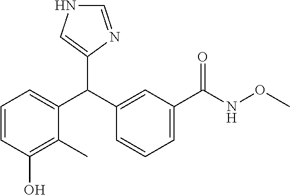 [SMILES: CONC(=O)c1cccc(C(c2c[nH]cn2)c2cccc(O)c2C)c1] |
| 8 | 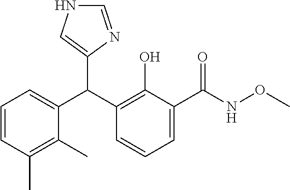 [SMILES: CONC(=O)c1cccc(C(c2c[nH]cn2)c2cccc(C)c2C)c1O] |
| 9 | 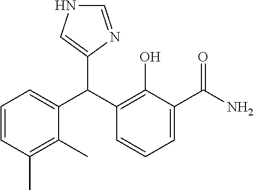 [SMILES: Cc1cccc(C(c2c[nH]cn2)c2cccc(C(N)=O)c2O)c1C] |
| 10 | 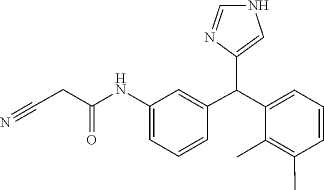 [SMILES: Cc1cccc(C(c2cccc(NC(=O)CC#N)c2)c2c[nH]cn2)c1C] |
| 11 | 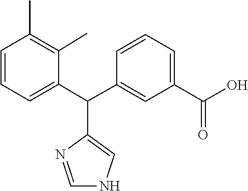 [SMILES: Cc1cccc(C(c2cccc(C(=O)O)c2)c2c[nH]cn2)c1C] |
| 12 | 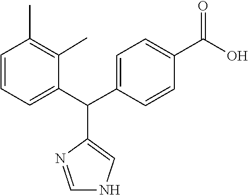 [SMILES: Cc1cccc(C(c2ccc(C(=O)O)cc2)c2c[nH]cn2)c1C] |
| 13 | 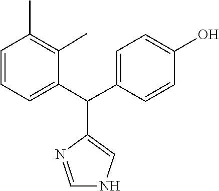 [SMILES: Cc1cccc(C(c2ccc(O)cc2)c2c[nH]cn2)c1C] |
| 14 | 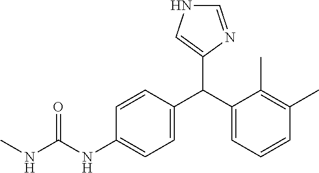 |
| 15 | 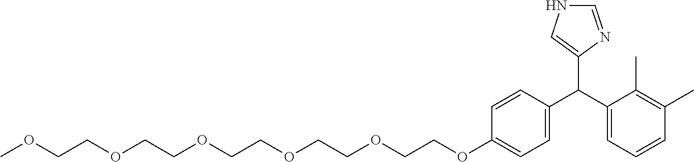 |
| 16 | 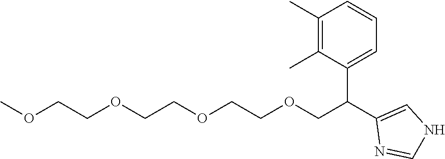 [SMILES: COCCOCCOCCOCC(c1c[nH]cn1)c1cccc(C)c1C] |
| 17 | 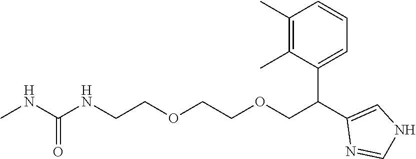 [SMILES: CNC(=O)NCCOCCOCC(c1c[nH]cn1)c1cccc(C)c1C] |
| 18 | 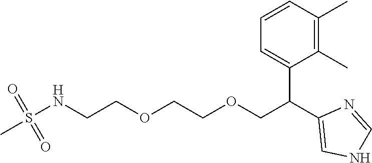 [SMILES: Cc1cccc(C(COCCOCCNS(C)(=O)=O)c2c[nH]cn2)c1C] |
| 19 | 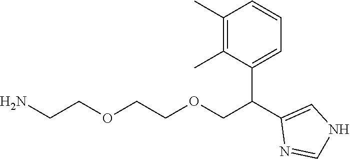 [SMILES: Cc1cccc(C(COCCOCCN)c2c[nH]cn2)c1C] |
| 20 | 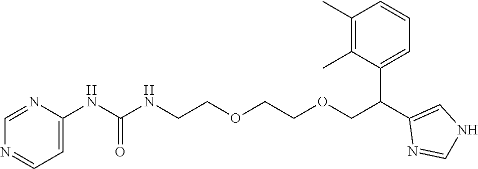 [SMILES: Cc1cccc(C(COCCOCCNC(=O)Nc2ccncn2)c2c[nH]cn2)c1C] |
| 21 | 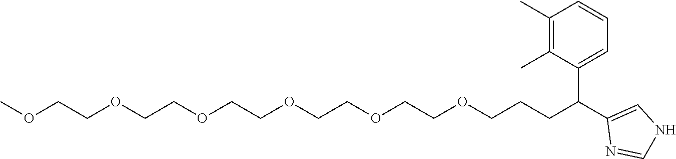 [SMILES: COCCOCCOCCOCCOCCOCCCC(c1c[nH]cn1)c1cccc(C)c1C] |
| 22 | 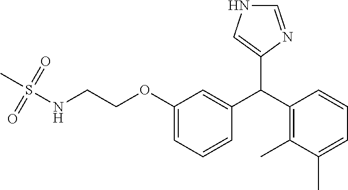 [SMILES: Cc1cccc(C(c2cccc(OCCNS(C)(=O)=O)c2)c2c[nH]cn2)c1C] |
| 23 | 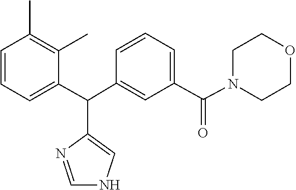 [SMILES: Cc1cccc(C(c2cccc(C(=O)N3CCOCC3)c2)c2c[nH]cn2)c1C] |
| 24 | 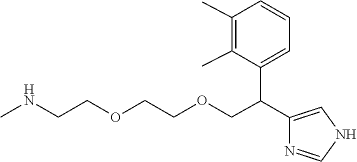 [SMILES: CNCCOCCOCC(c1c[nH]cn1)c1cccc(C)c1C] |
| 25 | 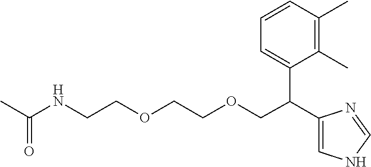 [SMILES: CC(=O)NCCOCCOCC(c1c[nH]cn1)c1cccc(C)c1C] |
| 26 | 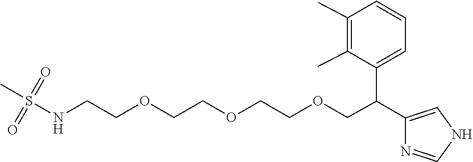 [SMILES: Cc1cccc(C(COCCOCCOCCNS(C)(=O)=O)c2c[nH]cn2)c1C] |
| 27 | 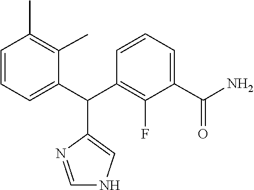 [SMILES: Cc1cccc(C(c2c[nH]cn2)c2cccc(C(N)=O)c2F)c1C] |
| 28 | 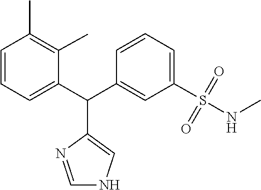 [SMILES: CNS(=O)(=O)c1cccc(C(c2c[nH]cn2)c2cccc(C)c2C)c1] |
| 29 | 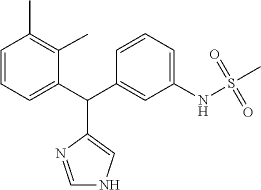 [SMILES: Cc1cccc(C(c2cccc(NS(C)(=O)=O)c2)c2c[nH]cn2)c1C] |
| 30 | 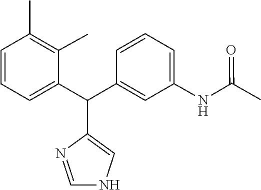 [SMILES: CC(=O)Nc1cccc(C(c2c[nH]cn2)c2cccc(C)c2C)c1] |
| 31 | 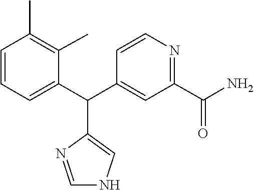 [SMILES: Cc1cccc(C(c2ccnc(C(N)=O)c2)c2c[nH]cn2)c1C] |
| 32 | 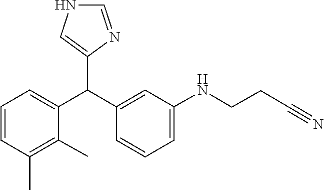 [SMILES: Cc1cccc(C(c2cccc(NCCC#N)c2)c2c[nH]cn2)c1C] |
| 33 | 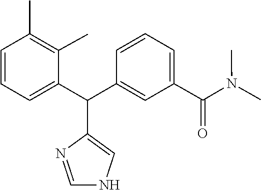 [SMILES: Cc1cccc(C(c2cccc(C(=O)N(C)C)c2)c2c[nH]cn2)c1C] |
| 34 | 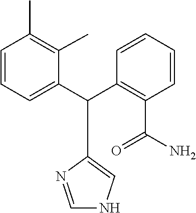 [SMILES: Cc1cccc(C(c2c[nH]cn2)c2ccccc2C(N)=O)c1C] |
| 35 | 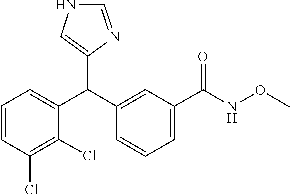 [SMILES: CONC(=O)c1cccc(C(c2c[nH]cn2)c2cccc(Cl)c2Cl)c1] |
| 36 | 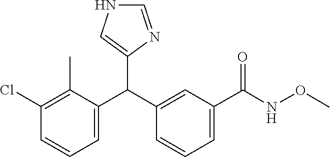 [SMILES: CONC(=O)c1cccc(C(c2c[nH]cn2)c2cccc(Cl)c2C)c1] |
| 37 | 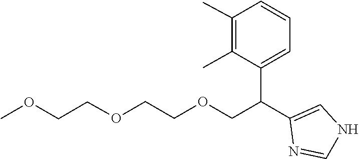 [SMILES: COCCOCCOCC(c1c[nH]cn1)c1cccc(C)c1C] |
| 38 | 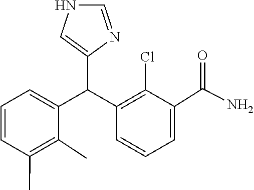 [SMILES: Cc1cccc(C(c2c[nH]cn2)c2cccc(C(N)=O)c2Cl)c1C] |
| 39 | 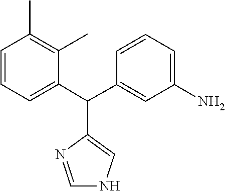 [SMILES: Cc1cccc(C(c2cccc(N)c2)c2c[nH]cn2)c1C] |
| 40 | 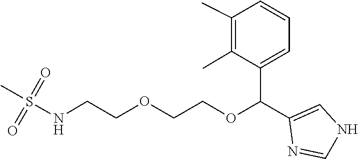 [SMILES: Cc1cccc(C(OCCOCCNS(C)(=O)=O)c2c[nH]cn2)c1C] |
| 41 | 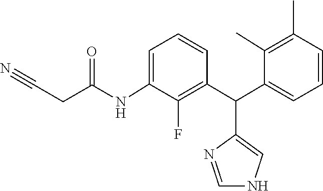 [SMILES: Cc1cccc(C(c2c[nH]cn2)c2cccc(NC(=O)CC#N)c2F)c1C] |
| 42 | 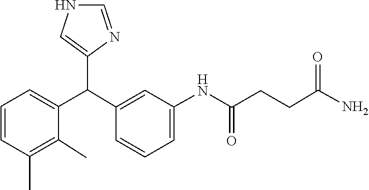 [SMILES: Cc1cccc(C(c2cccc(NC(=O)CCC(N)=O)c2)c2c[nH]cn2)c1C] |
| 43 | 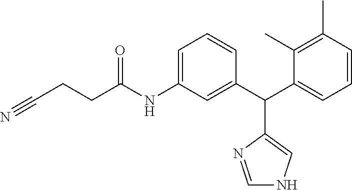 [SMILES: Cc1cccc(C(c2cccc(NC(=O)CCC#N)c2)c2c[nH]cn2)c1C] |
| 44 | 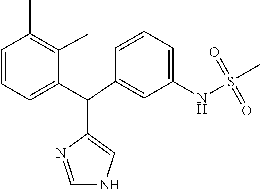 [SMILES: Cc1cccc(C(c2cccc(NS(C)(=O)=O)c2)c2c[nH]cn2)c1C] |
| 45 | 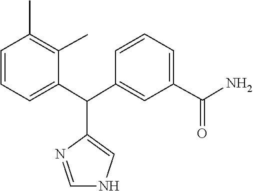 [SMILES: Cc1cccc(C(c2cccc(C(N)=O)c2)c2c[nH]cn2)c1C] |
| 46 | 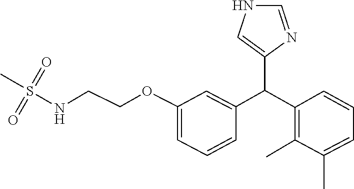 [SMILES: Cc1cccc(C(c2cccc(OCCNS(C)(=O)=O)c2)c2c[nH]cn2)c1C] |
| 47 | 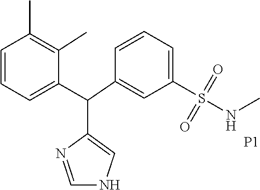 |
| 48 | 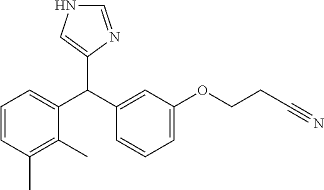 [SMILES: Cc1cccc(C(c2cccc(OCCC#N)c2)c2c[nH]cn2)c1C] |
| 49 | 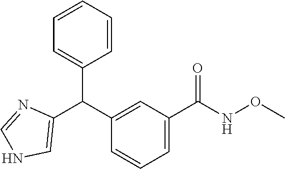 [SMILES: CONC(=O)c1cccc(C(c2ccccc2)c2c[nH]cn2)c1] |
| 50 | 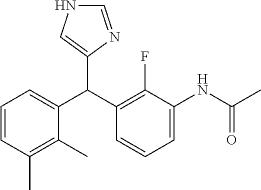 [SMILES: CC(=O)Nc1cccc(C(c2c[nH]cn2)c2cccc(C)c2C)c1F] |
| 51 | 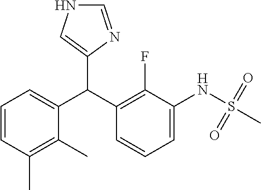 [SMILES: Cc1cccc(C(c2c[nH]cn2)c2cccc(NS(C)(=O)=O)c2F)c1C] |
| 52 | 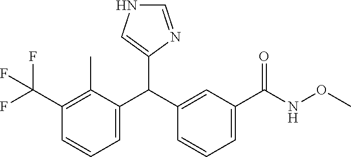 [SMILES: CONC(=O)c1cccc(C(c2c[nH]cn2)c2cccc(C(F)(F)F)c2C)c1] |
| 53 | 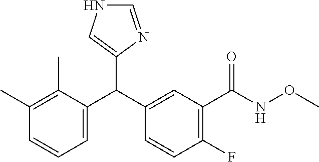 |
| 54 | 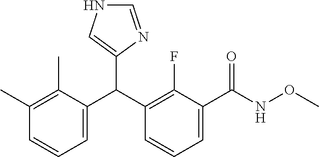 |
| 55 | 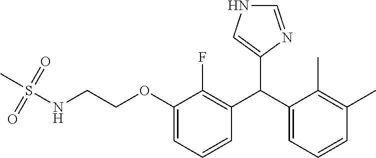 |
| 56 | 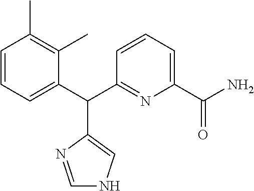 [SMILES: Cc1cccc(C(c2c[nH]cn2)c2cccc(C(N)=O)n2)c1C] |
| 57 | 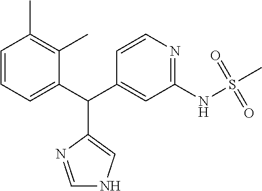 [SMILES: Cc1cccc(C(c2ccnc(NS(C)(=O)=O)c2)c2c[nH]cn2)c1C] |
| 58 | 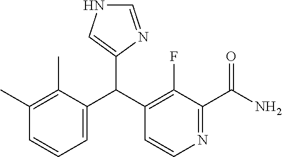 [SMILES: Cc1cccc(C(c2c[nH]cn2)c2ccnc(C(N)=O)c2F)c1C] |
| 59 | 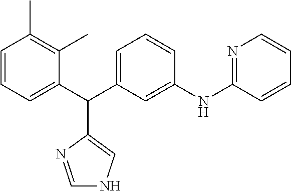 [SMILES: Cc1cccc(C(c2cccc(Nc3ccccn3)c2)c2c[nH]cn2)c1C] |
| 60 | 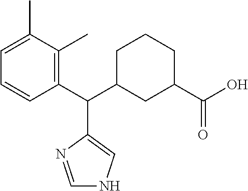 [SMILES: Cc1cccc(C(c2c[nH]cn2)C2CCCC(C(=O)O)C2)c1C] |
| 61 | 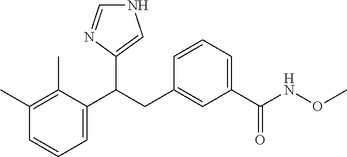 |
| 62 | 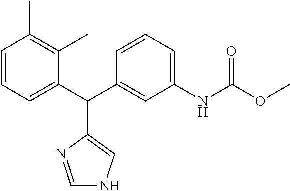 [SMILES: COC(=O)Nc1cccc(C(c2c[nH]cn2)c2cccc(C)c2C)c1] |
| 63 | 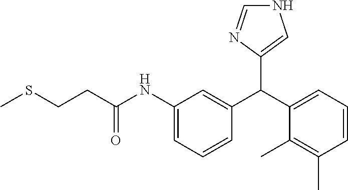 [SMILES: CSCCC(=O)Nc1cccc(C(c2c[nH]cn2)c2cccc(C)c2C)c1] |
| 64 | 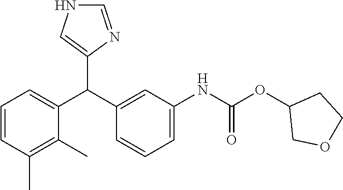 [SMILES: Cc1cccc(C(c2cccc(NC(=O)OC3CCOC3)c2)c2c[nH]cn2)c1C] |
| 65 | 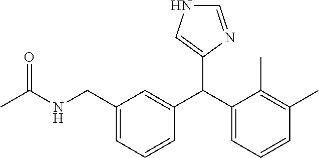 [SMILES: CC(=O)NCc1cccc(C(c2c[nH]cn2)c2cccc(C)c2C)c1] |
| 66 | 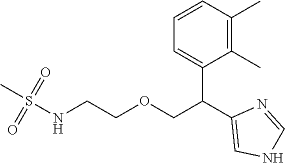 [SMILES: Cc1cccc(C(COCCNS(C)(=O)=O)c2c[nH]cn2)c1C] |
| 67 | 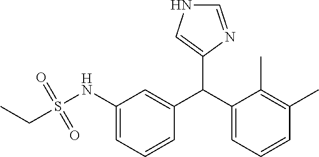 [SMILES: CCS(=O)(=O)Nc1cccc(C(c2c[nH]cn2)c2cccc(C)c2C)c1] |
| 68 | 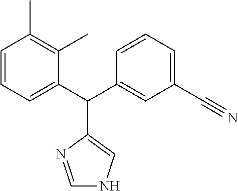 [SMILES: Cc1cccc(C(c2cccc(C#N)c2)c2c[nH]cn2)c1C] |
| 69 | 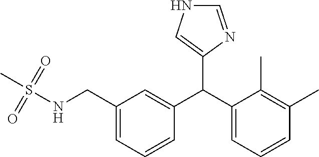 |
| 70 | 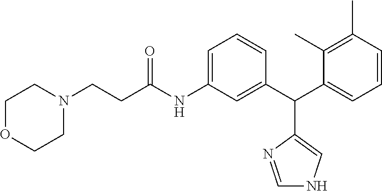 [SMILES: Cc1cccc(C(c2cccc(NC(=O)CCN3CCOCC3)c2)c2c[nH]cn2)c1C] |
| 71 | 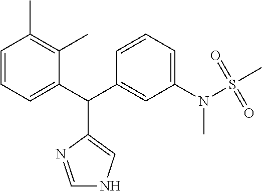 [SMILES: Cc1cccc(C(c2cccc(N(C)S(C)(=O)=O)c2)c2c[nH]cn2)c1C] |
| 72 | 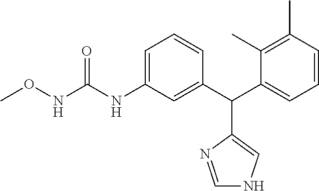 [SMILES: CONC(=O)Nc1cccc(C(c2c[nH]cn2)c2cccc(C)c2C)c1] |
| 73 | 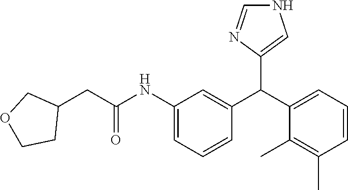 [SMILES: Cc1cccc(C(c2cccc(NC(=O)CC3CCOC3)c2)c2c[nH]cn2)c1C] |
| 74 | 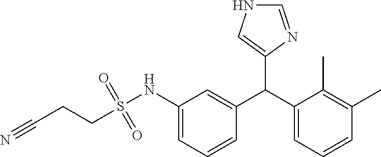 [SMILES: Cc1cccc(C(c2cccc(NS(=O)(=O)CCC#N)c2)c2c[nH]cn2)c1C] |
| 75 | 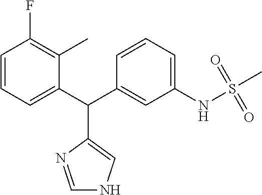 [SMILES: Cc1c(F)cccc1C(c1cccc(NS(C)(=O)=O)c1)c1c[nH]cn1] |
| 76 | 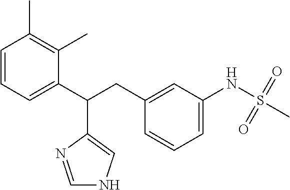 [SMILES: Cc1cccc(C(Cc2cccc(NS(C)(=O)=O)c2)c2c[nH]cn2)c1C] |
| 77 | 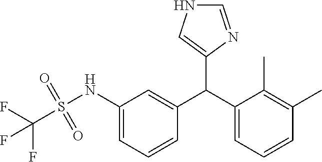 [SMILES: Cc1cccc(C(c2cccc(NS(=O)(=O)C(F)(F)F)c2)c2c[nH]cn2)c1C] |
| 78 | 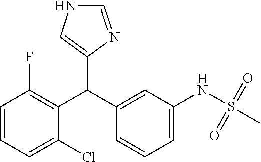 [SMILES: CS(=O)(=O)Nc1cccc(C(c2c[nH]cn2)c2c(F)cccc2Cl)c1] |
| 79 | 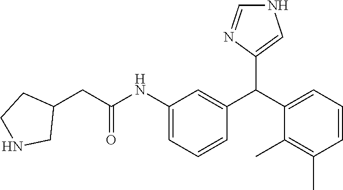 [SMILES: Cc1cccc(C(c2cccc(NC(=O)CC3CCNC3)c2)c2c[nH]cn2)c1C] |
| 80 | 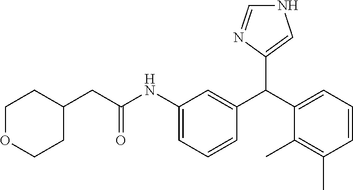 [SMILES: Cc1cccc(C(c2cccc(NC(=O)CC3CCOCC3)c2)c2c[nH]cn2)c1C] |
| 81 | 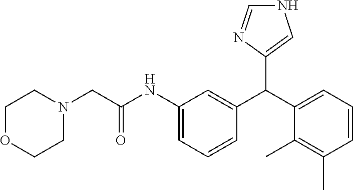 [SMILES: Cc1cccc(C(c2cccc(NC(=O)CN3CCOCC3)c2)c2c[nH]cn2)c1C] |
| 82 | 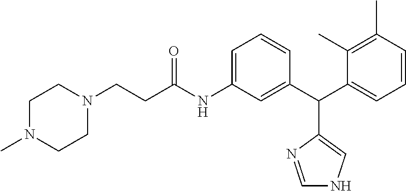 [SMILES: Cc1cccc(C(c2cccc(NC(=O)CCN3CCN(C)CC3)c2)c2c[nH]cn2)c1C] |
| 83 | 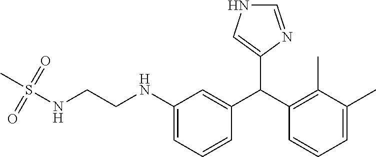 |
| 84 | 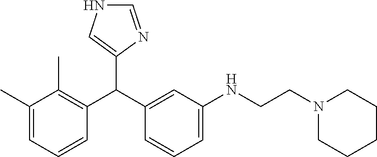 [SMILES: Cc1cccc(C(c2cccc(NCCN3CCCCC3)c2)c2c[nH]cn2)c1C] |
| 85 | 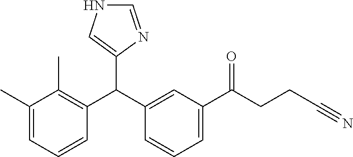 [SMILES: Cc1cccc(C(c2cccc(C(=O)CCC#N)c2)c2c[nH]cn2)c1C] |
| 86 | 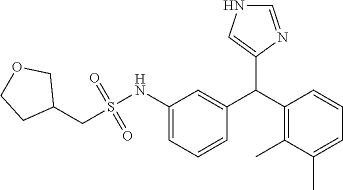 [SMILES: Cc1cccc(C(c2cccc(NS(=O)(=O)CC3CCOC3)c2)c2c[nH]cn2)c1C] |
| 87 | 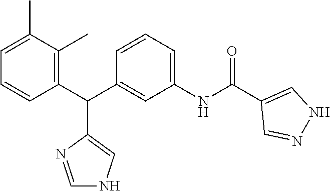 [SMILES: Cc1cccc(C(c2cccc(NC(=O)c3cn[nH]c3)c2)c2c[nH]cn2)c1C] |
| 88 | 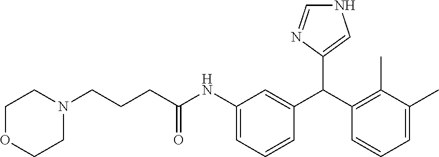 [SMILES: Cc1cccc(C(c2cccc(NC(=O)CCCN3CCOCC3)c2)c2c[nH]cn2)c1C] |
| 89 | 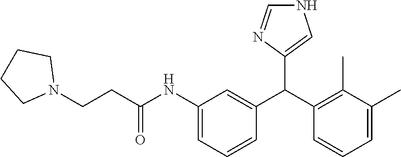 [SMILES: Cc1cccc(C(c2cccc(NC(=O)CCN3CCCC3)c2)c2c[nH]cn2)c1C] |
| 90 | 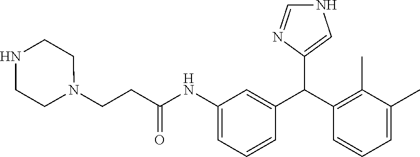 [SMILES: Cc1cccc(C(c2cccc(NC(=O)CCN3CCNCC3)c2)c2c[nH]cn2)c1C] |
| 91 | 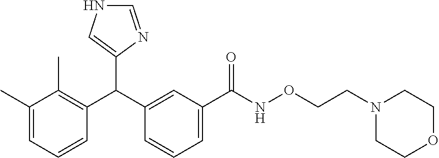 [SMILES: Cc1cccc(C(c2cccc(C(=O)NOCCN3CCOCC3)c2)c2c[nH]cn2)c1C] |
| 92 | 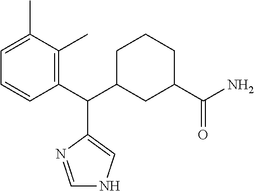 [SMILES: Cc1cccc(C(c2c[nH]cn2)C2CCCC(C(N)=O)C2)c1C] |
| 93 | 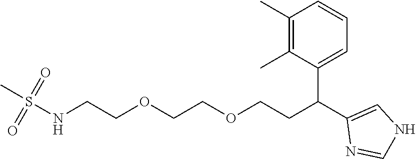 [SMILES: Cc1cccc(C(CCOCCOCCNS(C)(=O)=O)c2c[nH]cn2)c1C] |
| 94 | 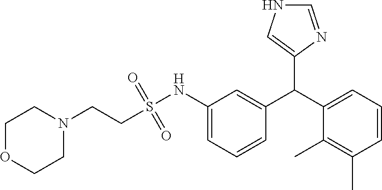 [SMILES: Cc1cccc(C(c2cccc(NS(=O)(=O)CCN3CCOCC3)c2)c2c[nH]cn2)c1C] |
| 95 | 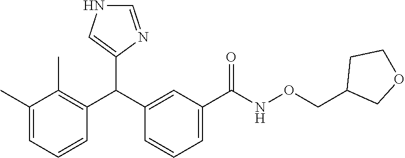 [SMILES: Cc1cccc(C(c2cccc(C(=O)NOCC3CCOC3)c2)c2c[nH]cn2)c1C] |
| 96 | 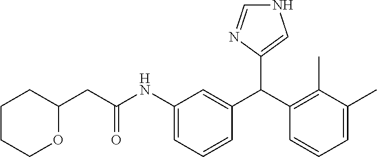 [SMILES: Cc1cccc(C(c2cccc(NC(=O)CC3CCCCO3)c2)c2c[nH]cn2)c1C] |
| 97 | 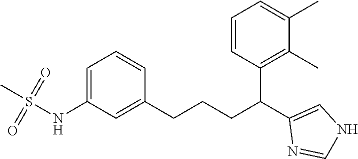 [SMILES: Cc1cccc(C(CCCc2cccc(NS(C)(=O)=O)c2)c2c[nH]cn2)c1C] |
| 98 | 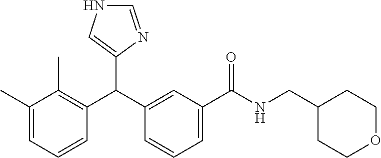 [SMILES: Cc1cccc(C(c2cccc(C(=O)NCC3CCOCC3)c2)c2c[nH]cn2)c1C] |
| 99 | 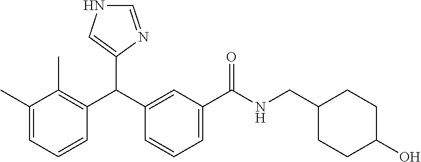 [SMILES: Cc1cccc(C(c2cccc(C(=O)NCC3CCC(O)CC3)c2)c2c[nH]cn2)c1C] |
| 100 | 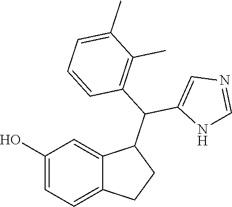 [SMILES: Cc1cccc(C(c2cnc[nH]2)C2CCc3ccc(O)cc32)c1C] |
| 101 | 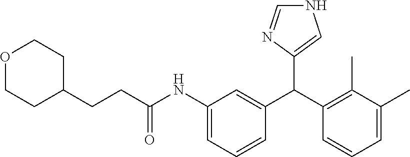 [SMILES: Cc1cccc(C(c2cccc(NC(=O)CCC3CCOCC3)c2)c2c[nH]cn2)c1C] |
| 102 | 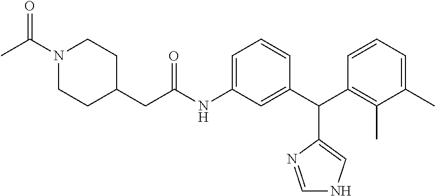 [SMILES: CC(=O)N1CCC(CC(=O)Nc2cccc(C(c3c[nH]cn3)c3cccc(C)c3C)c2)CC1] |
| 103 | 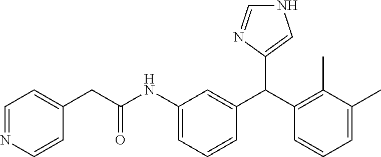 [SMILES: Cc1cccc(C(c2cccc(NC(=O)Cc3ccncc3)c2)c2c[nH]cn2)c1C] |
| 104 | 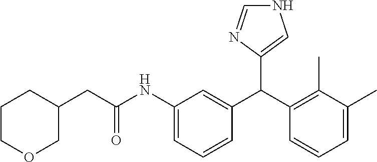 [SMILES: Cc1cccc(C(c2cccc(NC(=O)CC3CCCOC3)c2)c2c[nH]cn2)c1C] |
| 105 | 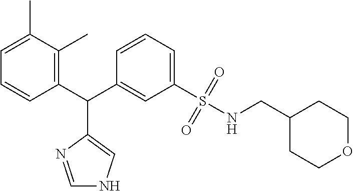 [SMILES: Cc1cccc(C(c2cccc(S(=O)(=O)NCC3CCOCC3)c2)c2c[nH]cn2)c1C] |
| 106 | 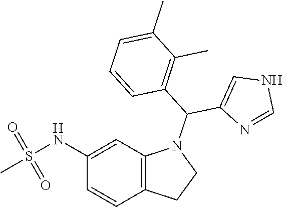 [SMILES: Cc1cccc(C(c2c[nH]cn2)N2CCc3ccc(NS(C)(=O)=O)cc32)c1C] |
| 107 | 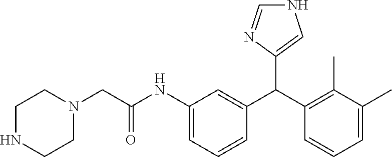 [SMILES: Cc1cccc(C(c2cccc(NC(=O)CN3CCNCC3)c2)c2c[nH]cn2)c1C] |
| 108 | 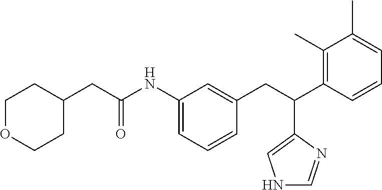 [SMILES: Cc1cccc(C(Cc2cccc(NC(=O)CC3CCOCC3)c2)c2c[nH]cn2)c1C] |
| 109 | 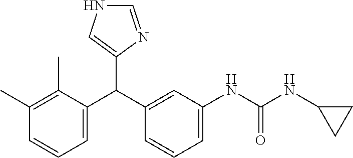 [SMILES: Cc1cccc(C(c2cccc(NC(=O)NC3CC3)c2)c2c[nH]cn2)c1C] |
| 110 | 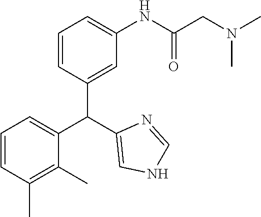 [SMILES: Cc1cccc(C(c2cccc(NC(=O)CN(C)C)c2)c2c[nH]cn2)c1C] |
| 111 | 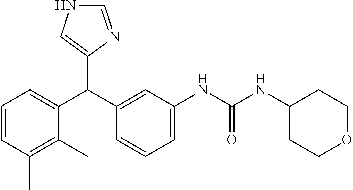 [SMILES: Cc1cccc(C(c2cccc(NC(=O)NC3CCOCC3)c2)c2c[nH]cn2)c1C] |
| 112 | 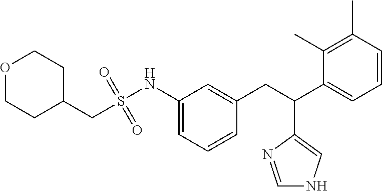 [SMILES: Cc1cccc(C(Cc2cccc(NS(=O)(=O)CC3CCOCC3)c2)c2c[nH]cn2)c1C] |
| 113 | 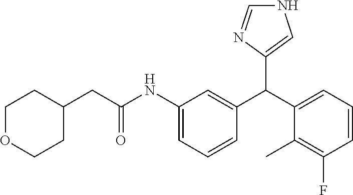 [SMILES: Cc1c(F)cccc1C(c1cccc(NC(=O)CC2CCOCC2)c1)c1c[nH]cn1] |
| 114 | 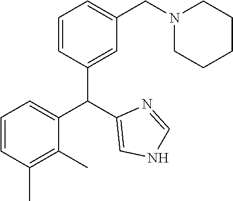 [SMILES: Cc1cccc(C(c2cccc(CN3CCCCC3)c2)c2c[nH]cn2)c1C] |
| 115 | 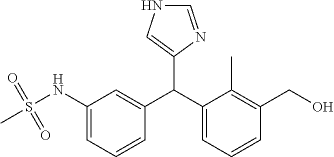 [SMILES: Cc1c(CO)cccc1C(c1cccc(NS(C)(=O)=O)c1)c1c[nH]cn1] |
| 116 | 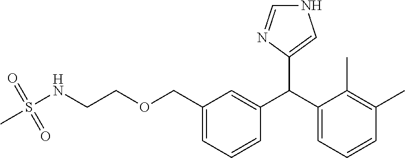 |
| 117 | 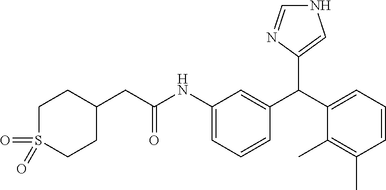 [SMILES: Cc1cccc(C(c2cccc(NC(=O)CC3CCS(=O)(=O)CC3)c2)c2c[nH]cn2)c1C] |
| 118 | 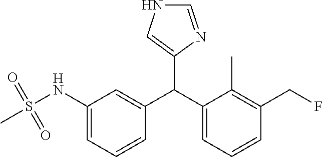 [SMILES: Cc1c(CF)cccc1C(c1cccc(NS(C)(=O)=O)c1)c1c[nH]cn1] |
| 119 | 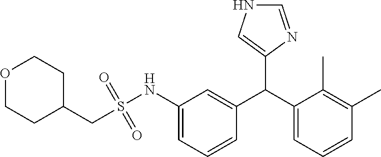 [SMILES: Cc1cccc(C(c2cccc(NS(=O)(=O)CC3CCOCC3)c2)c2c[nH]cn2)c1C] |
| 120 | 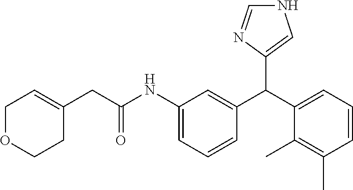 [SMILES: Cc1cccc(C(c2cccc(NC(=O)CC3=CCOCC3)c2)c2c[nH]cn2)c1C] |
| 121 | 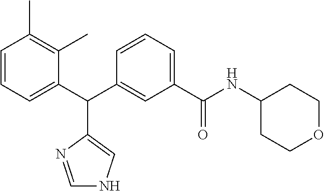 [SMILES: Cc1cccc(C(c2cccc(C(=O)NC3CCOCC3)c2)c2c[nH]cn2)c1C] |
| 122 | 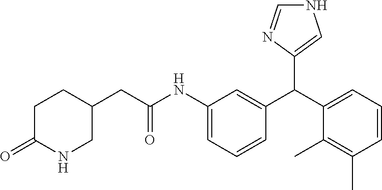 [SMILES: Cc1cccc(C(c2cccc(NC(=O)CC3CCC(=O)NC3)c2)c2c[nH]cn2)c1C] |
| 123 | 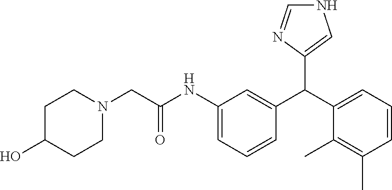 [SMILES: Cc1cccc(C(c2cccc(NC(=O)CN3CCC(O)CC3)c2)c2c[nH]cn2)c1C] |
| 124 | 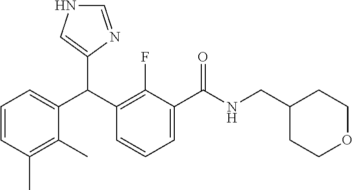 [SMILES: Cc1cccc(C(c2c[nH]cn2)c2cccc(C(=O)NCC3CCOCC3)c2F)c1C] |
| 125 | 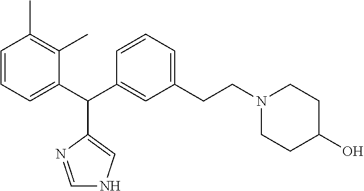 [SMILES: Cc1cccc(C(c2cccc(CCN3CCC(O)CC3)c2)c2c[nH]cn2)c1C] |
| 126 | 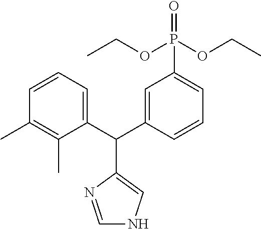 [SMILES: CCOP(=O)(OCC)c1cccc(C(c2c[nH]cn2)c2cccc(C)c2C)c1] |
| 127 | 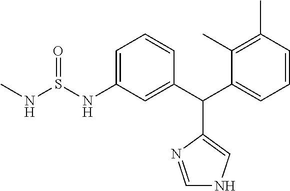 [SMILES: CNS(=O)Nc1cccc(C(c2c[nH]cn2)c2cccc(C)c2C)c1] |
| 128 | 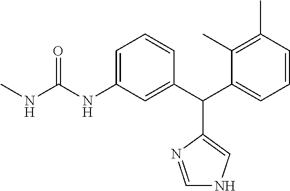 [SMILES: CNC(=O)Nc1cccc(C(c2c[nH]cn2)c2cccc(C)c2C)c1] |
| 129 | 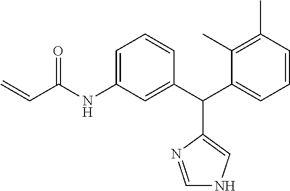 [SMILES: C=CC(=O)Nc1cccc(C(c2c[nH]cn2)c2cccc(C)c2C)c1] |
| 130 | 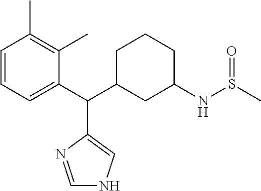 [SMILES: Cc1cccc(C(c2c[nH]cn2)C2CCCC(NS(C)=O)C2)c1C] |
| 131 | 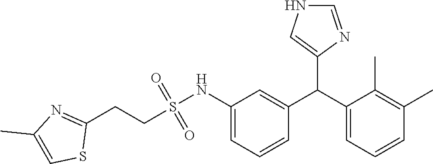 |
| 132 | 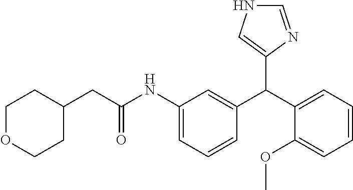 [SMILES: COc1ccccc1C(c1cccc(NC(=O)CC2CCOCC2)c1)c1c[nH]cn1] |
| 133 | 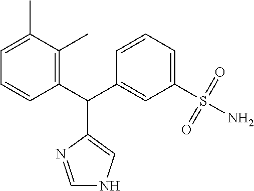 [SMILES: Cc1cccc(C(c2cccc(S(N)(=O)=O)c2)c2c[nH]cn2)c1C] |
| 134 | 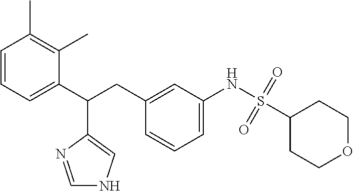 [SMILES: Cc1cccc(C(Cc2cccc(NS(=O)(=O)C3CCOCC3)c2)c2c[nH]cn2)c1C] |
| 135 | 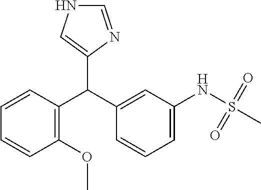 [SMILES: COc1ccccc1C(c1cccc(NS(C)(=O)=O)c1)c1c[nH]cn1] |
| 136 | 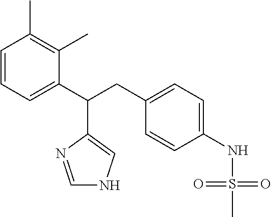 [SMILES: Cc1cccc(C(Cc2ccc(NS(C)(=O)=O)cc2)c2c[nH]cn2)c1C] |
| 137 | 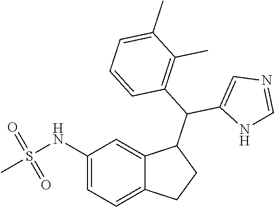 [SMILES: Cc1cccc(C(c2cnc[nH]2)C2CCc3ccc(NS(C)(=O)=O)cc32)c1C] |
| 138 | 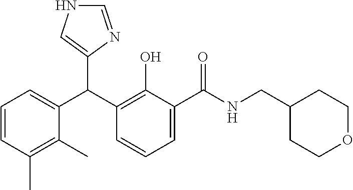 [SMILES: Cc1cccc(C(c2c[nH]cn2)c2cccc(C(=O)NCC3CCOCC3)c2O)c1C] |
| 139 | 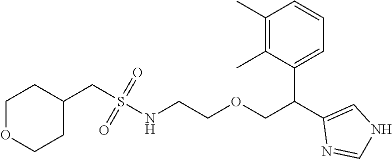 [SMILES: Cc1cccc(C(COCCNS(=O)(=O)CC2CCOCC2)c2c[nH]cn2)c1C] |
| 140 | 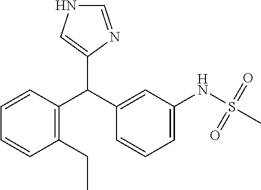 [SMILES: CCc1ccccc1C(c1cccc(NS(C)(=O)=O)c1)c1c[nH]cn1] |
| 141 | 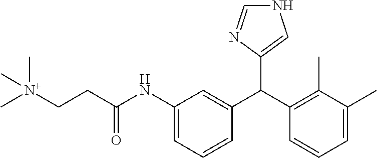 |
| 142 | 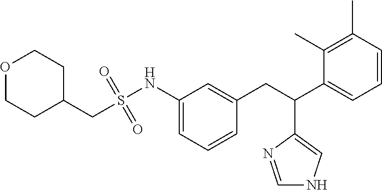 [SMILES: Cc1cccc(C(Cc2cccc(NS(=O)(=O)CC3CCOCC3)c2)c2c[nH]cn2)c1C] |
| 143 | 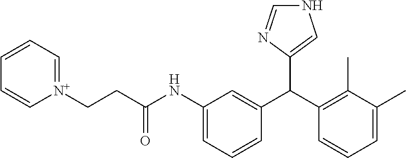 [SMILES: Cc1cccc(C(c2cccc(NC(=O)CC[n+]3ccccc3)c2)c2c[nH]cn2)c1C] |
| 144 | 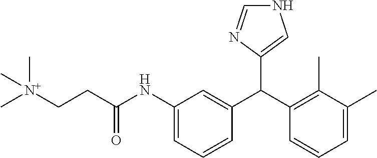 |
| 145 | 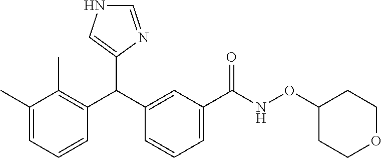 [SMILES: Cc1cccc(C(c2cccc(C(=O)NOC3CCOCC3)c2)c2c[nH]cn2)c1C] |
| 146 | 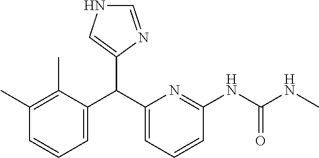 |
| 147 | 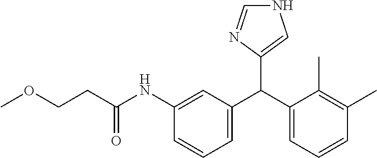 |
| 148 | 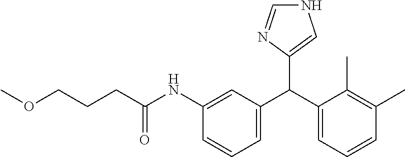 |
| 149 | 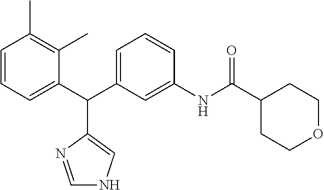 [SMILES: Cc1cccc(C(c2cccc(NC(=O)C3CCOCC3)c2)c2c[nH]cn2)c1C] |
| 150 | 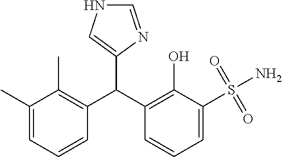 [SMILES: Cc1cccc(C(c2c[nH]cn2)c2cccc(S(N)(=O)=O)c2O)c1C] |
| 151 | 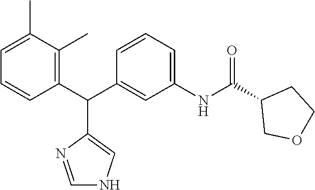 [SMILES: Cc1cccc(C(c2cccc(NC(=O)[C@@H]3CCOC3)c2)c2c[nH]cn2)c1C] |
| 152 | 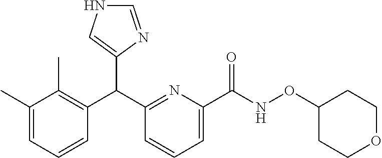 [SMILES: Cc1cccc(C(c2c[nH]cn2)c2cccc(C(=O)NOC3CCOCC3)n2)c1C] |
| 153 | 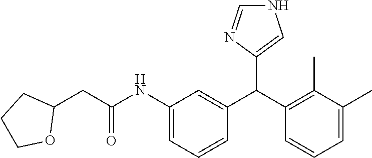 [SMILES: Cc1cccc(C(c2cccc(NC(=O)CC3CCCO3)c2)c2c[nH]cn2)c1C] |
| 154 | 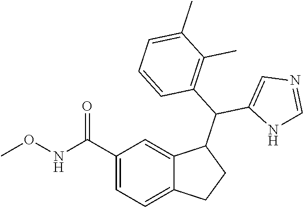 [SMILES: CONC(=O)c1ccc2c(c1)C(C(c1cnc[nH]1)c1cccc(C)c1C)CC2] |
| 155 | 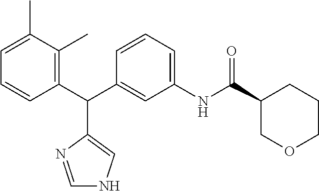 [SMILES: Cc1cccc(C(c2cccc(NC(=O)[C@H]3CCCOC3)c2)c2c[nH]cn2)c1C] |
| 156 | 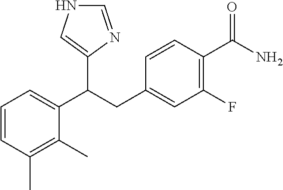 [SMILES: Cc1cccc(C(Cc2ccc(C(N)=O)c(F)c2)c2c[nH]cn2)c1C] |
| 157 | 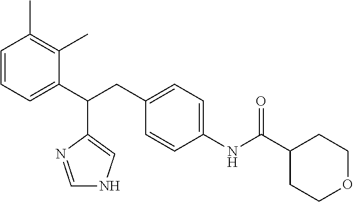 [SMILES: Cc1cccc(C(Cc2ccc(NC(=O)C3CCOCC3)cc2)c2c[nH]cn2)c1C] |
| 158 | 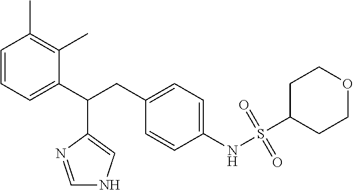 [SMILES: Cc1cccc(C(Cc2ccc(NS(=O)(=O)C3CCOCC3)cc2)c2c[nH]cn2)c1C] |
| 159 | 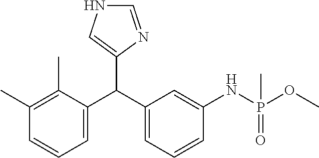 |
| 160 | 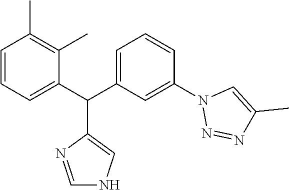 [SMILES: Cc1cn(-c2cccc(C(c3c[nH]cn3)c3cccc(C)c3C)c2)nn1] |
| 161 | 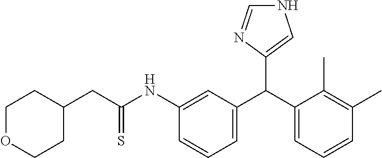 [SMILES: Cc1cccc(C(c2cccc(NC(=S)CC3CCOCC3)c2)c2c[nH]cn2)c1C] |
| 162 | 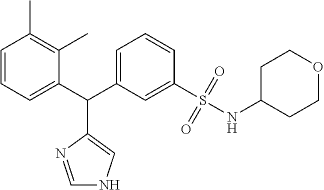 [SMILES: Cc1cccc(C(c2cccc(S(=O)(=O)NC3CCOCC3)c2)c2c[nH]cn2)c1C] |
| 163 | 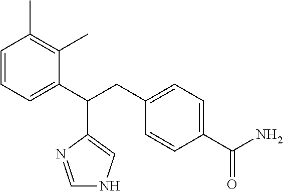 [SMILES: Cc1cccc(C(Cc2ccc(C(N)=O)cc2)c2c[nH]cn2)c1C] |
| 164 | 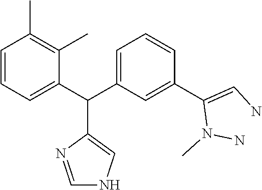 [SMILES: Cc1cccc(C(c2cccc(/C(=C/N)N(C)N)c2)c2c[nH]cn2)c1C] |
| 165 | 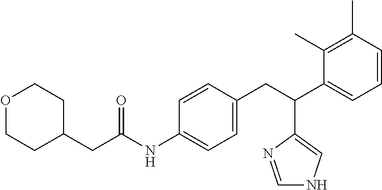 [SMILES: Cc1cccc(C(Cc2ccc(NC(=O)CC3CCOCC3)cc2)c2c[nH]cn2)c1C] |
| 166 | 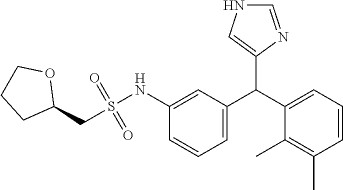 [SMILES: Cc1cccc(C(c2cccc(NS(=O)(=O)C[C@H]3CCCO3)c2)c2c[nH]cn2)c1C] |
| 167 | 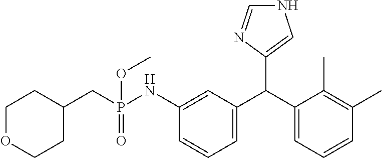 [SMILES: COP(=O)(CC1CCOCC1)Nc1cccc(C(c2c[nH]cn2)c2cccc(C)c2C)c1] |
| 168 | 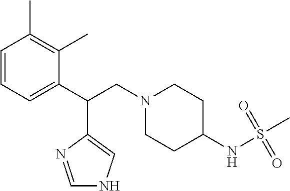 [SMILES: Cc1cccc(C(CN2CCC(NS(C)(=O)=O)CC2)c2c[nH]cn2)c1C] |
| 169 | 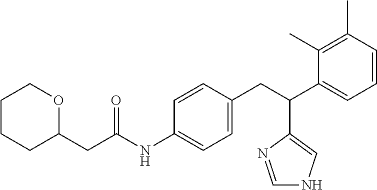 [SMILES: Cc1cccc(C(Cc2ccc(NC(=O)CC3CCCCO3)cc2)c2c[nH]cn2)c1C] |
| 170 | 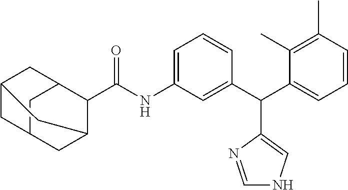 [SMILES: Cc1cccc(C(c2cccc(NC(=O)C3C4CC5CC(C4)CC3C5)c2)c2c[nH]cn2)c1C] |
| 171 | 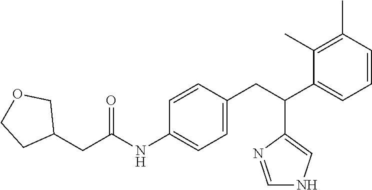 [SMILES: Cc1cccc(C(Cc2ccc(NC(=O)CC3CCOC3)cc2)c2c[nH]cn2)c1C] |
| 172 | 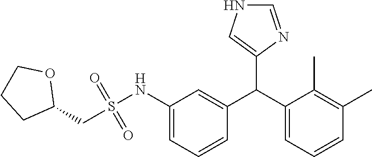 [SMILES: Cc1cccc(C(c2cccc(NS(=O)(=O)C[C@@H]3CCCO3)c2)c2c[nH]cn2)c1C] |
| 173 | 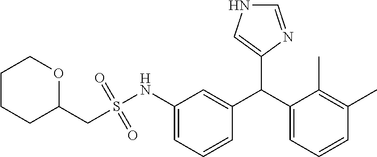 [SMILES: Cc1cccc(C(c2cccc(NS(=O)(=O)CC3CCCCO3)c2)c2c[nH]cn2)c1C] |
| 174 | 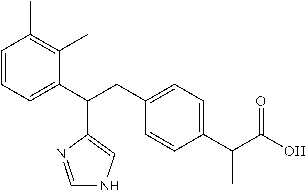 [SMILES: Cc1cccc(C(Cc2ccc(C(C)C(=O)O)cc2)c2c[nH]cn2)c1C] |
| 175 | 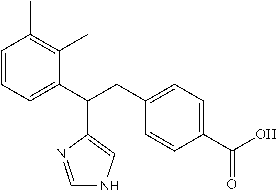 [SMILES: Cc1cccc(C(Cc2ccc(C(=O)O)cc2)c2c[nH]cn2)c1C] |
| 176 | 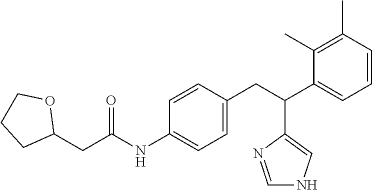 [SMILES: Cc1cccc(C(Cc2ccc(NC(=O)CC3CCCO3)cc2)c2c[nH]cn2)c1C] |
| 177 | 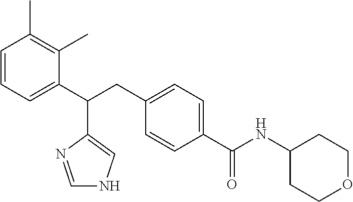 [SMILES: Cc1cccc(C(Cc2ccc(C(=O)NC3CCOCC3)cc2)c2c[nH]cn2)c1C] |
| 178 | 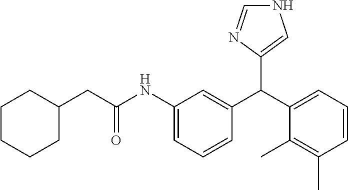 [SMILES: Cc1cccc(C(c2cccc(NC(=O)CC3CCCCC3)c2)c2c[nH]cn2)c1C] |
| 179 | 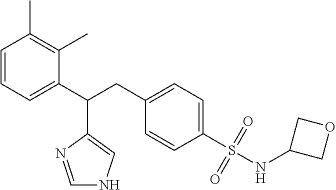 [SMILES: Cc1cccc(C(Cc2ccc(S(=O)(=O)NC3COC3)cc2)c2c[nH]cn2)c1C] |
| 180 | 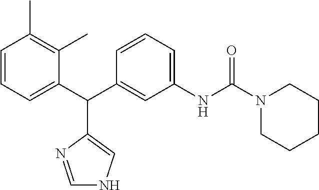 [SMILES: Cc1cccc(C(c2cccc(NC(=O)N3CCCCC3)c2)c2c[nH]cn2)c1C] |
| 181 | 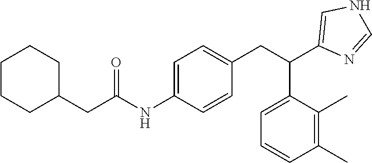 [SMILES: Cc1cccc(C(Cc2ccc(NC(=O)CC3CCCCC3)cc2)c2c[nH]cn2)c1C] |
| 182 | 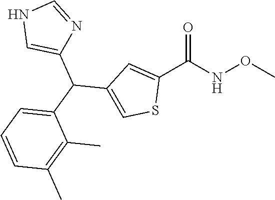 [SMILES: CONC(=O)c1cc(C(c2c[nH]cn2)c2cccc(C)c2C)cs1] |
| 183 | 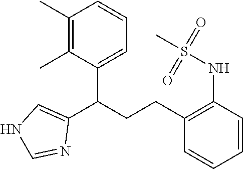 [SMILES: Cc1cccc(C(CCc2ccccc2NS(C)(=O)=O)c2c[nH]cn2)c1C] |
| 184 | 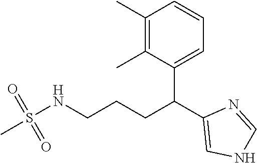 [SMILES: Cc1cccc(C(CCCNS(C)(=O)=O)c2c[nH]cn2)c1C] |
| 185 | 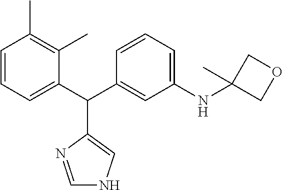 [SMILES: Cc1cccc(C(c2cccc(NC3(C)COC3)c2)c2c[nH]cn2)c1C] |
| 186 | 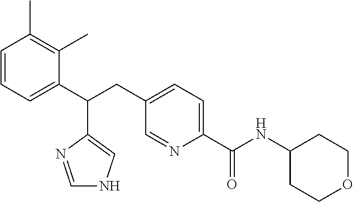 [SMILES: Cc1cccc(C(Cc2ccc(C(=O)NC3CCOCC3)nc2)c2c[nH]cn2)c1C] |
| 187 | 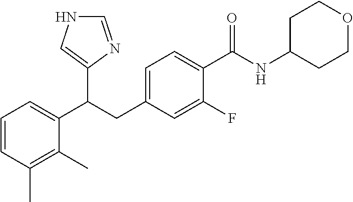 [SMILES: Cc1cccc(C(Cc2ccc(C(=O)NC3CCOCC3)c(F)c2)c2c[nH]cn2)c1C] |
| 188 | 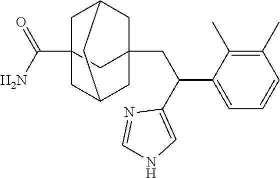 [SMILES: Cc1cccc(C(CC23CC4CC(C2)CC(C(N)=O)(C4)C3)c2c[nH]cn2)c1C] |
| 189 | 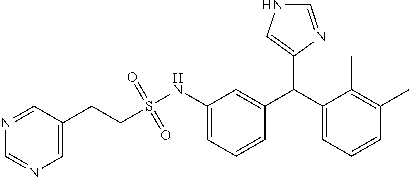 [SMILES: Cc1cccc(C(c2cccc(NS(=O)(=O)CCc3cncnc3)c2)c2c[nH]cn2)c1C] |
| 190 | 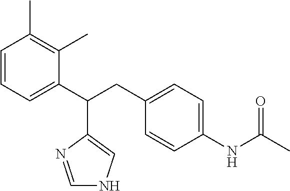 [SMILES: CC(=O)Nc1ccc(CC(c2c[nH]cn2)c2cccc(C)c2C)cc1] |
| 191 | 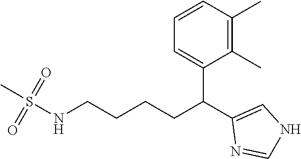 [SMILES: Cc1cccc(C(CCCCNS(C)(=O)=O)c2c[nH]cn2)c1C] |
| 192 | 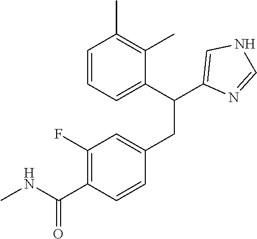 [SMILES: CNC(=O)c1ccc(CC(c2c[nH]cn2)c2cccc(C)c2C)cc1F] |
| 193 | 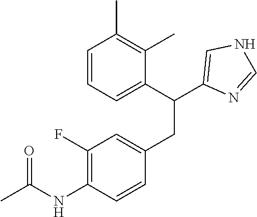 [SMILES: CC(=O)Nc1ccc(CC(c2c[nH]cn2)c2cccc(C)c2C)cc1F] |
| 194 | 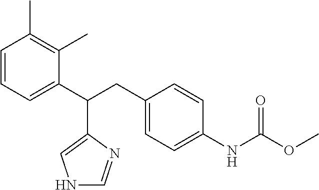 [SMILES: COC(=O)Nc1ccc(CC(c2c[nH]cn2)c2cccc(C)c2C)cc1] |
| 195 | 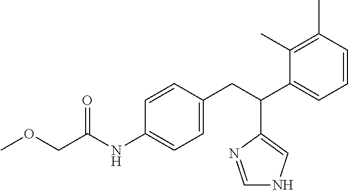 [SMILES: COCC(=O)Nc1ccc(CC(c2c[nH]cn2)c2cccc(C)c2C)cc1] |
| 196 | 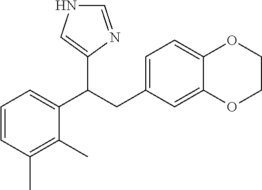 [SMILES: Cc1cccc(C(Cc2ccc3c(c2)OCCO3)c2c[nH]cn2)c1C] |
| 197 | 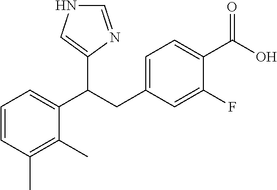 [SMILES: Cc1cccc(C(Cc2ccc(C(=O)O)c(F)c2)c2c[nH]cn2)c1C] |
| 198 | 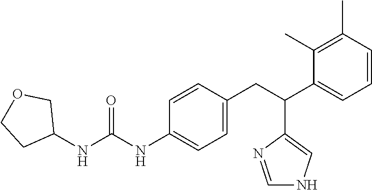 [SMILES: Cc1cccc(C(Cc2ccc(NC(=O)NC3CCOC3)cc2)c2c[nH]cn2)c1C] |
| 199 | 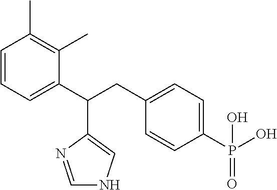 [SMILES: Cc1cccc(C(Cc2ccc(P(=O)(O)O)cc2)c2c[nH]cn2)c1C] |
| 200 | 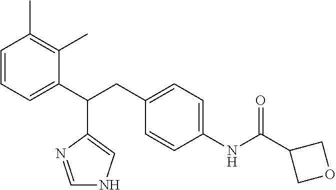 [SMILES: Cc1cccc(C(Cc2ccc(NC(=O)C3COC3)cc2)c2c[nH]cn2)c1C] |
| 201 | 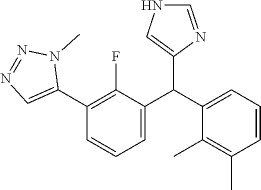 [SMILES: Cc1cccc(C(c2c[nH]cn2)c2cccc(-c3cnnn3C)c2F)c1C] |
| 202 | 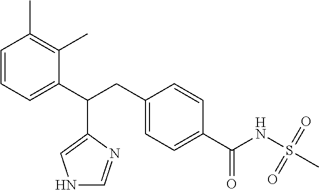 [SMILES: Cc1cccc(C(Cc2ccc(C(=O)NS(C)(=O)=O)cc2)c2c[nH]cn2)c1C] |
| 203 | 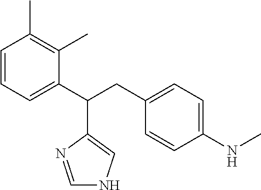 [SMILES: CNc1ccc(CC(c2c[nH]cn2)c2cccc(C)c2C)cc1] |
| 204 | 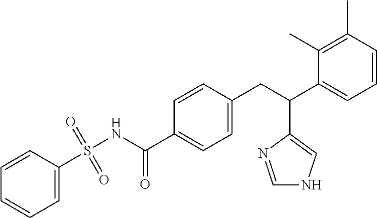 [SMILES: Cc1cccc(C(Cc2ccc(C(=O)NS(=O)(=O)c3ccccc3)cc2)c2c[nH]cn2)c1C] |
| 205 | 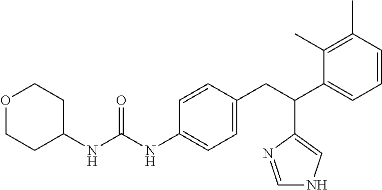 [SMILES: Cc1cccc(C(Cc2ccc(NC(=O)NC3CCOCC3)cc2)c2c[nH]cn2)c1C] |
| 206 | 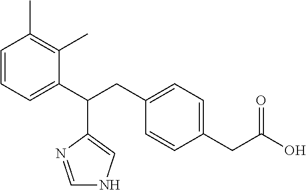 [SMILES: Cc1cccc(C(Cc2ccc(CC(=O)O)cc2)c2c[nH]cn2)c1C] |
| 207 | 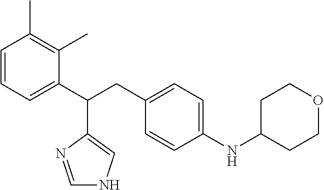 [SMILES: Cc1cccc(C(Cc2ccc(NC3CCOCC3)cc2)c2c[nH]cn2)c1C] |
| 208 | 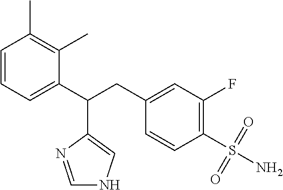 [SMILES: Cc1cccc(C(Cc2ccc(S(N)(=O)=O)c(F)c2)c2c[nH]cn2)c1C] |
| 209 | 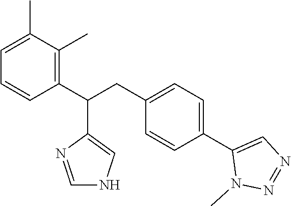 [SMILES: Cc1cccc(C(Cc2ccc(-c3cnnn3C)cc2)c2c[nH]cn2)c1C] |
| 210 | 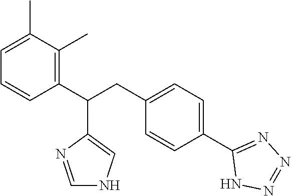 [SMILES: Cc1cccc(C(Cc2ccc(-c3nnn[nH]3)cc2)c2c[nH]cn2)c1C] |
| 211 | 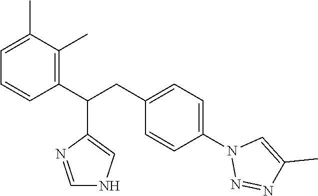 [SMILES: Cc1cn(-c2ccc(CC(c3c[nH]cn3)c3cccc(C)c3C)cc2)nn1] |
| 212 | 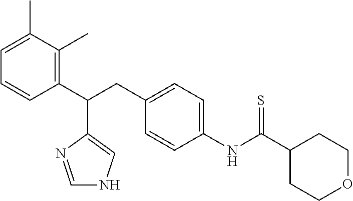 [SMILES: Cc1cccc(C(Cc2ccc(NC(=S)C3CCOCC3)cc2)c2c[nH]cn2)c1C] |
| 213 | 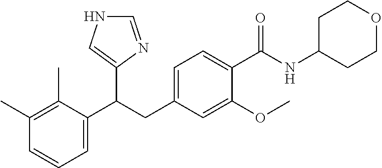 [SMILES: COc1cc(CC(c2c[nH]cn2)c2cccc(C)c2C)ccc1C(=O)NC1CCOCC1] |
| 214 | 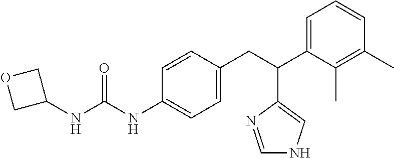 [SMILES: Cc1cccc(C(Cc2ccc(NC(=O)NC3COC3)cc2)c2c[nH]cn2)c1C] |
| 215 | 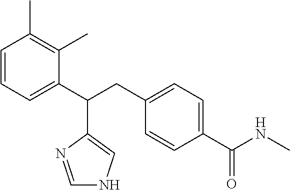 [SMILES: CNC(=O)c1ccc(CC(c2c[nH]cn2)c2cccc(C)c2C)cc1] |
| 216 | 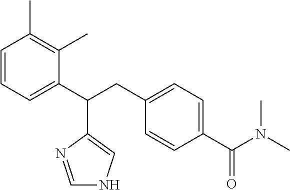 [SMILES: Cc1cccc(C(Cc2ccc(C(=O)N(C)C)cc2)c2c[nH]cn2)c1C] |
| 217 | 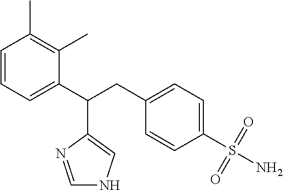 [SMILES: Cc1cccc(C(Cc2ccc(S(N)(=O)=O)cc2)c2c[nH]cn2)c1C] |
| 218 | 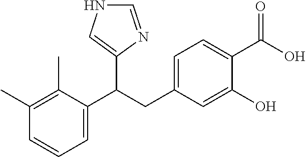 [SMILES: Cc1cccc(C(Cc2ccc(C(=O)O)c(O)c2)c2c[nH]cn2)c1C] |
| 219 | 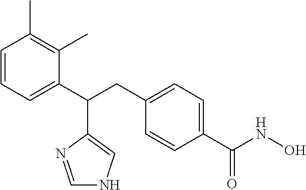 [SMILES: Cc1cccc(C(Cc2ccc(C(=O)NO)cc2)c2c[nH]cn2)c1C] |
| 220 | 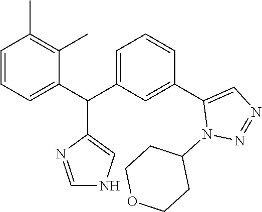 [SMILES: Cc1cccc(C(c2cccc(-c3cnnn3C3CCOCC3)c2)c2c[nH]cn2)c1C] |
| 221 | 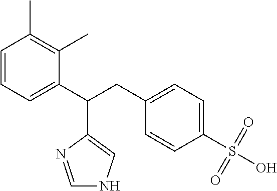 [SMILES: Cc1cccc(C(Cc2ccc(S(=O)(=O)O)cc2)c2c[nH]cn2)c1C] |
| 222 | 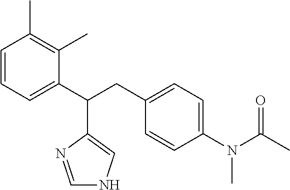 [SMILES: CC(=O)N(C)c1ccc(CC(c2c[nH]cn2)c2cccc(C)c2C)cc1] |
| 223 | 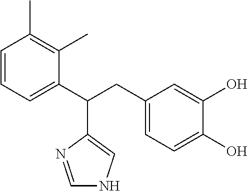 [SMILES: Cc1cccc(C(Cc2ccc(O)c(O)c2)c2c[nH]cn2)c1C] |
| 224 | 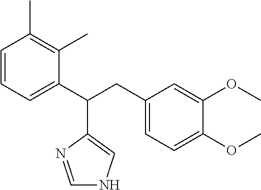 [SMILES: COc1ccc(CC(c2c[nH]cn2)c2cccc(C)c2C)cc1OC] |
| 225 | 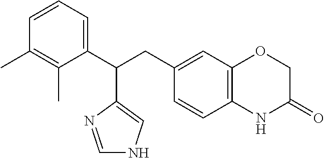 [SMILES: Cc1cccc(C(Cc2ccc3c(c2)OCC(=O)N3)c2c[nH]cn2)c1C] |
| 226 | 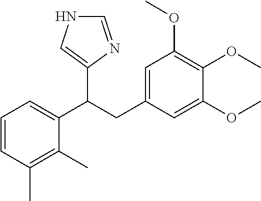 [SMILES: COc1cc(CC(c2c[nH]cn2)c2cccc(C)c2C)cc(OC)c1OC] |
| 227 | 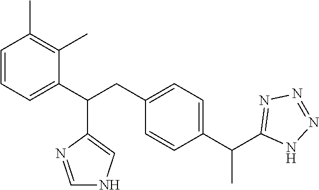 [SMILES: Cc1cccc(C(Cc2ccc(C(C)c3nnn[nH]3)cc2)c2c[nH]cn2)c1C] |
| 228 | 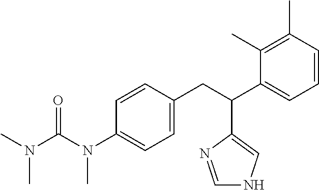 [SMILES: Cc1cccc(C(Cc2ccc(N(C)C(=O)N(C)C)cc2)c2c[nH]cn2)c1C] |
| 229 | 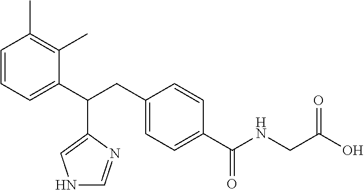 [SMILES: Cc1cccc(C(Cc2ccc(C(=O)NCC(=O)O)cc2)c2c[nH]cn2)c1C] |
| 230 | 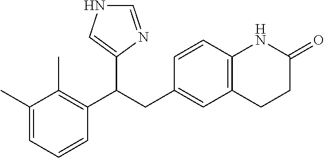 [SMILES: Cc1cccc(C(Cc2ccc3c(c2)CCC(=O)N3)c2c[nH]cn2)c1C] |
| 231 | 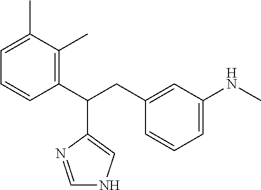 [SMILES: CNc1cccc(CC(c2c[nH]cn2)c2cccc(C)c2C)c1] |
| 232 | 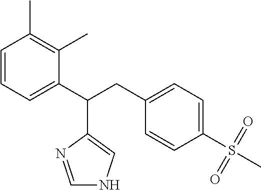 [SMILES: Cc1cccc(C(Cc2ccc(S(C)(=O)=O)cc2)c2c[nH]cn2)c1C] |
| 233 | 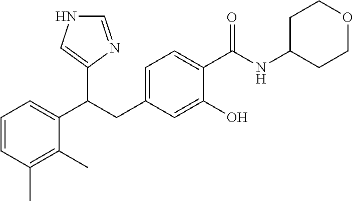 [SMILES: Cc1cccc(C(Cc2ccc(C(=O)NC3CCOCC3)c(O)c2)c2c[nH]cn2)c1C] |
| 234 | 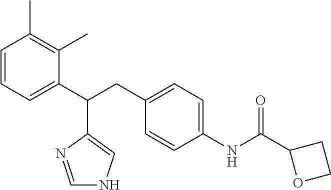 [SMILES: Cc1cccc(C(Cc2ccc(NC(=O)C3CCO3)cc2)c2c[nH]cn2)c1C] |
| 235 | 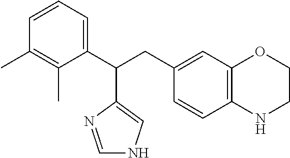 [SMILES: Cc1cccc(C(Cc2ccc3c(c2)OCCN3)c2c[nH]cn2)c1C] |
| 236 | 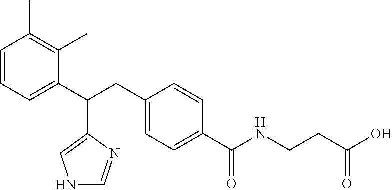 [SMILES: Cc1cccc(C(Cc2ccc(C(=O)NCCC(=O)O)cc2)c2c[nH]cn2)c1C] |
| 237 | 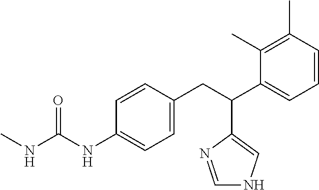 [SMILES: CNC(=O)Nc1ccc(CC(c2c[nH]cn2)c2cccc(C)c2C)cc1] |
| 238 | 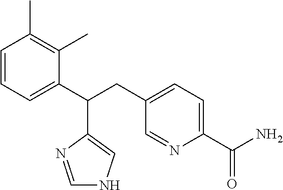 [SMILES: Cc1cccc(C(Cc2ccc(C(N)=O)nc2)c2c[nH]cn2)c1C] |
| 239 | 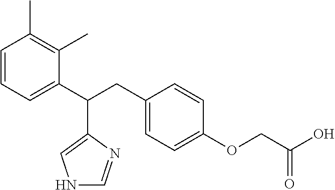 [SMILES: Cc1cccc(C(Cc2ccc(OCC(=O)O)cc2)c2c[nH]cn2)c1C] |
| 240 | 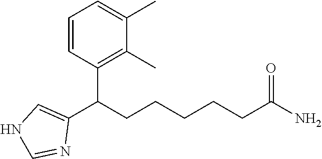 [SMILES: Cc1cccc(C(CCCCCC(N)=O)c2c[nH]cn2)c1C] |
| 241 | 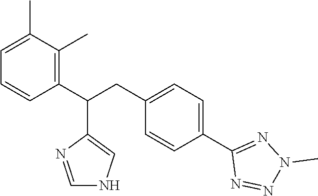 |
| 242 | 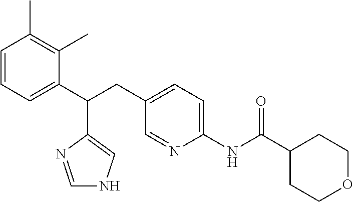 [SMILES: Cc1cccc(C(Cc2ccc(NC(=O)C3CCOCC3)nc2)c2c[nH]cn2)c1C] |
| 243 | 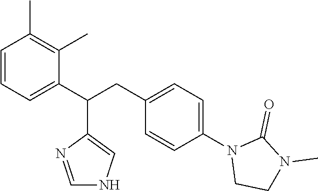 [SMILES: Cc1cccc(C(Cc2ccc(N3CCN(C)C3=O)cc2)c2c[nH]cn2)c1C] |
| 244 | 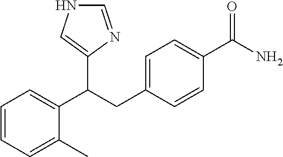 [SMILES: Cc1ccccc1C(Cc1ccc(C(N)=O)cc1)c1c[nH]cn1] |
| 245 | 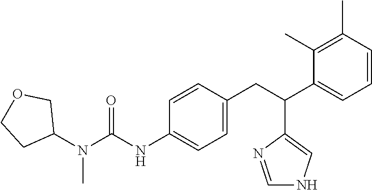 [SMILES: Cc1cccc(C(Cc2ccc(NC(=O)N(C)C3CCOC3)cc2)c2c[nH]cn2)c1C] |
| 246 | 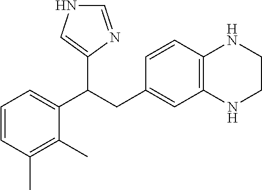 [SMILES: Cc1cccc(C(Cc2ccc3c(c2)NCCN3)c2c[nH]cn2)c1C] |
| 247 | 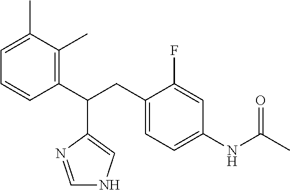 [SMILES: CC(=O)Nc1ccc(CC(c2c[nH]cn2)c2cccc(C)c2C)c(F)c1] |
| 248 | 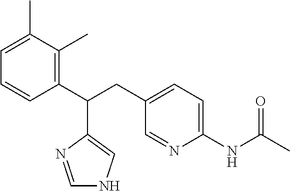 [SMILES: CC(=O)Nc1ccc(CC(c2c[nH]cn2)c2cccc(C)c2C)cn1] |
| 249 | 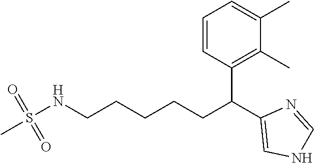 [SMILES: Cc1cccc(C(CCCCCNS(C)(=O)=O)c2c[nH]cn2)c1C] |
| 250 | 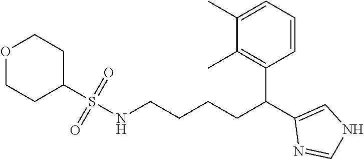 [SMILES: Cc1cccc(C(CCCCNS(=O)(=O)C2CCOCC2)c2c[nH]cn2)c1C] |
| 251 | 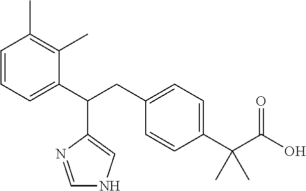 [SMILES: Cc1cccc(C(Cc2ccc(C(C)(C)C(=O)O)cc2)c2c[nH]cn2)c1C] |
| 401 | 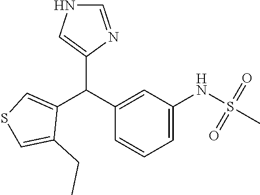 [SMILES: CCc1cscc1C(c1cccc(NS(C)(=O)=O)c1)c1c[nH]cn1] |
| 402 | 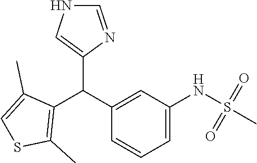 [SMILES: Cc1csc(C)c1C(c1cccc(NS(C)(=O)=O)c1)c1c[nH]cn1] |
| 403 | 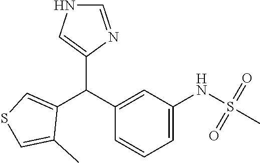 [SMILES: Cc1cscc1C(c1cccc(NS(C)(=O)=O)c1)c1c[nH]cn1] |
| 501 | 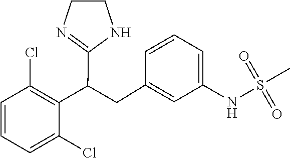 [SMILES: CS(=O)(=O)Nc1cccc(CC(C2=NCCN2)c2c(Cl)cccc2Cl)c1] |
| 502 | 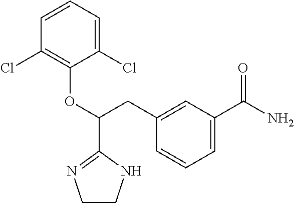 [SMILES: NC(=O)c1cccc(CC(Oc2c(Cl)cccc2Cl)C2=NCCN2)c1] |
| 503 | 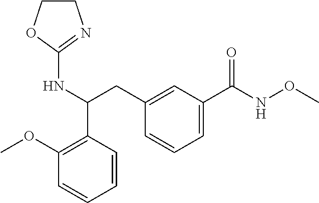 |
| 504 | 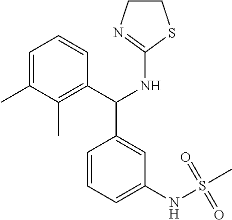 |
| 505 | 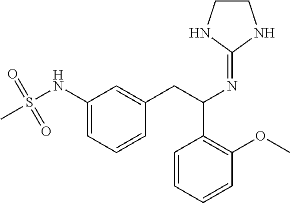 |
| 506 | 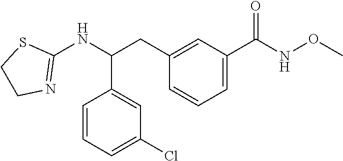 [SMILES: CONC(=O)c1cccc(CC(NC2=NCCS2)c2cccc(Cl)c2)c1] |
| 507 | 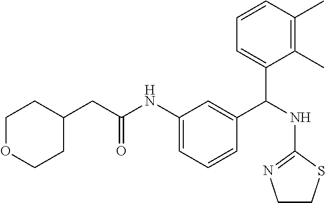 [SMILES: Cc1cccc(C(NC2=NCCS2)c2cccc(NC(=O)CC3CCOCC3)c2)c1C] |
| 508 | 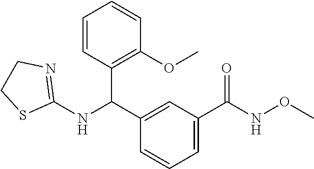 [SMILES: CONC(=O)c1cccc(C(NC2=NCCS2)c2ccccc2OC)c1] |
| 509 | 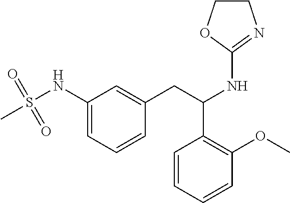 |
| 510 | 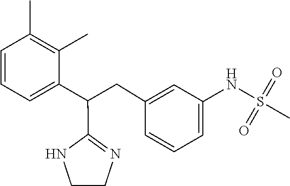 [SMILES: Cc1cccc(C(Cc2cccc(NS(C)(=O)=O)c2)C2=NCCN2)c1C] |
| 511 | 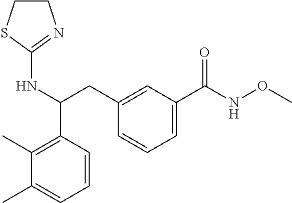 |
| 512 | 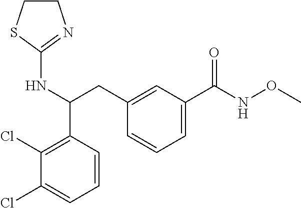 [SMILES: CONC(=O)c1cccc(CC(NC2=NCCS2)c2cccc(Cl)c2Cl)c1] |
| 513 | 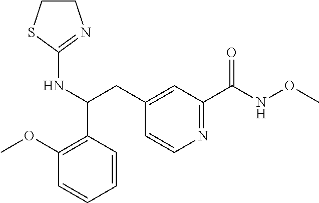 |
| 514 | 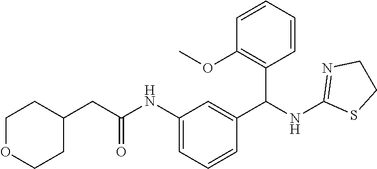 [SMILES: COc1ccccc1C(NC1=NCCS1)c1cccc(NC(=O)CC2CCOCC2)c1] |
| 515 | 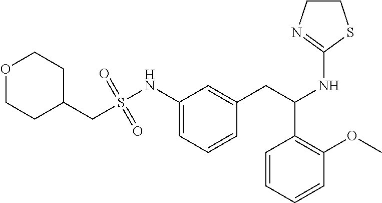 [SMILES: COc1ccccc1C(Cc1cccc(NS(=O)(=O)CC2CCOCC2)c1)NC1=NCCS1] |
| 516 | 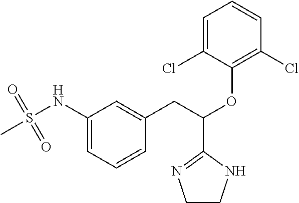 [SMILES: CS(=O)(=O)Nc1cccc(CC(Oc2c(Cl)cccc2Cl)C2=NCCN2)c1] |
| 517 | 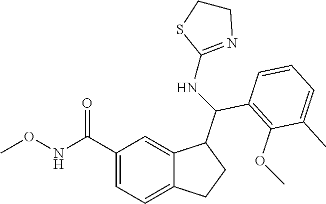 |
| 518 | 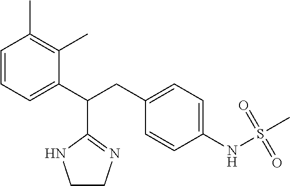 [SMILES: Cc1cccc(C(Cc2ccc(NS(C)(=O)=O)cc2)C2=NCCN2)c1C] |
| 519 | 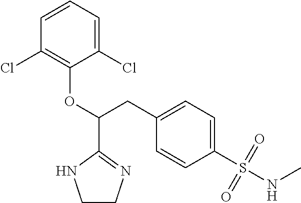 [SMILES: CNS(=O)(=O)c1ccc(CC(Oc2c(Cl)cccc2Cl)C2=NCCN2)cc1] |
| 520 | 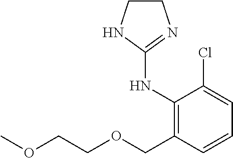 [SMILES: COCCOCc1cccc(Cl)c1NC1=NCCN1] |
| 601 | 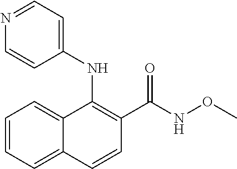 [SMILES: CONC(=O)c1ccc2ccccc2c1Nc1ccncc1] |
| 602 | 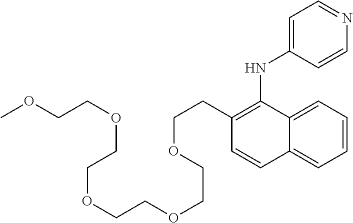 [SMILES: COCCOCCOCCOCCOCCc1ccc2ccccc2c1Nc1ccncc1] |

Exemplary R_{T} in formula (I-A). (I-B). (I-C). (I-D), or (II) include, but are not limited to, the following:

TABLE 1a
Exemplary R_{T}

| No. | structure |
|---|---|
| 1 | 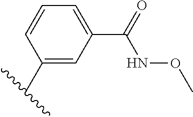 [SMILES: *c1cccc(C(=O)NOC)c1] |
| 2 | 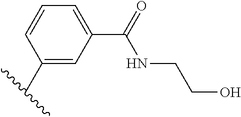 [SMILES: *c1cccc(C(=O)NCCO)c1] |
| 3 | 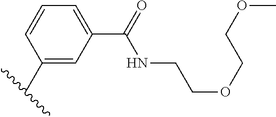 [SMILES: *c1cccc(C(=O)NCCOCCOC)c1] |
| 4 | 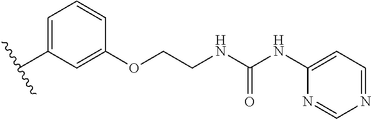 [SMILES: *c1cccc(OCCNC(=O)Nc2ccncn2)c1] |
| 5 | 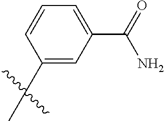 [SMILES: *c1cccc(C(N)=O)c1] |
| 6 | 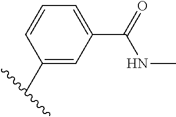 [SMILES: *c1cccc(C(=O)NC)c1] |
| 8 | 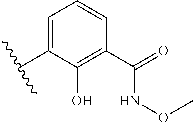 [SMILES: *c1cccc(C(=O)NOC)c1O] |
| 9 | 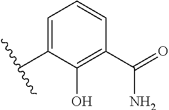 [SMILES: *c1cccc(C(N)=O)c1O] |
| 10 | 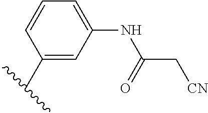 [SMILES: *c1cccc(NC(=O)CC#N)c1] |
| 11 | 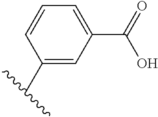 [SMILES: *c1cccc(C(=O)O)c1] |
| 14 | 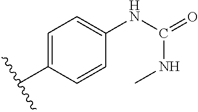 [SMILES: *c1ccc(NC(=O)NC)cc1] |
| 15 | 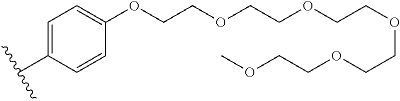 [SMILES: *c1ccc(OCCOCCOCCOCCOCCOC)cc1] |
| 16 | 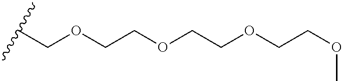 [SMILES: *COCCOCCOCCOC] |
| 17 | 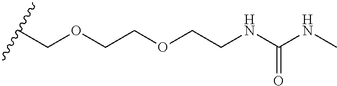 [SMILES: *COCCOCCNC(=O)NC] |
| 18 | 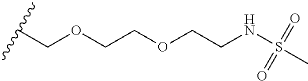 [SMILES: *COCCOCCNS(C)(=O)=O] |
| 19 | 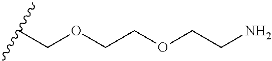 [SMILES: *COCCOCCN] |
| 20 | 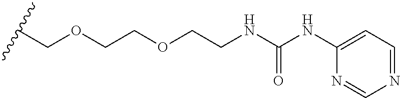 [SMILES: *COCCOCCNC(=O)Nc1ccncn1] |
| 21 | 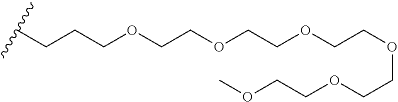 [SMILES: *CCCOCCOCCOCCOCCOCCOC] |
| 22 | 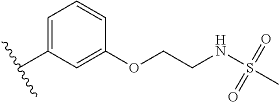 [SMILES: *c1cccc(OCCNS(C)(=O)=O)c1] |
| 23 | 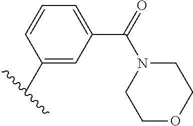 [SMILES: *c1cccc(C(=O)N2CCOCC2)c1] |
| 24 | 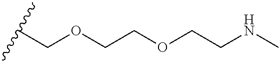 [SMILES: *COCCOCCNC] |
| 25 | 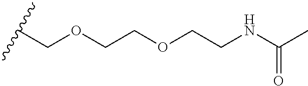 [SMILES: *COCCOCCNC(C)=O] |
| 26 | 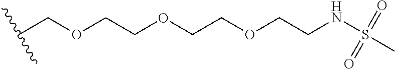 [SMILES: *COCCOCCOCCNS(C)(=O)=O] |
| 27 | 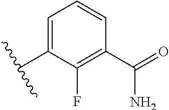 [SMILES: *c1cccc(C(N)=O)c1F] |
| 28 | 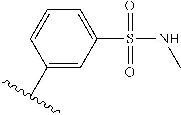 [SMILES: *c1cccc(S(=O)(=O)NC)c1] |
| 29 | 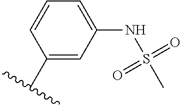 [SMILES: *c1cccc(NS(C)(=O)=O)c1] |
| 30 | 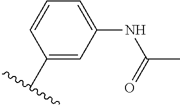 [SMILES: *c1cccc(NC(C)=O)c1] |
| 31 | 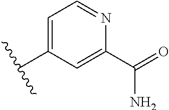 [SMILES: *c1ccnc(C(N)=O)c1] |
| 32 | 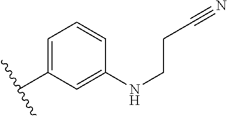 [SMILES: *c1cccc(NCCC#N)c1] |
| 33 | 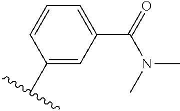 [SMILES: *c1cccc(C(=O)N(C)C)c1] |
| 37 | 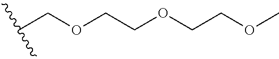 [SMILES: *COCCOCCOC] |
| 38 | 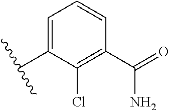 [SMILES: *c1cccc(C(N)=O)c1Cl] |
| 39 | 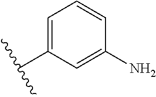 [SMILES: *c1cccc(N)c1] |
| 40 | 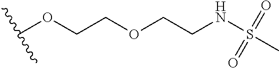 [SMILES: *OCCOCCNS(C)(=O)=O] |
| 41 | 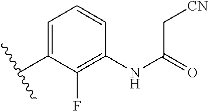 [SMILES: *c1cccc(NC(=O)CC#N)c1F] |
| 42 | 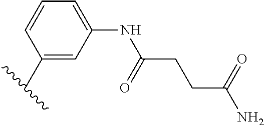 [SMILES: *c1cccc(NC(=O)CCC(N)=O)c1] |
| 43 | 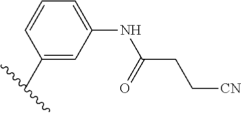 [SMILES: *c1cccc(NC(=O)CCC#N)c1] |
| 48 | 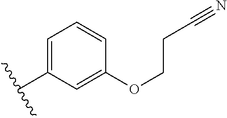 [SMILES: *c1cccc(OCCC#N)c1] |
| 50 | 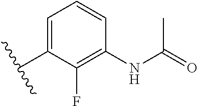 [SMILES: *c1cccc(NC(C)=O)c1F] |
| 51 | 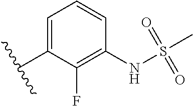 [SMILES: *c1cccc(NS(C)(=O)=O)c1F] |
| 53 | 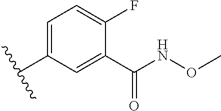 [SMILES: *c1ccc(F)c(C(=O)NOC)c1] |
| 54 | 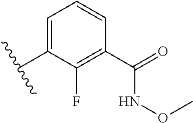 [SMILES: *c1cccc(C(=O)NOC)c1F] |
| 55 | 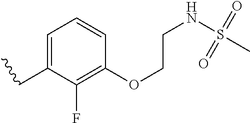 |
| 56 | 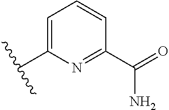 [SMILES: *c1cccc(C(N)=O)n1] |
| 57 | 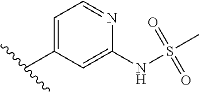 [SMILES: *c1ccnc(NS(C)(=O)=O)c1] |
| 58 | 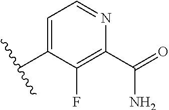 [SMILES: *c1ccnc(C(N)=O)c1F] |
| 59 | 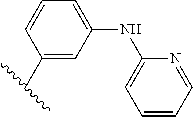 [SMILES: *c1cccc(Nc2ccccn2)c1] |
| 60 | 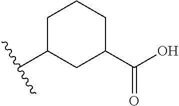 [SMILES: *C1CCCC(C(=O)O)C1] |
| 61 | 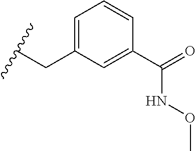 [SMILES: *Cc1cccc(C(=O)NOC)c1] |
| 62 | 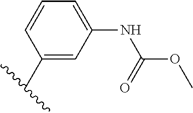 [SMILES: *c1cccc(NC(=O)OC)c1] |
| 63 | 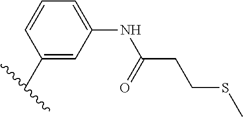 [SMILES: *c1cccc(NC(=O)CCSC)c1] |
| 64 | 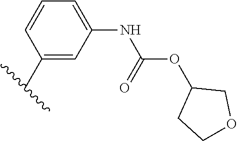 [SMILES: *c1cccc(NC(=O)OC2CCOC2)c1] |
| 65 | 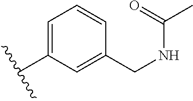 [SMILES: *c1cccc(CNC(C)=O)c1] |
| 66 | 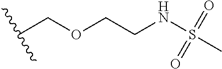 [SMILES: *COCCNS(C)(=O)=O] |
| 67 | 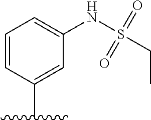 [SMILES: *c1cccc(NS(=O)(=O)CC)c1] |
| 68 | 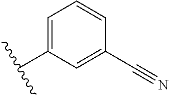 [SMILES: *c1cccc(C#N)c1] |
| 69 | 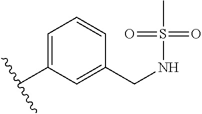 [SMILES: *c1cccc(CNS(C)(=O)=O)c1] |
| 70 | 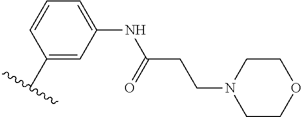 [SMILES: *c1cccc(NC(=O)CCN2CCOCC2)c1] |
| 71 | 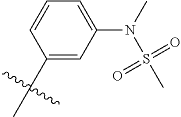 [SMILES: *c1cccc(N(C)S(C)(=O)=O)c1] |
| 72 | 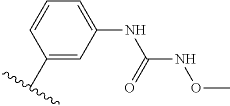 [SMILES: *c1cccc(NC(=O)NOC)c1] |
| 73 | 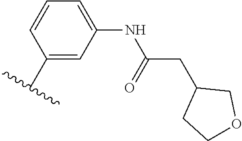 [SMILES: *c1cccc(NC(=O)CC2CCOC2)c1] |
| 74 | 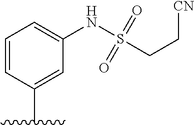 [SMILES: *c1cccc(NS(=O)(=O)CCC#N)c1] |
| 76 | 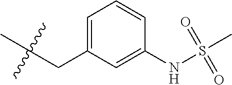 [SMILES: *Cc1cccc(NS(C)(=O)=O)c1] |
| 77 | 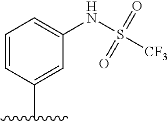 [SMILES: *c1cccc(NS(=O)(=O)C(F)(F)F)c1] |
| 79 | 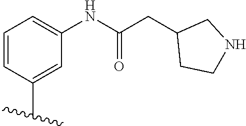 [SMILES: *c1cccc(NC(=O)CC2CCNC2)c1] |
| 80 | 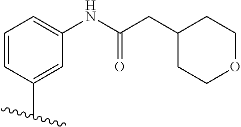 [SMILES: *c1cccc(NC(=O)CC2CCOCC2)c1] |
| 81 | 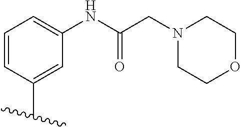 [SMILES: *c1cccc(NC(=O)CN2CCOCC2)c1] |
| 82 | 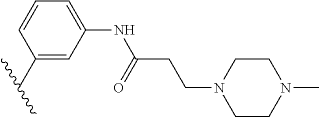 [SMILES: *c1cccc(NC(=O)CCN2CCN(C)CC2)c1] |
| 83 | 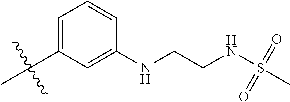 [SMILES: *c1cccc(NCCNS(C)(=O)=O)c1] |
| 84 | 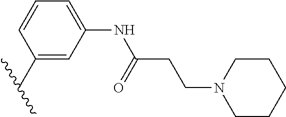 [SMILES: *c1cccc(NC(=O)CCN2CCCCC2)c1] |
| 85 | 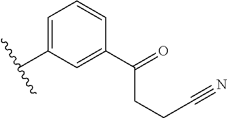 [SMILES: *c1cccc(C(=O)CCC#N)c1] |
| 86 | 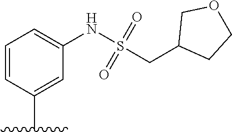 [SMILES: *c1cccc(NS(=O)(=O)CC2CCOC2)c1] |
| 87 | 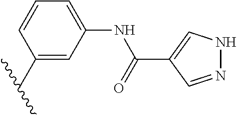 [SMILES: *c1cccc(NC(=O)c2cn[nH]c2)c1] |
| 88 | 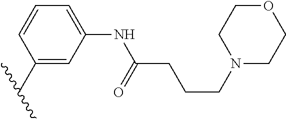 [SMILES: *c1cccc(NC(=O)CCCN2CCOCC2)c1] |
| 89 | 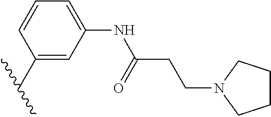 [SMILES: *c1cccc(NC(=O)CCN2CCCC2)c1] |
| 90 | 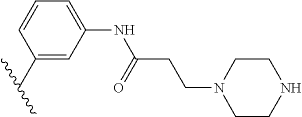 [SMILES: *c1cccc(NC(=O)CCN2CCNCC2)c1] |
| 91 | 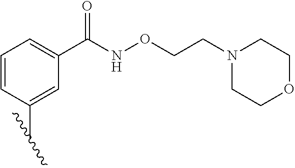 [SMILES: *c1cccc(C(=O)NOCCN2CCOCC2)c1] |
| 92 | 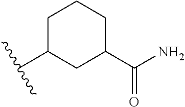 [SMILES: *C1CCCC(C(N)=O)C1] |
| 93 | 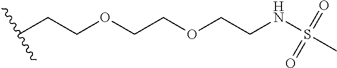 [SMILES: *CCOCCOCCNS(C)(=O)=O] |
| 94 | 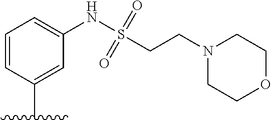 [SMILES: *c1cccc(NS(=O)(=O)CCN2CCOCC2)c1] |
| 95 | 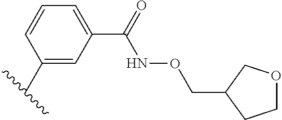 [SMILES: *c1cccc(C(=O)NOCC2CCOC2)c1] |
| 96 | 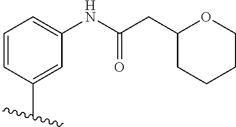 [SMILES: *c1cccc(NC(=O)CC2CCCCO2)c1] |
| 97 | 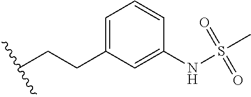 [SMILES: *CCc1cccc(NS(C)(=O)=O)c1] |
| 98 | 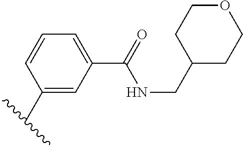 [SMILES: *c1cccc(C(=O)NCC2CCOCC2)c1] |
| 99 | 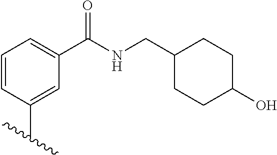 [SMILES: *c1cccc(C(=O)NCC2CCC(O)CC2)c1] |
| 101 | 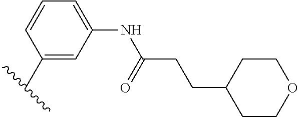 [SMILES: *c1cccc(NC(=O)CCC2CCOCC2)c1] |
| 102 | 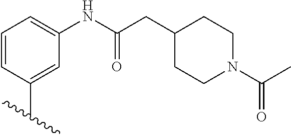 [SMILES: *c1cccc(NC(=O)CC2CCN(C(C)=O)CC2)c1] |
| 103 | 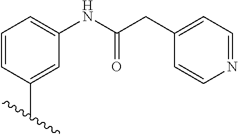 [SMILES: *c1cccc(NC(=O)Cc2ccncc2)c1] |
| 104 | 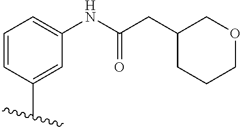 [SMILES: *c1cccc(NC(=O)CC2CCCOC2)c1] |
| 105 | 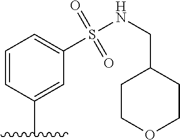 [SMILES: *c1cccc(S(=O)(=O)NCC2CCOCC2)c1] |
| 106 | 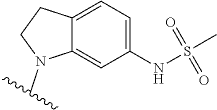 [SMILES: *N1CCc2ccc(NS(C)(=O)=O)cc21] |
| 107 | 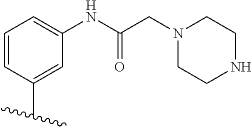 [SMILES: *c1cccc(NC(=O)CN2CCNCC2)c1] |
| 108 | 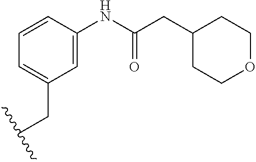 [SMILES: *Cc1cccc(NC(=O)CC2CCOCC2)c1] |
| 109 | 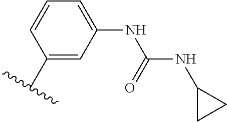 [SMILES: *c1cccc(NC(=O)NC2CC2)c1] |
| 110 | 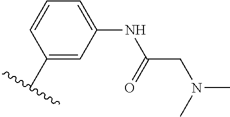 [SMILES: *c1cccc(NC(=O)CN(C)C)c1] |
| 111 | 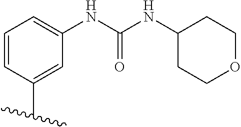 [SMILES: *c1cccc(NC(=O)NC2CCOCC2)c1] |
| 112 | 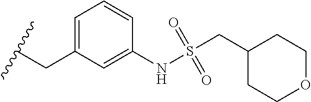 [SMILES: *Cc1cccc(NS(=O)(=O)CC2CCOCC2)c1] |
| 114 | 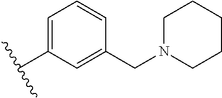 [SMILES: *c1cccc(CN2CCCCC2)c1] |
| 116 | 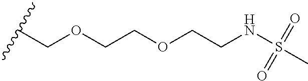 [SMILES: *COCCOCCNS(C)(=O)=O] |
| 117 | 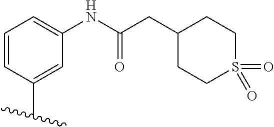 [SMILES: *c1cccc(NC(=O)CC2CCS(=O)(=O)CC2)c1] |
| 119 | 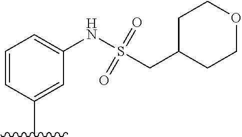 [SMILES: *c1cccc(NS(=O)(=O)CC2CCOCC2)c1] |
| 121 | 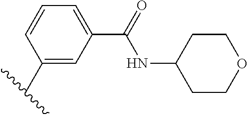 [SMILES: *c1cccc(C(=O)NC2CCOCC2)c1] |
| 120 | 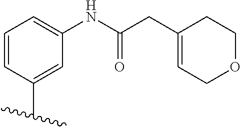 [SMILES: *c1cccc(NC(=O)CC2=CCOCC2)c1] |
| 122 | 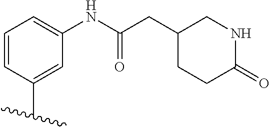 [SMILES: *c1cccc(NC(=O)CC2CCC(=O)NC2)c1] |
| 123 | 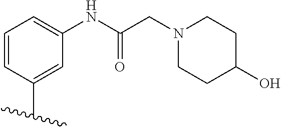 [SMILES: *c1cccc(NC(=O)CN2CCC(O)CC2)c1] |
| 124 | 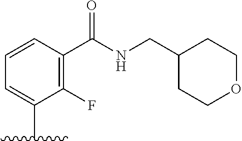 [SMILES: *c1cccc(C(=O)NCC2CCOCC2)c1F] |
| 125 | 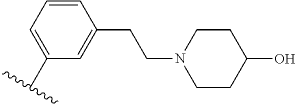 [SMILES: *c1cccc(CCN2CCC(O)CC2)c1] |
| 126 | 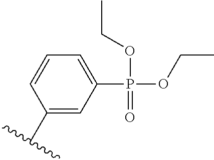 [SMILES: *c1cccc(P(=O)(OCC)OCC)c1] |
| 127 | 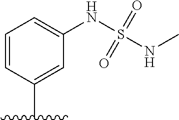 [SMILES: *c1cccc(NS(=O)(=O)NC)c1] |
| 128 | 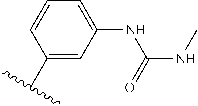 [SMILES: *c1cccc(NC(=O)NC)c1] |
| 129 | 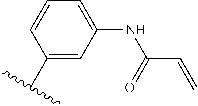 [SMILES: *c1cccc(NC(=O)C=C)c1] |
| 130 | 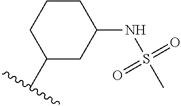 [SMILES: *C1CCCC(NS(C)(=O)=O)C1] |
| 131 | 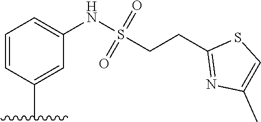 [SMILES: *c1cccc(NS(=O)(=O)CCc2nc(C)cs2)c1] |
| 133 | 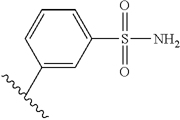 [SMILES: *c1cccc(S(N)(=O)=O)c1] |
| 134 | 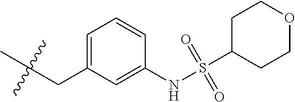 [SMILES: *Cc1cccc(NS(=O)(=O)C2CCOCC2)c1] |
| 136 | 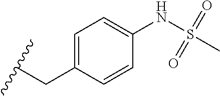 [SMILES: *Cc1ccc(NS(C)(=O)=O)cc1] |
| 137 | 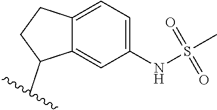 [SMILES: *C1CCc2ccc(NS(C)(=O)=O)cc21] |
| 138 | 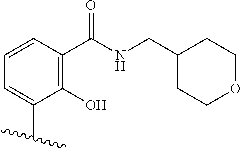 [SMILES: *c1cccc(C(=O)NCC2CCOCC2)c1O] |
| 139 | 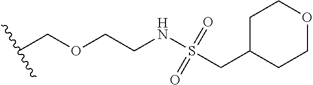 [SMILES: *COCCNS(=O)(=O)CC1CCOCC1] |
| 141 | 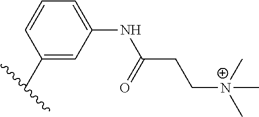 [SMILES: *c1cccc(NC(=O)CC[N+](C)(C)C)c1] |
| 142 | 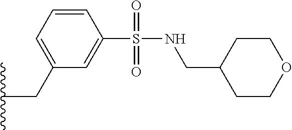 [SMILES: *Cc1cccc(S(=O)(=O)NCC2CCOCC2)c1] |
| 143 | 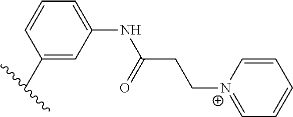 [SMILES: *c1cccc(NC(=O)CC[n+]2ccccc2)c1] |
| 144 | 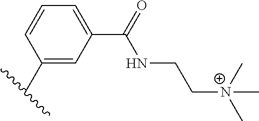 [SMILES: *c1cccc(C(=O)NCC[N+](C)(C)C)c1] |
| 145 | 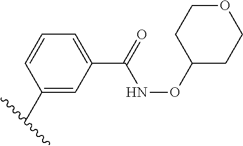 [SMILES: *c1cccc(C(=O)NOC2CCOCC2)c1] |
| 146 | 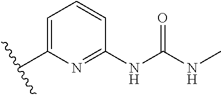 [SMILES: *c1cccc(NC(=O)NC)n1] |
| 147 | 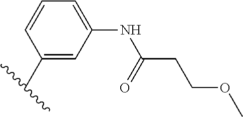 [SMILES: *c1cccc(NC(=O)CCOC)c1] |
| 148 | 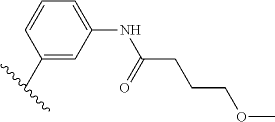 [SMILES: *c1cccc(NC(=O)CCCOC)c1] |
| 149 | 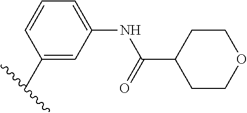 [SMILES: *c1cccc(NC(=O)C2CCOCC2)c1] |
| 150 | 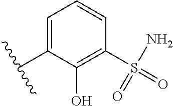 [SMILES: *c1cccc(S(N)(=O)=O)c1O] |
| 151 | 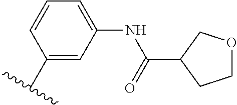 [SMILES: *c1cccc(NC(=O)C2CCOC2)c1] |
| 152 | 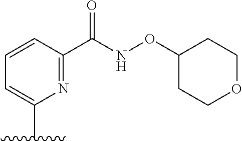 [SMILES: *c1cccc(C(=O)NOC2CCOCC2)n1] |
| 153 | 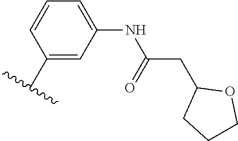 [SMILES: *c1cccc(NC(=O)CC2CCCO2)c1] |
| 154 | 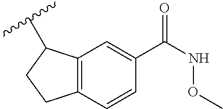 [SMILES: *C1CCc2ccc(C(=O)NOC)cc21] |
| 155 | 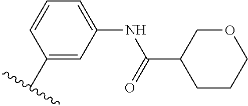 [SMILES: *c1cccc(NC(=O)C2CCCOC2)c1] |
| 156 | 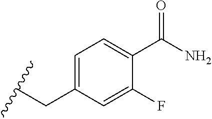 [SMILES: *Cc1ccc(C(N)=O)c(F)c1] |
| 157 | 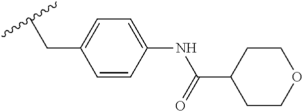 [SMILES: *Cc1ccc(NC(=O)C2CCOCC2)cc1] |
| 158 | 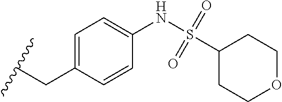 [SMILES: *Cc1ccc(NS(=O)(=O)C2CCOCC2)cc1] |
| 159 | 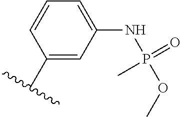 [SMILES: *c1cccc(NP(C)(=O)OC)c1] |
| 160 | 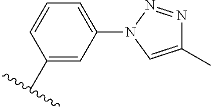 [SMILES: *c1cccc(-n2cc(C)nn2)c1] |
| 161 | 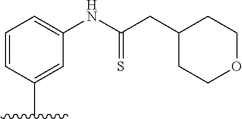 [SMILES: *c1cccc(NC(=S)CC2CCOCC2)c1] |
| 162 | 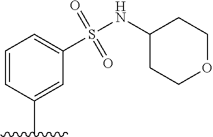 [SMILES: *c1cccc(S(=O)(=O)NC2CCOCC2)c1] |
| 163 | 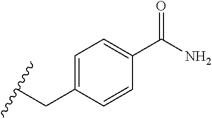 [SMILES: *Cc1ccc(C(N)=O)cc1] |
| 164 | 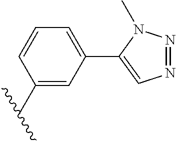 [SMILES: *c1cccc(-c2cnnn2C)c1] |
| 165 | 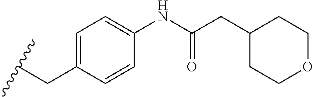 [SMILES: *Cc1ccc(NC(=O)CC2CCOCC2)cc1] |
| 166 | 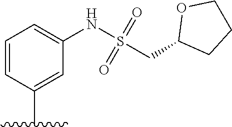 [SMILES: *c1cccc(NS(=O)(=O)C[C@H]2CCCO2)c1] |
| 167 | 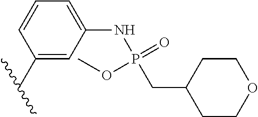 [SMILES: *c1cccc(NP(=O)(CC2CCOCC2)OC)c1] |
| 168 | 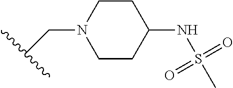 [SMILES: *CN1CCC(NS(C)(=O)=O)CC1] |
| 169 | 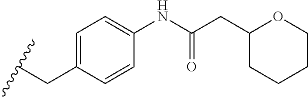 [SMILES: *Cc1ccc(NC(=O)CC2CCCCO2)cc1] |
| 170 | 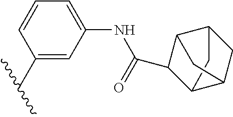 [SMILES: *c1cccc(NC(=O)C2C3CC4CC3CC42)c1] |
| 171 | 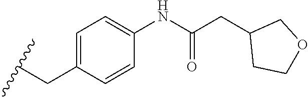 [SMILES: *Cc1ccc(NC(=O)CC2CCOC2)cc1] |
| 172 | 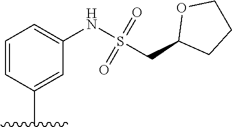 [SMILES: *c1cccc(NS(=O)(=O)C[C@@H]2CCCO2)c1] |
| 173 | 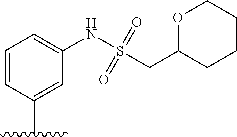 [SMILES: *c1cccc(NS(=O)(=O)CC2CCCCO2)c1] |
| 174 | 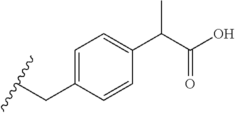 [SMILES: *Cc1ccc(C(C)C(=O)O)cc1] |
| 175 | 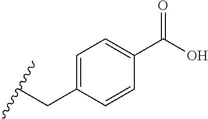 [SMILES: *Cc1ccc(C(=O)O)cc1] |
| 176 | 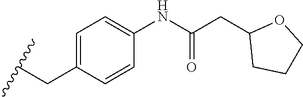 [SMILES: *Cc1ccc(NC(=O)CC2CCCO2)cc1] |
| 177 | 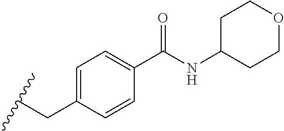 [SMILES: *Cc1ccc(C(=O)NC2CCOCC2)cc1] |
| 178 | 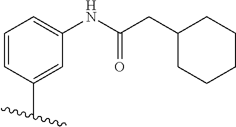 [SMILES: *c1cccc(NC(=O)CC2CCCCC2)c1] |
| 179 | 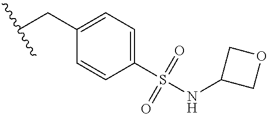 [SMILES: *Cc1ccc(S(=O)(=O)NC2COC2)cc1] |
| 180 | 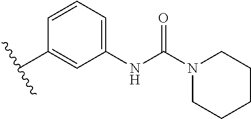 [SMILES: *c1cccc(NC(=O)N2CCCCC2)c1] |
| 181 | 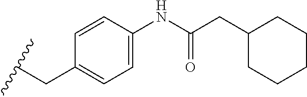 [SMILES: *Cc1ccc(NC(=O)CC2CCCCC2)cc1] |
| 182 | 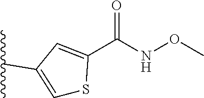 [SMILES: *c1csc(C(=O)NOC)c1] |
| 183 | 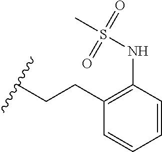 [SMILES: *CCc1ccccc1NS(C)(=O)=O] |
| 184 | 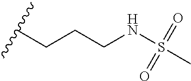 [SMILES: *CCCNS(C)(=O)=O] |
| 185 | 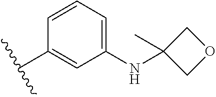 [SMILES: *c1cccc(NC2(C)COC2)c1] |
| 186 | 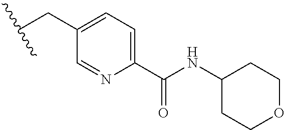 [SMILES: *Cc1ccc(C(=O)NC2CCOCC2)nc1] |
| 187 | 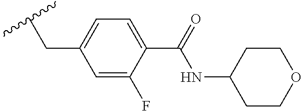 [SMILES: *Cc1ccc(C(=O)NC2CCOCC2)c(F)c1] |
| 188 | 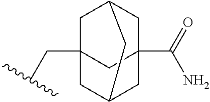 [SMILES: *CC12CC3CC(C1)CC(C(N)=O)(C3)C2] |
| 189 | 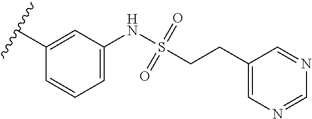 [SMILES: *c1cccc(NS(=O)(=O)CCc2cncnc2)c1] |
| 190 | 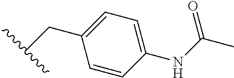 [SMILES: *Cc1ccc(NC(C)=O)cc1] |
| 191 | 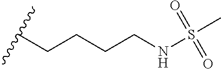 [SMILES: *CCCCNS(C)(=O)=O] |
| 192 | 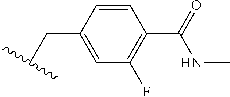 [SMILES: *Cc1ccc(C(=O)NC)c(F)c1] |
| 193 | 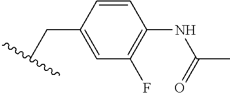 [SMILES: *Cc1ccc(NC(C)=O)c(F)c1] |
| 194 | 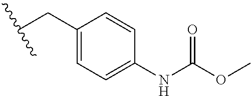 [SMILES: *Cc1ccc(NC(=O)OC)cc1] |
| 195 | 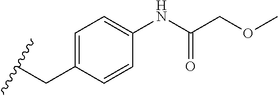 [SMILES: *Cc1ccc(NC(=O)COC)cc1] |
| 196 | 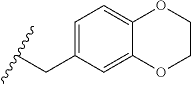 [SMILES: *Cc1ccc2c(c1)OCCO2] |
| 197 | 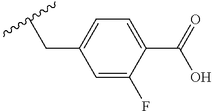 [SMILES: *Cc1ccc(C(=O)O)c(F)c1] |
| 198 | 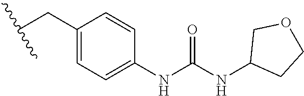 [SMILES: *Cc1ccc(NC(=O)NC2CCOC2)cc1] |
| 199 | 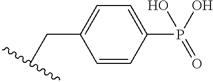 [SMILES: *Cc1ccc(P(=O)(O)O)cc1] |
| 200 | 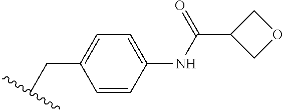 [SMILES: *Cc1ccc(NC(=O)C2COC2)cc1] |
| 201 | 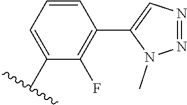 [SMILES: *c1cccc(-c2cnnn2C)c1F] |
| 202 | 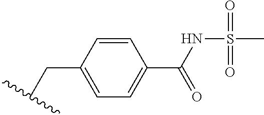 [SMILES: *Cc1ccc(C(=O)NS(C)(=O)=O)cc1] |
| 203 | 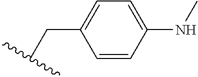 [SMILES: *Cc1ccc(NC)cc1] |
| 204 | 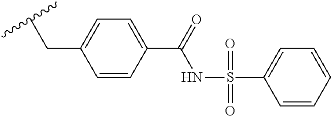 [SMILES: *Cc1ccc(C(=O)NS(=O)(=O)c2ccccc2)cc1] |
| 205 | 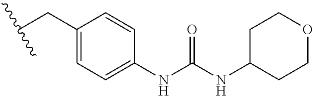 [SMILES: *Cc1ccc(NC(=O)NC2CCOCC2)cc1] |
| 206 | 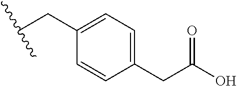 [SMILES: *Cc1ccc(CC(=O)O)cc1] |
| 207 | 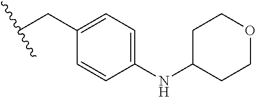 [SMILES: *Cc1ccc(NC2CCOCC2)cc1] |
| 208 | 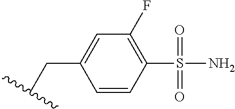 [SMILES: *Cc1ccc(S(N)(=O)=O)c(F)c1] |
| 209 | 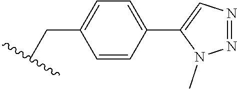 [SMILES: *Cc1ccc(-c2cnnn2C)cc1] |
| 210 | 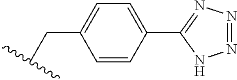 [SMILES: *Cc1ccc(-c2nnn[nH]2)cc1] |
| 211 | 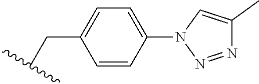 [SMILES: *Cc1ccc(-n2cc(C)nn2)cc1] |
| 212 | 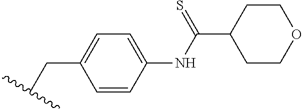 [SMILES: *Cc1ccc(NC(=S)C2CCOCC2)cc1] |
| 213 | 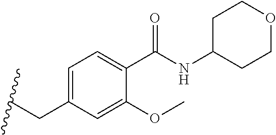 [SMILES: *Cc1ccc(C(=O)NC2CCOCC2)c(OC)c1] |
| 214 | 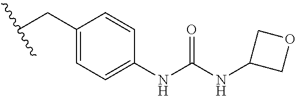 [SMILES: *Cc1ccc(NC(=O)NC2COC2)cc1] |
| 215 | 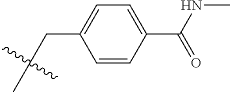 [SMILES: *Cc1ccc(C(=O)NC)cc1] |
| 216 | 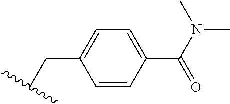 [SMILES: *Cc1ccc(C(=O)N(C)C)cc1] |
| 217 | 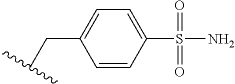 [SMILES: *Cc1ccc(S(N)(=O)=O)cc1] |
| 218 | 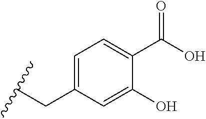 [SMILES: *Cc1ccc(C(=O)O)c(O)c1] |
| 219 | 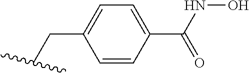 [SMILES: *Cc1ccc(C(=O)NO)cc1] |
| 220 | 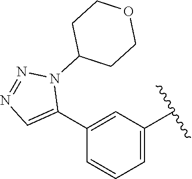 [SMILES: *c1cccc(-c2cnnn2C2CCOCC2)c1] |
| 221 | 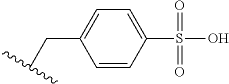 [SMILES: *Cc1ccc(S(=O)(=O)O)cc1] |
| 222 | 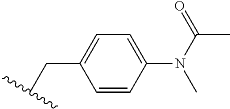 [SMILES: *Cc1ccc(N(C)C(C)=O)cc1] |
| 223 | 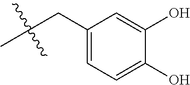 [SMILES: *Cc1ccc(O)c(O)c1] |
| 224 | 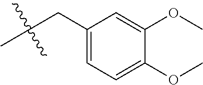 [SMILES: *Cc1ccc(OC)c(OC)c1] |
| 225 | 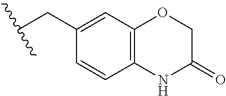 [SMILES: *Cc1ccc2c(c1)OCC(=O)N2] |
| 226 | 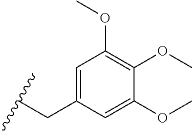 [SMILES: *Cc1cc(OC)c(OC)c(OC)c1] |
| 227 | 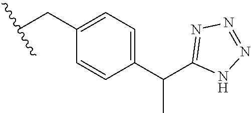 [SMILES: *Cc1ccc(C(C)c2nnn[nH]2)cc1] |
| 228 | 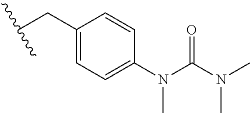 [SMILES: *Cc1ccc(N(C)C(=O)N(C)C)cc1] |
| 229 | 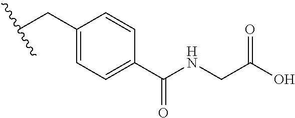 [SMILES: *Cc1ccc(C(=O)NCC(=O)O)cc1] |
| 230 | 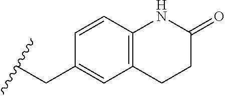 [SMILES: *Cc1ccc2c(c1)CCC(=O)N2] |
| 231 | 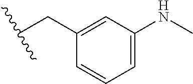 [SMILES: *Cc1cccc(NC)c1] |
| 232 | 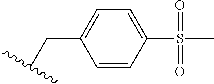 [SMILES: *Cc1ccc(S(C)(=O)=O)cc1] |
| 233 | 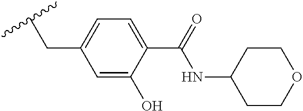 [SMILES: *Cc1ccc(C(=O)NC2CCOCC2)c(O)c1] |
| 234 | 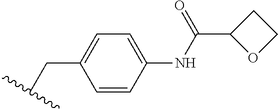 [SMILES: *Cc1ccc(NC(=O)C2CCO2)cc1] |
| 235 | 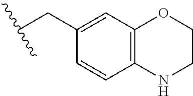 [SMILES: *Cc1ccc2c(c1)OCCN2] |
| 236 | 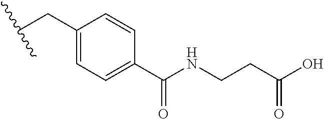 [SMILES: *Cc1ccc(C(=O)NCCC(=O)O)cc1] |
| 237 | 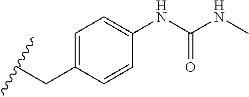 [SMILES: *Cc1ccc(NC(=O)NC)cc1] |
| 238 | 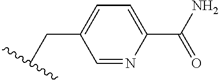 [SMILES: *Cc1ccc(C(N)=O)nc1] |
| 239 | 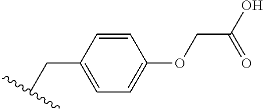 [SMILES: *Cc1ccc(OCC(=O)O)cc1] |
| 240 | 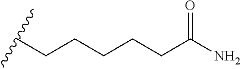 [SMILES: *CCCCCC(N)=O] |
| 241 | 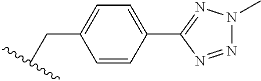 [SMILES: *Cc1ccc(-c2nnn(C)n2)cc1] |
| 242 | 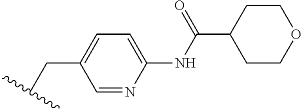 [SMILES: *Cc1ccc(NC(=O)C2CCOCC2)nc1] |
| 243 | 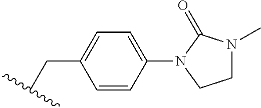 [SMILES: *Cc1ccc(N2CCN(C)C2=O)cc1] |
| 245 | 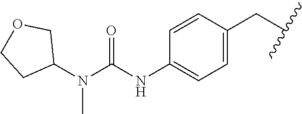 [SMILES: *Cc1ccc(NC(=O)N(C)C2CCOC2)cc1] |
| 246 | 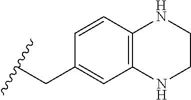 [SMILES: *Cc1ccc2c(c1)NCCN2] |
| 247 | 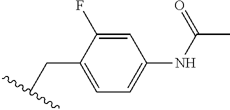 [SMILES: *Cc1ccc(NC(C)=O)cc1F] |
| 248 | 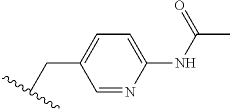 [SMILES: *Cc1ccc(NC(C)=O)nc1] |
| 249 | 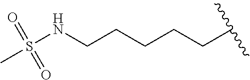 [SMILES: *CCCCCNS(C)(=O)=O] |
| 250 | 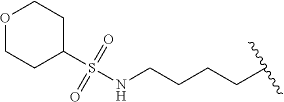 [SMILES: *CCCCNS(=O)(=O)C1CCOCC1] |
| 251 | 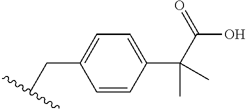 [SMILES: *Cc1ccc(C(C)(C)C(=O)O)cc1] |

### Methods of Preparation

Compounds described herein can be prepared by any number of processes as described generally below and more specifically illustrated by the exemplary compounds which follow in the Examples section herein. The compounds provided herein as prepared in the processes described below can be synthesized in the form of mixtures of stereoisomers (e.g., enantiomers, diastereomers), including racemic mixtures of enantiomers, which can be separated from one another using art-known resolution procedures, for instance including liquid chromatography using a chiral stationary phase. Additionally or alternatively, stereochemically pure isomeric forms of the compounds described herein can be derived from the corresponding stereochemically pure isomeric forms of the appropriate starting materials, intermediates, or reagents. For example, if a specific stereoisomer is desired, the compound can be synthesized by stereospecific methods of preparation, which typically employ stereochemically pure starting materials or intermediate compounds.

Pharmaceutically acceptable salts of compounds described herein can be synthesized from the parent compound containing an acidic or basic moiety by conventional chemical methods. Generally, such salts can be prepared by reacting the free acid or base forms of these compounds with a stoichiometric amount of the appropriate acid or base in water or in an organic solvent, or in a mixture of the two. Examples of suitable organic solvents include, but are not limited to, ether, ethyl acetate (EtOAc), ethanol, isopropanol, or acetonitrile.

By way of illustration, but not as a limitation, compounds of formula (I-A), (I-B), (I-C), (I-D), or (II) described herein can be prepared according to the following general preparation procedures shown in Scheme 1 as well as the examples shown in the present disclosure. One of ordinary skill in the art will recognize that, to obtain various compounds of formula (I-A), (I-B), (I-C), (I-D), or (II) as described herein, starting materials can be suitably selected so that the ultimately desired substituent groups will be carried through (i.e., be stable over the course of the synthesis) the reaction scheme with or without protection as appropriate to yield the desired product. Alternatively, it may be necessary or desirable to employ, in place of the ultimately desired substituent, a suitable group that may be carried through (i.e., be stable over the course of the synthesis) the reaction scheme and replaced as appropriate with the desired substituent.

If no temperature or temperature range is stated, it is to be understood that the reaction is to be conducted at room temperature.

When isomerically pure samples are desired, isomeric mixtures of compounds synthesized according to Scheme 1 can be separated by chiral supercritical fluid chromatography (SFC) or high performance liquid chromatography (HPLC).

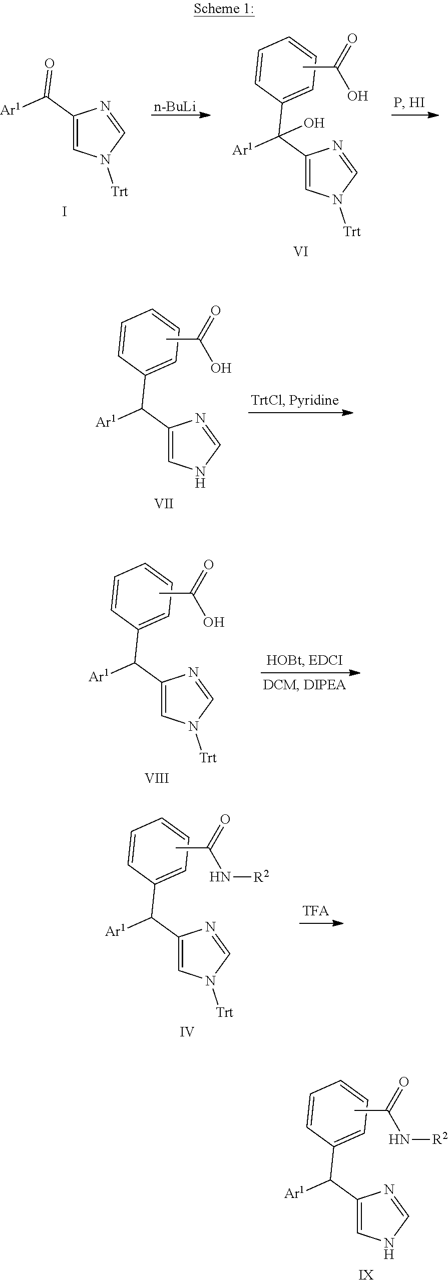

### Compositions

In another general aspect, the present disclosure provides a pharmaceutical composition comprising: (a) a means for increasing the activation of α2AR of the peripheral nervous system of a subject, and (b) a pharmaceutically acceptable carrier.

In some embodiments, the pharmaceutical composition comprises:
(a) a molecule comprising a moiety having formula

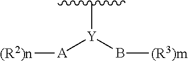

covalently linked to a means for increasing the distribution of said molecule to the peripheral nervous system of a subject, wherein A and B are defined as above in formula (I-A), and
(b) a pharmaceutically acceptable carrier.

In some embodiments, the pharmaceutical composition comprises:
(a) a molecule comprising a moiety having formula

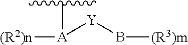

covalently linked to a means for increasing the distribution of said molecule to the peripheral nervous system of a subject, wherein A and B are defined as above in formula (I-B), and
(b) a pharmaceutically acceptable carrier.

In some embodiments, the pharmaceutical composition comprises:
(a) a molecule comprising a moiety having formula

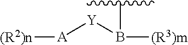

covalently linked to a means for increasing the distribution of said molecule to the peripheral nervous system of a subject, wherein A and B are defined as above in formula (I-C), and
(b) a pharmaceutically acceptable carrier.

In some embodiments, the pharmaceutical composition comprises:
(a) a molecule comprising a moiety having formula

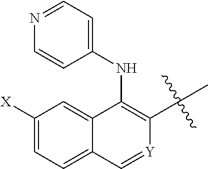

covalently linked to a means for increasing the distribution of said molecule to the peripheral nervous system of a subject, wherein A and B are defined as above in formula (I-C), and
(b) a pharmaceutically acceptable carrier.

In some embodiments, the pharmaceutical composition comprises:
(a) a molecule comprising a moiety having formula

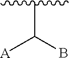

covalently linked to a means for increasing the distribution of said molecule to the peripheral nervous system of a subject, wherein A and B are defined as above in formula (II), and
(b) a pharmaceutically acceptable carrier.

In some embodiments, the pharmaceutical composition comprises:
(a) a molecule comprising a moiety having formula

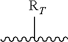

covalently linked to a means for activating α2 adrenergic receptor (α2AR), wherein R_{T} is defined as above in formula (I-A), (I-B), (I-C), (I-D), or (II), and
(b) a pharmaceutically acceptable carrier.

In another general aspect, the present disclosure provides a pharmaceutical composition comprising: (a) a molecule comprising (i) a first means for activating α2AR, and (ii) a second means for increasing distribution of the molecule to the peripheral nervous system of a subject, wherein the first means is covalently linked to the second means, and (b) a pharmaceutically acceptable carrier.

In one general aspect, the present disclosure provides a means for activating α2 adrenergic receptor (α2AR).

In some embodiments, the means for activating α2AR is an α2AR agonist.

In another general aspect, the present disclosure provides a means for increasing distribution of a molecule to the peripheral nervous system of a subject.

In some embodiments, R_{T} in formula (I-A), (I-B), (I-C), (I-D), or (II) is a means for increasing distribution of the molecule to the peripheral nervous system of a subject.

In another aspect, provided is a pharmaceutical composition comprising a compound of formula (I-A), (I-B), (I-C), (I-D), or (II) or a stereoisomer, tautomer, pharmaceutically acceptable salt or solvate thereof, as described herein.

Compositions can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions can also be formulated for other routes of administration including transmucosal, ocular, rectal, long acting implantation, sublingual administration, under the tongue, from oral mucosa bypassing the portal circulation, inhalation, or intranasal.

The form of the pharmaceutical compositions, the route of administration, the dosage and the regimen depend upon the condition to be treated, such as the severity of the illness, the age, weight, and sex of the patient. Pharmaceutical compositions can be formulated for different modes of administration such as for topical, oral, intranasal, parenteral, intraocular, intravenous, intramuscular, or subcutaneous administration.

In yet another aspect, provided is a method of preparing a pharmaceutical composition comprising combining a compound of formula (I-A), (I-B), (I-C), (I-D), or (II), or a stereoisomer, tautomer, pharmaceutically acceptable salt or solvate thereof, with at least one pharmaceutically acceptable carrier. Pharmaceutical compositions can be prepared by any method known in the art in view of the present disclosure, and one of ordinary skill in the art will be familiar with such techniques used to prepare pharmaceutical compositions. For example, a pharmaceutical composition according to the present disclosure can be prepared by mixing a compound of formula (I-A), (I-B), (I-C), (I-D), or (II), with one or more pharmaceutically acceptable carriers according to conventional pharmaceutical compounding techniques, including but not limited to, conventional admixing, dissolving, granulating, emulsifying, encapsulating, entrapping or lyophilizing processes.

### Methods of Use

In one general aspect, provided are methods of treating or preventing pain in a subject in need thereof, the method comprising administering to the subject a therapeutically effective amount of a peripherally selective α2AR agonist, wherein treating with the peripherally selective α2AR agonist causes less side effects than treating with a non-peripherally selective α2AR agonist, such as at similar or comparable dosage.

In some embodiments, the peripherally selective α2AR agonist comprises an α2AR activation moiety covalently linked to a peripheral distribution moiety.

In another general aspect, provided are methods of treating or preventing a disease in a subject in need thereof, the method comprising administering to the subject a therapeutically effective amount of a peripherally selective α2AR agonist, wherein the peripherally selective α2AR agonist comprises an α2AR activation moiety covalently linked to a peripheral distribution moiety.

The following embodiments apply to the above two general aspects of methods of use.

In some embodiments, the α2AR activation moiety has formula of

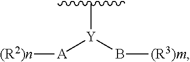

wherein Y, A, B, R², R³, m, and n are defined as in formula (I-A).

In some embodiments, the α2AR activation moiety has formula of

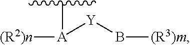

wherein Y, A, B, R², R³, m, and n are defined as in formula (I-B).

In some embodiments, the α2AR activation moiety has formula of

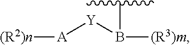

wherein Y, A, B, R², R³, m, and n are defined as in formula (I-C).

In some embodiments, the α2AR activation moiety has formula of

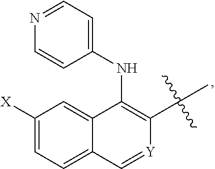

wherein X and Y are defined as in formula (I-D).

In some embodiments, the α2AR activation moiety has formula of

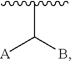

wherein A and B are defined as in formula (II).

In some embodiments, the peripheral distribution moiety has formula of

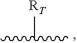

wherein R_{T} is defined as in formula (I-A).

In some embodiments, the peripheral distribution moiety has formula of

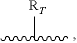

wherein R_{T} is defined as in formula (II).

In another general aspect, provided are methods of activating α2AR and methods of treating or preventing a disease in a subject, using the compounds described herein or the composition containing the compounds with one or more acceptable pharmaceutical carriers, describe herein.

In some embodiments, the compounds of formula (I-A), (I-B), (I-C), (I-D), or (II) can be useful for activating α2AR.

In some embodiments, provided is a method of activating α2AR in a subject in need thereof, comprising administering to the subject a compound or composition described herein, e.g., administering an effective amount of a compound or composition described herein.

In some embodiments, provided is a method of treating or preventing a disease in human or in animal.

In some embodiments, provided is a method of treating or preventing a disease in a subject in need thereof, comprising administering to the subject a compound or composition described herein, e.g., administering an effective amount of a compound or composition described herein.

In some embodiments, the disease is glaucoma, pain, spasticity, nasal congestion, rosacea, rhinitis, anesthesia, presbyopia, acute kidney injury, insomnia, inflammatory disease, cancer, etc. In some embodiments, the disease is pain.

In some embodiments, the pain is nociceptive pain, neuropathic pain such as peripheral neuropathic pain, or mixed pain. Examples of peripheral neuropathic pain include, but not limited to diabetic neuropathy, postherpetic neuralgia, HIV-associated pain, chemotherapy-induced peripheral neuropathy, and post-surgical neuropathic pain.

In some embodiments, the compounds and pharmaceutical compositions described herein cause less side effects when treating pain, such as sedation, decreasing heart rate, and decreasing blood pressure in the treated subject.

In certain embodiments, the compounds and pharmaceutical compositions described herein do not cause sedative response in the treated subject.

structures and/or materials

| | | Exemplified structures and/or materials |
|---|---|---|
| | | and/or acts disclosed in the Specification of |
| Claim | Recited function | the application filed herewith |
| Claim 1 | “means for increasing the activation | Table 1, and any tautomer, stereoisomer, |
| | of α2 adrenergic receptors of the | pharmaceutically acceptable salt or solvate |
| | peripheral nervous system of a subject” | thereof |
| Claims 2, | “means for increasing the | Table 1a |
| 4, and 9 | distribution of said molecule to the | |
| | peripheral nervous system of a subject” | |
| Claims 3 | “means for activating α2 adrenergic | (R)-3-nitrobiphenyline, A-193080, ADX- |
| and 4 | receptors” | 415, AGN 192836, AGN-191103, AGN- |
| | | 197075, AGN-201781, AGN-241622, |
| | | amitraz, Apraclonidine, AR-08, |
| | | Bethanidine, Brimonidine, BRL-48962, |
| | | Bromocriptine, Cirazoline, Clonidine, |
| | | Detomidine, Detomidine carboxylic acid, |
| | | Dexmedetomidine, Dipivefrin, DL- |
| | | Methylephedrine, Droxidopa, Epinephrine, |
| | | ergotamine, etilefrine, Etomidate, |
| | | Fadolmidine, Guanabenz, Guanethidine, |
| | | Guanfacine, Guanoxabenz, indanidine, |
| | | Lofexidine, Medetomidine, |
| | | mephentermine, Metamfetamine, |
| | | metaraminol, methoxamine, Methyldopa, |
| | | Methyldopate, Methyldopate |
| | | hydrochloride, Methylnorepinephrine, |
| | | mivazerol, Moxonidine, naphazoline, |
| | | Norepinephrine, norfenefrine, octopamine, |
| | | ODM-105, Oxymetazoline, Pergolide, |
| | | phenylpropanolamine, Povafonidine, |
| | | propylhexedrine, Pseudoephedrine, |
| | | Racepinephrine, rezatomidine, rilmenidine, |
| | | romifidine, synephrine, talipexole, |
| | | tasipimidine, Tiamenidine, Tizanidine, |
| | | Xylazine, Xylometazoline, and any |
| | | tautomer, stereoisomer, pharmaceutically |
| | | acceptable salt or solvate thereof |

### EXAMPLES

The following examples are to further illustrate the nature of the present disclosure. It should be understood that the following examples do not limit the disclosure and the scope of the present disclosure is to be determined by the appended claims.

### Methods of Synthesis

Unless indicated otherwise, the abbreviations for chemical reagents and synthesis conditions have their ordinary meaning known in the art as follows:
“ACN” refers to acetonitrile;
“LDA” refers to lithium diisopropyl amide;
“EA” or “EtOAc” refers to ethyl acetate;
“PE” refers to petroleum ether;
“r.t.” and “rt” refer to room temperature;
“THF” refers to tetrahydrofuran;
“DIPEA” refers to diisopropylethylamine;
“DCM” refers to dichloromethane;
“HOBT” refers to hydroxybenzotriazole;
“TLC” refers to thin layer chromatography;
“TLC” refers to thin layer chromatography;
“DMF” refers to dimethylformamide;
“h” refers to hours;
“min” refers to minutes;
“EDCI” refers to 1-Ethyl-3-(3-dimethylaminopropyl) carbodiimide;
“DMAP” refers to 4-Dimethylaminopyridine;
“Prep-HPLC” refers to preparative high performance liquid chromatography;
“DPPF” refers to 1,1′-Bis(diphenylphosphino) ferrocene; and
“NCS” refers to N-chlorosuccinimide.
“TEA” refers to triethylamine.
“TES” refers to triethyl silane.
“Trt” refers to trityl group or triphenylmethyl group.
“MeOH” refers to methanol.
“EtOH” refers to ethanol.
“t-BuXphos” refers to tert-butyl-Xantphos
“TMAI” refers to trimethylaluminum
“Xantphos” refers to 9,9-dimethyl-4,5-bis(diphenylphosphino) xanthene
“Pd(PPH3)4” refers to tetrakis(triphenylphosphine) palladium (0)

### Example 1. Synthesis of Compound 1

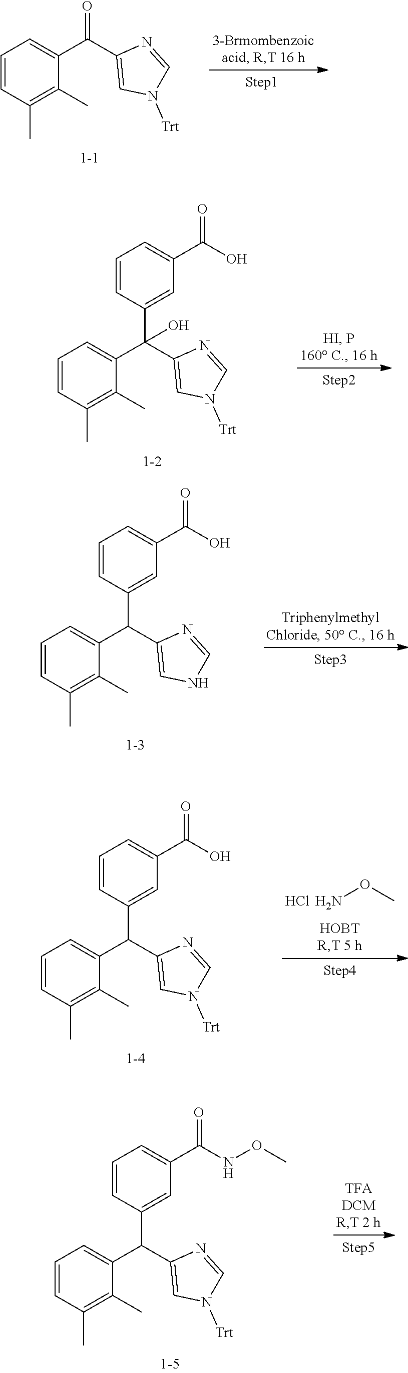

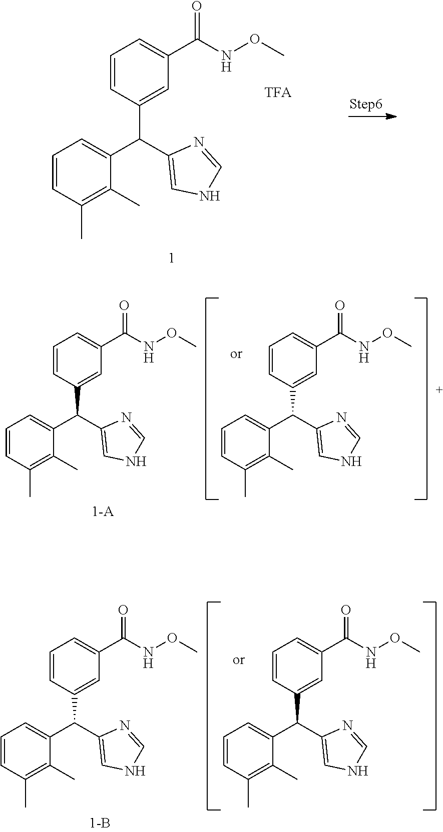

Step 1: 400 mL THF and 36 g (0.18 mol, 4.0 eq) of 3-bromobenzoic acid were added into a 500 mL reaction flask. Following cooling to −65° C., 135 mL (4 mol/L, 0.428 mol, 7.5 eq) of n-Butyllithium was added. The mixture was stirred at −65° C. for 2 hours before 20 g (0.057 mol, 1.0 eq) of compound 1-1 and an additional 400 mL of THF were introduced. After stirring at −65° C. for 30 minutes, the mixture was allowed to warm to room temperature over 16 hours. Completion was confirmed by LC-MS, and 270 mL of saturated ammonium chloride was added. The organic phase was then separated, vacuum concentrated, and the residue was column chromatographed (DCM-DCM:MeOH=92:8) to yield 13 g of compound 1-2, with a 40.4% yield.

Step 2:150 ml of 55% HI, 7.5 g (13.3 mmol, 1.0 eq) of compound 1-2, and 4.1 g (133 mmol, 10.0 eq) of red phosphorus were added into a 200 mL sealing tube The mixture was stirred at 160° C. for 16 hours until LC-MS indicated completion. Following vacuum concentration, the residue was collected to produce 7.3 g of compound 1-3, achieving a 100% yield.

Step 3:240 ml of pyridine, 12.1 g (39.5 mmol, 1.0 eq) of compound 1-3, and 55.1 g (197.5 mmol, 3.0 eq) of triphenylmethyl chloride were added to a 50 mL reaction flask. The mixture was stirred at 50° C. for 2 hours until LC-MS confirmed completion. After vacuum concentration, the residue underwent column chromatography (DCM-DCM:MeOH=92:8) to obtain 4.5 g of compound 1-4, with a 20.8% yield.

Step 4: 52 ml DCM, and then 1.3 g (2.37 mmol, 1.0 eq) of compound 1-4, 594 mg (7.11 mmol, 3.0 eq) of methoxyammonium chloride, 2.45 g (18.96 mmol, 8.0 eq) of DIPEA, 640 mg (4.74 mmol, 2.0 eq) of HOBT, and 999 mg (5.21 mmol, 2.2 eq) of EDCI were added to a 100 mL reaction flask. The mixture was stirred at room temperature for 5 hours until LC-MS confirmed the reaction's completion. After vacuum concentration, the residue was purified by column chromatography (DCM-DCM:MeOH=91:9) to yield 900 mg of compound 1-5, with a yield of 65.8%.

Step 5: 18 mL DCM and 900 mg (1.56 mmol, 1.0 eq) of compound 1-5, along with 9 mL of TFA, were added to a 50 mL reaction flask. The reaction was stirred at room temperature for 2 hours until LC-MS showed completion. Following vacuum concentration, the residue underwent column chromatography to yield 670 mg of compound 1, achieving a 98.5% yield.

1H NMR: (400 MHz DMSO) δ 14.33 (s, 2H), 11.79 (s, 1H), 9.00 (d, J=0.9 Hz, 1H), 7.67 (d, J=7.8 Hz, 1H), 7.60 (s, 1H), 7.46 (t, J=7.7 Hz, 1H), 7.35 (d, J=7.7 Hz, 1H), 7.17-7.06 (m, 2H), 6.94 (s, 1H), 6.70 (d, J=7.2 Hz, 1H), 5.91 (s, 1H), 3.69 (s, 3H), 2.26 (s, 3H), 2.13 (s, 3H). LC-MS: [M-TFA+1]+=336.2

Step 6: Compound 1 was separated by chiral HPLC to afford compound 1-A and compound 1-B. A column with the dimensions 30×

250 mm packed with CHIRALPAK® IG (10 μm particle size) was used as the chiral stationary phase. A mixture of 60% volume mobile phase A and 40% volume mobile phase B was used as the mobile phase.
Mobile phase A: Hexane+0.2% NH₃ in MeOH
Mobile phase B: EtOH+0.2% NH₃ in MeOH

The operation conditions were as follows:
Temperature: Ambient temperature
Flow rate: 25 mL/min
Detection: UV 214 nm

500 mg of compound 1 was separated on the column. The first eluting enantiomer (compound 1-A) with a retention time of 4.18 min was isolated from the eluent with an enantiomeric excess of 100% in 80% yield. The second eluting enantiomer (compound 1-B) with a retention time of 5.83 min was isolated from the eluent with an enantiomeric excess of 99.2% in 81% yield. In this application, the naming convention for separated enantiomers is systematic. “A” denotes the first eluting product from the chromatography, and “B” indicates the second. For compounds where chirality leads to four distinct products, they are labeled as “A,” “B,” “C,” and “D,” based on their elution order. Consequently, if a compound is named as X, the separated products would be systematically named “X-A,” “X—B,” “X—C,” and “X-D.”

### Example 2. Synthesis of Compound 8

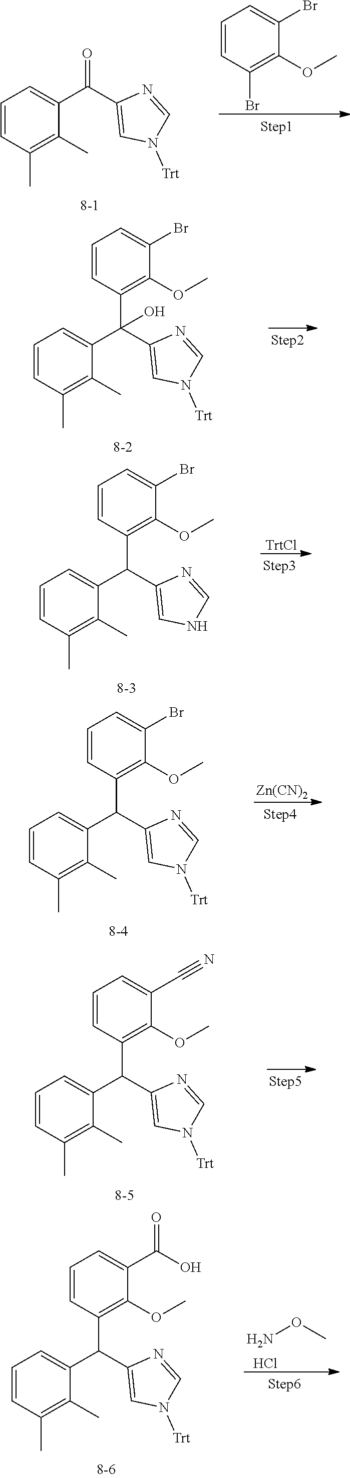

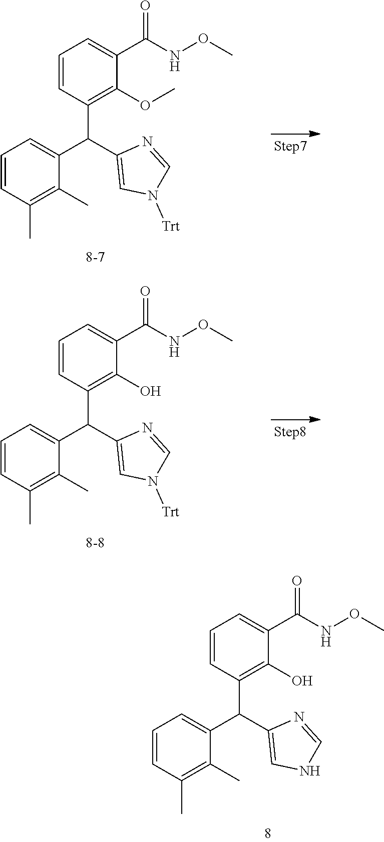

Step 1: 200 mL of THF and 9.10 g (34.1 mmol, 1.5 eq) of 2,6-dibromo-1-methoxybenzene were added in a 500 mL three-necked round bottom flask under nitrogen atmosphere. At 0° C., 27 mL (34.1 mmol, 1.5 eq) of isopropylmagnesium chloride lithium chloride complex was introduced. The mixture was stirred for 6 hours at 0° C. before adding 10 g (22.7 mmol, 1 eq) of compound 8-1, still under 0° C. Stirring continued for 16 hours at room temperature (25° C.). Then, it was poured into water, washed with ethyl acetate, dried using Na₂SO₄, and fast silica gel column purification yielded 11.3 g of compound 8-2 (79%).

Step 2: A 250 mL three-necked round bottom flask received 113 ml of DCM, 11.3 g (18 mmol, 1.0 eq) of compound 8-2, HSiEt₃ (21 g, 180 mmol, 10 eq), and TFA (21 g, 180 mmol, 10 eq) under nitrogen at 0° C. It was stirred until reaching room temperature over 16 hours. Concentration under vacuum produced compound 8-3 (17 g, crude).

Step 3:17 g (18 mmol, 1.0 eq) of compound 8-3, TrtCl (12.6 g, 45 mmol, 2.5 eq), 170 ml of DCM and Et3N (9.1 g, 90 mmol, 5 eq) were mixed in a 500 ml three-necked round bottom flask under nitrogen. After stirring for 16 hours at 25° C., completion was confirmed by LC-MS. The product was processed similarly to previous steps to yield 7.1 g of compound 8-4 (64%).

Step 4: 100 ml of DMF, 6 g (9.8 mmol, 1.0 eq) of compound 8-4, Zn(CN)₂ (1.26 g, 10.8 mmol, 1.1 eq), and Pd(PPh₃)₄ (1.26 g, 1.1 mmol, 0.11 eq) were added under nitrogen to a 250 ml three-necked round bottom flask. After stirring at 120° C. for 2 hours, LC-MS confirmed completion. Following the standard work-up, 5.1 g of compound 8-5 (93%) was obtained.

Step 5: 60 ml of EtOH, 2 g (3.6 mmol, 1.0 eq) of compound 8-5, and 12 mL of 30% KOH were added under nitrogen into a 100 ml single-mouth flask. The mixture was refluxed for 72 hours. After concentration under vacuum and subsequent work-up, 1.9 g of compound 8-6 (91%) was purified.

Step 6: 20 ml of DCM, 1 g (1.73 mmol, 1.0 eq) of compound 8-6, EDCI (0.432 g, 2.25 mmol, 1.3 eq), DIPEA (0.893 g, 6.92 mmol, 4 eq), HOBt (0.234 g, 1.73 mmol, 1.0 eq), and methoxyammonium chloride (0.174 g, 2.08 mmol, 1.2 eq) were combined in a 100 mL single-mouth flask under nitrogen. Stirred for 16 hours at 25° C. and then processed as before, this yielded 0.43 g of compound 8-7 (41%).

Step 7: 10 ml of DCM, 0.430 g (0.71 mmol, 1 eq) of compound 8-7, and BBr₃ (0.435 g, 1.775 mmol, 2.5 eq) were mixed in a 25 ml single-mouth flask at 0° C. under nitrogen. Stirring continued for 3 hours at 0° C. until LC-MS indicated completion, proceeding directly to the next step.

Step 8: The mixture from Step 7 and 10 ml of MeOH were added to a 50 ml single-mouth flask under nitrogen. Heated to reflux for 16 hours, completion was verified by LC-MS. After concentration under vacuum and further purification steps, including the addition of 10 mL saturated NaHCO₃ solution and washing with ethyl acetate, drying over Na₂SO₄, and purification using Liquid Phase Method, 39 mg of compound 8 was obtained, marking a 16% yield. 1H NMR: (400 MHz DMSO) δ 12.25 (s, 3H), 7.57 (s, 1H), 7.52 (d, J=7.8 Hz, 1H), 6.98 (dt, J=15.0, 6.6 Hz, 3H), 6.79 (t, J=7.7 Hz, 1H), 6.73 (d, J=7.2 Hz, 1H), 6.37 (s, 1H), 5.87 (s, 1H), 3.35 (s, 1H), 2.22 (s, 3H), 2.08 (s, 3H).

LC-MS: [M+1]⁺=352.2

### Example 3. Synthesis of Compound 17

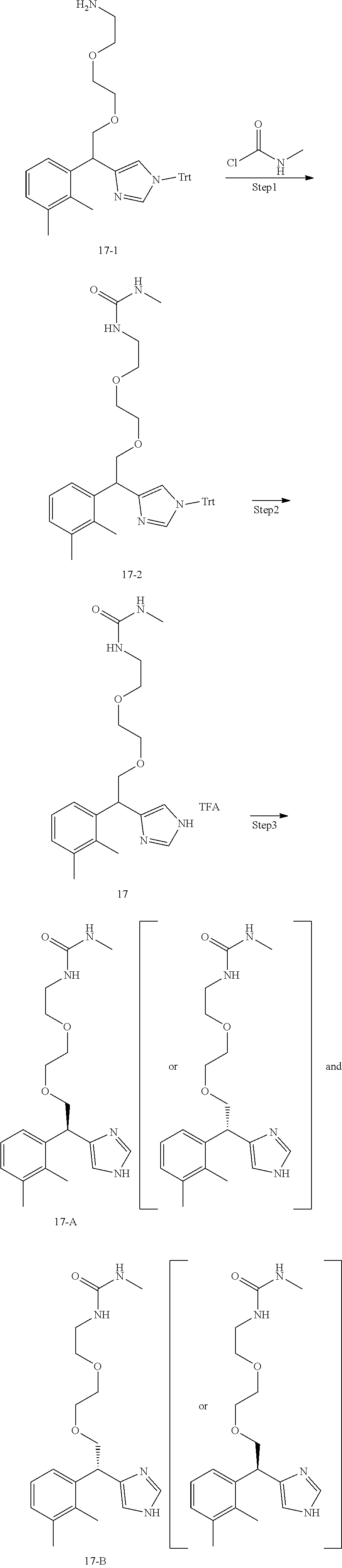

Step 1: a 10 ml single-mouth flask was initially charged under N₂ at 0° C. with 4 ml of DCM, 180 mg (0.33 mmol, 1 eq) of compound 17-1, 110 mg (1.00 mmol, 3 eq) of TEA, and 47 mg (0.5 mmol, 1.5 eq) of methylaminoformyl chloride. The reaction mixture, after being allowed to reach 25° C., was stirred for 16 hours. Once LC-MS confirmed the reaction's completion, it was concentrated under vacuum and then purified using a fast silica gel column, resulting in 150 mg of compound 17-2 at a 75% yield.

Step 2: 3 ml of DCM, 150 mg (0.25 mmol, 1 eq) of compound 17-2, and 1.5 ml of TFA were added to a 10 ml single-mouth flask under N₂ at 25° C. After stirring for 2 hours and confirmation of completion by LC-MS, the reaction mixture was vacuum concentrated and subjected to purification through a fast silica gel column, yielding 33 mg of compound 17, which corresponds to a 28% yield.

Step 3: 438 mg of compound 17 underwent separation using a chiral column, leading to the collection of compound 17-A (133 mg), which after preparative liquid chromatography with a neutral method yielded 101 mg (Yield=23.06%), and compound 17-B (133 mg), which also resulted in 101 mg after similar purification, with a yield of 23.06%.

¹H NMR: (400 MHz CDCl₃) δ 8.66 (s, 1H), 7.19-7.02 (m, 2H), 6.96 (d, J=7.2 Hz, 1H), 6.68 (s, 1H), 5.93 (s, 1H), 4.61 (dd, J=9.3, 4.1 Hz, 1H), 3.99-3.87 (m, 2H), 3.84-3.77 (m, 1H), 3.65 (ddd, J=17.3, 13.3, 7.2 Hz, 5H), 3.46-3.31 (m, 2H), 2.66 (s, 3H), 2.31 (s, 3H), 2.22 (s, 3H).

LC-MS: [M-TFA+1]+=361.3

### Example 4. Synthesis of Compound 22

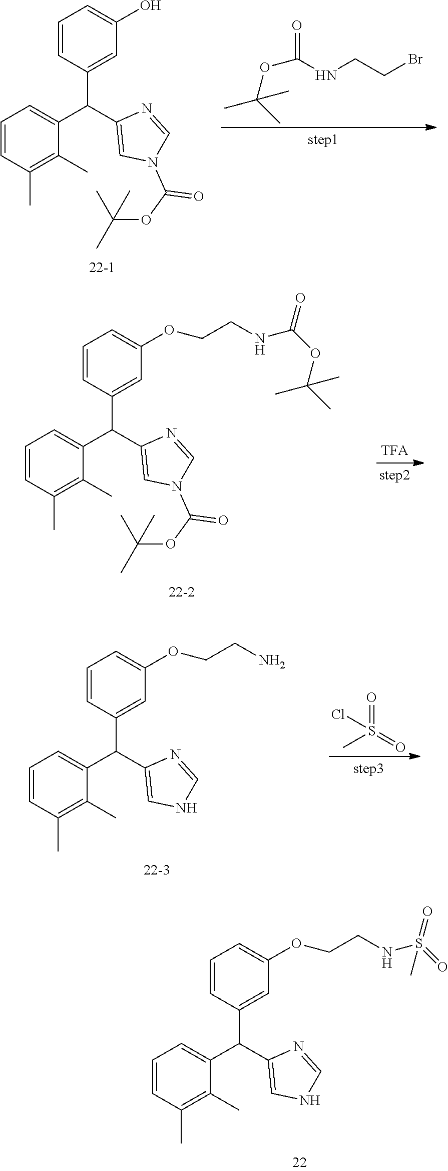

Step 1: a 50 mL three-necked flask received 10 ml ACN, 300 mg (0.793 mmol, 1 eq) of compound 22-1, 335 mg (1.58 mmol, 2 eq) of tert-butyl N-(2-bromoethyl) carbamate, and 387 mg (1.189 mmol, 1.5 eq) of Cs2CO3. The mixture was stirred at 60° C. for 12 hours. Upon completion, as indicated by LC-MS, it was poured into water and extracted with EtOAc. The organic layer was dried over Na₂SO₄, vacuum concentrated, and yielded 300 mg of compound 22-2 as a white solid, which was carried forward without further purification, yielding 72.5%.

Step 2: 300 mg of compound 22-2, 10 ml DCM, and 5 ml TFA were added to a 50 mL three-necked flask. This mixture was stirred at room temperature (25° C.) for 12 hours. LC-MS confirmed completion; the mixture was then diluted with water, adjusted to pH 10, and extracted with DCM. The organic phase was dried over Na₂SO₄, vacuum concentrated, and the resulting residue was column chromatographed on silica gel to obtain 85 mg of compound 22-3 as a yellow solid, with a yield of 45.9%.

Step 3: a 10 ml three-necked flask was charged with 5 ml DMF, 75 mg (0.233 mmol, 1 eq) of compound 22-3, 75 mg (0.583 mmol, 2.5 eq) of DIPEA, and 29 mg (0.257 mmol, 1.1 eq) of methanesulfonyl chloride. Stirring continued at 25° C. for 2 hours until LC-MS analysis confirmed the reaction's completion. The mixture was then diluted with water, extracted with EA, and the organic phase was dried over Na₂SO₄ and vacuum concentrated. Purification by silica gel column chromatography yielded 14 mg of compound 22 as a white solid, achieving a 13.2% yield.

Overall yield=4.4%.

LC-MS: [M+1]+=400.2

¹H NMR (400 MHZ, DMSO) δ 13.46 (s, 1H), 8.27 (s, 1H), 7.24 (dd, J=13.6, 5.7 Hz, 2H), 7.04 (d, J=11.2 Hz, 2H), 6.93-6.49 (m, 5H), 5.68 (s, 1H), 3.96 (s, 2H), 3.29 (d, J=4.5 Hz, 2H), 2.91 (s, 3H), 2.24 (s, 3H), 2.12 (s, 3H).

### Example 5. Synthesis of Compound 27

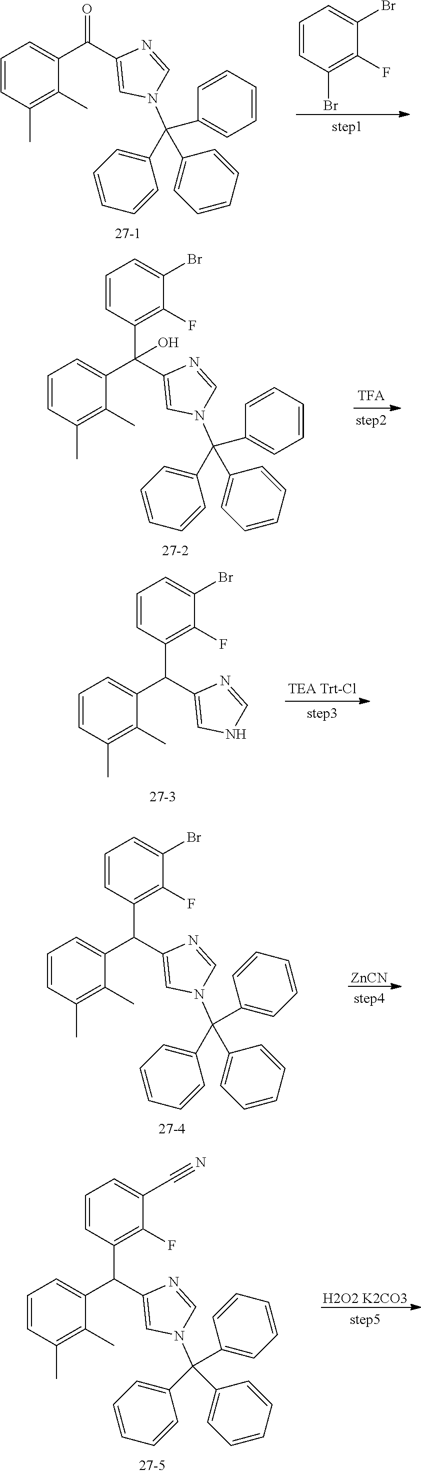

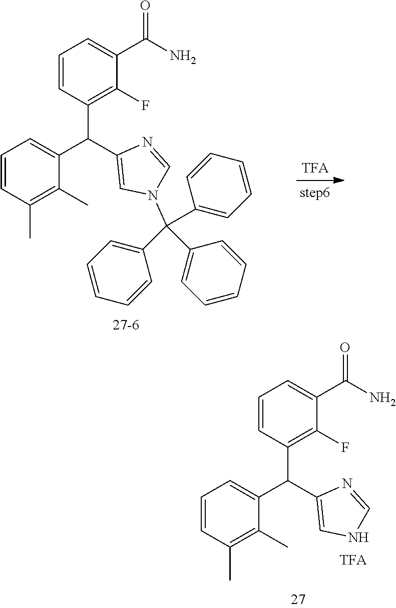 [SMILES: Cc1cccc(C(c2cn(C(c3ccccc3)(c3ccccc3)c3ccccc3)cn2)c2cccc(C(N)=O)c2F)c1C ; Cc1cccc(C(c2c[nH]cn2)c2cccc(C(N)=O)c2F)c1C]

Step 1: In a 50 mL reaction flask, 10 mL of THF and 860 mg (3.39 mmol, 1.5 eq) of 1,3-dibromo-2-fluorobenzene were combined and cooled to −65° C. Next, 1.4 mL (3.39 mmol, 1.5 eq) of n-butyllithium was added. The mixture was stirred at this temperature for 2 hours before adding 1 g (2.26 mmol, 1 eq) of compound 27-1 and another 10 mL of THF. It was stirred for an additional 30 minutes at −65° C., then allowed to warm to room temperature over 16 hours. Completion was verified by LC-MS, and 20 mL of saturated ammonium chloride was added. The organic phase was then separated, concentrated under vacuum, and purified via column chromatography, yielding 600 mg of compound 27-2 with a yield of 43%.

Step 2: A 50 mL three-necked flask was charged with 600 mg (0.971 mmol, 1 eq) of compound 27-2, 1.1 g (9.71 mmol, 10 eq) of triethylsilane, and 1.1 g (9.71 mmol, 10 eq) of TFA. The mixture was stirred at 25° C. for 1 hour. Upon completion, confirmed by LC-MS, it was poured into water, adjusted to pH=10, extracted with EA, dried over Na₂SO4, and concentrated under vacuum. Purification via column chromatography on silica gel led to the isolation of 170 mg of compound 27-3 as a yellow solid, yielding 48.7%.

Step 3: Into a 25 mL reaction flask, 10 ml of DMF, 170 mg (0.473 mmol, 1 eq) of compound 27-3, 158 g (0.568 mmol, 1.2 eq) of triphenylmethyl chloride, and 96 mg (0.946 mmol, 2 eq) of TEA were added. The mixture was stirred at 25° C. for 12 hours until LC-MS indicated the reaction had completed. After pouring into water, extracting with EA, drying over Na₂SO₄, and concentrating, the crude was purified by column chromatography, yielding 220 mg of compound 27-4 with a 77.3% yield.

Step 4: A 25 mL reaction flask was prepared with 10 ml DMF, 170 mg (0.283 mmol, 1 eq) of compound 27-4, 100 mg (0.848 mmol, 3 eq) of ZnCN, and 98 mg (0.0848 mmol, 0.3 eq) of tetrakis(triphenylphosphine) palladium. Stirring was conducted at 150° C. for 30 minutes in a microwave. After completion, confirmed by LC-MS, the mixture was worked up and purified by column chromatography to yield 130 mg of compound 27-5, an 84% yield.

Step 5: To a 25 mL reaction flask, 10 mL of DMSO and 110 mg (0.201 mmol, 1 eq) of compound 27-5 were added and cooled to 0° C. Then, 3 ml of 30% H₂O₂ was added, and the mixture was stirred at 0° C. for 1 hour. Following LC-MS confirmation of completion, water was added, and the organic phase was separated and concentrated under vacuum. Column chromatography purification yielded 100 mg of compound 27-6, an 88.1% yield.

Step 6: In a 25 mL three-port flask, 10 ml of DCM and 110 mg of compound 27-6 were combined, and 5 ml of TFA was added at 0° C. The mixture was allowed to reach room temperature naturally and stirred for 2 hours. Completion was indicated by LC-MS. The mixture was then concentrated under reduced pressure, and the residue was purified by TLC to obtain 25 mg of compound 27 as a white solid, with a yield of 29.4%.

Overall yield=3.5%.

LC-MS: [M-C₂HF₃O₂+1]+=324.2

¹H NMR (400 MHZ, DMSO) δ 14.36 (s, 5H), 8.97 (s, 3H), 7.77 (s, 3H), 7.64 (s, 3H), 7.60 (t, J=6.7 Hz, 3H), 7.24 (t, J=7.7 Hz, 3H), 7.15 (d, J=7.3 Hz, 3H), 7.13-6.98 (m, 9H), 6.71 (d, J=7.5 Hz, 3H), 6.01 (s, 3H), 2.27 (s, 9H), 2.12 (s, 9H)

### Example 6. Synthesis of Compound 28

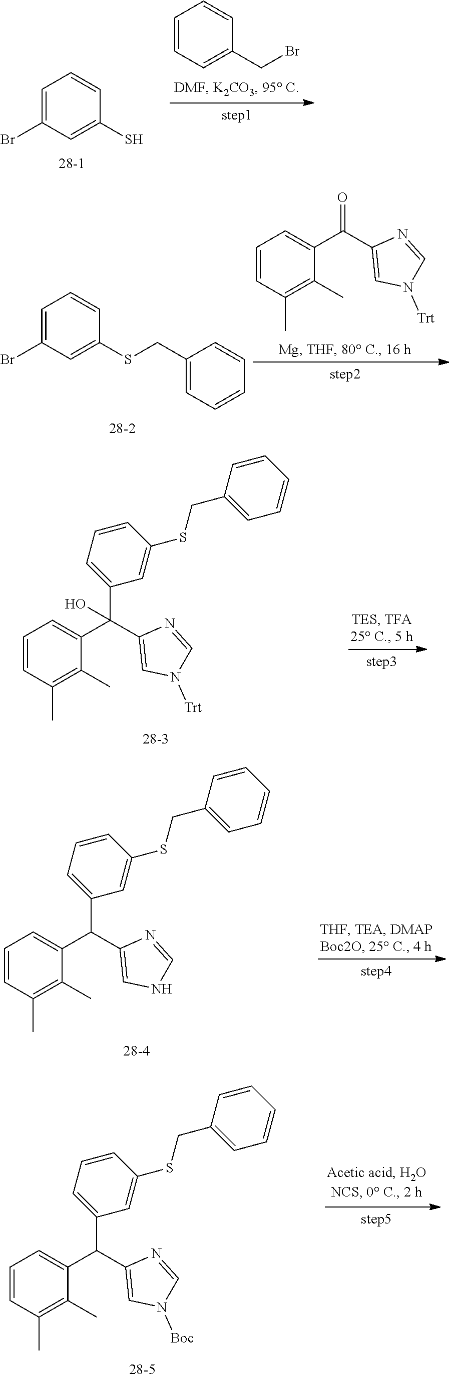

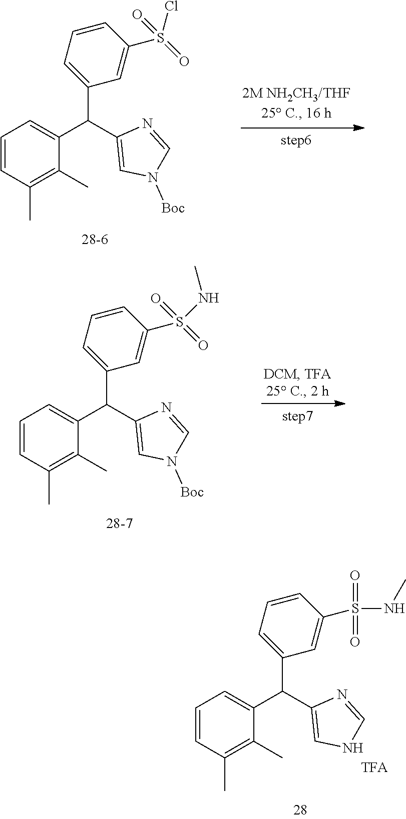 [SMILES: Cc1cccc(C(c2cccc(S(=O)(=O)Cl)c2)c2cn(C(=O)OC(C)(C)C)cn2)c1C ; CNS(=O)(=O)c1cccc(C(c2cn(C(=O)OC(C)(C)C)cn2)c2cccc(C)c2C)c1 ; CNS(=O)(=O)c1cccc(C(c2c[nH]cn2)c2cccc(C)c2C)c1]

Step 1: In a 500 ml reaction flask, 220 ml DMF, 24.5 g (0.13 mol, 1 eq) of compound 28-1, 26.6 g (0.16 mol, 1.2 eq) of benzyl bromide, and 21.5 g (1.2 mol, 1.2 eq) of K2CO₃ were combined. The mixture was heated to 95° C. for 16 hours. GC-MS confirmed the reaction's completion. After filtration and concentration, column chromatography purification yielded 32 g of compound 28-2 with an 88.5% yield.

Step 2: Into a 25 ml reaction flask, 5 ml THF, 185 mg (7.6 mmol, 2.1 eq) of magnesium chips, and 2 g (7.2 mmol, 2.0 eq) of compound 28-2 were added. The mixture was stirred at 65° C. for 1 hour before cooling to room temperature for the next step. A 50 mL reaction flask received 20 mL THF and 1.59 g (3.6 mmol, 1.0 eq) of (2,3-Dimethylphenyl)-[1-(trityl)-1H-imidazol-4-yl]methanone. The previously prepared Grignard reagent was added, and the reaction was heated to 80° C. for 16 hours. Completion was verified by LC-MS. After quenching with 10 mL water, extraction with EA, and drying with Na₂SO₄, the mixture was concentrated. Column chromatography purification yielded 1.6 g of compound 28-3, a 69.2% yield.

Step 3: A 100 mL reaction flask was charged with 14 mL DCM, 1.4 g (2.18 mmol, 1.0 eq) of compound 28-3, and 2.53 g (21.8 mmol, 10 eq) of TES. After cooling to 0° C., 2.48 g (21.8 mmol, 10 eq) of TFA was added. The mixture was warmed to 25° C. for 5 hours, then concentrated under vacuum after LC-MS confirmed completion. The residue was column chromatographed to yield 500 mg of compound 28-4, a 68.4% yield.

Step 4: In a 5 mL reaction flask, 2 mL THF, 100 mg (0.29 mmol, 1.0 eq) of compound 28-4, 7 mg (0.06 mmol, 0.2 eq) of DMAP, 94 mg (0.43 mmol, 1.5 eq) of Boc₂O, and 44 mg (0.43 mmol, 1.5 eq) of TEA were mixed. The reaction was held at 25° C. for 4 hours, confirmed by LC-MS. After vacuum concentration, the residue was purified by column chromatography, yielding 120 mg of compound 28-5, an 85.5% yield.

Step 5: A 10 mL reaction flask was loaded with 1.5 mL acetic acid, 0.5 ml water, and 120 mg (0.25 mmol, 1.0 eq) of compound 28-5. After chilling to 0° C., 165 mg (1.24 mmol, 5 eq) of NCS was added. The mixture was stirred at 0° C. for 2 hours until LC-MS confirmed completion, then moved to the next step without purification. The yield was recorded as 100%.

Step 6: To a 50 mL reaction flask, 10 mL of 2M NH₂CH₃/THF was added and cooled to 0° C. before introducing the crude compound 28-6. Stirring proceeded at 25° C. for 16 hours, as evidenced by LC-MS. After concentration under vacuum, the mixture was purified by column chromatography to yield 40 mg of compound 28-7, a 40% yield.

Step 7: In a 5 mL reaction flask, 1 mL DCM and 40 mg (0.088 mmol, 1.0 eq) of compound 28-7 were combined. The mixture was cooled to 0° C. before adding 0.5 ml TFA, then allowed to warm to 25° C. for 2 hours, completion shown by LC-MS. The concentrated mixture was purified through prep-HPLC to obtain 14 mg of compound 28, with a yield of 44.8%.

Overall yield=6.4%.

LC-MS: [M+1-TFA]⁺=356.1

¹H NMR (400 MHZ, DMSO) δ 14.32 (s, 2H), 9.01 (s, 1H), 7.71 (d, J=7.8 Hz, 1H), 7.64-7.58 (m, 2H), 7.46 (dt, J=17.1, 6.3 Hz, 2H), 7.12 (dt, J=15.1, 7.4 Hz, 2H), 6.95 (s, 1H), 6.68 (d, J=7.4 Hz, 1H), 2.37 (d, J=4.9 Hz, 3H), 2.26 (s, 3H), 2.12 (s, 3H).

### Example 7. Synthesis of Compound 31

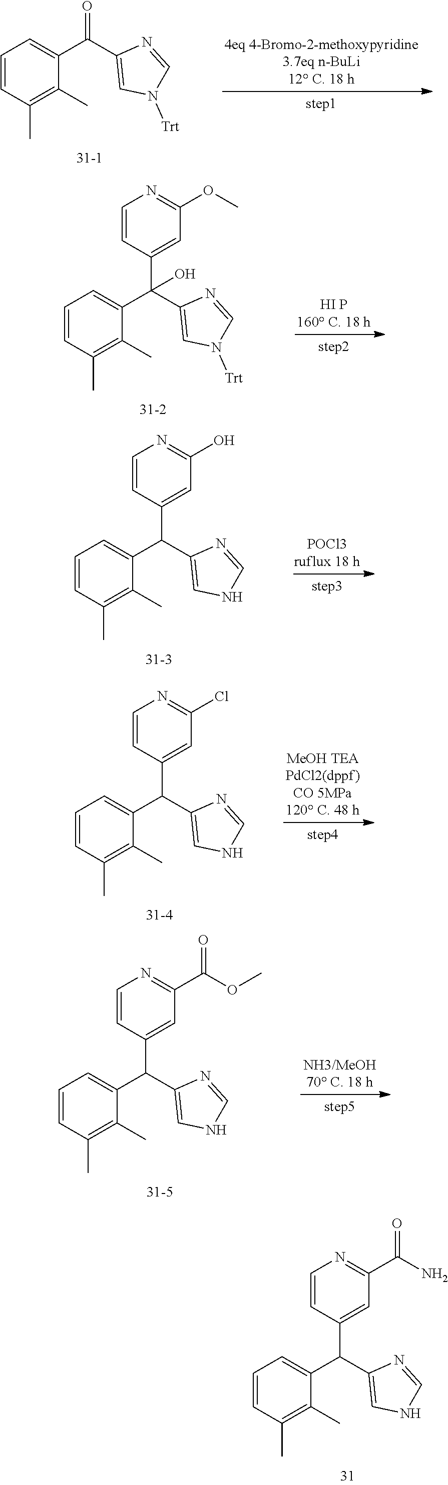

Step 1: Into a 250 mL three-port reaction bottle, 120 ml of THF, 5.08 g (27.15 mmol, 4.0 eq) of 4-bromo-2-methoxypyridine, and 10 mL of 2.5N n-butyl lithium in n-hexane (25.1 mmol, 3.7 eq) were added dropwise at −65° C. The solution was maintained at −65° C. for 1 hour before adding 3 g (6.79 mmol, 1 eq) of compound 31-1. After another 0.5 hours at −65° C., the reaction was left to proceed overnight at room temperature. Completion was verified by LC-MS. The reaction was quenched with 100 ml of saturated ammonium chloride, and the organic layer was separated and concentrated. The residue was mixed with 50 mL DCM, stirred for 5 minutes, filtered, and dried with an infrared lamp to yield 2.88 g of compound 31-2 (77% yield).

Step 2: A 50 ml closed tank received 20 ml of 57 wt. % HI, 2.35 g (4.599 mmol, 1.0 eq) of compound 31-2, and 1.43 g (45.99 mmol, 10 eq) of red phosphorus. Stirred at 160° C. overnight and checked by LC-MS for completion, the mixture was cooled to room temperature and concentrated to yield 5 g of crude compound 31-3 (100% yield).

Step 3: Compound 31-3 (1 g, crude) and 15 mL of POCl₃ were added to a 5 mL reaction flask. After refluxing overnight and verifying completion with LC-MS, the mixture was cooled, concentrated under vacuum, and the residue was neutralized to pH=8 with saturated sodium bicarbonate. Extraction with 40 mL EA (three times), drying with anhydrous sodium sulfate, and concentration provided 174 mg of compound 31-4 via column chromatography (30% yield). Step 4: Into a 200 mL high-pressure reactor, 174 mg (0.586 mmol, 1 eq) of compound 31-4, 10 mL of MeOH, 296 mg (2.93 mmol, 5 eq) of TEA, and 48 mg (0.0586 mmol, 0.1 eq) of PdCl₂ (dppf) were introduced. The reaction, under 5 MPa of carbon monoxide at 120° C. for 48 hours, left 5% of the starting material, as shown by LC-MS. After filtration and concentration, 270 mg of compound 31-5 was isolated by column chromatography (100% yield).

Step 5: A 50 mL closed tank was charged with 100 mg (0.312 mmol, 1 eq) of compound 31-5 and 5 mL of MeOH/NH₃ (15M/L). The mixture was stirred at 68° C. overnight, cooled to room temperature, concentrated under vacuum, and then purified to obtain 10 mg of compound 31 through pre-HPLC (10% yield).

LC-MS: [M+1]⁺=307.2

¹H NMR (400 MHZ, DMSO) δ 12.53 (s, 1H), 8.52 (d, J=4.7 Hz, 1H), 8.10 (s, 1H), 7.86 (s, 1H), 7.79 (s, 1H), 7.63 (s, 1H), 7.35 (d, J=3.8 Hz, 1H), 7.10-7.00 (m, 2H), 6.81 (d, J=7.2 Hz, 1H), 6.66 (s, 1H), 5.80 (s, 1H), 2.24 (s, 3H), 2.11 (s, 3H).

### Example 8. Synthesis of Compound 32

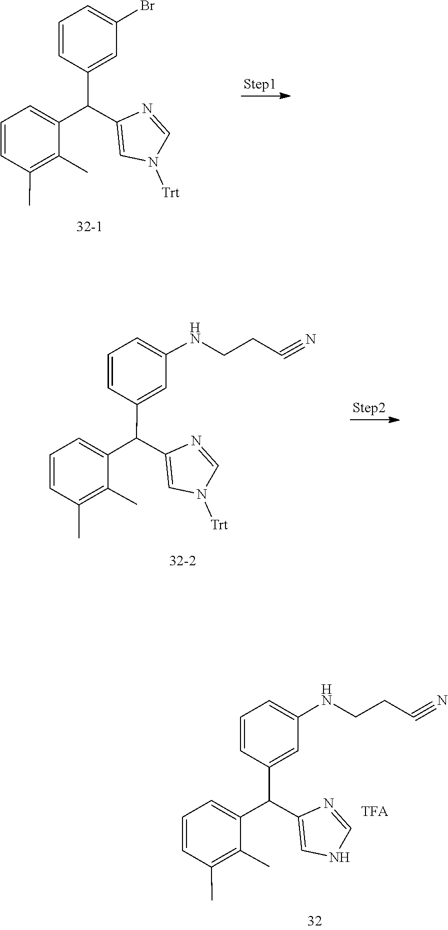

Step 1: Into the bottom of a 50 mL single-mouth flask, 300 mg (0.51 mmol, 1 eq) of compound 32-1, 108 mg (1.54 mmol, 3 eq) of 2-cyanoethylamine, 213 mg (1.54 mmol, 3 eq) of K₂CO₃, 47 mg (0.051 mmol, 0.1 eq) of Pd₂(dba)₃, and 55 mg (0.10 mmol, 0.2 eq) of brettphos were added. The mixture was stirred under nitrogen at 120° C. for 1 hour. The completion of the reaction was indicated by TLC. The mixture was then transferred into 100 ml of water and extracted three times with 50 mL of ethyl acetate. After washing the organic layer with 50 mL of brine, it was dried over Na₂SO₄ and concentrated under vacuum to yield 1 g of a crude yellow oil. This was purified via column chromatography (Petroleum Ether:Ethyl Acetate=1:0 to 1:1) to obtain 216 mg of compound 32-2 as a yellow powder, resulting in a yield of 73.36%.

Step 2: A 50 mL single-mouth flask was charged with 200 mg (0.35 mmol, 1 eq) of compound 32-2, 5 mL of dichloromethane, and 1 mL of trifluoroacetic acid. The mixture was stirred at room temperature for 1 hour, with TLC confirming the reaction's completion. The solution was then vacuum-concentrated, and the crude product was subjected to column chromatography (Dichloromethane:Methanol=1:0 to 90:10) to produce 120 mg of compound 32. Further purification by preparative HPLC yielded 93 mg of compound 32 as a yellow powder, with an overall yield of 59.92%.

Overall yield=43.96%.

LC-MS: [M+1]+=331.2.

1H NMR (400 MHZ, DMSO) δ 14.18 (s, 2H), 8.91 (s, 1H), 7.15-7.01 (m, 3H), 6.90 (s, 1H), 6.70 (d, J=7.2 Hz, 1H), 6.52 (d, J=8.0 Hz, 1H), 6.41 (s, 1H), 6.36 (d, J=7.6 Hz, 1H), 5.97 (s, 1H), 5.65 (s, 1H), 3.49-3.13 (m, 5H), 2.67 (t, J=6.5 Hz, 2H), 2.25 (s, 3H), 2.13 (s, 3H). Y=59.92%. Total yield=43.96%.

### Example 9. Synthesis of Compound 58

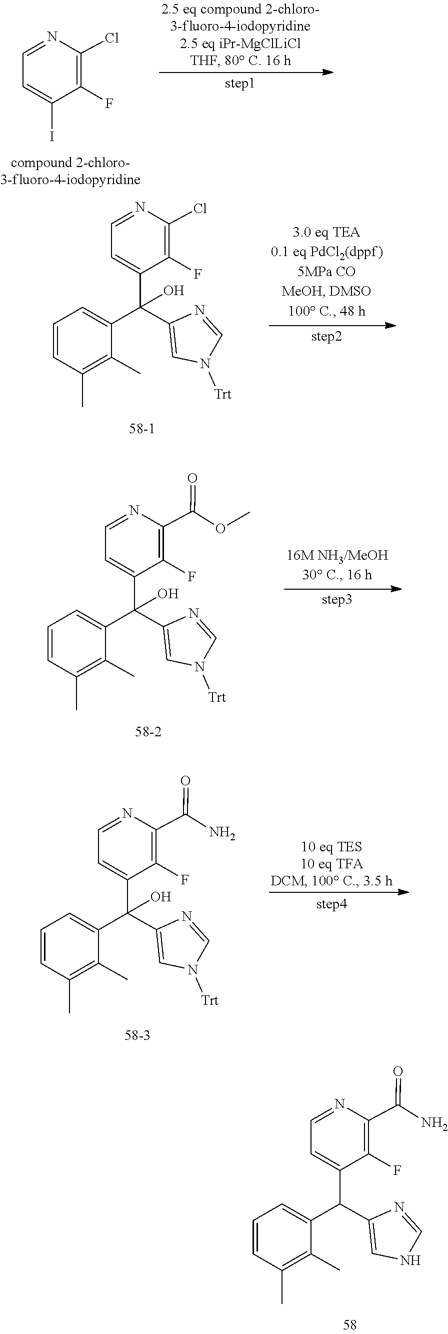

Step 1: In a 100 mL reaction flask, 40 mL of THF and 4 g (15.5 mmol, 2.5 eq) of 2-chloro-3-fluoro-4-iodopyridine were added. After cooling the mixture to 0° C., 12 mL (15.5 mmol, 2.5 eq) of iPr-MgClLiCl was introduced. The reaction was stirred at 0° C. for 3 hours, then 1.16 g (2.63 mmol, 1.0 eq) of 2,3-dimethylphenyl) [1-(trityl)-1H-imidazol-4-yl]methanone was added and the reaction was stirred at 80° C. for 16 hours. Following completion, confirmed by LC-MS, the reaction was quenched with 40 mL water and extracted with EA. The organic layer was dried over Na₂SO₄ and concentrated under vacuum. The residue was purified by column chromatography to yield 2.6 g of compound 58-1, achieving a 29.3% yield.

Step 2: To a 200 mL high-pressure reactor, 1 g (1.74 mmol, 1.0 eq) of 58-1, 40 mL of MeOH, 40 mL of DMSO, 530 mg (5.24 mmol, 3.0 eq) of TEA, and 148 mg (0.17 mmol, 0.1 eq) of PdCl₂ (dppf) were added. The mixture was reacted with carbon monoxide at 5 MPa and 100° C. for 48 hours, with LC-MS indicating 5% remaining raw material. After concentration, the residue was purified by column chromatography to yield 53 mg of compound 58-2, a 51.2% yield.

Step 3: A 50 mL sealed tube received 25 mL of 16M NH₃/MeOH and 530 mg (0.89 mol, 1.0 eq) of compound 58-2. Stirred at 30° C. for 16 hours until LC-MS confirmed completion, the residue was then purified by column chromatography to yield 360 mg of compound 58-3, a 67.8% yield. Step 4: Into a 10 mL reaction flask, 3 mL DCM, 100 mg (0.17 mmol, 1.0 eq) of compound 58-3, and 195 mg (1.7 mmol, 10 eq) of TES were added. After cooling to 0° C., 191 mg (1.7 mmol, 10 eq) of TFA was introduced. The reaction was then warmed to 100° C. for 3.5 hours. LC-MS showed completion, and after concentration under vacuum, the residue was purified by prep-HPLC to yield 16 mg of compound 58, with a 28.8% yield.

LC-MS: [M+1]⁺=325.2

¹H NMR (400 MHZ, CD₃OD) δ 8.36 (d, J=4.8 Hz, 1H), 7.73 (d, J=0.9 Hz, 1H), 7.13 (dt, J=22.7, 10.1 Hz, 2H), 7.03 (t, J=7.6 Hz, 1H), 6.72 (d, J=7.7 Hz, 1H), 6.53 (s, 1H), 6.05 (s, 1H), 2.32 (s, 3H), 2.21 (s, 3H).

### Example 10. Synthesis of Compound 60

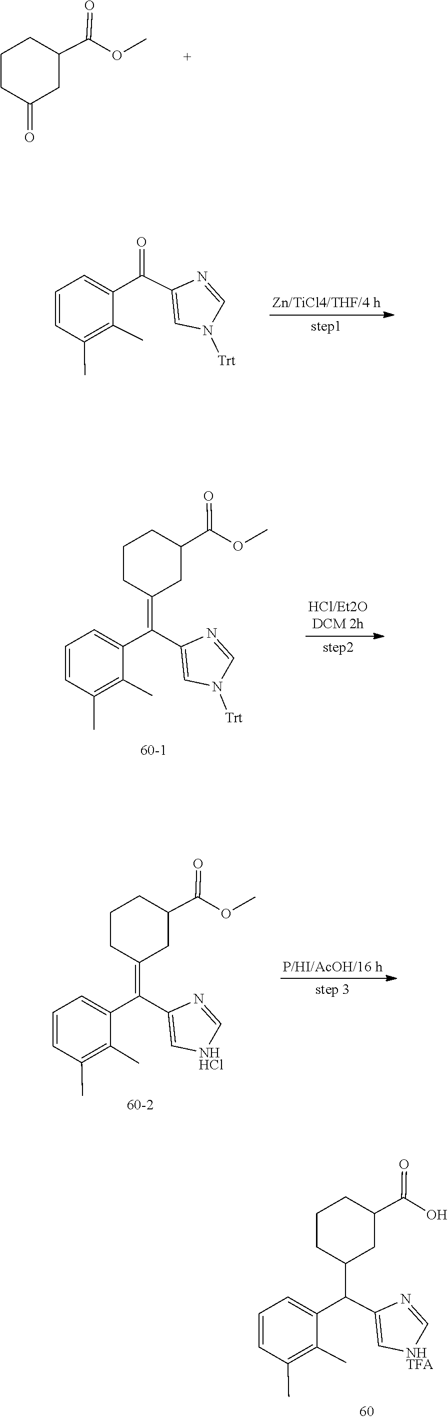

Step 1: Into a 100 mL three-necked flask, 29 mL of THF and 2.9 g (43.9 mmol, 7.5 eq) of zinc were added. This mixture was cooled to −10° C. under a nitrogen atmosphere while stirring. Then, 4.1 g (21.6 mmol, 3.7 eq) of titanium tetrachloride was added dropwise at −10° C. The reaction mixture was stirred at 70° C. for 16 hours, followed by the addition of 950 mg (6.08 mol, 1.04 eq) of methyl 3-oxocyclohexanecarboxylate and 2.6 g (5.85 mol, 1 eq) of (2,3-dimethylphenyl) (1-trityl-4-imidazolyl) methanone. Stirring continued for 4 hours at 80° C. Completion was confirmed by LC-MS. The reaction was then diluted with 100 ml of water and 100 mL of EA, filtered, and the filtrate was extracted with EA. After washing with brine and drying over Na₂SO₄, the organic layers were concentrated under reduced pressure to yield 950 mg of compound 60-1 as crude, with a 50% yield.

Step 2: To a 25 mL three-necked flask, 10 mL of DCM and 0.5 g (1.54 mmol, 1 eq) of compound 60-1, along with 10 mL of HCl/Et2O, were added. The mixture was stirred at room temperature for 3 hours. LC-MS indicated the reaction was complete. Concentrating under reduced pressure yielded 400 mg of compound 60-2 as crude, with a 100% yield.

Step 3: A 250 mL three-necked flask received 1.2 mL of AcOH, 0.9 mL of hydriodic acid (55%-58%), 50 mg (0.15 mmol, 1.0 eq) of compound 60-2, and 167 mg (5.4 mmol, 35 eq) of phosphorus. The reaction mixture was stirred at 100° C. for 16 hours. Completion was confirmed by LC-MS. The mixture was then added to water, adjusted to pH=7, and extracted with EA. The combined organic layers were washed with brine, dried over Na₂SO₄, filtered, and concentrated under reduced pressure to obtain 70 mg of crude. Purification by liquid chromatography yielded 6 mg of compound 60, with a 10% yield.

Overall yield: 5%

LC-MS: [M-TFA-1]−=311.2

1H NMR (400 MHZ, DMSO) δ 14.20 (s, 2H), 12.02 (s, 1H), 9.02-8.89 (m, 1H), 7.75-7.56 (m, 1H), 7.13 (ddt, J=22.9, 16.8, 9.0 Hz, 3H), 4.26-4.06 (m, 1H), 2.28-2.08 (m, 8H), 1.91-0.73 (m, 8H).

### Example 11. Synthesis of Compound 61

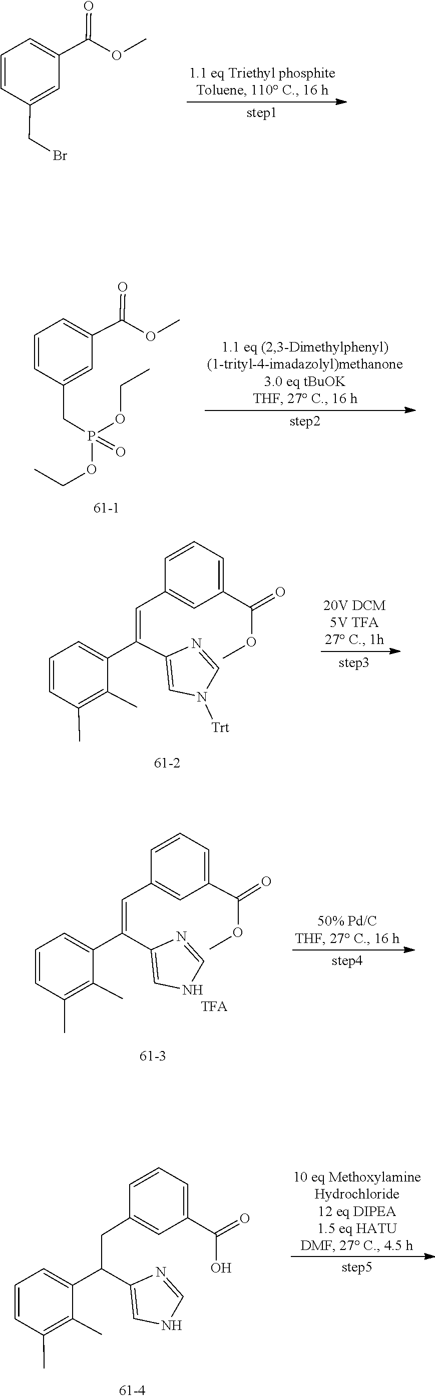

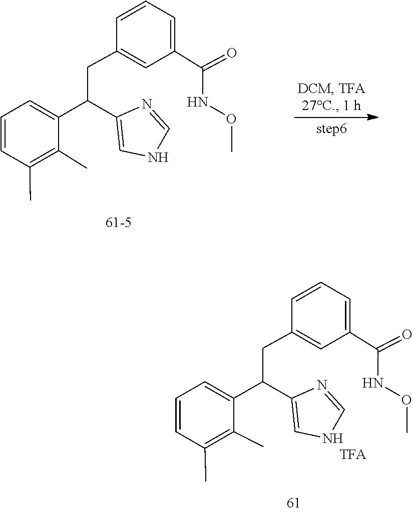 [SMILES: CONC(=O)c1cccc(CC(c2c[nH]cn2)c2cccc(C)c2C)c1 ; CONC(=O)c1cccc(CC(c2c[nH]cn2)c2cccc(C)c2C)c1]

Step 1: A 50 mL reaction flask received 20 mL toluene, 3.8 g (16.7 mmol, 1.0 eq) of methyl 3-(bromomethyl)benzoate, and 3.04 g (18.3 mmol, 1.1 eq) of triethyl phosphite. The mixture was stirred at 110° C. for 16 hours. Upon completion, confirmed by LC-MS, the solution was concentrated. The residue underwent column chromatography, yielding 5.3 g of compound 61-1 with a 99% yield.

Step 2: Into a 100 mL reaction flask, 40 mL THF, 2 g (7.0 mmol, 1.0 eq) of compound 61-1, and 3.4 g (7.7 mmol, 1.1 eq) of (2,3-dimethylphenyl) (1-trityl-4-imidazolyl) methanone were combined and cooled to 0° C. before adding 2.35 g (21 mmol, 3.0 eq) of potassium tert-butoxide. After stirring at 27° C. for 16 hours and confirmation of completion by LC-MS, the solution was concentrated and purified by column chromatography to yield 1.03 g of compound 61-2, a 26.3% yield.

Step 3: A 25 mL reaction flask was charged with 10 mL DCM and 500 mg of compound 61-2, followed by the addition of 2.5 mL TFA. The reaction mixture was stirred at 27° C. for 1 hour. LC-MS indicated completion, and after concentration, the residue was purified by column chromatography to yield 240 mg of compound 61-3, an 84.5% yield.

Step 4: In a 10 mL reaction flask, 3 mL THF, 240 mg of compound 61-3, and 120 mg (50%) of Pd/C were added. Stirred at 27° C. for 16 hours and confirmed by LC-MS, the solution was filtered. The organic phase was concentrated, and the residue was purified by column chromatography to yield 150 mg of compound 61-4, a 62.1% yield.

Step 5: To a 10 mL reaction flask, 3 mL DMF, 80 mg (0.25 mmol, 1.0 eq) of compound 61-4, 209 mg (2.5 mmol, 10 eq) of methoxyammonium chloride, cooled to 0° C., then 386 mg (3 mmol, 12 eq) of DIPEA and 142 mg (0.37 mmol, 1.5 eq) of HATU were added. Stirred at 27° C. for 4.5 hours, LC-MS showed 40% remaining raw material. The mixture was concentrated under vacuum to yield 160 mg of compound 61-5, achieving a 100% yield.

Step 6: A 10 mL reaction flask was prepared with 1 mL DCM and 160 mg of compound 61-5, and 0.5 mL TFA was added. Stirred at 27° C. for 1 hour, completion was confirmed by LC-MS. After concentration under vacuum, the residue was purified by prep-HPLC to yield 25 mg of compound 61, a 15.3% yield. Overall yield: 2.1%.

LC-MS: [M-C₂HF₃O₂+1]+=350.2

1H NMR (400 MHZ, DMSO) δ 14.20 (s, 2H), 11.69 (s, 1H), 8.96 (d, J=0.9 Hz, 1H), 7.61 (d, J=4.6 Hz, 2H), 7.55-7.48 (m, 1H), 7.31 (t, J=6.4 Hz, 2H), 7.07 (dt, J=12.8, 4.6 Hz, 3H), 4.74 (t, J=7.9 Hz, 1H), 3.70 (s, 3H), 3.39 (d, J=8.5 Hz, 1H), 3.19 (dd, J=13.8, 7.3 Hz, 1H), 2.21 (s, 3H), 2.12 (s, 3H).

### Example 12. Synthesis of Compound 139

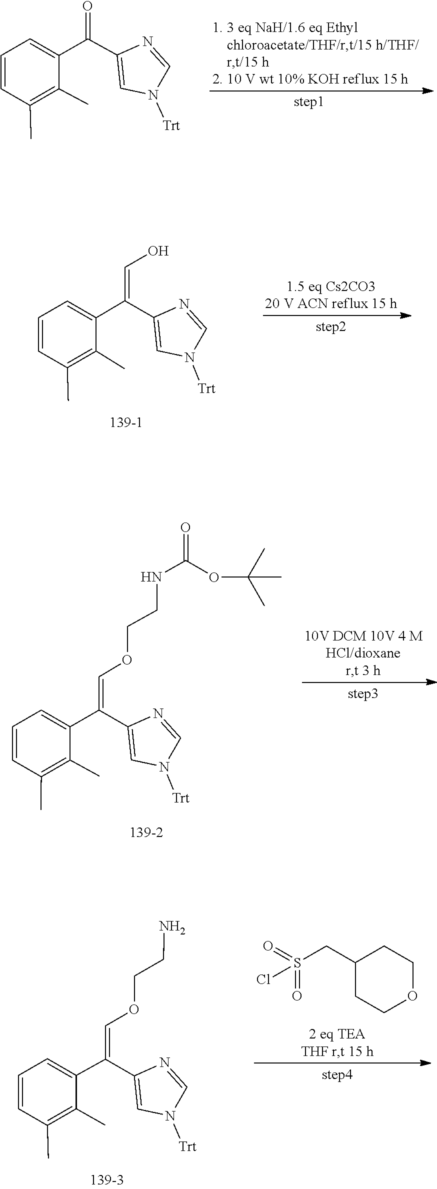

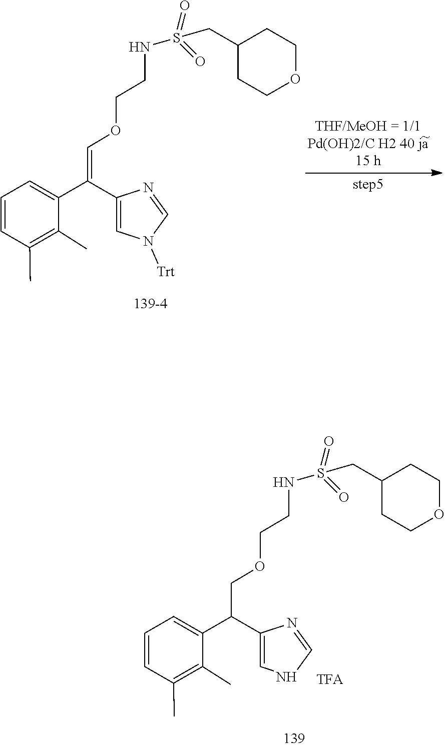

Step 1: In a 100 ml single-mouth flask, 50 ml of THF, 5 g (11.29 mmol, 1.0 eq) of (2,3-dimethylphenyl) (1-trityl-4-imidazolyl) methanone, 2.3 g (18.1 mmol, 1.6 eq) of ethyl chloroacetate, and 1.35 g (33.9 mmol, 3 eq, 60% wt) of NaH were combined under a nitrogen atmosphere. The mixture was stirred at 25° C. for 16 hours, confirmed complete by LC-MS, and concentrated under vacuum. After adding 50 ml of 10% KOH, it was stirred for another 16 hours at 100° C., then worked up and purified via a fast silica gel column to yield 3.4 g of 139-1, with a 65.9% yield.

Step 2: A 100 mL three-necked flask received 60 ml of ACN, 3.2 g (7.01 mmol, 1 eq) of 139-1, 3.14 g (14.02 mmol, 2 eq) of CAS 39684-80-5, and 3.42 g (10.51 mmol, 1.5 eq) of Cs2CO₃. Stirred at 60° C. for 12 hours and confirmed complete by LC-MS, the reaction was worked up and purified to give 930 mg of 139-2, a 22.1% yield.

Step 3: To a 25 mL three-necked flask was added 300 mg of 139-2, 3 ml of DCM, and 3 ml of 4M HCl in dioxane. After stirring at 25° C. for 3 hours and confirmation of completion by LC-MS, the reaction was neutralized to pH=10, extracted, and purified to yield 130 mg of 139-3 as a white solid, a 52% yield.

Step 4: A 25 mL three-necked flask was charged with 5 ml of THF, 130 mg (0.260 mmol, 1 eq) of 139-3, 40 mg (0.390 mmol, 1.5 eq) of TEA, and 57 mg (0.286 mmol, 1.1 eq) of (Tetrahydro-2H-pyran-4-yl) methanesulfonyl chloride (CAS 264608-29-9). Stirred at 25° C. for 18 hours and verified complete by LC-MS, the mixture was worked up and purified to give 80 mg of 139-4 as a white solid, yielding 46.5%.

Step 5:139-4 (80 mg) was combined with Pd(OH)₂/C (80 mg), 5 mL of methanol, and 5 mL of THF, stirred at 40° C. for 18 hours under a hydrogen atmosphere. The catalyst was filtered off, and the filtrate was concentrated, mixed with 10 mL of DCM and 5 mL of TFA, stirred for ten minutes, and dried. The residue was purified by preparative HPLC to yield 30 mg of 139 as a white solid, with a 46.3% yield.

Overall yield=1.63%.

LC-MS: [M-C₂HF₃O₂+1]+=422.2

1H NMR (400 MHz, DMSO) δ 14.20 (s, 2H), 9.00 (s, 1H), 7.58 (s, 1H), 7.21-6.95 (m, 3H), 6.86 (d, J=7.5 Hz, 1H), 4.65 (dd, J=8.2, 5.3 Hz, 1H), 3.90 (t, J=9.3 Hz, 1H), 3.78 (dd, J=16.0, 8.9 Hz, 3H), 3.50 (d, J=5.1 Hz, 2H), 3.28 (t, J=11.2 Hz, 2H), 3.09 (dd, J=11.1, 5.4 Hz, 2H), 2.92 (d, J=6.3 Hz, 2H), 2.28 (s, 6H), 2.00 (d, J=4.4 Hz, 1H), 1.72 (d, J=12.9 Hz, 2H), 1.28 (qd, J=12.2, 4.1 Hz, 2H).

### Example 13. Synthesis of Compound 156

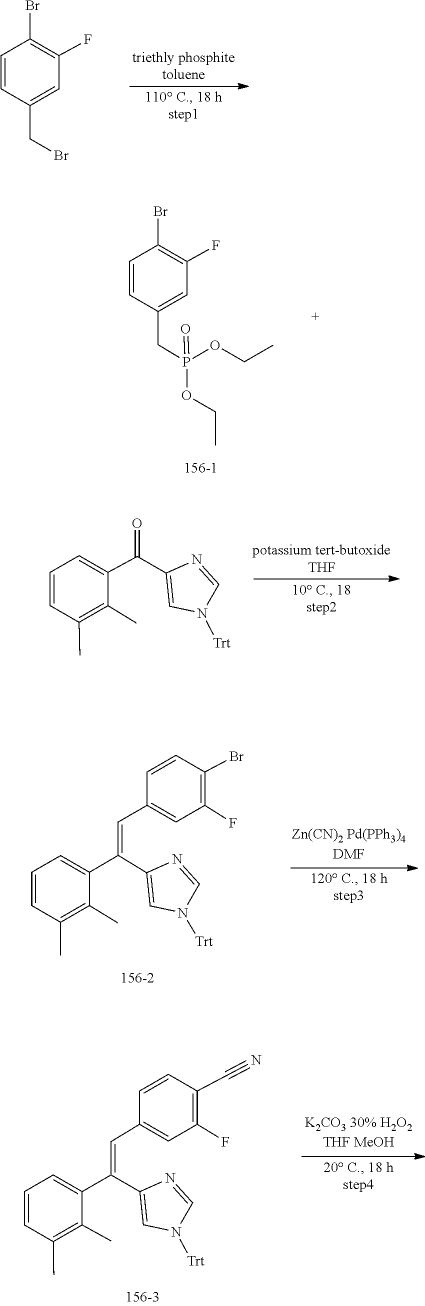

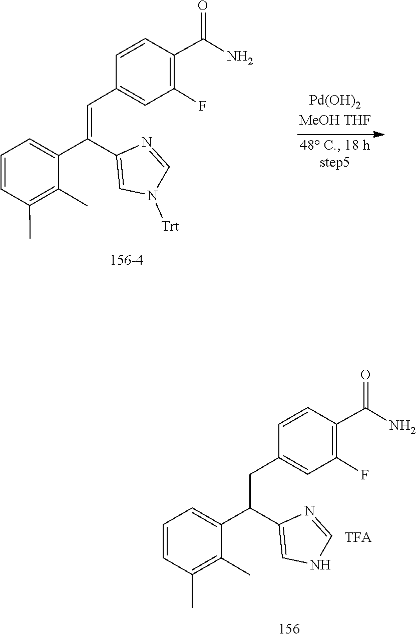

Step 1: A 50 mL reaction flask was charged with 25 mL of toluene, 5 g (0.0188 mmol, 1.0 eq) of 3-fluoro-4-bromobenzyl bromide, and 3.44 g (0.0207 mmol, 1.1 eq) of triethyl phosphite. Stirred at 110° C. for 18 hours, completion was confirmed by LC-MS. The reaction mixture was concentrated and the residue was purified by column chromatography to yield 5.69 g of compound 156-1, with a 93.4% yield.

Step 2: To a 50 mL reaction flask, 20 mL of THF, 1 g (3.08 mmol, 1.0 eq) of compound 156-1, and 1.36 g (3.08 mmol, 1.0 eq) of (2,3-dimethylphenyl) (1-trityl-4-imidazolyl) methanone were added and cooled to 0° C. Then, 1.04 g (9.24 mmol, 3.0 eq) of potassium tert-butoxide was introduced. After stirring at 10° C. for 18 hours and confirmation of completion by LC-MS, the reaction was filtered, concentrated, and the residue was purified by column chromatography to yield 1.31 g of 156-2, a 69.5% yield.

Step 3: A 10 mL reaction flask received 5 mL of DMF, 500 mg (0.817 mmol, 1.0 eq) of 156-2, 192 mg (1.634 mmol, 2.0 eq) of zinc cyanide, and 95 mg (0.0817 mmol, 0.1 eq) of Pd(PPh3)₄. Stirred at 120° C. for 18 hours, TLC indicated 50% of the raw materials remained. The mixture was diluted with 40 mL of ice water, extracted three times with 20 mL of EA, dried over anhydrous sodium sulfate, filtered, and concentrated under vacuum. Purification by column chromatography yielded 220 mg of 156-3, an 83.5% yield.

Step 4: Into a 5 mL reaction flask, 1.5 mL of THF, 1.5 mL of MeOH, 110 mg (0.197 mmol, 1.0 eq) of 156-3, 54 mg (0.394 mmol, 2.0 eq) of K2CO3, and 45 mg (0.394 mmol, 2.0 eq) of 30% H₂O₂ were combined under nitrogen. The mixture was stirred overnight at 20° C. for 18 hours. Following completion, confirmed by LC-MS, the mixture was poured into 10 ml of water, extracted three times with 10 mL of EA, dried over Na₂SO₄, and concentrated under vacuum. Purification by column chromatography yielded 77 mg of 156-4, a 67.7% yield.

Step 5: In a 5 mL reaction flask, 3 mL of MeOH:THF (1:1), 90 mg (0.156 mmol, 1.0 eq) of 156-4, and 87 mg of Pd(OH)₂ were stirred at 48° C. overnight under a hydrogen environment. LC-MS confirmed the reaction's completion. The reaction was filtered, concentrated, and the crude product was further processed with 2 mL of DCM and 0.5 mL of TFA, stirred for 0.5 hours, then concentrated. Purification by pre-HPLC yielded 41 mg of compound 156, with a 58.3% yield. Overall yield: 21.4%

LC-MS: [M-C₂HF₃O₂+1]+=338.2

1H NMR (400 MHZ, DMSO) δ 14.25 (s, 2H), 8.97 (s, 1H), 7.65-7.48 (m, 4H), 7.09 (dt, J=14.4, 7.2 Hz, 5H), 4.78 (t, J=7.9 Hz, 1H), 3.42 (dd, J=13.9, 8.5 Hz, 1H), 3.22 (dd, J=13.9, 7.3 Hz, 1H), 2.23 (s, 3H), 2.16 (s, 3H).

### Example 14. Synthesis of Compound 182

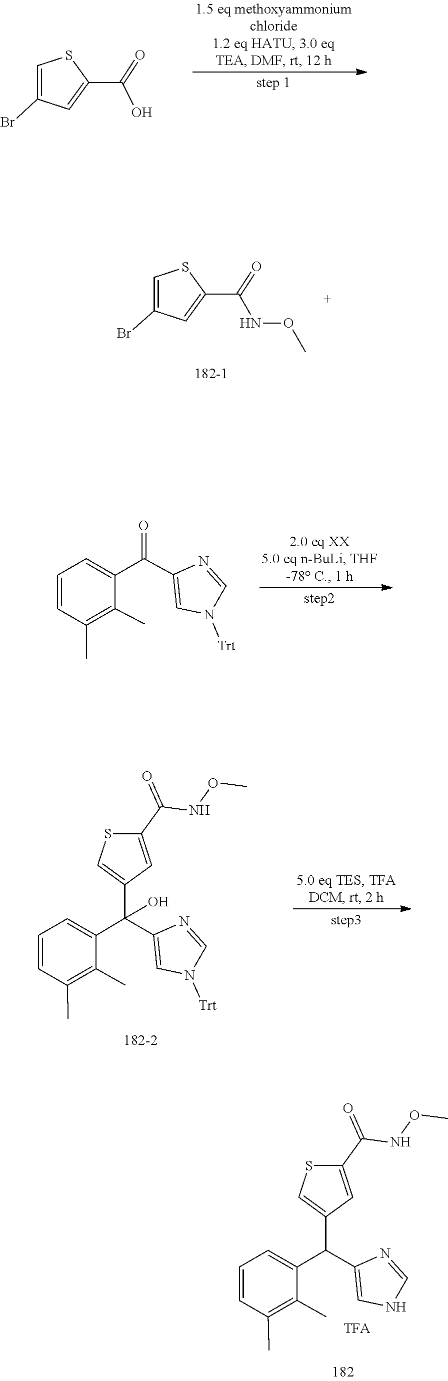

Step 1: In a 50 mL flask, 20 mL DMF, 2 g (9.66 mmol, 1.0 eq) of 4-bromo-2-thiophenecarboxylic acid, 2.93 g (28.98 mmol, 3.0 eq) of TEA, and 1.21 g (14.49 mmol, 1.5 eq) of methoxyammonium chloride were combined. After adding 4.41 g (11.59 mmol, 1.2 eq) of HATU, the mixture was stirred at room temperature for 12 hours. Following completion, confirmed by LC-MS, the reaction was quenched with water and extracted with ethyl acetate. After drying over Na₂SO₄ and filtration, the concentrate was purified by column chromatography (PE:EA=67:33) to yield 1.25 g of 182-1 as a white solid, a 54.8% yield.

Step 2: In a 25 mL three-necked flask, 598 mg (2.53 mmol, 2.0 eq) of 182-1 was dissolved in 6 mL THF and cooled to −80° C. under nitrogen. n-BuLi (2.5M, 2.5 mL, 6.33 mmol, 5.0 eq) was added, stirred for 40 minutes, then a mixture of 560 mg (1.27 mmol, 1.0 eq) of (2,3-dimethylphenyl) (1-trityl-4-imidazolyl) methanone in 6 mL THF was introduced. After stirring for 1 hour and quenching with water, the mixture was warmed and extracted with ethyl acetate. The organic phase was dried, filtered, and concentrated, then purified by column chromatography (DCM:MeOH=90:10) and further recrystallized with PE:MTBE=2:1 to yield 67 mg of 182-2 as a light yellow solid, an 8.8% yield.

Step 3: A 10 mL flask received 1 mL DCM, 1 mL TFA, 65 mg (0.11 mmol, 1.0 eq) of 182-2, and 38 mg (0.33 mmol, 3.0 eq) of TES. After stirring for 2 hours, confirmed complete by LC-MS, the mixture was concentrated and purified by preparative HPLC to afford 20 mg of 182 as a white solid, yielding 42.1%.

Overall yield=2.03%.

LC-MS: [M-C₂HF3O2+1]+=342.1

1H NMR (400 MHZ, DMSO) § 14.36 (s, 2H), 11.75 (s, 1H), 9.06 (s, 1H), 7.46 (s, 2H), 7.12 (p, J=7.4 Hz, 3H), 6.75 (d, J=7.3 Hz, 1H), 5.88 (s, 1H), 3.67 (s, 3H), 2.28 (s, 3H), 2.18 (s, 3H).

### Example 15. Synthesis of Compound 188

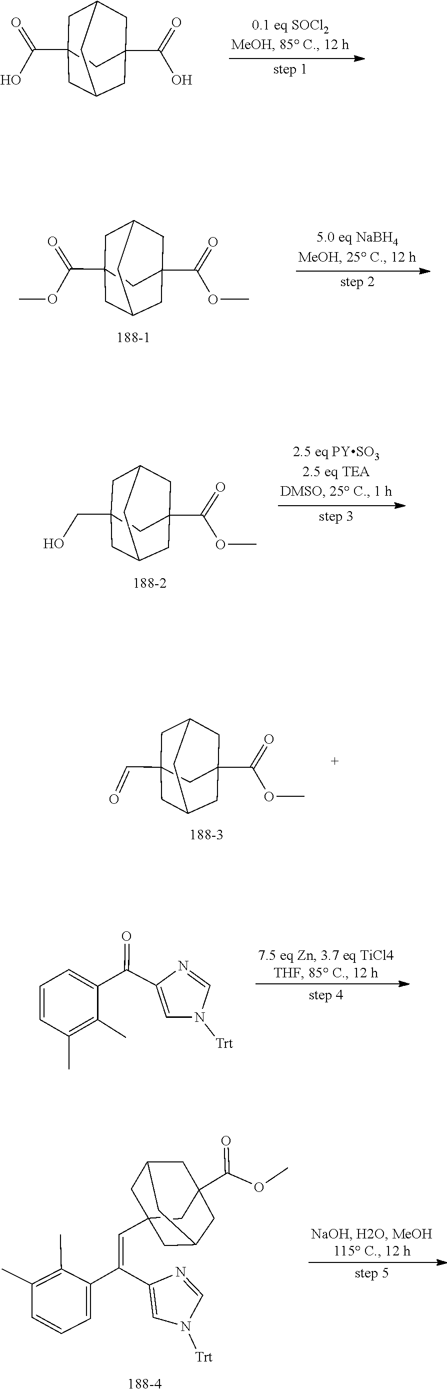

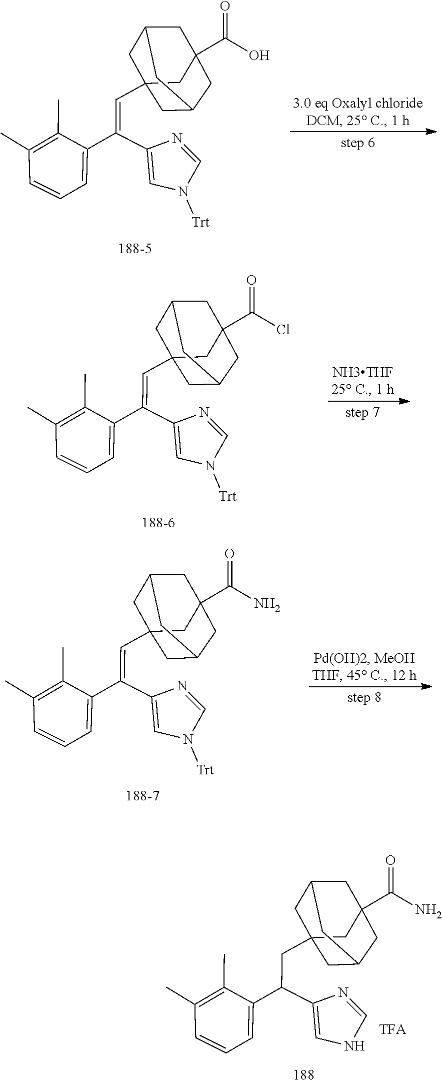

Step 1: Thionyl chloride (0.582 mL, 8.03 mol, 0.1 eq) was added to a methanol solution of adamantane-1,3-dicarboxylic acid (18 g, 80.27 mmol, 1.0 eq) at 0° C. Stirred at 85° C. for 12 hours, the reaction completion was confirmed by LC-MS. The concentrated mixture was diluted with water (50 mL), neutralized to pH=8 with saturated NaHCO₃, extracted with ethyl acetate (30 mL×3), dried, and concentrated to yield 20 g of 188-1 as a white solid (100% yield).

Step 2:188-1 (20 g, 79.27 mmol, 1.0 eq) was dissolved in methanol (350 mL), and NaBH₄ (14.99 g, 0.39 mol, 5.0 eq) was added at 0° C. After stirring at 25° C. for 12 hours and confirming the completion, the reaction was concentrated, diluted with ethyl acetate, extracted with water, dried, and purified to obtain 5 g of 188-2 as a white solid (11.7% yield).

Step 3: DMSO (50 mL), 188-2 (4 g, 17.83 mmol, 1.0 eq), PySO₃ (7.1 g, 44.58 mmol, 2.5 eq), and TEA (4.51 g, 44.58 mmol, 2.5 eq) were added to a 250 mL flask. Stirred at 25° C. for an hour and verified by LC-MS, the mixture was processed and purified to yield 2 g of 188-3 as a white solid (51.3% yield).

Step 4: In a 50 mL flask, THF (20 mL) was combined with Zn (1.1 g, 16.87 mmol, 7.5 eq) and TiCl4 (1.58 g, 8.32 mmol, 3.7 eq) at −70° C., followed by 188-3 (500 mg, 2.25 mmol, 1.0 eq) and a specified compound in THF. Stirred at 80° C. for 2 hours, the reaction was completed, worked up, and purified to yield 270 mg of 188-4 as a yellow solid (19.1% yield).

Step 5:188-4 (200 mg) was reacted with NaOH (5 mL, 8M) in methanol at 20° C., heated to 115° C. for 12 hours, cooled, adjusted to pH=4, extracted, and concentrated to yield 80 mg of 188-5 as a yellow solid (40.8% yield).

Step 6:188-5 (60 mg, 0.096 mmol, 1.0 eq) was mixed with oxalyl dichloride (36.92 mg, 0.291 mmol, 3.0 eq) in DCM (1 mL) at 0° C., stirred at 15° C. for an hour, concentrated to yield 60 mg of 188-6 as a white solid (100% yield), and used directly in the next step.

Step 7:188-6 (60 mg) was dissolved in THF (0.5 mL) and treated with NH₃. THF (8 mL) at 0° C., stirred at 15° C. for an hour, concentrated to yield 60 mg of 188-7 as a white solid (100% yield), and used directly in the next step.

Step 8:188-7 (60 mg) was combined with Pd(OH)₂ (60 mg) in MeOH:THF (16 mL, 1:1), stirred at 45° C. under hydrogen, concentrated, treated with DCM (1 mL) and TFA (0.5 mL), concentrated, and purified to yield 20 mg of 188 as a white solid (43.4% yield).

Overall yield=0.2%.

LC-MS: [M-C₂HF3O2+1]+=378.2

1H NMR (400 MHZ, DMSO) δ 14.20 (s, 2H), 8.96 (s, 1H), 7.66 (s, 1H), 7.03 (t, J=6.2 Hz, 3H), 6.89 (s, 1H), 6.67 (s, 1H), 4.53 (dd, J=8.5, 3.6 Hz, 1H), 2.27 (d, J=16.9 Hz, 6H), 2.06 (dd, J=14.4, 9.0 Hz, 1H), 1.95 (s, 2H), 1.68-1.21 (m, 13H).

### Example 16. Synthesis of Compound 196

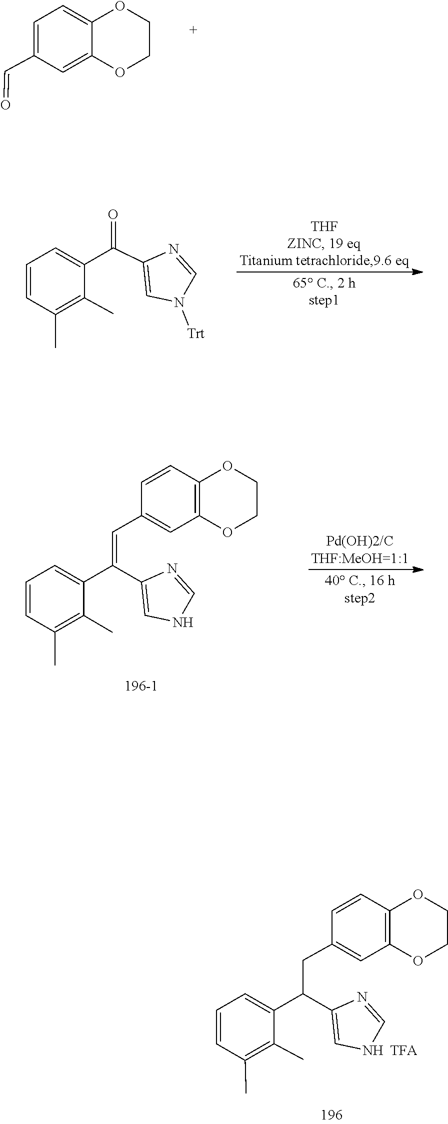

Step 1: Zinc (5.6 g, 0.085 mol, 19 eq) was added to a solution of THF (20 mL), followed by dropwise addition of TiCl4 (8.2 g, 0.043 mol, 9.6 eq) at 0° C. The reaction was then heated and refluxed at 70° C. for 1 hour. After cooling to 30° C., a THF solution containing 2,3-dihydro-benzo[1,4]dioxin-6-carbaldehyde (1.8 g, 0.0108 mol, 2.4 eq) and (2,3-Dimethylphenyl) (1-trityl-4-imidazolyl) methanone (2 g, 0.0045 mol, 1 eq) was introduced and refluxed at 65° C. for 2 hours under nitrogen. Completion was confirmed by LC-MS. The reaction was quenched with water, extracted with ethyl acetate, dried, and concentrated. Purification via column chromatography yielded 2.1 g of 196-1 as a white solid (100% yield).

Step 2:196-1 (100 mg, 0.37 mmol, 1 eq) was combined with Pd(OH)₂/C (100 mg) in a THF:MeOH (1:1) solution and stirred at 40° C. for 16 hours under hydrogen. Following LC-MS confirmation of completion, the reaction was filtered and concentrated to give a crude product. DCM (2 mL) and TFA (1 mL) were added to the crude, which was then concentrated and purified by preparative HPLC to yield 12 mg of 196 as a white solid (9.7% yield).

Overall yield=9.7%

LCMS: [M-C₂HF3O2+1]+=335.2

1H NMR (400 MHZ, DMSO) δ 14.22 (s, 2H), 8.94 (s, 1H), 7.59 (s, 1H), 7.13-6.99 (m, 3H), 6.67 (dd, J=7.5, 4.9 Hz, 2H), 6.59 (dd, J=8.2, 1.6 Hz, 1H), 4.64 (t, J=7.7 Hz, 1H), 4.16 (s, 4H), 3.22 (dd, J=13.9, 8.8 Hz, 1H), 3.01 (dd, J=13.9, 6.8 Hz, 1H), 2.23 (s, 3H), 2.15 (s, 3H).

### Example 17. Synthesis of Compound 401

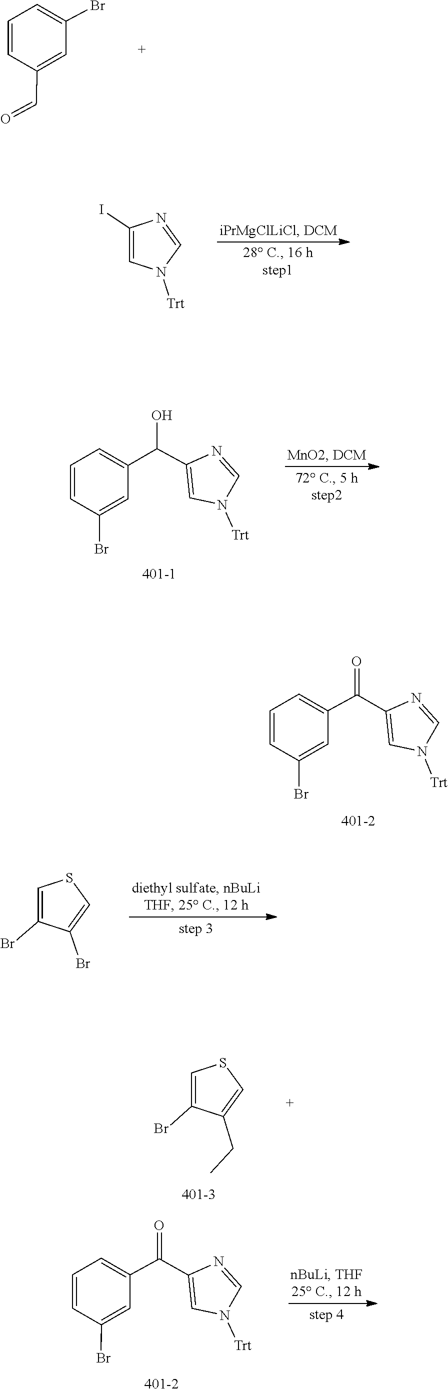

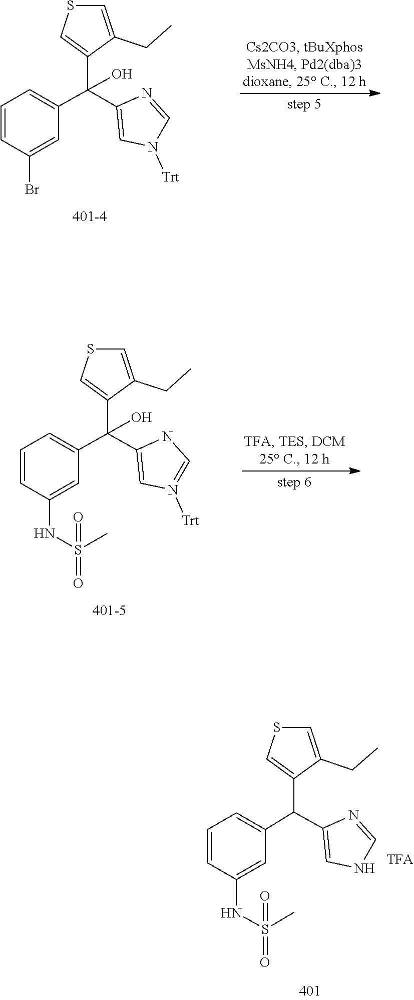

Step 1: In a 100 mL flask, 50 mL of dichloromethane and 4-Iodo-1-trityl-1H-imidazole (11.8 g, 0.027 mol, 1.0 eq) were combined. After cooling the mixture to 0° C., iPrMgCILiCl (1.3 mol/L, 20.7 mL, 0.027 mol, 1.0 eq) was added. The mixture was stirred at 0° C. for 2 hours, then 3-Bromobenzaldehyde (5 g, 0.027 mol, 1.0 eq) was introduced. Stirring continued at 28° C. for 16 hours until LC-MS confirmed the reaction's completion. After cooling back to 0° C. and quenching with 44 mL of saturated ammonium chloride, the organic layer was separated, concentrated, and purified via column chromatography using ethyl acetate to yield 7.1 g of 401-1, achieving a 53% yield.

Step 2: Into a 200 mL high-pressure tube, 180 mL of dichloromethane, compound 401-1 (6.1 g, 12.35 mmol, 1.0 eq), and MnO2 (6.44 g, 74.1 mmol, 6.0 eq) were added. The mixture was stirred at 72° C. for 5 hours. Completion was verified by LC-MS, and the mixture was then filtered to yield 5.6 g of compound 401-2, achieving an 83.9% yield.

Step 3: A 1 L reaction vessel was charged with 500 mL of diethyl ether and 20 g (82.67 mmol, 1 eq) of 3,4-Dibromothiophene (Cas: 3141-26-2). Upon cooling to −78° C., 36.37 mL (90.94 mmol, 1.1 eq) of n-BuLi was added dropwise. The mixture was stirred at −78° C. for 30 minutes before 14.02 g (90.94 mmol, 1.1 eq) of diethyl sulfate was added dropwise. Stirring continued at 25° C. for 5 hours until LC-MS confirmed the reaction's completion. After adding 25 mL of aqueous ammonia, the organic phase was separated, dried over anhydrous sodium sulfate, filtered, and concentrated to yield 10 g of compound 401-3, achieving a 63.3% yield.

Step 4: Into a 50 mL reaction flask, 5 mL of THF and 387.28 mg (2.03 mmol, 2 eq) of 401-3 were introduced. Cooled to −78° C., 0.81 mL (2.03 mmol, 2 eq) of n-BuLi was added dropwise. After stirring at −78° C. for 30 minutes, a solution of 500 mg (1.01 mmol, 1.0 eq) of 401-2 in 5 mL of THF was added. The reaction was then stirred at 25° C. for 12 hours, as indicated by LC-MS completion. The mixture was diluted with 20 mL water and the aqueous phase was extracted with EA (3 times, 5 mL each), the organic layers were combined, washed with brine (3 times, 5 mL each), dried over anhydrous sodium sulfate, filtered, and concentrated. Column chromatography purified the crude to yield 400 mg of compound 401-4, a 65.2% yield.

Step 5: To a 25 mL flask, 7 mL of dioxane, 340 mg (0.561 mmol, 1.0 eq) of 401-4, 64.08 mg (0.67 mmol, 1.2 eq) of MsNH₄, 10.28 mg (0.011 mmol, 0.02 eq) of Pd₂(dba)₃, 9.54 mg (0.022 mmol, 0.04 eq) of tBuxphos, and 365.85 mg (1.12 mmol, 2 eq) of Cs2CO3 were added. The mixture was stirred at 100° C. for 5 hours. After confirmation of completion by LC-MS, it was concentrated under vacuum and purified via column chromatography to obtain 100 mg of compound 401-5, yielding 35.2%.

Step 6: A 5 mL flask was prepared with 1 mL DCM, 0.1 mL TFA, 0.3 mL TES, and 100 mg (1.0 eq) of 401-5. The mixture was stirred at 25° C. for 2 hours, as shown by LC-MS completion. After concentration under vacuum, pre-HPLC purification yielded 10 mg of compound 401, a 17.2% yield.

Overall yield=2.5%

LCMS: [M-C₂HF₃O₂+1]+=362.1

1H NMR (400 MHZ, DMSO) δ 14.37 (s, 2H), 9.79 (s, 1H), 9.06 (d, J=0.7 Hz, 1H), 7.34 (dd, J=10.3, 5.4 Hz, 1H), 7.25 (d, J=3.0 Hz, 1H), 7.17-7.11 (m, 1H), 7.06 (s, 2H), 6.99-6.92 (m, 2H), 5.58 (s, 1H), 2.97 (s, 3H), 2.46-2.37 (m, 1H), 2.35-2.23 (m, 1H), 1.08 (t, J=7.4 Hz, 3H).

### Example 18. Synthesis of Compound 502

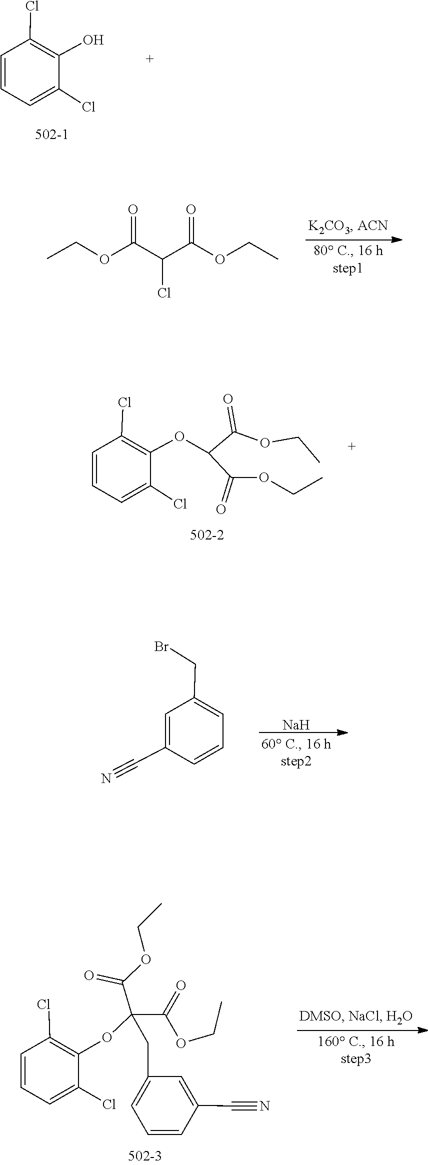

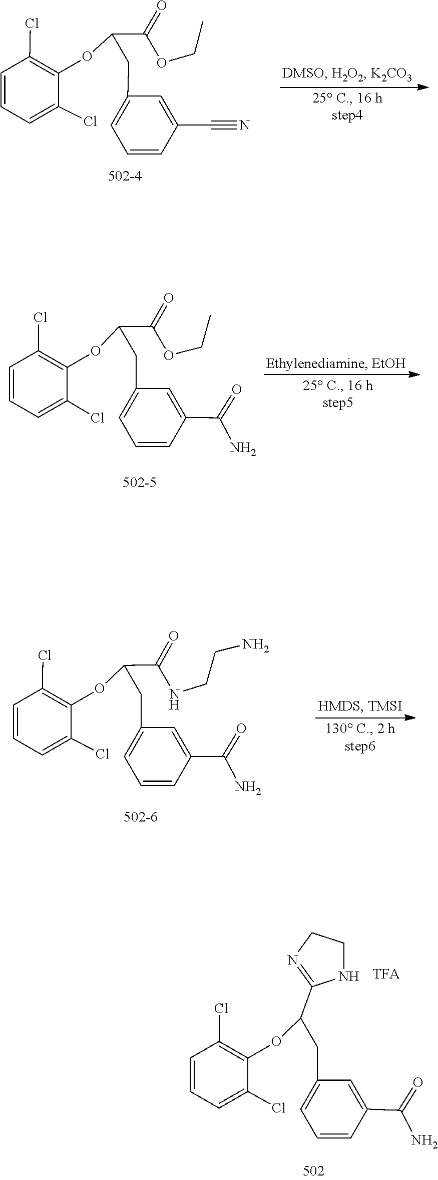 [SMILES: CCOC(=O)C(Cc1cccc(C#N)c1)Oc1c(Cl)cccc1Cl ; CCOC(=O)C(Cc1cccc(C(N)=O)c1)Oc1c(Cl)cccc1Cl ; NCCNC(=O)C(Cc1cccc(C(N)=O)c1)Oc1c(Cl)cccc1Cl ; NC(=O)c1cccc(CC(Oc2c(Cl)cccc2Cl)C2=NCCN2)c1]

Step 1: A 500 mL three-necked flask was loaded with 250 ml of ACN, 25 g (0.15 mol, 1 eq) of 502-1, 31.5 g (0.16 mol, 1.05 eq) of diethyl chloromalonate, and 43 g (0.31 mol, 2 eq) of K₂CO₃. The reaction mixture was refluxed at 80° C. overnight. After completion was confirmed by LC-MS, the mixture was concentrated under vacuum and purified via silica gel column chromatography to yield 40 g of 502-2. The yield was 83%.

Step 2: In a 1 L three-necked flask, 250 mL of DMF and 8 g (0.21 mol, 1.5 eq, 60%) of NaH were combined and cooled to 0° C. Then, 40 g (0.125 mol, 1 eq) of 502-2 dissolved in 100 mL of DMF was added at 0° C. and stirred for 1 hour. Next, 30 g (0.154 mol, 1.1 eq) of 3-(Bromomethyl)benzonitrile in 100 mL of DMF was added at 0° C., and the mixture was stirred at 58° C. overnight. After completion (confirmed by LC-MS), the reaction was quenched with water, extracted with EA, dried over Na₂SO₄, and concentrated. Purification by silica gel column chromatography yielded 36 g of 502-3 with a 64% yield.

Step 3: A 500 mL three-necked flask received 300 mL of DMSO, 36 g (0.08 mol, 1 eq) of 502-3, 9 g (0.15 mol, 2 eq) of NaCl, and 11 g (0.3 mol, 4 eq) of H₂O. The mixture was stirred at 150° C. overnight. LC-MS indicated the reaction was incomplete. The mixture was worked up similarly to previous steps and purified to yield 25 g of 502-4 with an 86% yield.

Step 4: To a 500 mL three-necked flask, 200 mL of DMSO, 20 g (0.055 mol, 1 eq) of 502-4, 13 g (0.11 mol, 2 eq) of H₂O₂, and 15 g (0.11 mol, 2 eq) of K₂CO₃ were added. The mixture was stirred at room temperature overnight and purified after standard work-up to yield 8 g of 502-05. The yield was 38%.

Step 5: A 100 mL three-necked flask was charged with 40 mL of EtOH, 8 g (0.021 mol, 1 eq) of 502-5, and 12.6 g (0.21 mol, 10 eq) of ethylenediamine. The mixture was stirred at room temperature overnight and purified to yield 7 g of 502-6 with an 84% yield.

Step 6: To a 25 mL single-necked flask, 1 g (2.53 mmol, 1 eq) of 502-6, 7.5 mL of HMDS, and 0.5 mL of TMSI were added. The mixture was stirred at 130° C. overnight, concentrated under vacuum, then added to 2 mL DCM and 1 mL TFA, stirred for 1 hour at room temperature, and concentrated. Purification yielded 10 mg of 502 with a final yield of 1%.

Overall yield=0.15%

LCMS: [M-C₂HF₃O₂+1]+=378.0

1H NMR (400 MHZ, DMSO) δ 10.45 (s, 2H), 7.98 (s, 1H), 7.85 (s, 1H), 7.80 (d, J=7.3 Hz, 1H), 7.55 (d, J=8.1 Hz, 2H), 7.43 (dt, J=14.9, 7.6 Hz, 3H), 7.26 (t, J=8.1 Hz, 1H), 5.23 (t, J=7.1 Hz, 1H), 3.81 (dd, J=14.5, 6.8 Hz, 4H), 3.60 (d, J=7.7 Hz, 1H), 3.50 (d, J=5.7 Hz, 1H).

### Example 19. Synthesis of Compound 503

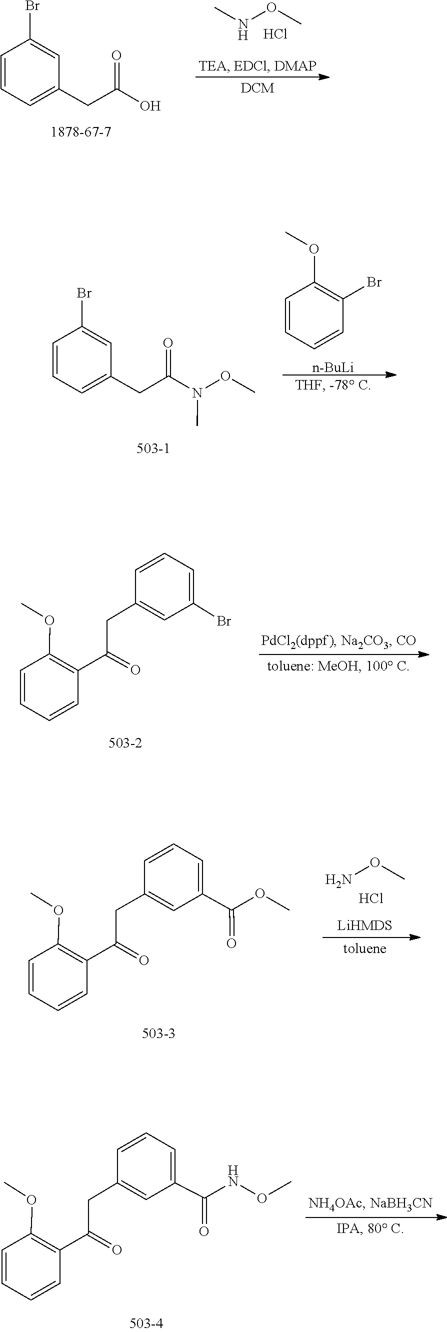

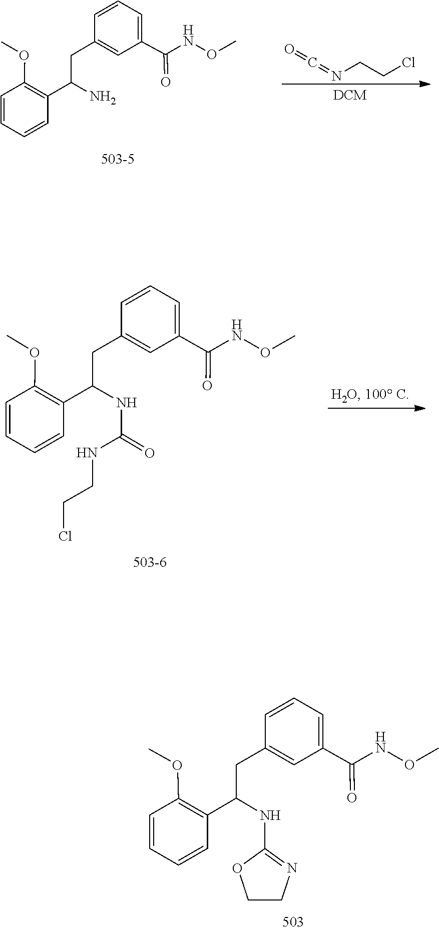

Step 1: Into a 500 mL three-necked flask, 250 mL of ACN, 25 g (0.15 mol, 1 eq) of (3-bromophenyl) acetic acid, 31.5 g (0.16 mol, 1.05 eq) of diethyl chloromalonate, and 43 g (0.31 mol, 2 eq) of K2CO3 were added. The mixture was refluxed overnight at 80° C. Following LC-MS confirmation of completion, it was concentrated and purified via silica gel chromatography, eluting with EtOAc/PE from 1/20 to 1/10 to yield 20 g of 2-(3-bromophenyl)-N-methoxy-N-methylacetamide (503-1) as a yellow oil, with a 73.60% yield.

Step 2: A solution of 1-bromo-2-methoxybenzene (28.99 g, 155 mmol) in dry THF (150 mL) was cooled to −78° C., to which n-BuLi (2.5M in hexane, 62 mL, 155 mmol) was added dropwise. After stirring at −78° C. for 30 minutes, a solution of 503-1 (20 g, 77.5 mmol) in dry THF (100 mL) was added dropwise. The solution was then allowed to warm to room temperature and stirred for 16 hours. The reaction was quenched with saturated NH₄Cl solution, extracted with EtOAc, and purified via silica gel chromatography, eluting with EtOAc/PE at 1/50 to yield 10 g of 2-(3-bromophenyl)-1-(2-methoxyphenyl) ethenone (503-2) as a yellow oil, with a 40.13% yield. Step 3: A solution of 503-2 (1.5 g, 4.9 mmol), PdCl₂ (dppf) (360 mg, 0.49 mmol), and sodium carbonate (1.04 g, 9.8 mmol) in toluene:MeOH (10 mL, 1:1 ratio) was heated at 100° C. for 3 days under CO atmosphere. The reaction was diluted with water, extracted with EtOAc, and purified via silica gel chromatography, eluting with EtOAc/PE from 1% to 10%, to yield 700 mg of methyl 3-[2-(2-methoxyphenyl)-2-oxoethyl]benzoate (503-3) as a yellow oil, with a 46.94% yield.

Step 4: To a solution of methyl 3-[2-(2-methoxyphenyl)-2-oxoethyl]benzoate (600 mg, 2.1 mmol) and O-methylhydroxylamine hydrochloride (264.38 mg, 3.16 mmol) in toluene (8 mL), LiHMDS (1M, 8.4 mL, 8.441 mmol) was added and stirred at 25° C. for 3 hours. After dilution with aqueous NH₄Cl and extraction with EtOAc, the product was purified via silica gel chromatography, eluting with DCM/MeOH at 1/20, to yield 400 mg of N-methoxy-3-[2-(2-methoxyphenyl)-2-oxoethyl]benzamide (503-4) as a yellow oil, with a 60.45% yield.

Step 5: A solution of N-methoxy-3-[2-(2-methoxyphenyl)-2-oxoethyl]benzamide (400 mg, 1.3364 mmol) and NH₄OAc (1.545 g, 20.046 mmol) in IPA (8.0 mL) was stirred at 25° C. for 30 minutes before adding NaBH₃CN (335.92 mg, 5.34 mmol) and heated at 80° C. for 3 hours. After adjusting the pH to 8 with 2M NaOH, the mixture was extracted with DCM and purified via silica gel chromatography, eluting with MeOH/DCM at 1/10, to yield 320 mg of 3-[2-amino-2-(2-methoxyphenyl)ethyl]-N-methoxybenzamide (503-5) as a white solid, with a 71.75% yield. Step 6: To a solution of 3-[2-amino-2-(2-methoxyphenyl)ethyl]-N-methoxybenzamide (300 mg, 0.9988 mmol, 1 eq) in DCM:DMF (5.0 mL, 10:1 ratio), 1-chloro-2-isocyanatoethane (421.59 mg, 3.9952 mmol) was stirred at 25° C. for 6 hours. After dilution with water and extraction with DCM, the combined organic phases were washed with brine, dried over sodium sulfate, and concentrated under vacuum. Without further purification, the crude product (250 mg, 46.26% yield) was obtained as a yellow oil.

Step 7: To a solution of 3-(2-{[(2-chloroethyl) carbamoyl]amino}-2-(2-methoxyphenyl)ethyl)-N-methoxybenzamide (200 mg, 0.4927 mmol) in water (5.0 mL), the mixture was heated at 100° C. for 3 hours. After cooling to room temperature, the mixture was purified by Biotage using a C18 column, eluting with 5% to 95% MeCN/H₂O containing 0.1% NH₄OH, to afford 48.25 mg of 3-[2-(4,5-dihydro-1,3-oxazol-2-ylamino)-2-(2-methoxyphenyl)ethyl]-N-methoxybenzamide (503) as a white solid, with a 24.65% yield.

Overall yield=11.40%

LCMS (ESI): m/z found 370.10 [M+H]+.

[M+1]⁺=370.10

1H NMR (400 MHZ, DMSO) δ 11.69 (s, 1H), 7.66 (s, 1H), 7.53 (d, J=6.8 Hz, 1H), 7.34 (dt, J=18.4, 7.6 Hz, 3H), 7.21 (t, J=8.0 Hz, 1H), 6.93 (dd, J=16.8, 8.4 Hz, 3H), 5.06 (d, J=6.4 Hz, 1H), 4.02 (t, J=8.4 Hz, 2H), 3.80 (s, 3H), 3.71 (s, 3H), 3.38 (t, J=8.4 Hz, 2H), 2.89 (dd, J=13.6, 4.0 Hz, 1H), 2.75 (dd, J=13.6, 10.0 Hz, 1H).

### Example 20. Synthesis of Compound 504

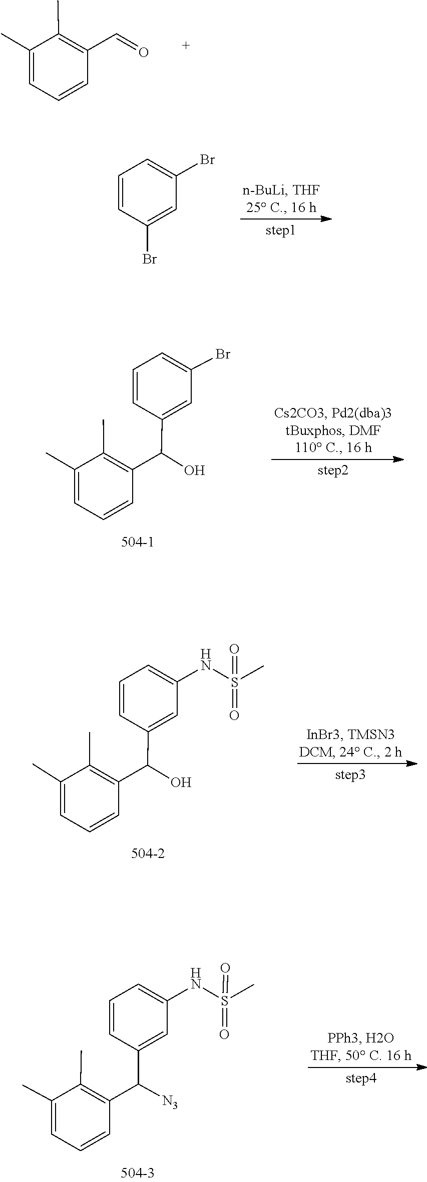

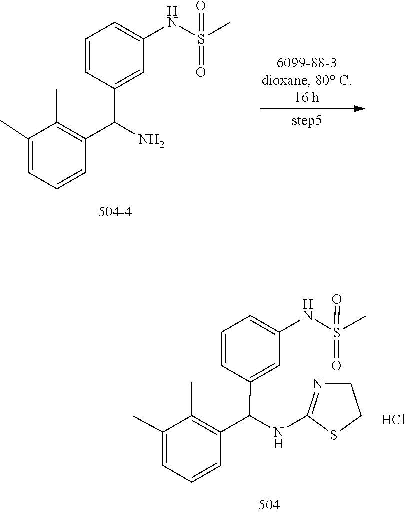

Step 1: In a 250 mL flask, 100 mL of THF and 13.1 g (0.056 mol, 1.5 eq) of 1,3-dibromobenzene were combined. After cooling to −78° C., 22.4 mL (0.056 mol, 1.5 eq) of 2.5M n-BuLi was added. Stirred at −78° C. for 1 hour, then a solution of 5 g (0.0373 mol, 1.0 eq) of 2,3-dimethylbenzaldehyde in 10 mL THF was introduced. The reaction continued at −78° C. for another hour before warming to room temperature overnight. Post-completion, verified by LC-MS, the reaction was quenched with 25 mL saturated ammonium chloride and the organic layer was separated, dried over Na₂SO₄, concentrated under vacuum, and purified via column chromatography (PE:EA=85:15) to yield 8.6 g of 504-1, a 77.8% yield Step 2: A 50 mL flask was charged with 20 mL DMF, 1 g (3.45 mmol, 1.0 eq) of 504-1, 492 mg (5.175 mmol, 1.5 eq) of MsNH₂, 2.25 g (6.9 mmol, 2.0 eq) of Cs₂CO₃, 316 mg (0.345 mmol, 0.1 eq) of Pd2 (dba)₃, and 293 mg (0.69 mmol, 0.2 eq) of tBuxphos. Stirred at 110° C. for 16 hours and completion confirmed by LC-MS, the reaction was quenched with ice water, extracted with EA, and dried over Na₂SO₄. Purification by column chromatography (PE:EA=1:1) yielded 180 mg of 504-2, a 17% yield.

Step 3: To a 5 mL flask, 1.5 mL DCM, 134 mg (0.439 mmol, 1.0 eq) of 504-2, 76 mg (0.659 mmol, 1.5 eq) of TMSN₃, and 31 mg (0.0878 mmol, 0.2 eq) of InBr₃ were added. Stirred at 17° C. for 2 hours, completion confirmed by LC-MS, concentrated under vacuum and purified by column chromatography (PE:EA=68:32) to yield 105 mg of 504-3, a 72.4% yield.

Step 4: A 5 mL flask received 1.5 mL THF, 0.3 mL water, 144 mg (0.436 mmol, 1.0 eq) of 504-3, and 229 mg (0.87 mmol, 2.0 eq) of PPh3. Stirred at 50° C. for 16 hours, completion confirmed by LC-MS, concentrated under vacuum and purified by TLC (DCM:MeOH=20:1) to yield 62 mg of 504-4, a 46.7% yield.

Step 5: To a 5 mL flask, 1 mL dioxane, 52 mg (0.171 mmol, 1.0 eq) of 504-4, and 60 mg (0.5 mmol, 2.9 eq) of 2-chloroethyl isothiocyanate were added. Stirred at 80° C. for 16 hours, completion confirmed by LC-MS, concentrated under vacuum and purified by pre-HPLC to yield 3.3 mg of compound 504, a 5% yield.

Overall yield: 2.2%

LCMS: [M−HCl+1]+=390.1

1H NMR (400 MHz, MeOD) δ 7.40 (t, J=7.9 Hz, 1H), 7.27-7.20 (m, 2H), 7.17 (t, J=7.6 Hz, 2H), 7.00 (dd, J=23.2, 7.5 Hz, 2H), 6.16 (s, 1H), 4.02 (t, J=7.5 Hz, 2H), 3.65 (t, J=7.6 Hz, 2H), 2.95 (s, 3H), 2.34 (s, 3H), 2.19 (s, 3H).

### Example 21. Synthesis of Compound 505

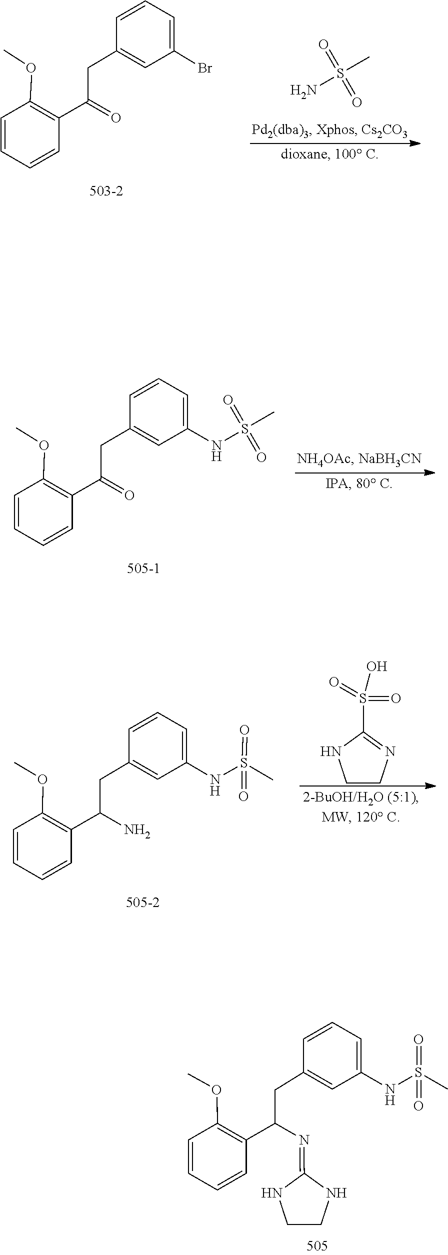

Step 1: A solution of 2-(3-bromophenyl)-1-(2-methoxyphenyl) ethenone (503-2, 2.0 g, 6.6 mmol), methanesulfonamide (0.75 g, 7.92 mmol), Pd(OAc)₂ (150 mg, 0.663 mmol), Xantphos (0.76 g, 1.32 mmol), and Cs2CO3 (4.30 g, 13.20 mmol) in dioxane (20.0 ml) was heated at 100° C. for 16 hours under nitrogen. After cooling, it was diluted with aqueous NH₄Cl and extracted with EtOAc. The organic layers were combined, washed with brine, dried over sodium sulfate, and concentrated. Purification by silica gel chromatography (MeOH/DCM, 1% to 10%) yielded N-{3-[2-(2-methoxyphenyl)-2-oxoethyl]phenyl}methanesulfonamide (505-1, 400 mg, 16.17% yield) as a yellow oil.

Step 2: A mixture of N-{3-[2-(2-methoxyphenyl)-2-oxoethyl]phenyl}methanesulfonamide (400 mg, 0.5323 mmol) and NH₄OAc (1.448 g, 18.785 mmol) in isopropanol (8.0 ml) was stirred at 25° C. for 0.5 hour, then NaBH₃CN (314.80 mg, 5.0096 mmol) was added and the mixture was heated at 80° C. for 4.5 hours. After cooling, the mixture was filtered through celite and concentrated. It was purified by preparative TLC (EtOAc/PE, 1/3) to obtain N-{3-[2-amino-2-(2-methoxyphenyl)ethyl]phenyl}methanesulfonamide (505-2, 240 mg, 53.82% yield) as a yellow oil.

Step 3: A solution of N-{3-[2-amino-2-(2-methoxyphenyl)ethyl]phenyl}methanesulfonamide (505-2, 160 mg, 0.4994 mmol) and 4,5-dihydro-1H-imidazole-2-sulfonic acid (224.96 mg, 1.4982 mmol) in butanol:water (5:1 ratio, 3.0 ml) was heated at 120° C. for 2 hours in a microwave reactor. After cooling, the mixture was concentrated, diluted with water, and extracted with EtOAc. The organic phases were combined, washed with brine, dried over sodium sulfate, concentrated, and purified by Biotage using a C18 column (eluting with 10% to 95% MeCN/H₂O, containing 0.1% TFA) to yield N-{3-[2-(imidazolidin-2-ylideneamino)-2-(2-methoxyphenyl)ethyl]phenyl}methanesulfonamide (505, 8.38 mg, 4.31% yield) as a white solid. LCMS: [M+1]⁺=388.38

1H NMR (400 MHZ, DMSO) δ 9.69 (s, 1H), 8.74 (d, J=9.2 Hz, 1H), 8.31 (s, 1H), 7.67 (s, 1H), 7.33-7.16 (m, 3H), 7.11 (s, 1H), 7.03 (dd, J=17.2, 8.4 Hz, 3H), 6.94 (t, J=7.6 Hz, 1H), 4.96-4.90 (m, 1H), 3.88 (s, 3H), 3.48 (s, 5H), 3.09 (dd, J=13.6, 4.8 Hz, 1H), 2.94-2.87 (m, 4H).

### Example 22. Synthesis of Compound 510

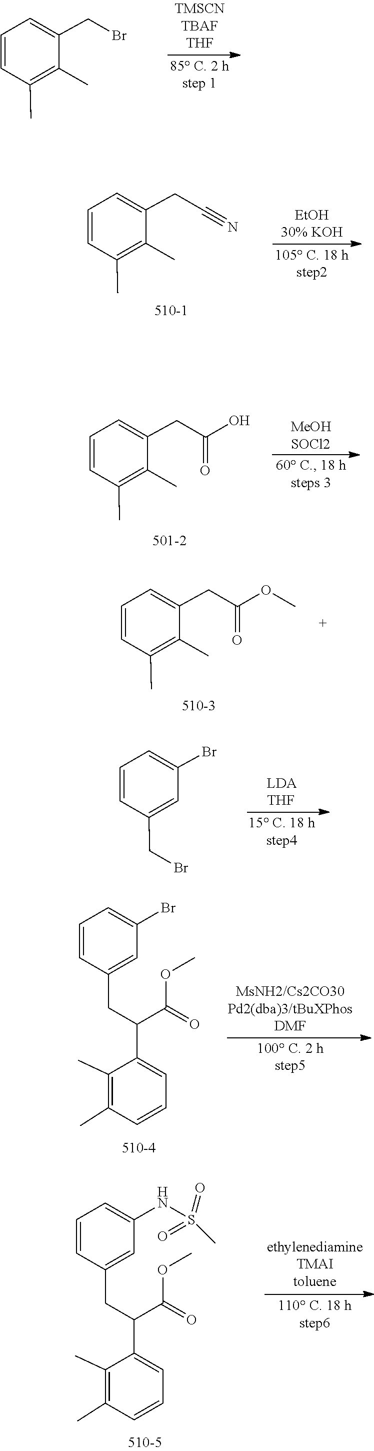

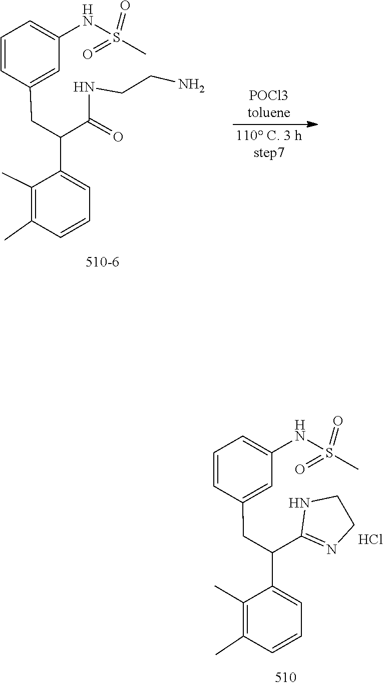 [SMILES: Cc1cccc(C(Cc2cccc(NS(C)(=O)=O)c2)C(=O)NCCN)c1C ; Cc1cccc(C(Cc2cccc(NS(C)(=O)=O)c2)C2=NCCN2)c1C ; Cl]

Step 1: In a 100 mL flask, 20 mL of ACN, 2 g (0.01 mol, 1.0 eq) of 2,3-Dimethylbenzyl bromide, 2 g (0.02 mmol, 2.0 eq) of TMSCN, and 20 mL (0.05 mmol, 2.0 eq) of TBAF in THF were combined and refluxed for 1.5 hours. After LC-MS confirmation of completion, the mixture was concentrated and purified by column chromatography (PE:EA=85:15) to yield 1.42 g of 510-1, a 97.9% yield.

Step 2: In a 100 mL flask, 28 mL of ethanol and 5.7 mL of 30% KOH solution were added to 1.42 g (0.0098 mol, 1.0 eq) of 510-1. The mixture was refluxed for 18 hours, concentrated, diluted with 20 mL water, adjusted to pH=2 with 6M HCl, filtered, and dried to yield 1.35 g of 510-2 as a solid, an 84.0% yield.

Step 3: A 50 mL flask received 15 mL of methanol and 1.3 g (0.008 mol, 1.0 eq) of 510-2, cooled to 0° C., then 1.89 g (0.0159 mol, 1.5 eq) of SOCl₂ was added. The reaction was stirred at 60° C. for 18 hours, concentrated, and purified by column chromatography (PE:EA=68:32) to yield 1.37 g of 510-3, a 96.2% yield.

Step 4: In a 25 ml bottle, 10 mL of THF, 497 mg (2.81 mmol, 1.0 eq) of 510-3 were cooled to −80° C. under nitrogen. LDA (3.37 mL, 3.37 mmol, 1.2 eq) was added, followed by 731 mg (2.95 mmol, 1.05 eq) of 823-78-9 in 2 mL THF. The mixture was warmed to room temperature overnight, quenched, and purified by column chromatography to yield 670 mg of 510-4, a 68.9% yield.

Step 5: A 50 ml bottle received 10 mL of DMF, 570 mg (1.65 mmol, 1.0 eq) of 510-4, 1.07 g (3.3 mmol, 2.0 eq) of Cs₂CO₃, 235 mg (2.47 mmol, 1.5 eq) of methanesulfonamide, 151 mg (0.165 mmol, 0.1 eq) of Pd₂(dba)₃, and 141 mg (0.33 mmol, 0.2 eq) of t-BuXphos. Stirred at 105° C. for 2 hours under nitrogen, the reaction was worked up and purified to yield 600 mg of 510-5, an 85.7% yield.

Step 6: In a 10 mL bottle, 5 mL of toluene, 200 mg (0.554 mmol, 1.0 eq) of 510-5, 166.5 mg (2.77 mmol, 5.0 eq) of ethylenediamine, and TMAl (1.39 mL, 2.77 mmol, 5.0 eq) were stirred at 110° C. overnight. After cooling and working up, the crude was purified to yield 29 mg of 510-6, a 13.5% yield.

Step 7: A 5 mL bottle received 1 mL of toluene and 29 mg (0.0745 mmol, 1.0 eq) of 510-6, then 57 mg (0.37 mmol, 5.0 eq) of POCl₃ was added. Stirred at 110° C. for 3 hours, the mixture was filtered and purified to yield 3.6 mg of 510, an 11.9% yield.

LC-MS=[M−HCl+1]+=372.2

1H NMR (400 MHZ, DMSO) δ 9.98 (s, 2H), 7.35 (d, J=7.1 Hz, 1H), 7.24 (t, J=7.7 Hz, 1H), 7.15 (dd, J=18.1, 7.2 Hz, 2H), 7.09-7.00 (m, 2H), 6.95 (d, J=7.4 Hz, 1H), 4.44 (t, J=7.5 Hz, 1H), 3.78 (s, 4H), 3.46-3.40 (m, 1H), 3.07 (dd, J=13.4, 7.1 Hz, 1H), 2.90 (s, 3H), 2.22 (s, 3H), 2.11 (s, 3H).

Other compounds were synthesized similarly as the above compounds. The characterization data of compounds are listed in the Table 2 below.

TABLE 2

| # | ¹H-NMR and MS |
|---|---|
| 1 | LC-MS: [M-TFA + 1] += 336.2, 1H NMR (400 MHz, DMSO) δ 14.33 (s, 2H), 11.79 (s, 1H), 9.00 (d, J = 0.9 Hz, 1H), |
| | 7.67 (d, J = 7.8 Hz, 1H), 7.60 (s, 1H), 7.46 (t, J = 7.7 Hz, 1H), 7.35 (d, J = 7.7 Hz, 1H), 7.17-7.06 (m, 2H), 6.94 (s, |
| | 1H), 6.70 (d, J = 7.2 Hz, 1H), 5.91 (s, 1H), 3.69 (s, 3H), 2.26 (s, 3H), 2.13 (s, 3H). |
| 1-A | M + 1] += 336.2, 1H NMR (400 MHz, DMSO) δ 11.98 (s, 1H), 11.71 (s, 1H), 7.61 (s, 1H), 7.55 (d, J = 7.5 Hz, 2H), |
| | 7.36 (t, J = 7.7 Hz, 1H), 7.29 (d, J = 7.7 Hz, 1H), 7.05-6.95 (m, 2H), 6.79 (d, J = 7.0 Hz, 1H), 6.44 (s, 1H), 5.65 (s, |
| | 1H), 3.67 (s, 3H), 2.23 (s, 3H), 2.11 (s, 3H). |
| 1-B | [M-HCl + 1] += 336.2, 1H NMR (400 MHz, DMSO) δ 14.60 (s, 2H), 11.93 (s, 1H), 9.13 (d, J = 1.1 Hz, 1H), 7.70 (d, |
| | J = 7.8 Hz, 1H), 7.61 (s, 1H), 7.46 (t, J = 7.7 Hz, 1H), 7.35 (d, J = 7.7 Hz, 1H), 7.18-7.05 (m, 2H), 7.00 (s, 1H), 6.72 (d, |
| | J = 7.1 Hz, 1H), 5.95 (s, 1H), 3.69 (s, 3H), 2.26 (s, 3H), 2.13 (s, 3H). |
| 2 | [M-TFA + 1] += 350.2, 1H NMR (400 MHz, DMSO) δ 14.42 (s, 2H), 9.03 (s, 1H), 8.48 (t, J = 5.5 Hz, 1H), 7.80 (d, |
| | J = 7.8 Hz, 1H), 7.70 (s, 1H), 7.45 (t, J = 7.7 Hz, 1H), 7.29 (d, J = 7.7 Hz, 1H), 7.11 (dt, J = 15.1, 7.4 Hz, 2H), 6.94 (s, |
| | 1H), 6.70 (d, J = 7.4 Hz, 1H), 5.90 (s, 1H), 4.73 (s, 1H), 3.49 (t, J = 6.2 Hz, 2H), 3.30 (q, J = 6.0 Hz, 2H), 2.26 (s, 3H), |
| | 2.13 (s, 3H). |
| 3 | [M-TFA + 1] += 408.3, 1H NMR (400 MHz, DMSO) δ 14.35 (s, 1H), 9.03 (s, 1H), 8.55 (t, J = 5.5 Hz, 1H), 7.79 (d, |
| | J = 7.8 Hz, 1H), 7.69 (s, 1H), 7.45 (t, J = 7.7 Hz, 1H), 7.31 (d, J = 7.7 Hz, 1H), 7.11 (dt, J = 15.1, 7.4 Hz, 2H), 6.95 (s, |
| | 1H), 6.70 (d, J = 7.4 Hz, 1H), 5.90 (s, 1H), 3.54-3.49 (m, 4H), 3.44-3.41 (m, 4H), 3.22 (s, 3H), 2.26 (s, 3H), 2.13 (s, 3H). |
| 4 | [M + 1] += 443.2, 1H NMR (400 MHz, DMSO) δ 11.85 (s, 1H), 9.68 (s, 1H), 8.67 (s, 1H), 8.46 (d, J = 5.9 Hz, 1H), 7.93 |
| | (d, J = 36.3 Hz, 1H), 7.56 (d, J = 4.7 Hz, 2H), 7.18 (s, 1H), 6.98 (s, 2H), 6.72 (d, J = 8.9 Hz, 4H), 6.37 (d, J = 94.6 Hz, |
| | 1H), 5.58 (d, J = 38.0 Hz, 1H), 3.97 (s, 2H), 3.51 (d, J = 5.0 Hz, 2H), 2.22 (s, 3H), 2.11 (s, 3H). |
| 5 | LC-MS: [M-TFA + 1] += 306.2, 1H NMR (400 MHz, DMSO) δ 14.29 (s, 2H), 8.97 (s, 1H), 7.99 (s, 1H), 7.81 (d, J = 7.4 |
| | Hz, 1H), 7.70 (s, 1H), 7.43 (dd, J = 14.7, 6.6 Hz, 2H), 7.31 (d, J = 7.3 Hz, 1H), 7.18-7.02 (m, 2H), 6.93 (s, 1H), 6.71 |
| | (d, J = 7.3 Hz, 1H), 5.88 (s, 1H), 2.26 (s, 3H), 2.13 (s, 3H). |
| 5-A | LC-MS = [M + 1] += 306.2, 1H NMR (400 MHz, DMSO) δ 11.90 (s, 1H), 7.92 (s, 1H), 7.69 (d, J = 8.9 Hz, 2H), 7.59 (s, |
| | 1H), 7.31 (dt, J = 25.6, 7.6 Hz, 3H), 7.00 (q, J = 7.7 Hz, 2H), 6.79 (d, J = 6.8 Hz, 1H), 6.42 (s, 1H), 5.64 (s, 1H), 2.23 |
| | (s, 3H), 2.11 (s, 3H). |
| 5-B | LC-MS = [M + 1] += 306.2, 1H NMR (400 MHz, DMSO) δ 11.92 (s, 1H), 7.92 (s, 1H), 7.70 (d, J = 9.0 Hz, 2H), 7.59 (s, |
| | 1H), 7.31 (dt, J = 25.9, 7.6 Hz, 3H), 7.00 (q, J = 7.7 Hz, 2H), 6.79 (d, J = 6.7 Hz, 1H), 6.42 (s, 1H), 5.64 (s, 1H), 2.23 |
| | (s, 3H), 2.11 (s, 3H). |
| 6 | LC-MS: [M-TFA + 1] += 320.2, 1H NMR (400 MHz, DMSO) δ 14.37 (s, 2H), 9.01 (s, 1H), 8.45 (d, J = 4.5 Hz, 1H), |
| | 7.76 (d, J = 7.8 Hz, 1H), 7.66 (s, 1H), 7.45 (t, J = 7.7 Hz, 1H), 7.30 (d, J = 7.7 Hz, 1H), 7.17-7.05 (m, 2H), 6.94 (s, |
| | 1H), 6.70 (d, J = 7.3 Hz, 1H), 5.89 (s, 1H), 2.76 (d, J = 4.5 Hz, 3H), 2.26 (s, 3H), 2.13 (s, 3H). |
| 7 | [M + 1] += 338.2 1H NMR (400 MHz, DMSO) δ 14.09 (s, 2H), 11.78 (s, 1H), 9.45 (s, 1H), 8.82 (s, 1H), 7.65 (d, J = 7.8 |
| | Hz, 1H), 7.58 (s, 1H), 7.44 (t, J = 7.7 Hz, 1H), 7.33 (d, J = 7.8 Hz, 1H), 6.98 (t, J = 7.9 Hz, 1H), 6.88 (s, 1H), 6.77 (d, |
| | J = 7.9 Hz, 1H), 6.31 (d, J = 7.6 Hz, 1H), 5.77 (s, 1H), 3.68 (s, 3H), 2.02 (s, 3H). |
| 8 | 1H NMR (400 MHz, DMSO) δ 12.25 (s, 3H), 7.57 (s, 1H), 7.52 (d, J = 7.8 Hz, 1H), 6.98 (dt, J = 15.0, 6.6 Hz, 3H), |
| | 6.79 (t, J = 7.7 Hz, 1H), 6.73 (d, J = 7.2 Hz, 1H), 6.37 (s, 1H), 5.87 (s, 1H), 3.35 (s, 1H), 2.22 (s, 3H), 2.08 (s, |
| | 3H)., [M + 1] += 352.2 |
| 9 | 1H NMR (400 MHz, DMSO) δ 13.74 (s, 1H), 11.82 (s, 1H), 8.46 (s, 1H), 7.95 (s, 1H), 7.72 (d, J = 7.6 Hz, 1H), 7.56 (s, |
| | 1H), 7.22-6.83 (m, 3H), 6.77 (t, J = 7.7 Hz, 2H), 6.40 (s, 1H), 5.87 (s, 1H), 2.22 (s, 3H), 2.09 (s, 3H)., [M + 1] += 322.2 |
| 10 | LC-MS: [M-C2HFO2 + 1] += 345.2, 1H NMR (400 MHz, DMSO) δ 13.33 (s, 2H), 10.27 (s, 1H), 8.19 (s, 1H), 7.49 (d, |
| | J = 7.7 Hz, 1H), 7.35-7.18 (m, 2H), 7.05 (dd, J = 19.4, 7.0 Hz, 2H), 6.89 (d, J = 7.3 Hz, 1H), 6.68 (d, J = 6.7 Hz, 1H), |
| | 6.58 (s, 1H), 5.70 (s, 1H), 3.84 (s, 2H), 2.24 (s, 3H), 2.10 (s, 3H). |
| 10-A | LC-MS = [M + 1] += 345.2, 1H NMR (400 MHz, DMSO) δ 11.88 (s, 1H), 10.20 (s, 1H), 7.57 (s, 1H), 7.49 (d, J = 7.9 |
| | Hz, 1H), 7.27-7.17 (m, 2H), 6.99 (q, J = 7.5 Hz, 2H), 6.88 (d, J = 7.6 Hz, 1H), 6.79 (d, J = 6.1 Hz, 1H), 6.42 (s, 1H), 5.56 |
| | (s, 1H), 3.82 (s, 2H), 2.22 (s, 3H), 2.11 (s, 3H). |
| 10-B | LC-MS = [M + 1] += 345.2, 1H NMR (400 MHz, DMSO) δ 11.87 (s, 1H), 10.20 (s, 1H), 7.57 (s, 1H), 7.50 (d, J = 7.9 |
| | Hz, 1H), 7.29-7.16 (m, 2H), 6.99 (q, J = 7.3 Hz, 2H), 6.88 (d, J = 7.6 Hz, 1H), 6.79 (s, 1H), 6.42 (s, 1H), 5.56 (s, 1H), |
| | 3.83 (s, 2H), 2.22 (s, 3H), 2.09 (d, J = 12.3 Hz, 3H). |
| 11 | LC-MS: [M-TFA + 1] += 307.2, 1H NMR (400 MHz, DMSO) δ 14.05 (s, 2H), 8.79 (s, 1H), 7.87 (d, J = 6.5 Hz, 1H), |
| | 7.74 (s, 1H), 7.60-7.37 (m, 2H), 7.10 (d, J = 13.8 Hz, 2H), 6.87 (s, 1H), 6.71 (d, J = 6.5 Hz, 1H), 5.92 (s, 1H), 2.26 |
| | (s, 3H), 2.12 (s, 3H). |
| 12 | LC-MS = [M + 1] += 307.36, 1H NMR (400 MHz, DMSO) δ 11.96 (s, 1H), 7.83 (d, J = 8.2 Hz, 2H), 7.58 (s, 1H), 7.19 |
| | (d, J = 8.2 Hz, 2H), 7.07-6.94 (m, 2H), 6.79 (d, J = 6.8 Hz, 1H), 6.44 (s, 1H), 5.66 (s, 1H), 2.23 (s, 3H), 2.11 (s, 3H). |
| 13 | [M + 1] += 279.2 1H NMR (400 MHz, DMSO) δ 11.21 (d, J = 18.6 Hz, 1H), 7.96-7.74 (m, 1H), 7.66 (d, J = 7.8 Hz, 1H), |
| | 6.40 (d, J = 25.1 Hz, 2H), 6.08-5.85 (m, 2H), 5.53 (dt, J = 19.2, 9.6 Hz, 1H), 4.41-4.24 (m, 1H), 4.10 (dd, J = 27.9, 21.9 |
| | Hz, 1H), 3.64 (s, 2H), 3.51 (s, 12H), 3.43-3.35 (m, 4H), 3.16 (dd, J = 19.1, 5.7 Hz, 5H), 2.99 (d, J = 6.1 Hz, 2H), 2.81 |
| | (dt, J = 21.6, 10.8 Hz, 1H), 2.59 (s, 1H), 2.07 (s, 2H), 1.66 (s, 3H), 1.56-1.37 (m, 3H), 1.31 (dd, J = 15.0, 6.7 Hz, 2H)., |
| 14 | LC-MS = [M + 1] += 335.41, 1H NMR (400 MHz, DMSO) δ 11.77 (t, J = 46.3 Hz, 1H), 8.64 (d, J = 38.2 Hz, 1H), 7.55 |
| | (s, 1H), 7.39-7.22 (m, 2H), 6.98 (t, J = 15.2 Hz, 4H), 6.89-6.60 (m, 1H), 6.52-6.07 (m, 2H), 5.50 (d, J = 37.5 Hz, |
| | 1H), 2.62 (d, J = 4.4 Hz, 3H), 2.22 (s, 3H), 2.10 (s, 3H). |
| 15 | [M + 1] += 513.3 1H NMR (400 MHz, DMSO) δ 11.84 (s, 1H), 7.56 (s, 1H), 7.00 (dd, J = 14.8, 8.2 Hz, 4H), 6.85 (d, |
| | J = 8.6 Hz, 2H), 6.76 (s, 1H), 6.36 (s, 1H), 5.53 (s, 1H), 4.11-3.98 (m, 2H), 3.80-3.67 (m, 2H), 3.64-3.55 (m, 2H), |
| | 3.56-3.45 (m, 12H), 3.42 (dd, J = 5.9, 3.5 Hz, 2H), 3.23 (s, 3H), 2.22 (s, 3H), 2.10 (s, 3H). |
| 16 | LCMS = [M-TFA + 1] += 363.3, 1H NMR (400 MHz, DMSO) δ 14.17 (s, 6H), 8.83 (s, 4H), 7.47 (s, 4H), 7.16-6.98 (m, |
| | 8H), 6.89 (d, J = 7.3 Hz, 4H), 4.64 (dd, J = 8.3, 5.5 Hz, 4H), 3.91 (t, J = 9.2 Hz, 4H), 3.77 (dd, J = 9.8, 5.5 Hz, 4H), |
| | 3.60-3.54 (m, 8H), 3.52-3.47 (m, 32H), 3.42-3.40 (m, 9H), 3.23 (s, 13H), 2.27 (s, 24H). |
| 17 | 1H NMR (400 MHz, CDCl3) δ 8.66 (s, 1H), 7.19-7.02 (m, 2H), 6.96 (d, J = 7.2 Hz, 1H), 6.68 (s, 1H), 5.93 (s, 1H), |
| | 4.61 (dd, J = 9.3, 4.1 Hz, 1H), 3.99-3.87 (m, 2H), 3.84-3.77 (m, 1H), 3.65 (ddd, J = 17.3, 13.3, 7.2 Hz, 5H), |
| | 3.46-3.31 (m, 2H), 2.66 (s, 3H), 2.31 (s, 3H), 2.22 (s, 3H)., [M-TFA + 1] += 361.3 |
| 17-A | 1H NMR (400 MHz, DMSO) δ 12.45 (s, 1H), 7.74 (s, 1H), 7.12-6.94 (m, 3H), 6.87 (s, 1H), 5.97-5.70 (m, 2H), |
| | 4.48 (t, J = 7.1 Hz, 1H), 3.97-3.84 (m, 1H), 3.84-3.70 (m, 1H), 3.51 (d, J = 5.0 Hz, 2H), 3.45 (t, J = 4.3 Hz, 2H), |
| | 3.31 (t, J = 5.7 Hz, 2H), 3.09 (q, J = 5.6 Hz, 2H), 2.52 (d, J = 4.9 Hz, 3H), 2.23 (d, J = 7.1 Hz, 6H)., [M + 1] += 361.3 |
| 17-B | 1H NMR (400 MHz, DMSO) δ 14.04 (s, 2H), 8.77 (s, 1H), 7.44 (s, 1H), 7.14-6.97 (m, 2H), 6.89 (d, J = 7.1 Hz, 1H), |
| | 6.00-5.69 (m, 2H), 4.72-4.47 (m, 1H), 3.90 (t, J = 9.1 Hz, 1H), 3.76 (dd, J = 9.8, 5.6 Hz, 1H), 3.62-3.52 (m, 2H), |
| | 3.49 (dt, J = 8.9, 4.0 Hz, 2H), 3.32 (t, J = 5.8 Hz, 3H), 3.11 (dd, J = 11.6, 5.8 Hz, 2H), 2.52 (d, J = 4.6 Hz, 3H), 2.26 |
| | (d, J = 2.8 Hz, 6H)., [M-TFA + 1] += 361.3 |
| 18 | [M + 1] += 382.2 1H NMR (400 MHz, DMSO) δ 14.12 (s, 2H), 8.85 (s, 1H), 7.49 (s, 1H), 7.10-6.99 (m, 3H), 6.88 (d, |
| | J = 7.3 Hz, 1H), 4.63 (dd, J = 8.3, 5.6 Hz, 1H), 3.91 (t, J = 9.2 Hz, 1H), 3.76 (dd, J = 9.8, 5.5 Hz, 1H), 3.58 (dt, J = 13.5, |
| | 4.1 Hz, 2H), 3.50 (t, J = 4.5 Hz, 2H), 3.41 (t, J = 5.8 Hz, 2H), 3.06 (q, J = 5.8 Hz, 2H), 2.87 (s, 3H), 2.26 (s, 6H). |
| 18-A | 1H NMR (400 MHz, DMSO) δ 12.64 (s, 1H), 7.83 (s, 1H), 7.07-6.96 (m, 4H), 6.91 (s, 1H), 4.49 (t, J = 7.0 Hz, 1H), |
| | 3.97-3.86 (m, 1H), 3.77 (dd, J = 9.6, 6.9 Hz, 1H), 3.56-3.50 (m, 2H), 3.48 (d, J = 4.9 Hz, 2H), 3.40 (t, J = 5.7 Hz, |
| | 2H), 3.05 (q, J = 5.7 Hz, 2H), 2.86 (s, 3H), 2.23 (d, J = 6.5 Hz, 6H)., [M + 1] += 382.2 |
| 18-B | 1H NMR (400 MHz, DMSO) δ 13.26 (s, 2H), 8.36 (d, J = 60.9 Hz, 1H), 7.25 (s, 1H), 7.04 (t, J = 7.4 Hz, 3H), 6.93 (d, |
| | J = 7.2 Hz, 1H), 4.57 (t, J = 6.9 Hz, 1H), 3.91 (t, J = 9.0 Hz, 1H), 3.76 (dd, J = 9.8, 6.2 Hz, 1H), 3.54 (t, J = 8.5 Hz, |
| | 2H), 3.50 (d, J = 4.4 Hz, 2H), 3.41 (t, J = 5.7 Hz, 2H), 3.06 (q, J = 5.7 Hz, 2H), 2.87 (s, 3H), 2.25 (d, J = 3.8 Hz, |
| | 6H)., [M-TFA + 1] += 382.2 |
| 19 | LC-MS: [M-2TFA + 1] += 304.2, 1H NMR (400 MHz, DMSO) δ 14.22 (s, 2H), 8.85 (s, 1H), 7.80 (s, 3H), 7.48 (s, 1H), |
| | 7.13-7.00 (m, 2H), 6.89 (d, J = 7.3 Hz, 1H), 4.63 (dd, J = 8.0, 5.8 Hz, 1H), 3.91 (t, J = 9.1 Hz, 1H), 3.77 (dd, J = 9.8, |
| | 5.7 Hz, 1H), 3.62-3.57 (m, 2H), 3.53 (dd, J = 9.8, 4.8 Hz, 4H), 2.94 (d, J = 4.9 Hz, 2H), 2.26 (d, J = 2.7 Hz, 6H). |
| 20 | 1H NMR (400 MHz, DMSO) δ 11.77 (s, 1H), 9.68 (s, 1H), 8.69 (s, 1H), 8.46 (d, J = 5.9 Hz, 1H), 7.74 (s, 1H), |
| | 7.64-7.42 (m, 2H), 6.97 (s, 2H), 6.75 (s, 1H), 4.45 (s, 1H), 3.93 (s, 1H), 3.80 (t, J = 8.2 Hz, 1H), 3.53 (s, 2H), 3.50 |
| | (s, 2H), 3.42 (t, J = 5.1 Hz, 2H), 3.27 (d, J = 5.1 Hz, 2H), 2.21 (d, J = 7.0 Hz, 6H), 1.24 (s, 2H)., [M + 1] += 425.2 |
| 21 | 1H NMR (400 MHz, DMSO) δ 7.47 (s, 1H), 7.11 (d, J = 7.4 Hz, 1H), 7.02-6.90 (m, 2H), 6.78 (s, 1H), 4.33 (s, 1H), |
| | 4.16-4.04 (m, 1H), 4.01 (t, J = 5.3 Hz, 2H), 3.70-3.57 (m, 2H), 3.56-3.44 (m, 14H), 3.45-3.39 (m, 2H), 3.36 (t, |
| | J = 6.5 Hz, 2H), 3.24 (s, 3H), 3.19 (t, J = 7.1 Hz, 1H), 2.22 (d, J = 8.4 Hz, 6H), 2.09-1.92 (m, 1H), 1.88-1.68 (m, |
| | 1H), 1.47-1.27 (m, 2H)., [M + 1] += 479.3 |
| 22 | [M + 1] += 400.2, 1H NMR (400 MHz, DMSO) δ 13.46 (s, 1H), 8.27 (s, 1H), 7.24 (dd, J = 13.6, 5.7 Hz, 2H), 7.04 (d, J = |
| | 11.2 Hz, 2H), 6.93-6.49 (m, 5H), 5.68 (s, 1H), 3.96 (s, 2H), 3.29 (d, J = 4.5 Hz, 2H), 2.91 (s, 3H), 2.24 (s, 3H), 2.12 (s, 3H). |
| 23 | LC-MS = [M-C2HF3O2 + 1] += 376.2, 1H NMR (400 MHz, DMSO) δ 14.24 (s, 2H), 8.90 (s, 1H), 7.44 (t, J = 7.6 Hz, |
| | 1H), 7.33 (d, J = 7.6 Hz, 1H), 7.26 (d, J = 7.7 Hz, 1H), 7.18-7.04 (m, 3H), 6.92 (s, 1H), 6.70 (d, J = 7.4 Hz, 1H), 5.88 |
| | (s, 1H), 3.44 (d, J = 102.9 Hz, 8H), 2.26 (s, 3H), 2.12 (s, 3H). |
| 24 | LC-MS = [M + 1-TFA] += 318.2, 1H NMR (400 MHz, DMSO) δ 14.43 (s, 2H), 9.00 (s, 1H), 8.52 (s, 2H), 7.56 (s, 1H), |
| | 7.13-7.01 (m, 2H), 6.87 (d, J = 7.2 Hz, 1H), 4.66 (dd, J = 7.9, 5.7 Hz, 1H), 3.91 (t, J = 9.1 Hz, 1H), 3.77 (dd, J = 9.8, |
| | 5.5 Hz, 1H), 3.63-3.50 (m, 6H), 3.05 (s, 2H), 2.54 (s, 3H), 2.27 (s, 6H). |
| 25 | LC-MS = [M + 1-TFA] += 346.2, 1H NMR (400 MHz, DMSO) δ 14.24 (s, 2H), 8.92 (s, 1H), 7.87 (s, 1H), 7.51 (s, 1H), |
| | 7.12-7.01 (m, 2H), 6.88 (d, J = 7.3 Hz, 1H), 4.64 (dd, J = 8.0, 5.6 Hz, 1H), 3.90 (t, J = 9.2 Hz, 1H), 3.76 (dd, J = 9.8, |
| | 5.4 Hz, 1H), 3.61-3.53 (m, 2H), 3.48 (t, J = 4.4 Hz, 2H), 3.35 (t, J = 5.9 Hz, 2H), 3.15 (q, J = 5.8 Hz, 2H), 2.26 (s, |
| | 6H), 1.78 (s, 3H). |
| 26 | 1H NMR (400 MHz, DMSO) δ 14.23 (s, 2H), 8.97 (s, 1H), 7.55 (s, 1H), 7.05 (dt, J = 19.1, 5.9 Hz, 3H), 6.86 (d, J = 7.3 |
| | Hz, 1H), 4.65 (dd, J = 8.2, 5.4 Hz, 1H), 3.90 (t, J = 9.2 Hz, 1H), 3.76 (dd, J = 9.8, 5.4 Hz, 1H), 3.60-3.54 (m, |
| | 2H), 3.53-3.47 (m, 6H), 3.44 (t, J = 5.8 Hz, 2H), 3.07 (q, J = 5.8 Hz, 2H), 2.89 (s, 3H), 2.27 (s, 6H)., |
| | [M-C2HF3O2 + 1] += 426.2 |
| 27 | LCMS: [M-C2HF3O2 + 1] += 324.2, 1H NMR (400 MHz, DMSO) δ 14.36 (s, 5H), 8.97 (s, 3H), 7.77 (s, 3H), 7.64 (s, |
| | 3H), 7.60 (t, J = 6.7 Hz, 3H), 7.24 (t, J = 7.7 Hz, 3H), 7.15 (d, J = 7.3 Hz, 3H), 7.13-6.98 (m, 9H), 6.71 (d, J = 7.5 |
| | Hz, 3H), 6.01 (s, 3H), 2.27 (s, 9H), 2.12 (s, 9H) |
| 28 | LC-MS = [M + 1-TFA] += 356.1, 1H NMR (400 MHz, DMSO) δ 14.32 (s, 2H), 9.01 (s, 1H), 7.71 (d, J = 7.8 Hz, 1H), |
| | 7.64-7.58 (m, 2H), 7.46 (dt, J = 17.1, 6.3 Hz, 2H), 7.12 (dt, J = 15.1, 7.4 Hz, 2H), 6.95 (s, 1H), 6.68 (d, J = 7.4 Hz, |
| | 1H), 2.37 (d, J = 4.9 Hz, 3H), 2.26 (s, 3H), 2.12 (s, 3H). |
| 29 | LC-MS = [M-C2HF3O2 + 1] += 356.1, 1H NMR (400 MHz, DMSO) δ 14.31 (s, 2H), 9.75 (s, 1H), 9.01 (s, 1H), 7.32 (t, |
| | J = 7.9 Hz, 1H), 7.13 (d, J = 8.0 Hz, 2H), 7.08 (t, J = 7.6 Hz, 1H), 7.01 (s, 1H), 6.95 (s, 1H), 6.90 (d, J = 7.6 Hz, 1H), |
| | 6.66 (d, J = 7.4 Hz, 1H), 5.81 (s, 1H), 2.96 (s, 3H), 2.26 (s, 3H), 2.13 (s, 3H). |
| 30 | LC-MS = [M-C2HF3O2 + 1] += 320.1, 1H NMR (400 MHz, DMSO) δ 14.36 (s, 2H), 9.95 (s, 1H), 9.06 (s, 1H), 7.54 (d, |
| | J = 8.1 Hz, 1H), 7.35 (s, 1H), 7.27 (t, J = 7.9 Hz, 1H), 7.10 (dt, J = 15.0, 7.3 Hz, 2H), 6.96 (s, 1H), 6.85 (d, J = 7.6 Hz, |
| | 1H), 6.66 (d, J = 7.4 Hz, 1H), 5.78 (s, 1H), 2.26 (s, 3H), 2.13 (s, 3H), 1.99 (s, 3H). |
| 31 | LC-MS = [M + 1] += 307.2, 1H NMR (400 MHz, DMSO) δ 12.53 (s, 1H), 8.52 (d, J = 4.7 Hz, 1H), 8.10 (s, 1H), 7.86 (s, |
| | 1H), 7.79 (s, 1H), 7.63 (s, 1H), 7.35 (d, J = 3.8 Hz, 1H), 7.10-7.00 (m, 2H), 6.81 (d, J = 7.2 Hz, 1H), 6.66 (s, 1H), |
| | 5.80 (s, 1H), 2.24 (s, 3H), 2.11 (s, 3H). |
| 32 | [M + 1] += 331.2. 1H NMR (400 MHz, DMSO) δ 14.18 (s, 2H), 8.91 (s, 1H), 7.15-7.01 (m, 3H), 6.90 (s, 1H), 6.70 (d, |
| | J = 7.2 Hz, 1H), 6.52 (d, J = 8.0 Hz, 1H), 6.41 (s, 1H), 6.36 (d, J = 7.6 Hz, 1H), 5.97 (s, 1H), 5.65 (s, 1H), 3.49-3.13 |
| | (m, 5H), 2.67 (t, J = 6.5 Hz, 2H), 2.25 (s, 3H), 2.13 (s, 3H). Y = 59.92%. Total yield = 43.96%. |
| 33 | [M-CF3COOH + 1] += 334.2 1H NMR (400 MHz, DMSO) δ 14.32 (s, 2H), 8.98 (s, 1H), 7.42 (t, J = 7.6 Hz, 1H), 7.32 |
| | (d, J = 7.6 Hz, 1H), 7.23 (d, J = 7.7 Hz, 1H), 7.18 (s, 1H), 7.16-7.03 (m, 2H), 6.93 (s, 1H), 6.70 (d, J = 7.4 Hz, 1H), |
| | 5.89 (s, 1H), 2.94 (s, 3H), 2.85 (s, 3H), 2.26 (s, 3H), 2.13 (s, 3H). |
| 34 | [M-C2HF3O2 + 1] += 306.2, 1H NMR (400 MHz, DMSO) δ 14.34 (s, 2H), 9.05 (d, J = 1.2 Hz, 1H), 7.80 (s, 1H), 7.53 |
| | (dt, J = 7.9, 3.6 Hz, 1H), 7.45-7.33 (m, 3H), 7.17-7.04 (m, 2H), 6.89 (dt, J = 6.9, 3.5 Hz, 1H), 6.80 (s, 1H), 6.66-6.57 |
| | (m, 2H), 2.25 (s, 3H), 2.07 (d, J = 6.5 Hz, 3H). |
| 35 | LC-MS = [M-C2HF3O2 + 1] += 376.1, 1H NMR (400 MHz, DMSO) δ 14.46 (s, 2H), 11.84 (s, 1H), 9.10 (d, J = 0.9 Hz, |
| | 1H), 7.72 (d, J = 7.8 Hz, 1H), 7.67 (dd, J = 8.1, 1.3 Hz, 1H), 7.60 (s, 1H), 7.50 (t, J = 7.7 Hz, 1H), 7.41 (t, J = 8.0 Hz, |
| | 2H), 7.11 (s, 1H), 7.03 (dd, J = 7.8, 1.2 Hz, 1H), 6.06 (s, 1H), 3.69 (s, 3H). |
| 36 | LCMS: [M-C2HF3O2 + 1] += 356.2/358.1, 1H NMR (400 MHz, DMSO) δ 14.45 (s, 2H), 11.83 (s, 1H), 9.08 (d, J = 5.9 |
| | Hz, 1H), 7.70 (d, J = 7.7 Hz, 1H), 7.60 (s, 1H), 7.51-7.42 (m, 2H), 7.37 (d, J = 7.6 Hz, 1H), 7.25 (t, J = 7.9 Hz, 1H), |
| | 7.02 (s, 1H), 6.84 (d, J = 7.8 Hz, 1H), 5.96 (d, J = 5.6 Hz, 1H), 3.69 (s, 3H), 2.29 (s, 3H). |
| 37 | [M-C2HF3O2 + 1] += 319.3, 1H NMR (400 MHz, DMSO) δ 14.31 (s, 2H), 9.00 (s, 1H), 7.57 (s, 1H), 7.15-6.96 (m, |
| | 2H), 6.86 (d, J = 7.4 Hz, 1H), 4.69-4.62 (m, 1H), 3.91 (t, J = 9.2 Hz, 1H), 3.76 (dd, J = 9.7, 5.2 Hz, 1H), 3.61-3.53 |
| | (m, 2H), 3.52-3.45 (m, 4H), 3.43-3.37 (m, 2H), 3.23 (s, 3H), 2.27 (s, 6H). |
| 38 | [M-2HF302 + 1] += 340.1, 1H NMR (400 MHz, DMSO) δ 14.41 (s, 2H), 9.08 (d, J = 1.2 Hz, 1H), 7.90 (s, 1H), 7.62 (s, |
| | 1H), 7.43-7.33 (m, 2H), 7.13 (dt, J = 11.6, 7.4 Hz, 2H), 6.98-6.90 (m, 2H), 6.64 (d, J = 7.4 Hz, 1H), 6.08 (s, 1H), |
| | 2.31-2.24 (m, 3H), 2.09 (s, 3H). |
| 39 | [M + 1] += 278.2., 1H NMR (400 MHz, DMSO) δ 7.55 (d, J = 1.0 Hz, 1H), 7.03-6.94 (m, 2H), 6.90 (t, J = 7.7 Hz, 1H), |
| | 6.79 (d, J = 6.9 Hz, 1H), 6.38 (dd, J = 6.0, 3.1 Hz, 2H), 6.33 (s, 1H), 6.27 (d, J = 7.6 Hz, 1H), 5.40 (s, 1H), 4.94 (s, |
| | 1H), 2.23 (s, 3H), 2.11 (s, 3H) |
| 40 | [M-C2HF3O2 + 1] += 368.2. 1H NMR (400 MHz, DMSO) δ 14.45 (s, 2H), 9.03 (s, 1H), 7.37 (s, 1H), 7.27 (dd, J = 6.3, |
| | 2.4 Hz, 1H), 7.19-7.14 (m, 2H), 7.05 (t, J = 5.9 Hz, 1H), 5.87 (s, 1H), 3.59 (s, 4H), 3.45 (t, J = 5.8 Hz, 2H), 3.09 (q, |
| | J = 5.8 Hz, 2H), 2.88 (s, 3H), 2.26 (s, 3H), 2.15 (s, 3H). |
| 41 | [M-C2HF3O2 + 1] += 363.2, 1H NMR (400 MHz, DMSO) δ 14.44 (s, 2H), 10.20 (s, 1H), 9.07 (s, 1H), 7.86 (t, J = 7.5 |
| | Hz, 1H), 7.17 (dd, J = 15.4, 7.5 Hz, 2H), 7.13-7.00 (m, 2H), 6.88-6.59 (m, 2H), 6.01 (s, 1H), 3.97 (s, 2H), 2.25 (d, |
| | J = 18.4 Hz, 3H), 2.14 (s, 3H). |
| 42 | [M-C2HF3O2 + 1] += 377.2, 1H NMR (400 MHz, DMSO) δ 14.33 (s, 2H), 9.95 (s, 1H), 9.07 (d, J = 1.1 Hz, 1H), 7.54 |
| | (d, J = 8.3 Hz, 1H), 7.40 (s, 1H), 7.32 (s, 1H), 7.28 (t, J = 7.9 Hz, 1H), 7.13 (d, J = 6.9 Hz, 1H), 7.09 (t, J = 7.5 Hz, |
| | 1H), 6.96 (s, 1H), 6.84 (d, J = 7.7 Hz, 1H), 6.75 (s, 1H), 6.67 (d, J = 7.1 Hz, 1H), 5.78 (s, 1H), 2.47 (d, J = 6.7 Hz, |
| | 2H), 2.35 (t, J = 7.0 Hz, 2H), 2.26 (s, 3H), 2.13 (s, 3H). |
| 43 | [M-C2HF3O2 + 1] += 359.2, 1H NMR (400 MHz, DMSO) δ 14.35 (s, 2H), 10.12 (s, 1H), 9.07 (d, J = 1.1 Hz, 1H), 7.54 |
| | (d, J = 9.1 Hz, 1H), 7.40 (s, 1H), 7.31 (t, J = 7.9 Hz, 1H), 7.14 (d, J = 6.7 Hz, 1H), 7.09 (t, J = 7.5 Hz, 1H), 6.96 (s, |
| | 1H), 6.89 (d, J = 7.7 Hz, 1H), 6.67 (d, J = 7.0 Hz, 1H), 5.80 (s, 1H), 2.74-2.61 (m, 4H), 2.26 (s, 3H), 2.13 (s, 3H). |
| 43-A | [M + 1] += 359.2., 1H NMR (400 MHz, MeOD) δ 7.65 (s, 1H), 7.56 (d, J = 8.0 Hz, 1H), 7.26 (dd, J = 9.8, 5.6 Hz, 2H), |
| | 7.05 (d, J = 7.3 Hz, 1H), 6.99 (t, J = 7.6 Hz, 1H), 6.89 (d, J = 7.7 Hz, 1H), 6.72 (d, J = 7.5 Hz, 1H), 6.38 (s, 1H), 5.68 |
| | (s, 1H), 2.79-2.65 (m, 4H), 2.29 (s, 3H), 2.17 (s, 3H). |
| 43-B | [M + 1] += 359.2., 1H NMR (400 MHz, MeOD) δ 7.65 (s, 1H), 7.61-7.51 (m, 1H), 7.25 (dd, J = 9.8, 5.8 Hz, 2H), 7.05 |
| | (d, J = 7.2 Hz, 1H), 6.99 (t, J = 7.6 Hz, 1H), 6.89 (d, J = 7.8 Hz, 1H), 6.72 (d, J = 7.5 Hz, 1H), 6.38 (s, 1H), 5.68 (s, |
| | 1H), 2.79-2.67 (m, 4H), 2.29 (s, 3H), 2.17 (s, 3H). |
| 44-A | [M + 1] += 356.2., 1H NMR (400 MHz, DMSO) δ 11.86 (s, 1H), 9.64 (s, 1H), 7.58 (d, J = 1.0 Hz, 1H), 7.23 (t, J = 7.8 |
| | Hz, 1H), 7.08-6.96 (m, 4H), 6.87 (d, J = 7.7 Hz, 1H), 6.79 (s, 1H), 6.43 (s, 1H), 5.55 (s, 1H), 2.93 (s, 3H), 2.23 (s, |
| | 3H), 2.12 (s, 3H). |
| 44-B | [M + 1] += 356.2., 1H NMR (400 MHz, DMSO) δ 11.86 (s, 1H), 9.64 (s, 1H), 7.58 (d, J = 1.0 Hz, 1H), 7.23 (t, J = 7.8 |
| | Hz, 1H), 7.08-6.96 (m, 4H), 6.87 (d, J = 7.7 Hz, 1H), 6.79 (s, 1H), 6.43 (s, 1H), 5.55 (s, 1H), 2.93 (s, 3H), 2.23 (s, |
| | 3H), 2.12 (s, 3H). |
| 45-A | [M + 1] += 324.1, 1H NMR (400 MHz, DMSO) δ 11.99 (s, 1H), 7.71 (s, 1H), 7.62 (d, J = 1.0 Hz, 1H), 7.57 (s, 1H), 7.50 |
| | (td, J = 7.3, 1.8 Hz, 1H), 7.17 (t, J = 7.6 Hz, 1H), 7.10 (t, J = 6.6 Hz, 1H), 7.07-6.96 (m, 2H), 6.77 (d, J = 7.3 Hz, |
| | 1H), 6.51 (s, 1H), 5.84 (s, 1H), 2.24 (s, 3H), 2.12 (s, 3H). |
| 45-B | [M + 1] += 324.2, 1H NMR (400 MHz, DMSO) δ 12.00 (s, 1H), 7.71 (s, 1H), 7.63 (d, J = 0.9 Hz, 1H), 7.57 (s, 1H), 7.50 |
| | (td, J = 7.3, 1.8 Hz, 1H), 7.17 (t, J = 7.6 Hz, 1H), 7.10 (t, J = 6.5 Hz, 1H), 7.07-6.96 (m, 2H), 6.77 (d, J = 7.3 Hz, |
| | 1H), 6.51 (s, 1H), 5.84 (s, 1H), 2.24 (s, 3H), 2.12 (s, 3H). |
| 46-A | [M + 1] += 400.2, 1H NMR (400 MHz, DMSO + D2O) δ 7.58 (d, J = 0.9 Hz, 1H), 7.20 (t, J = 7.7 Hz, 1H), 7.11-6.86 (m, |
| | 2H), 6.74 (dd, J = 26.0, 7.5 Hz, 3H), 6.64 (s, 1H), 6.44 (s, 1H), 5.56 (s, 1H), 3.93 (t, J = 5.3 Hz, 2H), 3.27 (t, J = 5.3 |
| | Hz, 2H), 2.89 (s, 3H), 2.20 (s, 3H), 2.09 (s, 3H). |
| 46-B | [M + 1] += 400.2, 1H NMR (400 MHz, DMSO + D2O) δ 7.58 (s, 1H), 7.20 (t, J = 7.7 Hz, 1H), 6.98 (d, J = 8.5 Hz, 2H), |
| | 6.73 (dd, J = 26.9, 7.4 Hz, 3H), 6.62 (s, 1H), 6.44 (s, 1H), 5.56 (s, 1H), 3.26 (t, J = 5.2 Hz, 2H), 2.89 (s, 3H), 2.19 (s, |
| | 3H), 2.08 (s, 3H). |
| 47-A | [M + 1] += 356.1, 1H NMR (400 MHz, DMSO) δ 11.90 (s, 1H), 7.61 (d, J = 4.3 Hz, 2H), 7.57 (s, 1H), 7.51 (t, J = 7.6 |
| | Hz, 1H), 7.41 (d, J = 7.5 Hz, 2H), 7.07-6.97 (m, 2H), 6.67 (d, J = 113.4 Hz, 2H), 5.72 (s, 1H), 2.37 (d, J = 5.0 Hz, |
| | 3H), 2.24 (s, 3H), 2.12 (s, 3H). |
| 47-B | [M + 1] += 356.1, 1H NMR (400 MHz, DMSO) δ 7.61 (dd, J = 9.5, 4.4 Hz, 2H), 7.55-7.45 (m, 2H), 7.38 (d, J = 7.7 Hz, |
| | 1H), 7.10-6.92 (m, 2H), 6.72 (s, 1H), 6.47 (s, 1H), 5.71 (s, 1H), 2.34 (s, 3H), 2.20 (s, 3H), 2.10-2.07 (m, 3H). |
| 48 | LCMS: [M-C2HF3O2 + 1] += 332.2, 1H NMR (400 MHz, DMSO) δ 14.58 (s, 1H), 9.48 (s, 1H), 9.08 (s, 1H), 7.21-6.99 |
| | (m, 4H), 6.74-6.59 (m, 3H), 6.53 (s, 1H), 5.72 (s, 1H), 4.42 (t, J = 6.5 Hz, 2H), 3.18 (t, J = 6.5 Hz, 2H), 2.25 (s, |
| | 3H), 2.11 (s, 3H). |
| 49 | [M-C2HF3O2 + 1] += 308.2, 1H NMR (400 MHz, DMSO) δ 14.30 (s, 2H), 11.79 (s, 1H), 9.04 (s, 1H), 7.72-7.60 (m, |
| | 2H), 7.51-7.22 (m, 7H), 7.15 (s, 1H), 5.74 (s, 1H), 3.69 (s, 3H). |
| 50 | LC-MS = [M-C2HF3O2 + 1] += 338.0, 1H NMR (400 MHz, DMSO) δ 14.43 (s, 2H), 9.75 (s, 1H), 9.07 (d, J = 1.2 Hz, |
| | 1H), 7.87 (t, J = 7.5 Hz, 1H), 7.17-7.06 (m, 4H), 6.69 (dd, J = 13.1, 7.0 Hz, 2H), 6.00 (s, 1H), 2.28 (s, 3H), 2.14 (s, |
| | 3H), 2.07 (s, 3H). |
| 51 | LC-MS = [M-C2HF3O2 + 1] += 374.0, 1H NMR (400 MHz, DMSO) δ 14.42 (s, 2H), 9.68 (s, 1H), 9.08 (d, J = 1.2 Hz, |
| | 1H), 7.39 (td, J = 7.9, 1.3 Hz, 1H), 7.22-7.14 (m, 2H), 7.09 (dd, J = 13.2, 5.6 Hz, 2H), 6.78 (dd, J = 10.5, 4.0 Hz, |
| | 1H), 6.69 (d, J = 7.4 Hz, 1H), 6.00 (s, 1H), 3.03 (s, 3H), 2.28 (s, 3H), 2.12 (s, 3H). |
| 52 | [M -C2HF3O2 + 1] += 390.2, 1H NMR (400 MHz, DMSO) δ 14.43 (s, 1.5H), 11.83 (s, 1H), 9.09 (d, J = 0.9 Hz, 1H), |
| | 7.70 (t, J = 6.7 Hz, 2H), 7.62 (s, 1H), 7.49 (t, J = 7.7 Hz, 1H), 7.44 (t, J = 7.9 Hz, 1H), 7.37 (d, J = 7.7 Hz, 1H), 7.19 |
| | (d, J = 7.7 Hz, 1H), 7.02 (s, 1H), 6.05 (s, 1H), 3.69 (s, 3H), 2.34 (d, J = 7.0 Hz, 3H). |
| 53 | [M-C2HF3O2 + 1] += 354.2., 1H NMR (400 MHz, DMSO) δ 14.39 (s, 2H), 11.58 (s, 1H), 9.07 (s, 1H), 7.48-7.39 (m, |
| | 1H), 7.38-7.26 (m, 2H), 7.17-7.06 (m, 2H), 6.97 (s, 1H), 6.68 (d, J = 7.3 Hz, 1H), 5.93 (s, 1H), 3.69 (s, 3H), 2.26 |
| | (s, 3H), 2.12 (s, 3H). |
| 54 | [M -C2HF3O2 + 1] += 390.2, 1H NMR (400 MHz, DMSO) δ 14.43 (s, 1.5H), 11.83 (s, 1H), 9.09 (d, J = 0.9 Hz, 1H), |
| | 7.70 (t, J = 6.7 Hz, 2H), 7.62 (s, 1H), 7.49 (t, J = 7.7 Hz, 1H), 7.44 (t, J = 7.9 Hz, 1H), 7.37 (d, J = 7.7 Hz, 1H), 7.19 |
| | (d, J = 7.7 Hz, 1H), 7.02 (s, 1H), 6.05 (s, 1H), 3.69 (s, 3H), 2.34 (d, J = 7.0 Hz, 3H). |
| 54 | [M-C2HF3O2 + 1] += 354.2, 1H NMR (400 MHz, DMSO) δ 14.37 (s, 2H), 11.65 (s, 1H), 9.05 (s, 1H), 7.55 (t, J = 6.5 |
| | Hz, 1H), 7.27 (t, J = 7.7 Hz, 1H), 7.17 (d, J = 7.3 Hz, 1H), 7.10 (dd, J = 16.2, 8.9 Hz, 3H), 6.71 (d, J = 7.5 Hz, 1H), |
| | 6.01 (s, 1H), 3.70 (s, 3H), 2.28 (s, 3H), 2.12 (s, 3H). |
| 55 | LCMS: [M-C2HF3O2 + 1] += 418.1, 1H NMR (400 MHz, DMSO) δ 8.95 (d, J = 1.2 Hz, 1H), 7.10 (ddd, J = 15.2, 10.7, |
| | 7.4 Hz, 4H), 6.99 (s, 1H), 6.65 (t, J = 12.9 Hz, 1H), 6.48 (dd, J = 8.8, 5.2 Hz, 1H), 5.96 (s, 1H), 4.08 (t, J = 5.3 Hz, |
| | 2H), 3.33 (t, J = 5.2 Hz, 2H), 2.92 (s, 3H), 2.24 (s, 3H), 2.09 (s, 3H). |
| 56 | [M + 1] += 307.1, 1H NMR (400 MHz, DMSO) δ 11.94 (s, 1H), 8.06-7.78 (m, 3H), 7.63 (d, J = 20.5 Hz, 2H), 7.40 |
| | (dd, J = 6.7, 1.9 Hz, 1H), 7.14-6.89 (m, 3H), 6.61 (s, 1H), 5.83 (s, 1H), 2.20 (d, J = 22.4 Hz, 6H). |
| 57 | [M + 1] += 357.1, 1H NMR (400 MHz, DMSO) δ 11.93 (s, 1H), 10.81 (s, 1H), 8.06 (s, 1H), 7.61 (s, 1H), 7.14-6.93 (m, |
| | 2H), 6.93-6.48 (m, 4H), 5.56 (s, 1H), 3.20 (s, 3H), 2.26 (s, 3H), 2.10 (s, 3H). |
| 58 | [M + 1] += 325.2, 1H NMR (400 MHz, CD3OD) δ 8.36 (d, J = 4.8 Hz, 1H), 7.73 (d, J = 0.9 Hz, 1H), 7.13 (dt, J = 22.7, |
| | 10.1 Hz, 2H), 7.03 (t, J = 7.6 Hz, 1H), 6.72 (d, J = 7.7 Hz, 1H), 6.53 (s, 1H), 6.05 (s, 1H), 2.32 (s, 3H), 2.21 (s, 3H). |
| 59 | [M + 1] += 355.15, 1H NMR (400 MHz, DMSO) δ 14.31 (s, 1H), 9.27 (s, 1H), 9.08 (s, 1H), 8.08 (d, J = 4.0 Hz, 1H), |
| | 7.68 (d, J = 8.0 Hz, 1H), 7.60 (t, J = 7.2 Hz, 1H), 7.34 (s, 1H), 7.28 (t, J = 8.0 Hz, 1H), 7.16-7.06 (m, 2H), 7.02 (s, |
| | 1H), 6.83 (d, J = 8.4 Hz, 1H), 6.80-6.74 (m, 2H), 6.71 (d, J = 7.2 Hz, 1H), 5.78 (s, 1H), 2.27 (s, 3H), 2.16 (s, 3H). |
| 60 | [M-TFA − 1] −= 311.2, 1H NMR (400 MHz, DMSO) δ 14.20 (s, 2H), 12.02 (s, 1H), 9.02-8.89 (m, 1H), 7.75-7.56 (m, |
| | 1H), 7.13 (ddt, J = 22.9, 16.8, 9.0 Hz, 3H), 4.26-4.06 (m, 1H), 2.28-2.08 (m, 8H), 1.91-0.73 (m, 8H). |
| 61 | [M -C2HF3O2 + 1] += 350.2, 1H NMR (400 MHz, DMSO) δ 14.20 (s, 2H), 11.69 (s, 1H), 8.96 (d, J = 0.9 Hz, 1H), 7.61 |
| | (d, J = 4.6 Hz, 2H), 7.55-7.48 (m, 1H), 7.31 (t, J = 6.4 Hz, 2H), 7.07 (dt, J = 12.8, 4.6 Hz, 3H), 4.74 (t, J = 7.9 Hz, |
| | 1H), 3.70 (s, 3H), 3.39 (d, J = 8.5 Hz, 1H), 3.19 (dd, J = 13.8, 7.3 Hz, 1H), 2.21 (s, 3H), 2.12 (s, 3H). |
| 62 | [M-C2HF3O2 + 1] += 336.2, 1H NMR (400 MHz, DMSO) δ 14.30 (s, 2H), 9.67 (s, 1H), 9.06 (d, J = 0.8 Hz, 1H), |
| | 7.40-7.31 (m, 2H), 7.27 (t, J = 7.8 Hz, 1H), 7.16-7.05 (m, 2H), 6.96 (s, 1H), 6.80 (d, J = 7.6 Hz, 1H), 6.67 (d, J = 7.2 Hz, |
| | 1H), 5.77 (s, 1H), 3.63 (s, 3H), 2.26 (s, 3H), 2.13 (s, 3H). |
| 63 | [M-C2HF3O2 + 1] += 380.1, 1H NMR (400 MHz, DMSO) δ 14.30 (s, 2H), 9.99 (s, 1H), 9.05 (d, J = 0.9 Hz, 1H), 7.55 |
| | (d, J = 8.2 Hz, 1H), 7.39 (s, 1H), 7.29 (t, J = 7.9 Hz, 1H), 7.16-7.05 (m, 2H), 6.95 (s, 1H), 6.86 (d, J = 7.7 Hz, 1H), |
| | 6.67 (d, J = 7.1 Hz, 1H), 5.78 (s, 1H), 2.70 (t, J = 7.0 Hz, 2H), 2.57 (t, J = 7.1 Hz, 2H), 2.25 (d, J = 10.4 Hz, 3H), 2.13 |
| | (s, 3H), 2.06 (s, 3H). |
| 64 | [M-C2HF3O2 + 1] += 392.2, 1H NMR (400 MHz, DMSO) δ 14.29 (s, 2H), 9.74 (s, 1H), 9.06 (d, J = 1.1 Hz, 1H), 7.34 |
| | (dd, J = 20.1, 15.2 Hz, 2H), 7.25 (d, J = 7.9 Hz, 1H), 7.19-7.03 (m, 2H), 6.96 (s, 1H), 6.81 (d, J = 7.5 Hz, 1H), 6.66 |
| | (d, J = 7.2 Hz, 1H), 5.76 (s, 1H), 5.21 (dd, J = 6.0, 4.4 Hz, 1H), 4.02-3.64 (m, 5H), 3.47 (s, 18H), 2.55-2.46 (m, |
| | 7H), 2.26 (s, 3H), 2.23-1.97 (m, 4H), 1.97-1.80 (m, 1H), 1.44-1.11 (m, 1H), 0.01 (s, 1H). |
| 65 | [M-C2HF3O2 + 1] += 334.2, 1H NMR (400 MHz, DMSO) δ 14.34 (s, 2H), 9.06 (d, J = 1.1 Hz, 1H), 8.34 (t, J = 5.8 Hz, |
| | 1H), 7.32 (t, J = 7.6 Hz, 1H), 7.21-7.15 (m, 1H), 7.15-6.99 (m, 4H), 6.94 (s, 1H), 6.69 (d, J = 7.2 Hz, 1H), 5.81 (s, |
| | 1H), 4.26-4.16 (m, 2H), 2.26 (s, 3H), 2.14 (s, 3H), 1.84 (s, 3H). |
| 66 | [M-C2HF3O2 + 1] += 338.1, 1H NMR (400 MHz, DMSO) δ 14.25 (s, 2H), 9.00 (d, J = 1.1 Hz, 1H), 7.58 (s, 1H), 7.07 |
| | (dt, J = 12.0, 7.0 Hz, 3H), 6.87 (d, J = 7.5 Hz, 1H), 4.66 (dd, J = 8.5, 5.3 Hz, 1H), 3.91 (t, J = 9.3 Hz, 1H), 3.76 (dd, |
| | J = 9.8, 5.3 Hz, 1H), 3.51 (d, J = 2.1 Hz, 2H), 3.09 (q, J = 5.8 Hz, 2H), 2.83 (s, 3H), 2.28 (s, 6H). |
| 67 | [M-C2HF3O2 + 1] += 370.2, 1H NMR (400 MHz, DMSO) δ 14.33 (s, 2H), 9.80 (s, 1H), 9.07 (s, 1H), 7.31 (t, J = 7.9 |
| | Hz, 1H), 7.17-7.05 (m, 3H), 7.02 (s, 1H), 6.97 (s, 1H), 6.89 (d, J = 7.7 Hz, 1H), 6.65 (d, J = 7.4 Hz, 1H), 5.81 (s, |
| | 1H), 3.05 (q, J = 7.3 Hz, 2H), 2.26 (s, 3H), 2.12 (s, 3H), 1.15 (t, J = 7.3 Hz, 3H). |
| 68 | [M-C2HF3O2 + 1] += 288.2, 1H NMR (400 MHz, DMSO) δ 14.39 (s, 2H), 9.06 (d, J = 0.9 Hz, 1H), 7.80 (d, J = 7.6 Hz, |
| | 1H), 7.66 (s, 1H), 7.59 (t, J = 7.7 Hz, 1H), 7.53 (d, J = 8.0 Hz, 1H), 7.18-7.08 (m, 2H), 7.03 (s, 1H), 6.69 (d, J = 7.3 |
| | Hz, 1H), 5.96 (s, 1H), 2.27 (s, 3H), 2.12 (s, 3H). |
| 69 | [M-C2HF3O2 + 1] += 370.1, 1H NMR (400 MHz, DMSO) δ 14.38 (s, 2H), 9.07 (d, J = 1.1 Hz, 1H), 7.56 (t, J = 6.3 Hz, |
| | 1H), 7.36 (t, J = 7.6 Hz, 1H), 7.28 (d, J = 7.7 Hz, 1H), 7.19 (s, 1H), 7.15-7.06 (m, 3H), 6.94 (s, 1H), 5.83 (s, 1H), |
| | 4.13 (d, J = 6.3 Hz, 2H), 2.80 (s, 3H), 2.26 (s, 3H), 2.14 (s, 3H). |
| 70 | [M-C4H2F604 + 1] += 419.2, 1H NMR (400 MHz, DMSO + D2O) δ 8.98 (s, 1H), 7.50 (d, J = 8.0 Hz, 1H), 7.38 (s, 1H), |
| | 7.32 (t, J = 7.9 Hz, 1H), 7.16-7.02 (m, 2H), 6.89 (d, J = 3.9 Hz, 2H), 6.64 (d, J = 7.4 Hz, 1H), 5.77 (s, 1H), 3.95 (s, |
| | 2H), 3.30 (dd, J = 62.3, 55.3 Hz, 6H), 2.78 (t, J = 7.1 Hz, 2H), 2.24 (s, 3H), 2.11 (s, 3H). |
| 71 | [M-C2HF3O2 + 1] += 370.1, 1H NMR (400 MHz, DMSO) δ 14.39 (s, 2H), 9.08 (d, J = 1.1 Hz, 1H), 7.46-7.33 (m, |
| | 2H), 7.27 (s, 1H), 7.17-7.05 (m, 3H), 6.96 (s, 1H), 6.70 (d, J = 7.2 Hz, 1H), 5.87 (s, 1H), 3.21 (s, 3H), 2.92 (s, 3H), |
| | 2.26 (s, 3H), 2.15 (s, 3H). |
| 72 | [M-C2HF3O2 + 1] += 351.2, 1H NMR (400 MHz, D2O) δ 8.56 (s, 1H), 7.28 (t, J = 7.8 Hz, 1H), 7.19 (d, J = 8.1 Hz, 1H), |
| | 7.12 (d, J = 10.3 Hz, 2H), 7.02 (t, J = 7.7 Hz, 1H), 6.95 (d, J = 7.7 Hz, 1H), 6.80 (s, 1H), 6.70 (d, J = 7.7 Hz, 1H), 5.81 |
| | (s, 1H), 3.62 (s, 3H), 2.19 (s, 3H), 2.08 (s, 3H). |
| 73 | [M -C2HF3O2 + 1] += 390.2, 1H NMR (400 MHz, DMSO) δ 14.32 (s, 2H), 9.96 (s, 1H), 9.07 (d, J = 0.9 Hz, 1H), 7.56 |
| | (d, J = 8.2 Hz, 1H), 7.40 (s, 1H), 7.28 (t, J = 7.9 Hz, 1H), 7.16-7.05 (m, 2H), 6.97 (s, 1H), 6.85 (d, J = 7.7 Hz, 1H), |
| | 6.67 (d, J = 7.3 Hz, 1H), 5.79 (s, 1H), 3.79 (d, J = 8.1 Hz, 1H), 3.75 (dd, J = 8.8, 3.4 Hz, 1H), 3.72-3.65 (m, 2H), |
| | 3.30 (dd, J = 8.2, 6.4 Hz, 1H), 2.58-2.52 (m, 1H), 2.42-2.32 (m, 2H), 2.26 (s, 3H), 2.13 (s, 3H), 2.00 (dtd, J = 12.9, |
| | 7.7, 5.5 Hz, 1H), 1.51 (td, J = 14.5, 7.1 Hz, 1H). |
| 73-A | [M-C2HF3O2 + 1] += 390.2, 1H NMR (400 MHz, DMSO) δ 14.34 (s, 2H), 9.95 (s, 1H), 9.07 (s, 1H), 7.56 (d, J = 8.1 |
| | Hz, 1H), 7.40 (s, 1H), 7.28 (t, J = 7.9 Hz, 1H), 7.11 (dt, J = 15.0, 7.3 Hz, 2H), 6.97 (s, 1H), 6.85 (d, J = 7.6 Hz, 1H), |
| | 6.68 (d, J = 7.4 Hz, 1H), 5.79 (s, 1H), 3.85-3.67 (m, 2H), 3.63 (q, J = 7.6 Hz, 1H), 3.38-3.21 (m, 1H), 2.59-2.52 |
| | (m, 1H), 2.43-2.31 (m, 2H), 2.27 (s, 3H), 2.14 (s, 3H), 2.00 (td, J = 12.8, 7.6 Hz, 1H), 1.51 (dq, J = 14.4, 7.2 Hz, 1H). |
| 73-B | [M-C2HF3O2 + 1] += 390.2 1H NMR (400 MHz, DMSO) δ 14.31 (s, 2H), 9.95 (s, 1H), 9.07 (s, 1H), 7.56 (d, J = 8.1 |
| | Hz, 1H), 7.40 (s, 1H), 7.28 (t, J = 7.9 Hz, 1H), 7.11 (dt, J = 15.1, 7.4 Hz, 2H), 6.97 (s, 1H), 6.85 (d, J = 7.7 Hz, 1H), |
| | 6.68 (d, J = 7.3 Hz, 1H), 5.79 (s, 1H), 3.75 (ddd, J = 13.4, 11.6, 6.7 Hz, 2H), 3.63 (dd, J = 15.3, 7.5 Hz, 1H), 3.35-3.25 |
| | (m, 1H), 2.42-2.33 (m, 2H), 2.27 (s, 3H), 2.14 (s, 3H), 2.00 (td, J = 12.9, 7.6 Hz, 1H), 1.51 (dq, J = 14.4, 7.2 Hz, 1H). |
| 74 | [M-C2HF3O2 + 1] += 395.1, 1H NMR (400 MHz, DMSO) δ 14.33 (s, 2H), 10.08 (s, 1H), 9.07 (d, J = 1.0 Hz, 1H), 7.34 |
| | (t, J = 7.9 Hz, 1H), 7.19-7.11 (m, 2H), 7.09 (t, J = 7.6 Hz, 1H), 7.00 (d, J = 14.5 Hz, 2H), 6.93 (d, J = 7.8 Hz, 1H), 6.66 |
| | (d, J = 7.3 Hz, 1H), 5.82 (s, 1H), 3.45 (t, J = 6.8 Hz, 2H), 2.89 (t, J = 6.8 Hz, 2H), 2.26 (s, 3H), 2.11 (d, J = 14.5 Hz, 3H). |
| 75 | [M-C2HF3O2 + 1] += 360.1, 1H NMR (400 MHz, DMSO) δ 14.36 (s, 2H), 9.79 (s, 1H), 9.08 (s, 1H), 7.35 (t, J = 7.9 Hz, |
| | 1H), 7.25 (dd, J = 14.2, 7.6 Hz, 1H), 7.15 (t, J = 8.1 Hz, 2H), 7.03 (d, J = 10.8 Hz, 2H), 6.93 (d, J = 7.6 Hz, 1H), 6.70 |
| | (d, J = 7.6 Hz, 1H), 5.82 (s, 1H), 2.97 (s, 3H), 2.14 (s, 3H). |
| 76 | [M-C2HF3O2 + 1] += 370.1, 1H NMR (400 MHz, DMSO) δ 14.28 (s, 2H), 9.64 (s, 1H), 8.97 (s, 1H), 7.62 (s, 1H), 7.18 |
| | (t, J = 8.1 Hz, 1H), 7.07 (dt, J = 10.8, 6.6 Hz, 3H), 6.95 (dd, J = 18.4, 7.0 Hz, 3H), 4.67 (t, J = 7.8 Hz, 1H), 3.37 (dd, |
| | J = 13.7, 8.2 Hz, 1H), 3.12 (dd, J = 13.7, 7.5 Hz, 1H), 2.83 (s, 3H), 2.21 (s, 3H), 2.11 (s, 3H). |
| 77 | [M -C2HF3O2 + 1] += 410.1, 1H NMR (400 MHz, DMSO) δ 14.30 (s, 1.5H), 9.07 (s, 1H), 7.38 (t, J = 7.9 Hz, 1H), |
| | 7.19-7.01 (m, 5H), 6.92 (s, 1H), 6.65 (d, J = 7.3 Hz, 1H), 5.86 (s, 1H), 2.26 (s, 3H), 2.11 (s, 3H). |
| 78 | [M-C2HF3O2 + 1] += 380.0, 1H NMR (400 MHz, DMSO) δ 14.42 (s, 2H), 9.81 (s, 1H), 9.08 (s, 1H), 7.52-7.43 (m, |
| | 2H), 7.38-7.26 (m, 2H), 7.21-7.15 (m, 2H), 7.11 (s, 1H), 6.97 (d, J = 7.7 Hz, 1H), 6.06 (s, 1H), 2.97 (s, 3H). |
| 79 | [M- C2HF3O2 + 1] += 389.2, 1H NMR (400 MHz, DMSO) δ 10.04 (s, 1H), 9.03 (s, 1H), 7.53 (d, J = 8.2 Hz, 1H), 7.37 |
| | (s, 1H), 7.29 (t, J = 7.9 Hz, 1H), 7.15-7.04 (m, 2H), 6.93 (s, 1H), 6.86 (d, J = 7.7 Hz, 1H), 6.66 (d, J = 7.4 Hz, 1H), |
| | 5.77 (s, 1H), 3.36 (dd, J = 11.4, 7.3 Hz, 1H), 3.27-3.18 (m, 1H), 3.15-3.04 (m, 1H), 2.79 (dd, J = 11.2, 8.9 Hz, 1H), |
| | 2.59-2.52 (m, 1H), 2.49-2.39 (m, 2H), 2.25 (s, 3H), 2.15-2.02 (m, 4H), 1.54 (dq, J = 13.0, 8.8 Hz, 1H). |
| 80 | [M-C2HF3O2 + 1] += 433.2, 1H NMR (400 MHz, DMSO) δ 14.32 (s, 2H), 9.92 (s, 1H), 9.06 (s, 1H), 7.57 (d, J = 8.2 |
| | Hz, 1H), 7.39 (s, 1H), 7.28 (t, J = 7.9 Hz, 1H), 7.17-7.05 (m, 2H), 6.97 (s, 1H), 6.85 (d, J = 7.7 Hz, 1H), 6.67 (d, |
| | J = 7.4 Hz, 1H), 5.78 (s, 1H), 3.81 (dd, J = 11.2, 2.7 Hz, 2H), 3.28 (t, J = 10.9 Hz, 2H), 2.26 (s, 3H), 2.21 (d, J = 7.1 Hz, |
| | 2H), 2.13 (s, 3H), 1.96 (ddd, J = 11.2, 7.4, 3.8 Hz, 1H), 1.55 (d, J = 11.6 Hz, 2H), 1.21 (qd, J = 12.2, 4.3 Hz, 2H). |
| 81 | [M-C2HF3O2 + 1] += 405.2, 1H NMR (400 MHz, DMSO) δ 14.40 (s, 1H), 10.56 (s, 1H), 9.07 (s, 1H), 7.56 (d, J = 8.8 |
| | Hz, 1H), 7.42-7.30 (m, 2H), 7.17-7.05 (m, 2H), 7.03-6.90 (m, 2H), 6.67 (d, J = 7.4 Hz, 1H), 5.84 (s, 1H), 4.03 (s, |
| | 2H), 3.84 (s, 4H), 3.24 (s, 4H), 2.26 (s, 3H), 2.13 (s, 3H). |
| 82 | [M-C2HF3O2 + 1] += 432.2, 1H NMR (400 MHz, DMSO) δ 14.41 (s, 2H), 10.07 (s, 1H), 9.08 (s, 1H), 7.55 (d, J = 8.4 |
| | Hz, 1H), 7.38 (s, 1H), 7.30 (t, J = 7.9 Hz, 1H), 7.11 (dt, J = 15.1, 7.3 Hz, 2H), 6.94 (s, 1H), 6.88 (d, J = 7.7 Hz, 1H), |
| | 6.67 (d, J = 7.4 Hz, 1H), 5.79 (s, 1H), 3.54-2.86 (m, 10H), 2.76 (s, 3H), 2.57 (s, 2H), 2.26 (s, 3H), 2.13 (s, 3H). |
| 83 | [M-C2HF3O2 + 1] += 399.2, 1H NMR (400 MHz, DMSO) δ 14.29 (s, 2H), 9.05 (s, 1H), 7.14-7.05 (m, 4H), 6.95 (s, |
| | 1H), 6.69 (d, J = 7.4 Hz, 1H), 6.50 (d, J = 9.4 Hz, 1H), 6.39 (s, 1H), 6.34 (d, J = 7.6 Hz, 1H), 5.66 (s, 1H), 3.13-3.03 |
| | (m, 4H), 2.88 (s, 3H), 2.26 (s, 3H), 2.14 (s, 3H). |
| 84 | [M-C2HF3O2 + 1] += 417.2, 1H NMR (400 MHz, DMSO) δ 9.01 (d, J = 1.1 Hz, 1H), 7.51 (d, J = 8.1 Hz, 1H), 7.37 (s, |
| | 1H), 7.32 (t, J = 7.9 Hz, 1H), 7.10 (dt, J = 15.1, 7.4 Hz, 2H), 6.90 (d, J = 9.8 Hz, 2H), 6.65 (d, J = 7.4 Hz, 1H), 5.78 (s, |
| | 1H), 3.40 (d, J = 12.0 Hz, 2H), 3.30 (t, J = 7.2 Hz, 2H), 2.88 (t, J = 11.2 Hz, 2H), 2.77 (t, J = 7.2 Hz, 2H), 2.24 (s, 3H), |
| | 2.11 (s, 3H), 1.81 (d, J = 14.3 Hz, 2H), 1.72-1.54 (m, 3H), 1.39 (t, J = 12.1 Hz, 1H). |
| 85 | [M-C2HF3O2 + 1] += 344.2, 1H NMR (400 MHz, DMSO) δ 14.35 (s, 2H), 9.05 (s, 1H), 7.95 (d, J = 7.7 Hz, 1H), 7.78 |
| | (s, 1H), 7.56 (t, J = 7.7 Hz, 1H), 7.46 (d, J = 7.7 Hz, 1H), 7.19-7.07 (m, 2H), 6.96 (s, 1H), 6.69 (d, J = 7.3 Hz, 1H), |
| | 5.96 (s, 1H), 3.47-3.34 (m, 2H), 2.71 (t, J = 6.6 Hz, 2H), 2.25 (s, 3H), 2.11 (s, 3H). |
| 86 | [M-C2HF3O2 + 1] += 426.2, 1H NMR (400 MHz, DMSO) δ 14.32 (s, 2H), 9.89 (s, 1H), 9.07 (s, 1H), 7.33 (t, J = 7.9 Hz, |
| | 1H), 7.14 (d, J = 7.4 Hz, 2H), 7.09 (t, J = 7.6 Hz, 1H), 7.03-6.95 (m, 2H), 6.91 (d, J = 7.5 Hz, 1H), 6.65 (d, J = 7.5 |
| | Hz, 1H), 5.82 (s, 1H), 3.82-3.75 (m, 1H), 3.71-3.66 (m, 1H), 3.61 (dd, J = 7.4, 1.9 Hz, 1H), 3.32-3.24 (m, 1H), |
| | 3.23-3.13 (m, 2H), 2.26 (s, 3H), 2.12 (s, 3H), 2.08-1.85 (m, 1H), 1.65-1.41 (m, 1H). |
| 87 | [M-C2HF3O2 + 1] += 372.2, 1H NMR (400 MHz, DMSO) δ 14.35 (s, 2H), 9.84 (s, 1H), 9.08 (d, J = 1.0 Hz, 1H), 8.19 |
| | (s, 2H), 7.68 (d, J = 8.2 Hz, 1H), 7.56 (s, 1H), 7.33 (t, J = 7.9 Hz, 1H), 7.17-7.06 (m, 2H), 7.00 (s, 1H), 6.89 (d, |
| | J = 7.7 Hz, 1H), 6.71 (d, J = 7.1 Hz, 1H), 5.81 (s, 1H), 2.27 (s, 3H), 2.16 (s, 3H). |
| 88 | [M-C2HF3O2 + 1] += 433.2, 1H NMR (400 MHz, DMSO) δ 14.42 (s, 2H), 10.05 (s, 2H), 9.05 (s, 1H), 7.58 (d, J = 8.5 |
| | Hz, 1H), 7.37 (s, 1H), 7.30 (t, J = 7.9 Hz, 1H), 7.16-7.07 (m, 2H), 6.93 (s, 1H), 6.88 (d, J = 7.7 Hz, 1H), 6.67 (d, |
| | J = 7.3 Hz, 1H), 5.79 (s, 1H), 3.96 (s, 2H), 3.55 (s, 4H), 3.19-2.91 (m, 4H), 2.43-2.34 (m, 2H), 2.26 (s, 3H), 2.13 (s, |
| | 3H), 1.98-1.87 (m, 2H). |
| 89 | [M-C2HF3O2 + 1] += 403.2, 1H NMR (400 MHz, DMSO) δ 14.33 (s, 2H), 10.19 (s, 1H), 9.49 (s, 1H), 9.07 (s, 1H), |
| | 7.56 (d, J = 8.9 Hz, 1H), 7.33 (dd, J = 15.6, 7.7 Hz, 2H), 7.19-7.05 (m, 2H), 6.92 (d, J = 9.4 Hz, 2H), 6.66 (d, J = 7.3 |
| | Hz, 1H), 5.79 (s, 1H), 3.40 (d, J = 6.1 Hz, 4H), 3.05 (d, J = 7.3 Hz, 2H), 2.77 (t, J = 7.1 Hz, 2H), 2.26 (s, 3H), 2.13 (s, |
| | 3H), 2.01 (s, 2H), 1.87 (dd, J = 7.3, 4.8 Hz, 2H). |
| 90 | [M-C2HF3O2 + 1] += 418.2, 1H NMR (400 MHz, DMSO) δ 14.42 (s, 2H), 10.12 (s, 1H), 9.00 (d, J = 60.9 Hz, 3H), 7.56 |
| | (d, J = 8.3 Hz, 1H), 7.37 (s, 1H), 7.30 (t, J = 7.9 Hz, 1H), 7.19-7.05 (m, 2H), 6.94 (s, 1H), 6.89 (d, J = 7.7 Hz, 1H), |
| | 6.67 (d, J = 7.3 Hz, 1H), 5.80 (s, 1H), 3.13 (d, J = 88.2 Hz, 10H), 2.63 (s, 2H), 2.26 (s, 3H), 2.13 (s, 3H). |
| 91 | [M -C2HF3O2 + 1] += 435.2, 1H NMR (400 MHz, DMSO) δ 9.02 (d, J = 0.9 Hz, 1H), 7.69 (d, J = 7.8 Hz, 1H), 7.60 (s, |
| | 1H), 7.50 (t, J = 7.7 Hz, 1H), 7.39 (d, J = 7.7 Hz, 1H), 7.17-7.05 (m, 2H), 6.93 (s, 1H), 6.67 (d, J = 7.2 Hz, 1H), 5.91 |
| | (s, 1H), 4.29-4.16 (m, 2H), 4.00 (s, 2H), 3.74 (s, 2H), 3.50 (s, 2H), 3.47-3.42 (m, 2H), 3.22 (d, J = 7.1 Hz, 2H), |
| | 2.25 (s, 3H), 2.11 (s, 3H). |
| 92 | [M-C2HF3O2 + 1] += 312.2, 1H NMR (400 MHz, DMSO) δ 14.27 (s, 2H), 8.97 (d, J = 0.8 Hz, 1H), 7.65 (s, 1H), 7.28 |
| | (d, J = 7.6 Hz, 1H), 7.15 (s, 1H), 7.12-6.99 (m, 2H), 6.62 (s, 1H), 4.12 (d, J = 10.6 Hz, 1H), 2.25 (d, J = 4.1 Hz, 6H), |
| | 2.14 (dd, J = 22.9, 11.7 Hz, 1H), 2.03 (t, J = 10.0 Hz, 1H), 1.80-1.65 (m, 2H), 1.45 (dd, J = 31.5, 12.5 Hz, 2H), |
| | 1.31-1.13 (m, 2H), 1.02-0.85 (m, 2H). |
| 93 | [M-C2HF3O2 + 1] += 396.2, 1H NMR (400 MHz, DMSO) δ 8.95 (s, 1H), 7.58 (s, 1H), 7.05 (dd, J = 7.3, 5.8 Hz, 2H), |
| | 6.98-6.89 (m, 1H), 4.53 (t, J = 7.5 Hz, 1H), 3.51-3.47 (m, 2H), 3.44 (dd, J = 7.2, 4.1 Hz, 4H), 3.34 (dd, J = 10.5, |
| | 4.9 Hz, 1H), 3.29-3.20 (m, 1H), 3.07 (t, J = 5.7 Hz, 2H), 2.90 (s, 3H), 2.28 (dd, J = 17.4, 4.5 Hz, 7H), 2.05 (td, |
| | J = 13.5, 6.0 Hz, 1H). |
| 94 | [M-C2HF3O2 + 1] += 455.2, 1H NMR (400 MHz, DMSO) δ 14.36 (s, 1H), 10.11 (s, 1H), 9.08 (d, J = 1.0 Hz, 1H), 7.35 |
| | (t, J = 7.9 Hz, 1H), 7.20-7.13 (m, 2H), 7.09 (t, J = 7.6 Hz, 1H), 7.04 (s, 1H), 6.95 (d, J = 8.7 Hz, 2H), 6.67 (d, J = 7.4 |
| | Hz, 1H), 5.82 (s, 1H), 3.69 (s, 4H), 3.52 (s, 2H), 3.13 (d, J = 111.8 Hz, 6H), 2.26 (s, 3H), 2.13 (s, 3H). |
| 95 | [M-C2HF3O2 + 1] += 406.2, 1H NMR (400 MHz, DMSO) δ 9.04 (d, J = 1.2 Hz, 1H), 7.66 (d, J = 7.8 Hz, 1H), 7.57 (s, |
| | 1H), 7.47 (t, J = 7.7 Hz, 1H), 7.35 (d, J = 7.8 Hz, 1H), 7.17-7.07 (m, 2H), 6.96 (s, 1H), 6.68 (d, J = 7.3 Hz, 1H), 5.90 |
| | (s, 1H), 3.87-3.80 (m, 1H), 3.72 (ddd, J = 13.7, 12.6, 7.2 Hz, 3H), 3.64-3.55 (m, 2H), 3.52-3.50 (m, 1H), 2.25 (s, |
| | 3H), 2.12 (s, 3H), 2.01-1.91 (m, 1H), 1.58 (td, J = 13.4, 7.1 Hz, 1H). |
| 96 | [M-C2HF3O2 + 1] += 404.2, 1H NMR (400 MHz, DMSO) δ 14.32 (s, 2H), 9.93 (s, 1H), 9.07 (d, J = 1.1 Hz, 1H), 7.56 |
| | (t, J = 7.0 Hz, 1H), 7.42 (d, J = 11.7 Hz, 1H), 7.28 (t, J = 7.9 Hz, 1H), 7.18-7.01 (m, 2H), 6.96 (s, 1H), 6.85 (dd, J = 7.5, |
| | 3.8 Hz, 1H), 6.67 (d, J = 7.5 Hz, 1H), 5.78 (s, 1H), 3.81 (d, J = 11.2 Hz, 1H), 3.69-3.65 (m, 1H), 3.30 (td, J = 11.0, |
| | 2.5 Hz, 1H), 2.42 (dd, J = 14.2, 8.2 Hz, 1H), 2.33 (dd, J = 13.8, 4.3 Hz, 1H), 2.26 (s, 3H), 2.13 (s, 3H), 1.80-1.71 (m, |
| | 1H), 1.60 (d, J = 12.5 Hz, 1H), 1.52-1.36 (m, 3H), 1.28-1.15 (m, 1H). |
| 97 | [M-C2HF3O2 + 1] += 384.2, 1H NMR (400 MHz, DMSO) δ 14.28 (s, 2H), 9.67 (s, 1H), 9.01 (d, J = 1.0 Hz, 1H), 7.64 |
| | (s, 1H), 7.25 (t, J = 7.8 Hz, 1H), 7.12-7.02 (m, 3H), 7.02-6.95 (m, 2H), 6.92 (d, J = 7.7 Hz, 1H), 4.35 (t, J = 7.4 Hz, |
| | 1H), 2.96 (s, 3H), 2.57 (dd, J = 8.9, 5.1 Hz, 1H), 2.47 (s, 1H), 2.36 (d, J = 8.6 Hz, 1H), 2.26 (s, 3H), 2.16-2.02 (m, 4H). |
| 98 | [M-C2HF3O2 + 1] += 404.2, 1H NMR (400 MHz, DMSO) δ 14.36 (s, 2H), 9.08 (d, J = 1.1 Hz, 1H), 8.52 (t, J = 5.7 Hz, |
| | 1H), 7.79 (d, J = 7.8 Hz, 1H), 7.68 (s, 1H), 7.45 (t, J = 7.7 Hz, 1H), 7.30 (d, J = 7.8 Hz, 1H), 7.17-7.06 (m, 2H), 6.96 |
| | (s, 1H), 6.69 (d, J = 7.3 Hz, 1H), 5.90 (s, 1H), 3.84 (dd, J = 11.3, 2.6 Hz, 2H), 3.25 (t, J = 10.9 Hz, 2H), 3.13 (t, J = 6.4 |
| | Hz, 2H), 2.26 (s, 3H), 2.13 (s, 3H), 1.77 (ddt, J = 14.9, 7.7, 3.8 Hz, 1H), 1.56 (d, J = 11.5 Hz, 2H), 1.18 (qd, J = 12.1, |
| | 4.4 Hz, 2H)). |
| 98-A | [M + 1] += 404.2, 1H NMR (400 MHz, MeOD) δ 7.68 (d, J = 4.1 Hz, 2H), 7.63 (s, 1H), 7.40 (t, J = 7.7 Hz, 1H), 7.28 (d, |
| | J = 7.7 Hz, 1H), 7.06 (d, J = 7.4 Hz, 1H), 7.00 (dd, J = 14.2, 6.7 Hz, 1H), 6.73 (d, J = 7.5 Hz, 1H), 6.40 (s, 1H), 5.77 |
| | (s, 1H), 3.95 (dd, J = 11.2, 3.2 Hz, 2H), 3.40 (dd, J = 19.1, 7.6 Hz, 2H), 3.25 (d, J = 7.0 Hz, 2H), 2.30 (s, 3H), 2.18 (s, |
| | 3H), 1.89 (dtd, J = 15.2, 7.5, 3.6 Hz, 1H), 1.67 (d, J = 12.9 Hz, 2H), 1.42-1.19 (m, 3H). |
| 98-B | [M + 1] += 404.2, 1H NMR (400 MHz, MeOD) δ 7.66 (t, J = 12.4 Hz, 3H), 7.40 (t, J = 7.7 Hz, 1H), 7.28 (d, J = 7.7 Hz, |
| | 1H), 7.11-6.97 (m, 2H), 6.73 (d, J = 7.6 Hz, 1H), 6.39 (s, 1H), 5.77 (s, 1H), 3.95 (dd, J = 11.2, 3.0 Hz, 2H), 3.41 (t, |
| | J = 11.2 Hz, 2H), 3.25 (d, J = 6.9 Hz, 2H), 2.30 (s, 3H), 2.18 (s, 3H), 1.93-1.83 (m, 1H), 1.67 (d, J = 12.8 Hz, 2H), |
| | 1.37-1.28 (m, 3H). |
| 99 | [M -C2HF3O2 + 1] += 418.2, 1H NMR (400 MHz, DMSO) δ 9.01 (s, 1H), 7.51 (d, J = 6.6 Hz, 1H), 7.41 (s, 1H), 7.27 |
| | (t, J = 7.9 Hz, 1H), 7.10 (dt, J = 15.1, 7.3 Hz, 2H), 6.95 (s, 1H), 6.83 (d, J = 7.6 Hz, 1H), 6.66 (d, J = 7.5 Hz, 1H), 5.76 |
| | (s, 1H), 3.68 (d, J = 20.5 Hz, 1H), 3.35-3.24 (m, 1H), 2.25 (s, 3H), 2.16 (t, J = 7.0 Hz, 1H), 2.12 (s, 4H), 1.78 (d, J = 8.7 |
| | Hz, 2H), 1.59 (dd, J = 37.0, 9.9 Hz, 3H), 1.39 (dd, J = 18.5, 8.5 Hz, 3H), 1.14-1.04 (m, 1H), 0.95 (q, J = 12.9 Hz, 1H). |
| 100-A | [M + 1] += 319.1, 1H NMR (400 MHz, DMSO) δ 12.03 (s, 1H), 8.71 (s, 1H), 8.15 (s, 1H), 7.48 (s, 1H), 7.42 (d, J = 8.0 |
| | Hz, 1H), 7.04 (t, J = 7.6 Hz, 1H), 6.98-6.96 (m, 1H), 6.90 (d, J = 8.0 Hz, 1H), 6.82 (s, 1H), 6.46-6.43 (m, 1H), 5.65 |
| | (s, 1H), 4.26 (d, J = 10.4 Hz, 1H), 3.79-3.74 (m, 1H), 2.78-2.67 (m, 1H), 2.61-2.55 (m, 1H), 2.19 (s, 3H), 1.98-1.92 |
| | (m, 1H), 1.89 (s, 3H), 1.85-1.75 (m, 1H). |
| 100-B | [M + 1] += 319.1, 1H NMR (400 MHz, DMSO) δ 11.96 (s, 1H), 8.81 (s, 1H), 8.15 (s, 1H), 7.54 (s, 1H), 7.46 (d, J = 7.6 |
| | Hz, 1H), 7.03 (t, J = 7.6 Hz, 1H), 6.93 (t, J = 6.4 Hz, 2H), 6.85 (s, 1H), 6.48-6.46 (m, 1H), 5.87-5.86 (m, 1H), 4.15-4.12 |
| | (m, 1H), 4.03-3.92 (m, 1H), 2.69-2.63 (m, 1H), 2.60-2.55 (m, 1H), 2.22 (s, 6H), 1.91-1.80 (m, 1H), 1.47-1.34 (m, 1H). |
| 101 | [M-C2HF3O2 + 1] += 418.3, 1H NMR (400 MHz, DMSO) δ 14.31 (s, 2H), 9.91 (s, 1H), 9.07 (s, 1H), 7.55 (d, J = 8.0 |
| | Hz, 1H), 7.39 (s, 1H), 7.28 (t, J = 7.9 Hz, 1H), 7.11 (dt, J = 15.0, 7.4 Hz, 2H), 6.97 (s, 1H), 6.85 (d, J = 7.6 Hz, 1H), |
| | 6.67 (d, J = 7.4 Hz, 1H), 5.78 (s, 1H), 3.82 (dd, J = 11.0, 3.2 Hz, 2H), 3.25 (t, J = 11.2 Hz, 2H), 2.31-2.23 (m, 5H), |
| | 2.13 (s, 3H), 1.50 (ddd, J = 19.0, 15.8, 9.5 Hz, 5H), 1.14 (qd, J = 12.4, 4.2 Hz, 2H). |
| 102 | [M-C2HF3O2 + 1] += 445.3, 1H NMR (400 MHz, DMSO) δ 9.96 (s, 1H), 9.04 (s, 2H), 7.54 (d, J = 8.1 Hz, 2H), 7.40 (s, |
| | 2H), 7.28 (t, J = 7.9 Hz, 2H), 7.10 (dt, J = 15.1, 7.4 Hz, 4H), 6.95 (s, 2H), 6.84 (d, J = 7.6 Hz, 2H), 6.66 (d, J = 7.5 Hz, |
| | 2H), 5.77 (s, 2H), 4.30 (d, J = 13.1 Hz, 2H), 3.76 (d, J = 13.2 Hz, 2H), 2.99 (t, J = 12.0 Hz, 2H), 2.47 (s, 2H), 2.25 (s, |
| | 6H), 2.20 (d, J = 7.0 Hz, 4H), 2.12 (s, 6H), 1.97 (s, 8H), 1.64 (t, J = 14.8 Hz, 4H), 1.18-1.05 (m, 2H), 0.98 (dt, |
| | J = 12.0, 8.6 Hz, 2H). |
| 103 | [M-C2HF3O2 + 1] += 397.2, 1H NMR (400 MHz, DMSO) δ 14.36 (s, 1H), 10.37 (s, 1H), 9.07 (s, 1H), 8.68 (d, J = 5.5 |
| | Hz, 2H), 7.64 (d, J = 5.4 Hz, 2H), 7.55 (d, J = 8.1 Hz, 1H), 7.39 (s, 1H), 7.31 (t, J = 7.9 Hz, 1H), 7.10 (dt, J = 15.1, 7.4 |
| | Hz, 2H), 6.97-6.86 (m, 2H), 6.66 (d, J = 7.4 Hz, 1H), 5.80 (s, 1H), 3.85 (s, 2H), 2.26 (s, 3H), 2.12 (s, 3H). |
| 104 | [M-C2HF3O2 + 1] += 404.2, 1H NMR (400 MHz, DMSO) δ 14.33 (s, 2H), 9.93 (s, 1H), 9.07 (s, 1H), 7.56 (d, J = 8.1 |
| | Hz, 1H), 7.39 (s, 1H), 7.28 (t, J = 7.9 Hz, 1H), 7.11 (dt, J = 15.0, 7.4 Hz, 2H), 6.97 (s, 1H), 6.85 (d, J = 7.6 Hz, 1H), |
| | 6.67 (d, J = 7.4 Hz, 1H), 5.79 (s, 1H), 3.72 (dd, J = 9.2, 5.8 Hz, 2H), 3.30 (td, J = 10.8, 2.6 Hz, 1H), 3.09-3.00 (m, |
| | 1H), 2.26 (s, 3H), 2.21-2.07 (m, 5H), 2.01-1.90 (m, 1H), 1.77 (d, J = 9.4 Hz, 1H), 1.60-1.40 (m, 2H), 1.27-1.13 (m, 1H). |
| 105 | [M-C2HF3O2 + 1] += 440.2, 1H NMR (400 MHz, DMSO) δ 14.37 (s, 2H), 9.09 (s, 1H), 7.73 (d, J = 7.9 Hz, 1H), 7.68 |
| | (t, J = 5.9 Hz, 1H), 7.64-7.56 (m, 2H), 7.45 (d, J = 7.7 Hz, 1H), 7.20-7.06 (m, 2H), 6.97 (s, 1H), 6.67 (d, J = 7.5 |
| | Hz, 1H), 6.01 (s, 1H), 3.80-3.74 (m, 3H), 3.16 (t, J = 11.2 Hz, 2H), 2.56 (dd, J = 11.9, 6.3 Hz, 2H), 2.27 (s, 3H), 2.10 |
| | (d, J = 17.5 Hz, 3H), 1.48 (dd, J = 20.5, 9.5 Hz, 3H), 1.07-0.92 (m, 2H). |
| 106 | [M-C2HF3O2 + 1] += 397.2, 1H NMR (400 MHz, DMSO) δ 14.40 (s, 2H), 9.34 (s, 1H), 9.09 (s, 1H), 7.49 (s, 1H), 7.19 |
| | (d, J = 7.4 Hz, 1H), 7.11 (t, J = 7.6 Hz, 1H), 6.98 (d, J = 8.0 Hz, 2H), 6.42 (d, J = 7.8 Hz, 1H), 6.13 (s, 1H), 6.00 (s, |
| | 1H), 3.35 (q, J = 8.7 Hz, 1H), 3.07 (dd, J = 15.7, 9.0 Hz, 1H), 2.89 (dd, J = 15.4, 6.9 Hz, 2H), 2.80 (s, 3H), 2.30 (s, |
| | 3H), 2.17 (d, J = 10.9 Hz, 3H). |
| 107 | [M-C2HF3O2 + 1] += 404.2, 1H NMR (400 MHz, DMSO) δ 14.41 (s, 2H), 9.95 (s, 1H), 9.08 (s, 1H), 8.73 (s, 2H), 7.57 |
| | (d, J = 8.1 Hz, 1H), 7.45 (s, 1H), 7.32 (t, J = 7.9 Hz, 1H), 7.11 (dt, J = 15.1, 7.4 Hz, 2H), 6.95 (s, 1H), 6.90 (d, J = 7.6 |
| | Hz, 1H), 6.67 (d, J = 7.5 Hz, 1H), 5.80 (s, 1H), 3.35 (s, 2H), 3.17 (s, 4H), 2.84 (s, 4H), 2.26 (s, 3H), 2.13 (s, 3H). |
| 108 | [M-C2HF3O2 + 1] += 418.2, 1H NMR (400 MHz, DMSO) δ 14.24 (s, 2H), 9.83 (s, 1H), 8.97 (s, 1H), 7.62 (s, 1H), 7.49 |
| | (s, 1H), 7.32 (d, J = 8.0 Hz, 1H), 7.12 (t, J = 7.8 Hz, 1H), 7.05 (dd, J = 9.1, 4.9 Hz, 3H), 6.83 (d, J = 7.5 Hz, 1H), 4.68 |
| | (t, J = 7.7 Hz, 1H), 3.86-3.81 (m, 2H), 3.38-3.23 (m, 3H), 3.09 (dd, J = 13.9, 6.9 Hz, 1H), 2.22 (d, J = 6.0 Hz, 5H), |
| | 2.16 (s, 3H), 1.97 (ddd, J = 11.3, 7.6, 4.0 Hz, 1H), 1.57 (d, J = 12.1 Hz, 2H), 1.23 (qd, J = 12.3, 4.2 Hz, 2H). |
| 109 | [M + 1] += 361.1, 1H NMR (400 MHz, DMSO) δ 11.89 (s, 1H), 8.22-8.19 (m, 1H), 7.55 (s, 1H), 7.30 (d, J = 8.0 Hz, |
| | 1H), 7.14-7.03 (m, 2H), 7.01-6.95 (m, 2H), 6.78-6.76 (m, 1H), 6.66 (d, J = 7.6 Hz, 1H), 6.40 (s, 1H), 6.25-6.24 |
| | (m, 1H), 5.49 (s, 1H), 2.47-2.46 (m, 1H), 2.22 (s, 3H), 2.11 (s, 3H), 0.66-0.52 (m, 2H), 0.44-0.25 (m, 2H). |
| 110 | [M + 1] += 362.9, 1H NMR (400 MHz, DMSO) δ 11.83 (d, J = 30.0 Hz, 1H), 9.62 (d, J = 21.4 Hz, 1H), 7.56 (s, 2H), |
| | 7.38 (s, 1H), 7.17 (t, J = 7.8 Hz, 1H), 6.97 (d, J = 7.0 Hz, 2H), 6.79 (dd, J = 36.8, 28.4 Hz, 2H), 6.37 (d, J = 92.8 Hz, |
| | 1H), 5.55 (d, J = 37.8 Hz, 1H), 3.00 (s, 2H), 2.23 (d, J = 7.0 Hz, 9H), 2.12 (s, 3H). |
| 111 | [M-C2HF3O2 + 1] += 405.2, 1H NMR (400 MHz, DMSO) δ 14.27 (s, 2H), 9.04 (s, 1H), 8.43 (s, 1H), 7.29 (d, J = 8.3 |
| | Hz, 1H), 7.20 (t, J = 7.8 Hz, 2H), 7.16-7.04 (m, 2H), 6.96 (s, 1H), 6.69 (dd, J = 16.0, 7.5 Hz, 2H), 6.22 (d, J = 7.6 |
| | Hz, 1H), 5.74 (s, 1H), 3.80 (dd, J = 8.2, 3.4 Hz, 2H), 3.68-3.57 (m, 1H), 3.37-3.32 (m, 2H), 2.26 (s, 3H), 2.13 (s, |
| | 3H), 1.74 (d, J = 11.1 Hz, 2H), 1.35 (td, J = 14.9, 4.3 Hz, 2H). |
| 112 | LC-MS = [M-C2HF3O2 + 1] += 454.2, 1H NMR (400 MHz, DMSO) δ 14.25 (s, 2H), 9.76 (s, 1H), 8.99 (d, J = 0.8 Hz, |
| | 1H), 7.62 (s, 1H), 7.17 (t, J = 7.8 Hz, 1H), 7.10-7.00 (m, 4H), 6.95 (d, J = 8.0 Hz, 1H), 6.89 (d, J = 7.6 Hz, 1H), 4.67 |
| | (t, J = 7.8 Hz, 1H), 3.82-3.75 (m, 2H), 3.37 (dd, J = 13.8, 8.1 Hz, 1H), 3.26 (t, J = 11.4 Hz, 2H), 3.12 (dd, J = 13.7, |
| | 7.6 Hz, 1H), 2.87 (d, J = 6.3 Hz, 2H), 2.21 (s, 3H), 2.12 (s, 3H), 2.06-1.96 (m, 1H), 1.70-1.60 (m, 2H), 1.30-1.16 (m, 2H). |
| 112-A | LC-MS = [M + 1] += 454.2, 1H NMR (400 MHz, DMSO) δ 11.79 (d, J = 47.0 Hz, 1H), 9.69 (s, 1H), 7.49 (s, 1H), 7.20 |
| | (s, 1H), 7.11 (t, J = 7.6 Hz, 1H), 7.03-6.88 (m, 4H), 6.85 (d, J = 7.2 Hz, 1H), 6.74 (s, 1H), 4.48 (s, 1H), 3.79 (d, J = 11.4 |
| | Hz, 2H), 3.26 (t, J = 11.7 Hz, 2H), 3.04 (dd, J = 13.7, 7.7 Hz, 1H), 2.84 (t, J = 10.8 Hz, 2H), 2.19 (s, 3H), 2.15 (s, |
| | 3H), 2.01 (dd, J = 9.4, 5.6 Hz, 1H), 1.64 (t, J = 12.0 Hz, 2H), 1.28-1.15 (m, 2H). |
| 112-B | LC-MS = [M + 1] += 454.2, 1H NMR (400 MHz, DMSO) δ 11.73 (s, 1H), 9.69 (s, 1H), 7.49 (s, 1H), 7.19 (s, 1H), 7.11 |
| | (t, J = 7.7 Hz, 1H), 7.04-6.88 (m, 4H), 6.85 (d, J = 7.3 Hz, 1H), 6.74 (s, 1H), 4.48 (s, 1H), 3.84-3.71 (m, 2H), 3.26 |
| | (t, J = 11.7 Hz, 2H), 3.04 (dd, J = 13.7, 7.6 Hz, 1H), 2.91-2.80 (m, 2H), 2.19 (s, 3H), 2.15 (s, 3H), 2.08-1.94 (m, |
| | 1H), 1.64 (t, J = 11.6 Hz, 2H), 1.31-1.15 (m, 2H). |
| 113 | [M-C2HF3O2 + 1] += 408.2, 1H NMR (400 MHz, DMSO) δ 14.38 (s, 2H), 9.95 (s, 1H), 9.09 (s, 1H), 7.58 (d, J = 8.0 |
| | Hz, 1H), 7.40 (s, 1H), 7.27 (dt, J = 14.0, 7.8 Hz, 2H), 7.15 (t, J = 8.9 Hz, 1H), 7.04 (s, 1H), 6.87 (d, J = 7.6 Hz, 1H), |
| | 6.70 (d, J = 7.6 Hz, 1H), 5.79 (s, 1H), 3.81 (dd, J = 11.2, 2.6 Hz, 2H), 3.28 (t, J = 10.9 Hz, 2H), 2.25-2.10 (m, 5H), |
| | 1.96 (ddd, J = 11.2, 7.3, 3.8 Hz, 1H), 1.55 (d, J = 11.3 Hz, 2H), 1.21 (qd, J = 12.3, 4.3 Hz, 2H). |
| 114 | [M-C2HF3O2 + 1] += 360.2, 1H NMR (400 MHz, DMSO) δ 14.49 (s, 2H), 9.70 (s, 1H), 9.07 (s, 1H), 7.51-7.40 (m, |
| | 2H), 7.33-7.23 (m, 2H), 7.17-7.06 (m, 2H), 6.97 (s, 1H), 6.69 (d, J = 7.4 Hz, 1H), 5.87 (s, 1H), 4.24 (s, 2H), 3.28 |
| | (s, 2H), 2.83 (s, 2H), 2.26 (s, 3H), 2.13 (s, 3H), 1.78 (s, 2H), 1.73-1.55 (m, 3H), 1.34 (d, J = 10.5 Hz, 1H). |
| 115 | [M-C2HF3O2 + 1] += 372.1, 1H NMR (400 MHz, DMSO) δ 14.31 (s, 2H), 9.77 (s, 1H), 9.07 (s, 1H), 7.41-7.26 (m, |
| | 2H), 7.22-7.10 (m, 2H), 7.00 (d, J = 16.8 Hz, 2H), 6.91 (d, J = 7.7 Hz, 1H), 6.74 (d, J = 7.5 Hz, 1H), 5.82 (s, 1H), |
| | 5.21 (d, J = 53.4 Hz, 1H), 4.51 (s, 2H), 2.97 (s, 3H), 2.15 (s, 3H). |
| 116 | [M + 1] += 414.10, 1H NMR (400 MHz, DMSO) δ 7.63 (s, 1H), 7.29-7.21 (m, 2H), 7.12 (s, 1H), 7.04-6.95 (m, 3H), |
| | 6.68 (d, J = 7.6 Hz, 1H), 6.32 (s, 1H), 5.68 (s, 1H), 4.49 (s 2H), 3.53 (t, J = 5.2 Hz, 2H), 3.23 (t, J = 5.6 Hz, 2H), 2.85 |
| | (s, 3H), 2.27 (s, 3H), 2.15 (s, 3H). |
| 117 | [M-C2HF3O2 + 1] += 452.2, 1H NMR (400 MHz, DMSO) δ 14.32 (s, 2H), 9.97 (s, 1H), 9.07 (d, J = 1.0 Hz, 1H), 7.57 |
| | (d, J = 8.2 Hz, 1H), 7.38 (s, 1H), 7.29 (t, J = 7.9 Hz, 1H), 7.16-7.05 (m, 2H), 6.97 (s, 1H), 6.86 (d, J = 7.7 Hz, 1H), |
| | 6.67 (d, J = 7.3 Hz, 1H), 5.79 (s, 1H), 3.15 (dd, J = 18.4, 7.9 Hz, 2H), 3.00 (d, J = 12.5 Hz, 2H), 2.28 (d, J = 8.9 Hz, |
| | 5H), 2.21-2.07 (m, 4H), 2.00 (d, J = 12.6 Hz, 2H), 1.66 (q, J = 11.6 Hz, 2H). |
| 118 | [M-C2HF3O2 + 1] += 374.1, 1H NMR (400 MHz, DMSO) δ 14.38 (s, 2H), 9.79 (s, 1H), 9.09 (d, J = 1.1 Hz, 1H), 7.35 |
| | (dd, J = 16.1, 8.0 Hz, 2H), 7.25 (t, J = 7.6 Hz, 1H), 7.14 (dt, J = 13.8, 6.9 Hz, 1H), 7.02 (d, J = 6.2 Hz, 2H), 6.91 (t, J = |
| | 7.6 Hz, 2H), 5.87 (s, 1H), 5.57 (q, J = 11.0 Hz, 1H), 5.45 (q, J = 11.1 Hz, 1H), 2.97 (s, 3H), 2.25 (d, J = 11.4 Hz, 3H). |
| 119 | [M-C2HF3O2 + 1] += 440.2, 1H NMR (400 MHz, DMSO) δ 14.33 (s, 2H), 9.86 (s, 1H), 9.08 (s, 1H), 7.33 (t, J = 7.9 |
| | Hz, 1H), 7.20-7.04 (m, 3H), 6.94 (dd, J = 19.3, 6.6 Hz, 3H), 6.64 (d, J = 7.5 Hz, 1H), 5.82 (s, 1H), 3.79-3.75 (m, |
| | 2H), 3.23 (t, J = 11.7 Hz, 2H), 2.97 (d, J = 6.3 Hz, 2H), 2.26 (s, 3H), 2.12 (s, 3H), 2.06-1.94 (m, 1H), 1.63 (t, J = |
| | 11.1 Hz, 2H), 1.29-1.15 (m, 2H). |
| 120 | [M-C2HF3O2 + 1] += 402.2, 1H NMR (400 MHz, DMSO) δ 14.30 (s, 2H), 10.00 (s, 1H), 9.07 (d, J = 0.9 Hz, 1H), 7.56 |
| | (d, J = 8.3 Hz, 1H), 7.39 (s, 1H), 7.29 (t, J = 7.9 Hz, 1H), 7.18-7.05 (m, 2H), 6.98 (s, 1H), 6.86 (d, J = 7.7 Hz, 1H), |
| | 6.67 (d, J = 7.3 Hz, 1H), 5.79 (s, 1H), 5.59 (s, 1H), 4.02 (s, 2H), 3.66 (t, J = 5.5 Hz, 2H), 2.99 (s, 2H), 2.26 (s, 3H), |
| | 2.13 (s, 3H), 2.05 (s, 2H). |
| 121 | [M -C2HF3O2 + 1] += 390.2, 1H NMR (400 MHz, DMSO + D2O) δ 9.02 (d, J = 1.1 Hz, 1H), 7.78 (d, J = 7.8 Hz, 1H), |
| | 7.67 (s, 1H), 7.45 (t, J = 7.7 Hz, 1H), 7.28 (d, J = 7.7 Hz, 1H), 7.16-7.06 (m, 2H), 6.93 (s, 1H), 6.67 (d, J = 7.4 Hz, |
| | 1H), 5.89 (s, 1H), 3.92 (ddd, J = 29.2, 18.1, 8.0 Hz, 3H), 3.36 (t, J = 11.1 Hz, 2H), 2.24 (s, 3H), 2.11 (s, 3H), |
| | 1.75-1.68 (m, 2H), 1.56 (qd, J = 11.9, 4.2 Hz, 2H). |
| 121-A | LC-MS: [M + 1] += 390.2, 1H NMR (400 MHz, DMSO) δ 8.73 (s, 1H), 8.31 (d, J = 7.7 Hz, 1H), 7.78 (d, J = 7.8 Hz, |
| | 1H), 7.69 (s, 1H), 7.43 (t, J = 7.7 Hz, 1H), 7.27 (d, J = 7.8 Hz, 2H), 7.17 (s, 1H), 7.13-7.02 (m, 3H), 6.82 (s, 1H), |
| | 6.71 (d, J = 7.3 Hz, 1H), 5.85 (s, 1H), 4.00-3.94 (m, 1H), 3.87 (d, J = 9.0 Hz, 2H), 3.37 (s, 2H), 2.26 (s, 3H), 2.12 (s, |
| | 3H), 1.73 (d, J = 10.1 Hz, 2H), 1.57 (qd, J = 12.0, 4.4 Hz, 2H). |
| 121-B | LC-MS: [M + 1] += 390.2, 1H NMR (400 MHz, DMSO) δ 8.26 (d, J = 7.7 Hz, 1H), 7.68 (dd, J = 13.3, 8.6 Hz, 3H), 7.36 |
| | (t, J = 7.6 Hz, 1H), 7.24 (d, J = 7.7 Hz, 1H), 7.06-6.98 (m, 2H), 6.78 (d, J = 7.1 Hz, 1H), 6.43 (s, 1H), 5.66 (s, 1H), |
| | 4.00-3.94 (m, 1H), 3.87 (d, J = 9.6 Hz, 2H), 3.37 (s, 2H), 2.24 (s, 3H), 2.12 (s, 3H), 1.73 (d, J = 10.4 Hz, 2H), |
| | 1.63-1.53 (m, 2H). |
| 122-A | [M-C2HF3O2 + 1] += 417.3, 1H NMR (400 MHz, DMSO) δ 14.33 (s, 2H), 9.97 (d, J = 19.3 Hz, 1H), 9.08 (d, J = 0.9 |
| | Hz, 1H), 7.57 (d, J = 8.1 Hz, 1H), 7.38 (t, J = 20.3 Hz, 2H), 7.29 (t, J = 7.9 Hz, 1H), 7.18-7.04 (m, 2H), 6.97 (s, 1H), |
| | 6.86 (d, J = 7.7 Hz, 1H), 6.67 (d, J = 7.3 Hz, 1H), 5.79 (s, 1H), 3.19 (d, J = 11.8 Hz, 1H), 2.91-2.79 (m, 1H), 2.30 |
| | (dd, J = 17.6, 10.6 Hz, 5H), 2.22-2.04 (m, 6H), 1.84-1.73 (m, 1H), 1.45 (dq, J = 13.0, 8.4 Hz, 1H). |
| 122-B | [M-C2HF3O2 + 1] += 417.3, 1H NMR (400 MHz, DMSO) δ 14.33 (s, 2H), 9.97 (d, J = 19.3 Hz, 1H), 9.08 (d, J = 0.9 |
| | Hz, 1H), 7.57 (d, J = 8.1 Hz, 1H), 7.38 (t, J = 20.3 Hz, 2H), 7.29 (t, J = 7.9 Hz, 1H), 7.18-7.04 (m, 2H), 6.97 (s, 1H), |
| | 6.86 (d, J = 7.7 Hz, 1H), 6.67 (d, J = 7.3 Hz, 1H), 5.79 (s, 1H), 3.19 (d, J = 11.8 Hz, 1H), 2.91-2.79 (m, 1H), 2.30 |
| | (dd, J = 17.6, 10.6 Hz, 5H), 2.22-2.04 (m, 6H), 1.84-1.73 (m, 1H), 1.45 (dq, J = 13.0, 8.4 Hz, 1H). |
| 123 | [M-C2HF3O2 + 1] += 419.2, 1H NMR (400 MHz, DMSO) δ 14.49 (s, 2H), 10.63 (s, 1H), 9.73 (s, 1H), 9.08 (s, 1H), 7.55 |
| | (d, J = 8.3 Hz, 1H), 7.37 (dd, J = 9.4, 6.3 Hz, 2H), 7.17-7.06 (m, 2H), 7.01 (d, J = 7.7 Hz, 1H), 6.94 (s, 1H), 6.67 (d, |
| | J = 7.3 Hz, 1H), 5.84 (s, 1H), 5.06 (s, 1H), 4.08 (s, 2H), 3.92 (s, 1H), 3.45 (s, 1H), 3.29 (s, 2H), 3.09 (s, 1H), 2.26 (s, |
| | 3H), 2.13 (s, 3H), 1.93 (d, J = 6.0 Hz, 2H), 1.71 (s, 2H). |
| 124 | [M-C2HF3O2 + 1] += 422.2, 1H NMR (400 MHz, DMSO) δ 14.38 (s, 2H), 9.07 (s, 1H), 8.43 (t, J = 5.6 Hz, 1H), 7.53 (t, |
| | J = 6.4 Hz, 1H), 7.25 (t, J = 7.7 Hz, 1H), 7.17 (d, J = 7.2 Hz, 1H), 7.07 (ddd, J = 22.1, 14.3, 7.4 Hz, 3H), 6.71 (d, |
| | J = 7.4 Hz, 1H), 6.02 (s, 1H), 3.85 (dd, J = 11.3, 2.6 Hz, 2H), 3.26 (t, J = 11.0 Hz, 2H), 3.19-3.07 (m, 2H), 2.28 (s, 3H), |
| | 2.12 (s, 3H), 1.75 (ddd, J = 11.1, 7.7, 3.9 Hz, 1H), 1.58 (d, J = 12.6 Hz, 2H), 1.19 (qd, J = 12.2, 4.4 Hz, 2H). |
| 124-B | LC-MS: [M + 1] += 422.2, 1H NMR (400 MHz, DMSO) δ 12.34 (s, 1H), 8.35 (s, 1H), 7.77 (s, 1H), 7.42 (t, J = 6.6 Hz, |
| | 1H), 7.16 (t, J = 7.6 Hz, 1H), 7.11-6.98 (m, 3H), 6.76 (d, J = 7.4 Hz, 1H), 6.56 (s, 1H), 5.85 (s, 1H), 3.88-3.77 (m, |
| | 2H), 3.23 (d, J = 11.2 Hz, 2H), 3.11 (t, J = 6.3 Hz, 2H), 2.24 (s, 3H), 2.11 (s, 3H), 1.82-1.69 (m, 1H), 1.57 (d, |
| | J = 12.4 Hz, 2H), 1.17 (qd, J = 12.4, 4.5 Hz, 2H). |
| 124-A | LC-MS: [M + 1] += 422.2, 1H NMR (400 MHz, DMSO) δ 13.20 (s, 1H), 8.36 (s, 1H), 8.14 (s, 1H), 7.45 (t, J = 6.6 Hz, |
| | 1H), 7.19 (t, J = 7.6 Hz, 1H), 7.14-6.99 (m, 3H), 6.79-6.66 (m, 2H), 5.89 (s, 1H), 3.90-3.77 (m, 2H), 3.24 (d, J = 11.3 |
| | Hz, 2H), 3.12 (t, J = 6.3 Hz, 2H), 2.25 (s, 3H), 2.09 (d, J = 16.2 Hz, 3H), 1.75 (dd, J = 9.0, 5.4 Hz, 1H), 1.58 (d, |
| | J = 12.6 Hz, 2H), 1.18 (qd, J = 12.2, 4.1 Hz, 2H). |
| 125 | [M-C2HF3O2 + 1] += 390.2, 1H NMR (400 MHz, DMSO) δ 14.42 (s, 2H), 9.49 (s, 1H), 9.07 (s, 1H), 7.35 (t, J = 7.6 Hz, |
| | 1H), 7.22 (t, J = 6.3 Hz, 1H), 7.13 (d, J = 6.3 Hz, 2H), 7.07 (dd, J = 15.4, 7.7 Hz, 2H), 6.94 (s, 1H), 5.81 (s, 1H), 5.03 |
| | (s, 1H), 3.64 (d, J = 10.5 Hz, 1H), 3.51 (d, J = 11.8 Hz, 1H), 3.35 (d, J = 11.6 Hz, 1H), 3.25 (d, J = 8.4 Hz, 2H), 3.16 |
| | (d, J = 11.7 Hz, 1H), 3.04-2.89 (m, 3H), 2.26 (s, 3H), 2.13 (s, 3H), 1.97 (d, J = 12.4 Hz, 1H), 1.90-1.71 (m, 2H), |
| | 1.56 (dd, J = 23.4, 10.1 Hz, 1H). |
| 126 | [M-C2HF3O2 + 1] += 399.2, 1H NMR (400 MHz, DMSO) δ 14.37 (s, 2H), 9.09 (d, J = 1.0 Hz, 1H), 7.74-7.59 (m, 1H), |
| | 7.59-7.46 (m, 2H), 7.46-7.35 (m, 1H), 7.13 (dt, J = 15.1, 7.4 Hz, 2H), 6.96 (s, 1H), 6.66 (d, J = 7.4 Hz, 1H), 6.00 |
| | (s, 1H), 4.03-3.89 (m, 4H), 2.26 (s, 3H), 2.11 (s, 3H), 1.28-1.08 (m, 6H). |
| 127 | LC-MS = [M-C2HF3O2 + 1] += 371.1, 1H NMR (400 MHz, DMSO) δ 14.31 (s, 2H), 9.67 (s, 1H), 9.07 (d, J = 0.8 Hz, |
| | 1H), 7.28 (dt, J = 13.3, 6.3 Hz, 2H), 7.17-7.05 (m, 3H), 6.96 (d, J = 8.0 Hz, 2H), 6.79 (d, J = 7.7 Hz, 1H), 6.66 (d, |
| | J = 7.3 Hz, 1H), 5.76 (s, 1H), 2.43 (d, J = 4.9 Hz, 3H), 2.26 (s, 3H), 2.13 (s, 3H). |
| 128 | [M-C2HF3O2 + 1] += 335.2, 1H NMR (400 MHz, DMSO) δ 14.30 (s, 2H), 9.06 (d, J = 1.1 Hz, 1H), 8.58 (s, 1H), 7.30 |
| | (d, J = 8.1 Hz, 1H), 7.20 (dd, J = 13.9, 6.1 Hz, 2H), 7.14-7.04 (m, 2H), 6.96 (s, 1H), 6.68 (t, J = 7.6 Hz, 2H), 6.02 (d, |
| | J = 4.2 Hz, 1H), 5.73 (s, 1H), 2.59 (d, J = 3.7 Hz, 3H), 2.25 (s, 3H), 2.13 (s, 3H). |
| 129 | [M -C2HF3O2 + 1] += 332.2, 1H NMR (400 MHz, DMSO) δ 14.34 (s, 2H), 10.18 (s, 1H), 9.07 (s, 1H), 7.63 (d, J = 8.1 |
| | Hz, 1H), 7.47 (s, 1H), 7.32 (t, J = 7.9 Hz, 1H), 7.17-7.06 (m, 2H), 6.99 (s, 1H), 6.91 (d, J = 7.7 Hz, 1H), 6.68 (d, J = 7.3 |
| | Hz, 1H), 6.40 (dd, J = 16.9, 10.1 Hz, 1H), 6.23 (dd, J = 17.0, 1.8 Hz, 1H), 5.81 (s, 1H), 5.74 (dd, J = 10.1, 1.8 Hz, |
| | 1H), 2.27 (s, 3H), 2.14 (s, 3H). |
| 130-A | [M + 1] += 362.2., 1H NMR (400 MHz, DMSO) δ 11.95 (s, 1H), 7.53 (s, 1H), 7.36 (d, J = 7.6 Hz, 1H), 7.02 (t, J = 7.6 |
| | Hz, 1H), 6.93 (d, J = 7.2 Hz, 1H), 6.88 (d, J = 7.6 Hz, 1H), 6.84 (s, 1H), 3.91 (d, J = 10.2 Hz, 1H), 3.07-2.94 (m, |
| | 1H), 2.78 (s, 3H), 2.23 (d, J = 1.7 Hz, 6H), 2.14 (dd, J = 23.6, 12.8 Hz, 1H), 1.88 (d, J = 11.2 Hz, 1H), 1.64 (t, J = 14.8 |
| | Hz, 2H), 1.50 (d, J = 12.4 Hz, 1H), 1.27-1.16 (m, 1H), 1.13-1.00 (m, 1H), 0.88-0.68 (m, 2H) |
| 130-B | [M + 1] += 362.2., 1H NMR (400 MHz, DMSO) δ 11.89 (s, 1H), 7.51 (s, 1H), 7.33 (d, J = 7.6 Hz, 1H), 6.99 (dd, |
| | J = 15.4, 7.7 Hz, 2H), 6.92 (d, J = 7.2 Hz, 1H), 6.86 (s, 1H), 3.91 (d, J = 10.3 Hz, 1H), 3.05 (dd, J = 18.6, 15.0 Hz, 1H), |
| | 2.82 (s, 3H), 2.22 (d, J = 5.3 Hz, 6H), 2.18-2.10 (m, 1H), 1.84 (dd, J = 27.2, 11.6 Hz, 2H), 1.59 (d, J = 12.7 Hz, 1H), 1.32 |
| | (d, J = 12.5 Hz, 1H), 1.10 (dt, J = 22.1, 11.6 Hz, 2H), 0.89 (dd, J = 23.9, 11.9 Hz, 1H), 0.65 (dd, J = 23.1, 11.1 Hz, 1H) |
| 131 | [M-C2HF3O2 + 1] += 467.1, 1H NMR (400 MHz, DMSO) δ 14.34 (s, 2H), 9.99 (s, 1H), 9.08 (s, 1H), 7.32 (t, J = 7.9 Hz, |
| | 1H), 7.13 (d, J = 6.2 Hz, 3H), 7.10-7.03 (m, 2H), 6.99 (s, 1H), 6.92 (d, J = 7.7 Hz, 1H), 6.65 (d, J = 7.5 Hz, 1H), 5.82 |
| | (s, 1H), 3.57-3.47 (m, 2H), 3.33-3.24 (m, 2H), 2.27 (s, 3H), 2.24 (s, 3H), 2.11 (s, 3H). |
| 132 | [M-C2HF3O2 + 1] += 406.2 1H NMR (400 MHz, DMSO) δ 14.31 (s, 2H), 9.92 (s, 1H), 9.06 (s, 1H), 7.55 (d, J = 8.2 |
| | Hz, 1H), 7.40 (s, 1H), 7.30 (ddd, J = 22.5, 12.2, 4.8 Hz, 2H), 7.07 (d, J = 8.1 Hz, 2H), 6.93 (dt, J = 7.5, 6.8 Hz, 2H), |
| | 6.81 (d, J = 7.6 Hz, 1H), 5.81 (s, 1H), 3.81 (dd, J = 11.2, 2.7 Hz, 2H), 3.76 (s, 3H), 3.28 (t, J = 10.9 Hz, 2H), 2.21 (d, |
| | J = 7.1 Hz, 2H), 1.96 (ddd, J = 11.1, 7.3, 3.9 Hz, 1H), 1.55 (d, J = 11.5 Hz, 2H), 1.21 (qd, J = 12.1, 4.3 Hz, 2H). |
| 133 | [M-C2HF3O2 + 1] += 342.1, 1H NMR (400 MHz, DMSO) δ 14.39 (s, 2H), 9.08 (s, 1H), 7.78 (d, J = 7.8 Hz, 1H), 7.65 |
| | (s, 1H), 7.58 (t, J = 7.7 Hz, 1H), 7.47-7.33 (m, 3H), 7.23-7.07 (m, 2H), 6.96 (s, 1H), 6.70 (d, J = 7.5 Hz, 1H), 5.98 |
| | (s, 1H), 2.28 (s, 3H), 2.15 (s, 3H). |
| 134 | [M-C2HF3O2 + 1] += 440.2, 1H NMR (400 MHz, DMSO) δ 14.27 (s, 2H), 9.76 (s, 1H), 8.98 (s, 1H), 7.60 (s, 1H), 7.16 |
| | (t, J = 7.8 Hz, 1H), 7.13-7.03 (m, 4H), 7.01 (d, J = 8.0 Hz, 1H), 6.88 (d, J = 7.6 Hz, 1H), 4.68 (t, J = 7.8 Hz, 1H), |
| | 3.89 (dd, J = 11.4, 3.0 Hz, 2H), 3.37 (dd, J = 13.7, 8.3 Hz, 1H), 3.23 (t, J = 11.6 Hz, 2H), 3.12 (dd, J = 14.0, 7.2 Hz, |
| | 2H), 2.22 (s, 3H), 2.14 (s, 3H), 1.77 (d, J = 12.2 Hz, 2H), 1.60 (dt, J = 11.9, 8.2 Hz, 2H). |
| 135 | [M-C2HF3O2 + 1] += 358.1, 1H NMR (400 MHz, DMSO) δ 14.33 (s, 2H), 9.75 (s, 1H), 9.06 (s, 1H), 7.32 (dd, J = 12.4, |
| | 7.4 Hz, 2H), 7.13 (d, J = 8.0 Hz, 1H), 7.07 (d, J = 9.4 Hz, 2H), 7.01 (s, 1H), 6.96 (t, J = 7.4 Hz, 1H), 6.93-6.86 |
| | (m, 2H), 5.82 (s, 1H), 3.76 (s, 3H), 2.97 (s, 3H). |
| 136 | [M-C2HF3O2 + 1] += 370.1, 1H NMR (400 MHz, DMSO) δ 14.27 (s, 2H), 9.61 (s, 1H), 8.97 (s, 1H), 7.62 (s, 1H), 7.07 |
| | (dt, J = 12.6, 8.6 Hz, 7H), 4.67 (t, J = 7.8 Hz, 1H), 3.33 (dd, J = 13.7, 8.3 Hz, 1H), 3.09 (dd, J = 13.7, 7.4 Hz, 1H), |
| | 2.92 (s, 3H), 2.21 (s, 3H), 2.09 (s, 3H). |
| 136-A | LC-MS: [M + 1] += 370.2, 1H NMR (400 MHz, DMSO) δ 11.78 (s, 1H), 9.51 (s, 1H), 7.50 (s, 1H), 7.18 (d, J = 7.5 Hz, |
| | 1H), 7.10-6.89 (m, 6H), 6.76 (s, 1H), 4.48 (t, J = 7.6 Hz, 1H), 3.35 (s, 1H), 3.30 (s, 1H), 3.01 (dd, J = 13.7, 7.4 Hz, |
| | 3H), 2.19 (s, 3H), 2.12 (s, 3H). |
| 136-B | LC-MS: [M + 1] += 370.2, 1H NMR (400 MHz, DMSO) δ 11.72 (s, 1H), 9.52 (s, 1H), 7.50 (s, 1H), 7.18 (d, J = 7.6 Hz, |
| | 1H), 7.10-6.91 (m, 6H), 6.76 (s, 1H), 4.48 (t, J = 7.6 Hz, 1H), 3.31 (s, 1H), 3.01 (dd, J = 13.7, 7.4 Hz, 1H), 2.90 (s, |
| | 3H), 2.19 (s, 3H), 2.12 (s, 3H). |
| 137-A | [M-C2HF3O2 + 1] += 396.2, 1H NMR (400 MHz, DMSO) δ 14.28 (s, 2H), 9.31 (s, 1H), 9.01 (s, 1H), 7.74 (s, 1H), 7.27 |
| | (d, J = 7.6 Hz, 1H), 7.14 (dd, J = 17.1, 8.0 Hz, 2H), 7.06 (d, J = 7.2 Hz, 1H), 6.92 (dd, J = 8.0, 1.6 Hz, 1H), 6.07 (s, |
| | 1H), 4.48 (d, J = 10.8 Hz, 1H), 3.03-2.91 (m, 1H), 2.80-2.70 (m, 1H), 2.61 (s, 3H), 2.17 (s, 4H), 1.82 (s, 4H). |
| 137-B | [M-C2HF3O2 + 1] += 396.2, 1H NMR (400 MHz, DMSO) δ 14.32 (d, J = 34.6 Hz, 2H), 9.48 (s, 1H), 9.04 (s, 1H), 7.68 |
| | (s, 1H), 7.53 (d, J = 7.7 Hz, 1H), 7.24-7.13 (m, 2H), 7.08 (d, J = 7.4 Hz, 1H), 6.99 (dd, J = 8.0, 1.4 Hz, 1H), 6.32 (s, |
| | 1H), 4.30 (d, J = 11.3 Hz, 1H), 4.04 (dd, J = 12.9, 5.6 Hz, 1H), 2.90-2.77 (m, 4H), 2.75-2.66 (m, 1H), 2.23 (d, J = 16.0 |
| | Hz, 6H), 2.02 (dd, J = 13.0, 8.1 Hz, 1H), 1.61-1.48 (m, 1H). |
| 138 | [M-C2HF3O2 + 1] += 420.2, 1H NMR (400 MHz, DMSO) δ 14.28 (s, 2H), 13.74 (d, J = 23.1 Hz, 1H), 9.15-8.89 (m, |
| | 2H), 7.87 (d, J = 6.9 Hz, 1H), 7.22-6.99 (m, 2H), 6.99-6.80 (m, 3H), 6.68 (d, J = 7.4 Hz, 1H), 6.01 (s, 1H), 3.92-3.74 |
| | (m, 2H), 3.23 (dt, J = 12.4, 8.7 Hz, 4H), 2.26 (s, 3H), 2.07 (s, 3H), 1.93-1.73 (m, 1H), 1.59 (d, J = 12.3 Hz, 2H), |
| | 1.20 (qt, J = 35.5, 17.7 Hz, 2H). |
| 139 | [M-C2HF3O2 + 1] += 422.2, 1H NMR (400 MHz, DMSO) δ 14.20 (s, 2H), 9.00 (s, 1H), 7.58 (s, 1H), 7.21-6.95 (m, |
| | 3H), 6.86 (d, J = 7.5 Hz, 1H), 4.65 (dd, J = 8.2, 5.3 Hz, 1H), 3.90 (t, J = 9.3 Hz, 1H), 3.78 (dd, J = 16.0, 8.9 Hz, 3H), |
| | 3.50 (d, J = 5.1 Hz, 2H), 3.28 (t, J = 11.2 Hz, 2H), 3.09 (dd, J = 11.1, 5.4 Hz, 2H), 2.92 (d, J = 6.3 Hz, 2H), 2.28 (s, |
| | 6H), 2.00 (d, J = 4.4 Hz, 1H), 1.72 (d, J = 12.9 Hz, 2H), 1.28 (qd, J = 12.2, 4.1 Hz, 2H). |
| 140 | [M-C2HF3O2 + 1] += 356.2, 1H NMR (400 MHz, DMSO) δ 14.37 (s, 2H), 9.77 (s, 1H), 9.09 (s, 1H), 7.33 (t, J = 7.9 Hz, |
| | 1H), 7.28 (d, J = 4.5 Hz, 2H), 7.24-7.17 (m, 1H), 7.14 (d, J = 7.6 Hz, 1H), 7.01 (s, 2H), 6.89 (t, J = 7.4 Hz, 2H), 5.82 |
| | (s, 1H), 2.96 (s, 3H), 2.71-2.55 (m, 2H), 1.07 (t, J = 7.5 Hz, 3H). |
| 141 | [M -C2HF3O2] += 391.2, 1H NMR (400 MHz, DMSO) δ 14.20 (s, 2H), 10.20 (s, 1H), 8.92 (s, 1H), 7.56 (d, J = 8.3 Hz, |
| | 1H), 7.32 (t, J = 7.7 Hz, 2H), 7.10 (dt, J = 15.0, 7.5 Hz, 2H), 6.97-6.79 (m, 2H), 6.68 (d, J = 7.3 Hz, 1H), 5.78 (s, |
| | 1H), 3.61 (t, J = 7.6 Hz, 2H), 3.07 (s, 9H), 2.85 (t, J = 7.4 Hz, 2H), 2.26 (s, 3H), 2.13 (s, 3H). |
| 142 | [M-C2HF3O2 + 1] += 454.2, 1H NMR (400 MHz, DMSO) δ 8.93 (s, 1H), 7.61-7.49 (m, 3H), 7.47-7.35 (m, 2H), 7.07 |
| | (dt, J = 14.6, 7.7 Hz, 3H), 4.74 (t, J = 7.9 Hz, 1H), 3.78 (d, J = 8.4 Hz, 2H), 3.48 (dd, J = 13.9, 7.2 Hz, 1H), 3.22 (dt, |
| | J = 23.8, 10.1 Hz, 3H), 2.43 (d, J = 6.5 Hz, 2H), 2.17 (s, 3H), 2.04 (s, 3H), 1.49 (t, J = 14.8 Hz, 3H), 1.11-0.95 (m, 2H). |
| 143 | [M -C2HF3O2] += 411.2, 1H NMR (400 MHz, DMSO) δ 14.32 (s, 2H), 10.14 (s, 1H), 9.16-8.98 (m, 3H), 8.60 (t, |
| | J = 7.8 Hz, 1H), 8.15 (t, J = 6.9 Hz, 2H), 7.54 (d, J = 8.1 Hz, 1H), 7.28 (dd, J = 17.6, 9.7 Hz, 2H), 7.17-7.03 (m, 2H), |
| | 6.97-6.83 (m, 2H), 6.63 (t, J = 12.2 Hz, 1H), 5.78 (s, 1H), 4.87 (t, J = 6.2 Hz, 2H), 3.12 (t, J = 6.3 Hz, 2H), 2.26 (s, |
| | 3H), 2.14-2.08 (m, 3H). |
| 144 | [M-C2F3O2] += 391.2, 1H NMR (400 MHz, DMSO) δ 14.43 (s, 2H), 9.08 (s, 1H), 8.87 (t, J = 5.4 Hz, 1H), 7.79 (d, |
| | J = 7.8 Hz, 1H), 7.68 (s, 1H), 7.50 (t, J = 7.7 Hz, 1H), 7.37 (d, J = 7.7 Hz, 1H), 7.19-7.06 (m, 2H), 6.94 (s, 1H), 6.69 |
| | (d, J = 7.5 Hz, 1H), 5.93 (s, 1H), 3.66 (d, J = 5.8 Hz, 2H), 3.47 (t, J = 6.5 Hz, 2H), 3.13 (s, 9H), 2.27 (s, 3H), 2.10 |
| | (d, J = 18.2 Hz, 3H). |
| 145 | [M-C2F3O2 + 1] += 406.2, 1H NMR (400 MHz, DMSO) δ 14.32 (s, 2H), 11.58 (s, 1H), 9.07 (s, 1H), 7.70 (d, J = 7.6 |
| | Hz, 1H), 7.61 (s, 1H), 7.46 (t, J = 7.7 Hz, 1H), 7.34 (d, J = 7.5 Hz, 1H), 7.12 (dt, J = 15.1, 7.4 Hz, 2H), 6.97 (s, 1H), |
| | 6.70 (d, J = 7.4 Hz, 1H), 5.91 (s, 1H), 4.06 (s, 1H), 3.88-3.82 (m, 2H), 3.37 (t, J = 9.2 Hz, 2H), 2.27 (s, 3H), 2.13 (s, |
| | 3H), 1.89 (d, J = 10.6 Hz, 2H), 1.55 (d, J = 8.9 Hz, 2H). |
| 146 | [M + 1] += 336.1, 1H NMR (400 MHz, DMSO) δ 14.42 (s, 1H), 9.36 (s, 1H), 9.09 (d, J = 0.8 Hz, 1H), 7.96 (s, 1H), 7.67 |
| | (t, J = 8.0 Hz, 1H), 7.16-7.06 (m, 4H), 6.88 (d, J = 7.2 Hz, 1H), 6.74 (d, J = 6.8 Hz, 1H), 5.90 (s, 1H), 2.53 (d, J = 4.4 |
| | Hz, 3H), 2.26 (s, 3H), 2.16 (s, 3H). |
| 147 | [M + 1] += 363.80, 1H NMR (400 MHz, DMSO) δ 8.94 (s, 1H), 7.55-7.43 (m, 2H), 7.32 (t, J = 8.0 Hz, 1H), 7.16-7.08 |
| | (m, 2H), 6.96-6.82 (m, 2H), 6.68 (d, J = 7.4 Hz, 1H), 5.79 (s, 1H), 3.59 (t, J = 6.0 Hz, 2H), 3.22 (s, 3H), 2.53-2.49 |
| | (m, 2H), 2.26 (s, 3H), 2.14 (s, 3H). |
| 148 | [M + 1] += 377.80, 1H NMR (400 MHz, DMSO) δ 14.30 (s, 2H), 9.92 (s, 1H), 9.06 (s, 1H), 7.54 (d, J = 8.0 Hz, 1H), |
| | 7.40 (s, 1H), 7.27 (t, J = 8.0 Hz, 1H), 7.14-7.06 (m, 2H), 6.96 (s, 1H), 6.84 (d, J = 7.6 Hz, 1H), 6.66 (d, J = 7.2 Hz, |
| | 1H), 5.77 (s, 1H), 3.32 (t, J = 6.4 Hz, 2H), 3.21 (s, 3H), 2.34-2.29 (m, 2H), 2.26 (s, 3H), 2.13 (s, 3H), 1.80-1.72 (m, 2H). |
| 149 | [M + 1] += 390.1, 1H NMR (400 MHz, MeOD) δ 7.62 (s, 1H), 7.52-7.50 (m, 1H), 7.24-7.20 (m, 2H), 7.03-7.01 (m, |
| | 1H), 6.98-6.95 (m, 1H), 6.85 (d, J = 8.0 Hz, 1H), 6.70-6.68 (m, 1H), 6.35 (s, 1H), 5.65 (s, 1H), 3.99-3.95 (m, 2H), |
| | 3.47-3.41 (m, 2H), 2.61-2.53(m, 1H), 2.27 (s, 3H), 2.15 (s, 3H), 1.85-1.78 (m, 2H), 1.75-1.69 (m, 2H). |
| 150 | [M-C2HF3O2 + 1] += 358.1, 1H NMR (400 MHz, DMSO) δ 14.35 (s, 2H), 9.49 (d, J = 124.3 Hz, 1H), 9.06 (s, 1H), |
| | 7.82-7.45 (m, 3H), 7.15 (d, J = 7.4 Hz, 1H), 7.08 (dd, J = 15.2, 7.7 Hz, 1H), 7.05-6.91 (m, 3H), 6.67 (d, J = 7.6 Hz, 1H), |
| | 6.06 (s, 1H), 2.27 (s, 3H), 2.11 (d, J = 10.4 Hz, 3H). |
| 151-A | [M -C2HF3O2 + 1] += 376.2, 1H NMR (400 MHz, DMSO) δ 14.32 (s, 2H), 10.05 (s, 1H), 9.07 (s, 1H), 7.56 (d, J = 8.0 |
| | Hz, 1H), 7.42 (s, 1H), 7.30 (t, J = 7.9 Hz, 1H), 7.11 (dt, J = 15.1, 7.4 Hz, 2H), 6.97 (s, 1H), 6.87 (d, J = 7.6 Hz, 1H), |
| | 6.68 (d, J = 7.4 Hz, 1H), 5.79 (s, 1H), 3.90 (t, J = 8.2 Hz, 1H), 3.78-3.66 (m, 3H), 3.11 (p, J = 7.6 Hz, 1H), 2.27 (s, |
| | 3H), 2.14 (s, 3H), 2.04 (dd, J = 14.3, 7.1 Hz, 2H). |
| 151-B | [M-C2HF3O2 + 1] += 376.2, 1H NMR (400 MHz, DMSO) δ 14.32 (s, 2H), 10.05 (s, 1H), 9.06 (s, 1H), 7.56 (d, J = 8.0 |
| | Hz, 1H), 7.42 (s, 1H), 7.30 (t, J = 7.9 Hz, 1H), 7.15-7.06 (m, 2H), 6.97 (s, 1H), 6.87 (d, J = 7.6 Hz, 1H), 6.68 (d, J = |
| | 7.4 Hz, 1H), 5.79 (s, 1H), 3.90 (t, J = 8.2 Hz, 1H), 3.77-3.67 (m, 3H), 3.16-3.05 (m, 1H), 2.27 (s, 3H), 2.14 (s, 3H), |
| | 2.04 (dd, J = 14.4, 7.2 Hz, 2H). |
| 152 | [M-C2HF3O2 + 1] += 407.2 1H NMR (400 MHz, DMSO) δ 14.27 (s, 2H), 11.55 (s, 1H), 9.08 (s, 1H), 8.06-7.86 (m, 2H), |
| | 7.50 (d, J = 7.7 Hz, 1H), 7.38 (s, 1H), 7.18-7.05 (m, 2H), 6.96 (d, J = 7.3 Hz, 1H), 6.07 (s, 1H), 4.12 (dd, J = 8.5, 4.3 Hz, |
| | 1H), 3.91-3.86 (m, 2H), 3.38 (t, J = 9.4 Hz, 2H), 2.25 (d, J = 7.5 Hz, 6H), 1.89 (d, J = 12.3 Hz, 2H), 1.58 (d, J = 9.1 Hz, 2H). |
| 153 | [M-C2HF3O2 + 1] += 390.2, 1H NMR (400 MHz, DMSO) δ 8.97 (s, 1H), 7.49 (d, J = 8.0 Hz, 1H), 7.42 (s, 1H), 7.28 (t, |
| | J = 7.9 Hz, 1H), 7.09 (dt, J = 15.1, 7.4 Hz, 2H), 6.92 (s, 1H), 6.84 (d, J = 7.6 Hz, 1H), 6.65 (d, J = 7.4 Hz, 1H), 5.76 (s, |
| | 1H), 4.21-3.99 (m, 1H), 3.76-3.72 (m, 1H), 3.57 (dd, J = 14.6, 7.5 Hz, 1H), 2.47-2.36 (m, 2H), 2.24 (s, 3H), 2.11 |
| | (s, 3H), 1.96 (td, J = 12.4, 6.6 Hz, 1H), 1.88-1.71 (m, 2H), 1.48 (td, J = 15.5, 7.6 Hz, 1H). |
| 154-A | [M-C2HF3O2 + 1] += 376.2, 1H NMR (400 MHz, DMSO) δ 14.31 (s, 2H), 11.34 (s, 1H), 9.01 (s, 1H), 7.74 (s, 1H), |
| | 7.45 (d, J = 7.8 Hz, 1H), 7.27 (d, J = 7.8 Hz, 1H), 7.13 (t, J = 6.3 Hz, 2H), 7.10-7.05 (m, 1H), 6.39 (s, 1H), 4.50 (d, |
| | J = 10.3 Hz, 1H), 3.85-3.77 (m, 1H), 3.61 (s, 3H), 3.05 (dt, J = 16.3, 8.1 Hz, 1H), 2.90-2.76 (m, 1H), 2.27-2.17 (m, |
| | 1H), 2.13 (s, 3H), 1.98-1.87 (m, 1H), 1.65 (s, 3H). |
| 154-B | [M-C2HF3O2 + 1] += 376.2, 1H NMR (400 MHz, DMSO) δ 14.33 (s, 2H), 11.57 (s, 1H), 9.05 (s, 1H), 7.71 (s, 1H), 7.54 |
| | (d, J = 7.8 Hz, 1H), 7.48 (d, J = 7.7 Hz, 1H), 7.33 (d, J = 7.8 Hz, 1H), 7.17 (t, J = 7.6 Hz, 1H), 7.09 (d, J = 7.3 Hz, 1H), |
| | 6.86 (s, 1H), 4.36 (d, J = 10.9 Hz, 1H), 4.09 (t, J = 11.3 Hz, 1H), 3.66 (s, 3H), 2.95-2.84 (m, 1H), 2.82-2.75 (m, |
| | 1H), 2.25 (s, 3H), 2.18 (s, 3H), 2.03 (td, J = 15.9, 8.1 Hz, 1H), 1.64-1.52 (m, 1H). |
| 155-A | [M-C2HF3O2 + 1] += 390.2, 1H NMR (400 MHz, DMSO) δ 14.31 (s, 2H), 9.96 (s, 1H), 9.07 (s, 1H), 7.53 (d, J = 8.0 |
| | Hz, 1H), 7.43 (s, 1H), 7.28 (t, J = 7.9 Hz, 1H), 7.11 (dt, J = 15.0, 7.3 Hz, 2H), 6.96 (s, 1H), 6.86 (d, J = 7.5 Hz, 1H), |
| | 6.67 (d, J = 7.4 Hz, 1H), 5.78 (s, 1H), 3.92 (d, J = 11.1 Hz, 1H), 3.80 (d, J = 10.6 Hz, 1H), 3.41-3.24 (m, 2H), 2.58 |
| | (t, J = 10.8 Hz, 1H), 2.27 (s, 3H), 2.13 (s, 3H), 1.90 (d, J = 11.8 Hz, 1H), 1.61 (ddd, J = 40.6, 24.3, 12.0 Hz, 3H). |
| 155-B | LCMS = [M-C2HF3O2 + 1] += 390.2, 1H NMR (400 MHz, DMSO) δ 14.32 (s, 2H), 9.96 (s, 1H), 9.07 (s, 1H), 7.53 (d, |
| | J = 8.0 Hz, 1H), 7.43 (s, 1H), 7.28 (t, J = 7.9 Hz, 1H), 7.16-7.03 (m, 2H), 6.96 (s, 1H), 6.86 (d, J = 7.7 Hz, 1H), 6.67 |
| | (d, J = 7.3 Hz, 1H), 5.78 (s, 1H), 3.91 (d, J = 12.4 Hz, 1H), 3.80 (d, J = 10.8 Hz, 1H), 3.31 (ddd, J = 16.4, 13.4, 6.4 Hz, |
| | 2H), 2.63-2.54 (m, 1H), 2.27 (s, 3H), 2.13 (s, 3H), 1.90 (d, J = 12.1 Hz, 1H), 1.73-1.43 (m, 3H). |
| 156 | LC-MS = [M-C2HF3O2 + 1] += 338.2, 1H NMR (400 MHz, DMSO) δ 14.25 (s, 2H), 8.97 (s, 1H), 7.65-7.48 (m, 4H), |
| | 7.09 (dt, J = 14.4, 7.2 Hz, 5H), 4.78 (t, J = 7.9 Hz, 1H), 3.42 (dd, J = 13.9, 8.5 Hz, 1H), 3.22 (dd, J = 13.9, 7.3 Hz, |
| | 1H), 2.23 (s, 3H), 2.16 (s, 3H). |
| 156-A | LC-MS: [M + 1] += 338.2, 1H NMR (400 MHz, MeOD) δ 7.62 (t, J = 8.0 Hz, 1H), 7.57 (s, 1H), 7.17 (d, J = 7.5 Hz, 1H), |
| | 7.08-6.95 (m, 3H), 6.90 (d, J = 12.5 Hz, 1H), 6.73 (s, 1H), 4.63 (t, J = 7.8 Hz, 1H), 3.49 (dd, J = 13.4, 7.0 Hz, 1H), |
| | 3.21 (dd, J = 13.7, 8.6 Hz, 1H), 2.22 (s, 3H), 2.10 (s, 3H). |
| 156-B | LC-MS: [M + 1] += 338.2., 1H NMR (400 MHz, CDCl3) δ 7.91 (t, J = 8.2 Hz, 1H), 7.53 (d, J = 11.1 Hz, 1H), 7.22 (d, |
| | J = 7.6 Hz, 1H), 7.03 (ddd, J = 21.4, 15.4, 7.7 Hz, 3H), 6.81 (d, J = 13.1 Hz, 1H), 6.68-6.52 (m, 2H), 5.72 (s, 1H), |
| | 4.56 (t, J = 7.6 Hz, 1H), 3.57 (dd, J = 13.7, 7.7 Hz, 1H), 3.20 (dd, J = 13.7, 7.6 Hz, 1H), 2.24 (s, 3H), 2.10 (s, 3H). |
| 157 | [M-C2HF3O2 + 1] += 404.2, 1H NMR (400 MHz, DMSO) δ 14.23 (s, 2H), 9.79 (s, 1H), 8.95 (s, 1H), 7.58 (s, 1H), 7.44 |
| | (d, J = 8.4 Hz, 2H), 7.14-7.01 (m, 5H), 4.68 (t, J = 7.8 Hz, 1H), 3.90 (d, J = 11.0 Hz, 2H), 3.42-3.24 (m, 3H), 3.09 (dd, |
| | J = 13.8, 7.2 Hz, 1H), 2.56 (dd, J = 13.1, 7.8 Hz, 1H), 2.22 (s, 3H), 2.15 (s, 3H), 1.63 (ddd, J = 17.5, 10.7, 3.6 Hz, 4H). |
| 157-A | LC-MS: [M-HCl + 1] += 404.2, 1H NMR (400 MHz, DMSO) δ 9.86 (s, 1H), 8.87 (s, 1H), 7.53 (s, 1H), 7.42 (d, J = 8.2 |
| | Hz, 2H), 7.13-7.01 (m, 5H), 4.69 (t, J = 7.8 Hz, 1H), 3.89 (d, J = 11.2 Hz, 2H), 3.33 (ddd, J = 22.1, 12.6, 6.2 Hz, |
| | 3H), 3.10 (dd, J = 13.7, 7.5 Hz, 1H), 2.57 (d, J = 20.7 Hz, 1H), 2.18 (d, J = 30.3 Hz, 6H), 1.72-1.55 (m, 4H). |
| 157-B | LC-MS: [M-HCl + 1] += 404.3, 1H NMR (400 MHz, DMSO) δ 14.45 (s, 2H), 9.89 (s, 1H), 8.97 (s, 1H), 7.59-7.38 (m, |
| | 3H), 7.18-7.00 (m, 5H), 4.69 (t, J = 7.7 Hz, 1H), 3.89 (d, J = 11.0 Hz, 2H), 3.33 (dd, J = 13.5, 7.0 Hz, 3H), 3.08 (dd, |
| | J = 13.7, 7.0 Hz, 1H), 2.65-2.55 (m, 1H), 2.19 (d, J = 22.6 Hz, 6H), 1.71-1.55 (m, 4H). |
| 158 | [M-C2HF3O2 + 1] += 440.2, 1H NMR (400 MHz, DMSO) δ 14.26 (s, 2H), 9.75 (s, 1H), 8.97 (s, 1H), 7.62 (s, 1H), |
| | 7.17-6.92 (m, 7H), 4.66 (t, J = 7.8 Hz, 1H), 3.89 (dd, J = 11.1, 3.3 Hz, 2H), 3.38-3.16 (m, 4H), 3.07 (dd, J = 13.6, 7.6 |
| | Hz, 1H), 2.21 (s, 3H), 2.07 (s, 3H), 1.81 (d, J = 12.2 Hz, 2H), 1.62 (qd, J = 12.4, 4.5 Hz, 2H). |
| 159 | [M + 1] += 370.1, 1H NMR (400 MHz, DMSO) δ 14.44 (s, 2H), 9.07 (d, J = 1.2 Hz, 1H), 7.76 (d, J = 8.8 Hz, 1H), 7.19 |
| | (t, J = 8.0 Hz, 1H), 7.13-7.05 (m, 2H), 6.98 (d, J = 10.4 Hz, 2H), 6.80 (s, 1H), 6.67 (d, J = 7.2 Hz, 2H), 5.75 (s, 1H), |
| | 3.49 (dd, J = 11.2, 6.4 Hz, 3H), 2.26 (s, 3H), 2.13 (s, 3H), 1.44 (dd, J = 16.8, 8.0 Hz, 3H). |
| 160 | [M + 1] += 344.43, 1H NMR (400 MHz, DMSO) δ 11.89 (s, 1H), 8.46 (s, 1H), 7.70-7.61 (m, 2H), 7.60 (s, 1H), 7.47 (t, |
| | J = 8.0 Hz, 1H), 7.21 (d, J = 7.6 Hz, 1H), 7.05-6.97 (m, 2H), 6.86 (s, 1H), 6.51 (s, 1H), 5.72 (s, 1H), 2.30 (s, 3H), |
| | 2.23 (s, 3H), 2.15 (s, 3H). |
| 161 | [M + 1] += 419.90, 1H NMR (400 MHz, DMSO) δ 14.33 (s, 2H), 11.54 (s, 1H), 9.07 (s, 1H), 7.71 (d, J = 8.0 Hz, 1H), |
| | 7.63 (s, 1H), 7.39 (t, J = 8.0 Hz, 1H), 7.16-7.06 (m, 3H), 7.00 (s, 1H), 6.68 (d, J = 7.6 Hz, 1H), 5.85 (s, 1H), 3.86-3.78 (m, |
| | 2H), 3.27 (t, J = 11.2 Hz, 2H), 2.65 (d, J = 7.2 Hz, 2H), 2.26 (s, 3H), 2.15 (s, 3H), 1.58 (d, J = 12.8 Hz, 2H), 1.24 (m, 2H). |
| 162 | [M + 1] += 426.9, 1H NMR (400 MHz, MeOD) δ 8.91 (s, 1H), 7.84 (d, J = 7.6 Hz, 1H), 7.65 (s, 1H), 7.59 (t, J = 8.0 Hz, |
| | 1H), 7.48 (d, J = 7.6 Hz, 1H), 7.18-7.16 (m, 1H), 7.11 (t, J = 8.0 Hz, 1H), 6.84 (s, 1H), 6.69 (d, J = 7.6 Hz, 1H), 6.00 |
| | (s, 1H), 3.77-3.74 (m, 2H), 3.25-3.19 (m, 2H), 3.17-3.11 (m, 1H), 2.31 (s, 3H), 2.18 (s, 3H), 1.49-1.46 (m, 2H), |
| | 1.43-1.27 (m, 2H). |
| 163 | LC-MS: [M-C2HF3O2 + 1] += 320.2, 1H NMR (400 MHz, DMSO) δ 14.26 (s, 2H), 8.96 (s, 1H), 7.87 (s, 1H), 7.72 (d, |
| | J = 8.1 Hz, 2H), 7.61 (s, 1H), 7.25 (t, J = 13.1 Hz, 3H), 7.15-7.02 (m, 3H), 4.75 (t, J = 7.8 Hz, 1H), 3.42 (dd, J = 13.7, |
| | 8.4 Hz, 1H), 3.20 (dd, J = 13.7, 7.4 Hz, 1H), 2.22 (s, 3H), 2.13 (s, 3H). |
| 164 | [M + 1] += 344.43, 1H NMR (400 MHz, MeOD) δ 7.71 (s, 1H), 7.64 (s, 1H), 7.47 (t, J = 7.6 Hz, 1H), 7.40 (d, J = 6.8 |
| | Hz, 1H), 7.27 (d, J = 8.4 Hz, 1H), 7.23 (s, 1H), 7.06(d, J = 7.6 Hz, 1H), 7.00 (t, J = 7.6 Hz, 1H), 6.74 (d, J = 7.2 Hz, 1H), |
| | 6.42 (s, 1H), 5.80 (s, 1H), 3.97 (s, 3H), 2.29 (s, 3H), 2.19 (s, 3H). |
| 165 | LC-MS: [M-C2HF3O2 + 1] += 418.3, 1H NMR (400 MHz, DMSO) δ 14.23 (s, 2H), 9.81 (s, 1H), 8.95 (s, 1H), 7.59 (s, |
| | 1H), 7.42 (d, J = 8.3 Hz, 2H), 7.16-6.97 (m, 5H), 4.67 (t, J = 7.7 Hz, 1H), 3.82 (dd, J = 11.2, 2.4 Hz, 2H), 3.29 (t, J = 10.9 |
| | Hz, 3H), 3.09 (dd, J = 13.8, 7.1 Hz, 1H), 2.21 (d, J = 9.2 Hz, 5H), 2.15 (s, 3H), 1.96 (ddd, J = 11.2, 7.3, 3.9 Hz, |
| | 1H), 1.56 (d, J = 12.2 Hz, 2H), 1.22 (qd, J = 12.3, 4.2 Hz, 2H). |
| 166 | LC-MS: [M-C2HF3O2 + 1] += 426.2., 1H NMR (400 MHz, DMSO) δ 14.19 (s, 2H), 9.76 (d, J = 7.4 Hz, 1H), 9.00 (s, |
| | 1H), 7.31 (t, J = 7.9 Hz, 1H), 7.14 (d, J = 7.5 Hz, 2H), 7.08 (t, J = 7.5 Hz, 1H), 7.00 (s, 1H), 6.91 (d, J = 11.9 Hz, 2H), |
| | 6.66 (d, J = 7.7 Hz, 1H), 5.79 (s, 1H), 4.17-4.08 (m, 1H), 3.57 (ddd, J = 22.3, 15.0, 7.7 Hz, 2H), 3.21 (d, J = 5.9 Hz, |
| | 2H), 2.26 (s, 3H), 2.13 (s, 3H), 1.98 (s, 1H), 1.82-1.71 (m, 2H), 1.55 (dd, J = 16.7, 10.1 Hz, 1H). |
| 167 | [M + 1] += 454.2, 1H NMR (400 MHz, DMSO) δ 14.33 (s, 1H), 9.05 (s, 1H), 7.70 (dd, J = 8.0, 4.4 Hz, 1H), 7.19 (t, |
| | J = 8.0 Hz, 1H), 7.12 (d, J = 7.2 Hz, 1H), 7.07 (t, J = 7.6 Hz, 1H), 7.00-6.92 (m, 2H), 6.81-6.75 (m, 1H), 6.72-6.63 |
| | (m, 2H), 5.74 (s, 1H), 3.74 (dd, J = 13.6, 8.4 Hz, 2H), 3.50 (dd, J = 11.2, 6.4 Hz, 3H), 3.21 (m, 2H), 2.26 (s, 3H), 2.12 |
| | (d, J = 2.8 Hz, 3H), 1.86-1.57 (m, 5H), 1.17 (m, 2H). |
| 168 | [M + 1] += 377.20, 1H NMR (400 MHz, DMSO) δ 11.71 (s, 1H), 7.49 (s, 1H), 6.98 (m, 4H), 6.73 (s, 1H), 4.41 (t, |
| | J = 6.8 Hz, 1H), 3.05 (s, 1H), 2.98-2.89 (m, 2H), 2.88 (s, 3H), 2.82 (d, J = 11.6 Hz, 1H), 2.67-2.62 (m, 1H), 2.23 (s, |
| | 6H), 2.07-1.99 (m, 2H), 1.74 (d, J = 11.2 Hz, 2H), 1.40-1.23 (m, 3H). |
| 169 | LC-MS: [M-C2HF3O2 + 1] += 418.3., 1H NMR (400 MHz, DMSO) δ 14.21 (s, 2H), 9.81 (s, 1H), 8.94 (s, 1H), 7.59 (s, |
| | 1H), 7.43 (d, J = 8.2 Hz, 2H), 7.07 (t, J = 7.6 Hz, 5H), 4.67 (t, J = 7.7 Hz, 1H), 3.82 (d, J = 11.1 Hz, 1H), 3.68 (s, 1H), |
| | 3.35-3.26 (m, 2H), 3.08 (dd, J = 13.8, 7.1 Hz, 1H), 2.43 (dd, J = 14.1, 8.1 Hz, 1H), 2.32 (dd, J = 14.1, 4.8 Hz, 1H), 2.22 |
| | (s, 3H), 2.14 (s, 3H), 1.77 (d, J = 10.1 Hz, 1H), 1.61 (d, J = 12.6 Hz, 1H), 1.50-1.38 (m, 3H), 1.23 (t, J = 11.7 Hz, 1H). |
| 170 | LCMS = [M-C2HF3O2 + 1] += 440.3, 1H NMR (400 MHz, DMSO) δ 14.28 (s, 2H), 9.63 (s, 1H), 9.07 (s, 1H), 7.59 (d, |
| | J = 8.1 Hz, 1H), 7.45 (s, 1H), 7.27 (t, J = 7.9 Hz, 1H), 7.11 (dt, J = 15.0, 7.3 Hz, 2H), 6.98 (s, 1H), 6.81 (d, J = 7.7 Hz, |
| | 1H), 6.69 (d, J = 7.4 Hz, 1H), 5.77 (s, 1H), 2.62 (s, 1H), 2.27 (s, 3H), 2.19 (s, 2H), 2.15 (s, 3H), 2.08 (s, 2H), 1.89 (s, |
| | 1H), 1.79 (q, J = 12.6 Hz, 5H), 1.70 (s, 2H), 1.53 (d, J = 12.0 Hz, 2H). |
| 171 | LC-MS: [M-C2HF3O2 + 1] += 404.2, 1H NMR (400 MHz, DMSO) δ 14.20 (s, 2H), 9.84 (s, 1H), 8.95 (s, 1H), 7.59 (s, |
| | 1H), 7.42 (d, J = 8.3 Hz, 2H), 7.07 (d, J = 6.9 Hz, 5H), 4.67 (t, J = 7.8 Hz, 1H), 3.79 (t, J = 7.6 Hz, 1H), 3.72 (dd, J = 7.9, |
| | 5.5 Hz, 1H), 3.66-3.61 (m, 1H), 3.30 (dd, J = 15.6, 8.8 Hz, 2H), 3.09 (dd, J = 13.8, 7.2 Hz, 1H), 2.55 (dd, J = 13.6, 6.3 Hz, |
| | 1H), 2.39-2.33 (m, 2H), 2.22 (s, 3H), 2.14 (s, 3H), 2.00 (td, J = 12.8, 7.6 Hz, 1H), 1.52 (dq, J = 14.4, 7.3 Hz, 1H). |
| 172 | [M-C2HF3O2 + 1] += 426.2.1H NMR (400 MHz, DMSO) δ 14.26 (s, 2H), 9.76 (d, J = 7.6 Hz, 1H), 9.02 (s, 1H), 7.31 |
| | (t, J = 7.9 Hz, 1H), 7.18-7.06 (m, 3H), 7.00 (s, 1H), 6.97-6.86 (m, 2H), 6.66 (d, J = 7.5 Hz, 1H), 5.80 (s, 1H), 4.12 |
| | (p, J = 6.2 Hz, 1H), 3.63-3.54 (m, 2H), 3.21 (d, J = 4.9 Hz, 2H), 2.26 (s, 3H), 2.13 (s, 3H), 1.99 (dd, J = 11.8, 6.2 Hz, |
| | 1H), 1.76 (dd, J = 13.9, 7.1 Hz, 2H), 1.62-1.47 (m, 1H). |
| 173 | LC-MS: [M-C2HF3O2 + 1] += 440.2, 1H NMR (400 MHz, DMSO) δ 14.34 (s, 2H), 9.69 (s, 1H), 9.08 (s, 1H), 7.31 (t, |
| | J = 7.7 Hz, 1H), 7.14 (d, J = 7.4 Hz, 2H), 7.08 (t, J = 7.6 Hz, 1H), 6.94 (dd, J = 18.0, 11.3 Hz, 3H), 6.66 (d, J = 7.5 Hz, |
| | 1H), 5.80 (s, 1H), 3.77-3.58 (m, 2H), 3.31-3.06 (m, 3H), 2.26 (s, 3H), 2.13 (s, 3H), 1.71 (s, 1H), 1.58 (d, J = 13.1 |
| | Hz, 1H), 1.48-1.09 (m, 4H). |
| 174 | LC-MS: [M-C2HF3O2 + 1] += 349.2, 1H NMR (400 MHz, DMSO) δ 14.23 (s, 2H), 12.17 (s, 1H), 8.96 (s, 1H), 7.64 (s, |
| | 1H), 7.17-7.01 (m, 7H), 4.69 (t, J = 7.7 Hz, 1H), 3.60 (q, J = 7.0 Hz, 1H), 3.34 (dd, J = 13.9, 8.6 Hz, 1H), 3.10 (dd, |
| | J = 13.9, 7.0 Hz, 1H), 2.21 (s, 3H), 2.11 (s, 3H), 1.31 (d, J = 7.1 Hz, 3H). |
| 174-A | LC-MS: [M + 1] += 349.2, 1H NMR (400 MHz, DMSO) δ 7.50 (s, 1H), 7.17 (d, J = 7.9 Hz, 1H), 7.05 (dd, J = 19.6, 8.1 |
| | Hz, 4H), 6.98 (d, J = 7.6 Hz, 1H), 6.94 (d, J = 6.8 Hz, 1H), 6.75 (s, 1H), 4.51 (t, J = 7.7 Hz, 1H), 3.44 (q, J = 7.0 Hz, |
| | 1H), 3.32 (dd, J = 13.9, 8.4 Hz, 1H), 3.00 (dd, J = 14.0, 6.9 Hz, 1H), 2.17 (d, J = 18.9 Hz, 6H), 1.25 (d, J = 7.1 Hz, 3H). |
| 174-B | LC-MS: [M + 1] += 349.2, 1H NMR (400 MHz, DMSO) δ 7.50 (s, 1H), 7.17 (d, J = 7.2 Hz, 1H), 7.06 (dd, J = 17.5, 8.1 |
| | Hz, 4H), 7.00 (t, J = 7.5 Hz, 1H), 6.94 (d, J = 7.3 Hz, 1H), 6.75 (s, 1H), 4.52 (t, J = 7.5 Hz, 1H), 3.45 (q, J = 7.2 Hz, |
| | 1H), 3.32 (dd, J = 13.6, 8.3 Hz, 1H), 3.01 (dd, J = 14.0, 7.0 Hz, 1H), 2.17 (d, J = 19.2 Hz, 6H), 1.25 (d, J = 7.1 Hz, 3H) |
| 174-C | LC-MS: [M + 1] += 349.2, 1H NMR (400 MHz, DMSO) δ 7.51 (s, 1H), 7.17 (d, J = 7.5 Hz, 1H), 7.06 (dd, J = 18.2, 8.0 |
| | Hz, 4H), 6.99 (d, J = 7.7 Hz, 1H), 6.95 (d, J = 7.3 Hz, 1H), 4.53 (t, J = 7.7 Hz, 1H), 3.44 (dd, J = 14.1, 7.0 Hz, 1H), |
| | 3.31 (dd, J = 13.9, 8.0 Hz, 1H), 3.01 (dd, J = 14.0, 7.1 Hz, 1H), 2.17 (d, J = 18.2 Hz, 6H), 1.25 (d, J = 7.1 Hz, 3H). |
| 174-D | LC-MS: [M + 1] += 349.2, 1H NMR (400 MHz, DMSO) δ 7.50 (s, 1H), 7.17 (d, J = 7.3 Hz, 1H), 7.06 (dd, J = 22.6, 8.1 |
| | Hz, 4H), 6.99 (d, J = 7.5 Hz, 1H), 6.94 (d, J = 7.1 Hz, 1H), 4.52 (t, J = 7.6 Hz, 1H), 3.41 (q, J = 7.1 Hz, 1H), 3.31 (dd, |
| | J = 13.8, 8.1 Hz, 1H), 3.01 (dd, J = 13.9, 7.1 Hz, 1H), 2.17 (d, J = 17.8 Hz, 6H), 1.24 (d, J = 7.1 Hz, 3H). |
| 175 | LC-MS: [M-C2HF3O2 + 1] += 321.2, 1H NMR (400 MHz, DMSO) δ 14.25 (s, 2H), 8.96 (s, 1H), 7.79 (d, J = 8.0 Hz, |
| | 2H), 7.61 (s, 1H), 7.28 (d, J = 8.0 Hz, 2H), 7.14-7.04 (m, 3H), 4.76 (t, J = 7.8 Hz, 1H), 3.45 (dd, J = 13.6, 8.4 Hz, |
| | 1H), 3.23 (dd, J = 13.7, 7.4 Hz, 1H), 2.21 (s, 3H), 2.12 (s, 3H). |
| 175-A | LC-MS: [M + 1] += 321.2, 1H NMR (400 MHz, DMSO) δ 12.42 (s, 2H), 7.74 (d, J = 8.1 Hz, 2H), 7.62 (s, 1H), |
| | 7.27-7.17 (m, 3H), 7.04-6.91 (m, 2H), 6.82 (s, 1H), 4.56 (t, J = 7.7 Hz, 1H), 3.44 (dd, J = 13.5, 8.1 Hz, 1H), 3.14 (dd, |
| | J = 13.6, 7.3 Hz, 1H), 2.19 (s, 3H), 2.14 (s, 3H). |
| 175-B | LC-MS: [M + 1] += 321.2, 1H NMR (400 MHz, DMSO) δ 11.95 (s, 2H), 7.73 (d, J = 8.1 Hz, 2H), 7.50 (s, 1H), 7.22 (d, |
| | J = 8.0 Hz, 3H), 7.03-6.89 (m, 2H), 6.75 (s, 1H), 4.54 (t, J = 7.6 Hz, 1H), 3.44 (dd, J = 13.6, 8.2 Hz, 1H), 3.13 (dd, |
| | J = 13.7, 7.3 Hz, 1H), 2.19 (s, 3H), 2.14 (s, 3H). |
| 176 | LC-MS: [M-C2HF3O2 + 1] += 404.2, 1H NMR (400 MHz, DMSO) δ 14.24 (s, 2H), 9.82 (s, 1H), 8.95 (s, 1H), 7.59 (s, |
| | 1H), 7.43 (d, J = 8.3 Hz, 2H), 7.08 (t, J = 7.4 Hz, 5H), 4.67 (t, J = 7.8 Hz, 1H), 4.19-4.10 (m, 1H), 3.76 (dd, J = 14.3, |
| | 7.3 Hz, 1H), 3.59 (dd, J = 14.6, 7.5 Hz, 1H), 3.30 (dd, J = 13.7, 8.5 Hz, 1H), 3.09 (dd, J = 13.8, 7.2 Hz, 1H), 2.47 (d, |
| | J = 7.5 Hz, 1H), 2.40 (dd, J = 14.0, 5.7 Hz, 1H), 2.22 (s, 3H), 2.14 (s, 3H), 1.98 (dt, J = 12.0, 6.5 Hz, 1H), 1.91-1.75 |
| | (m, 2H), 1.52 (td, J = 15.8, 7.5 Hz, 1H). |
| 177 | LC-MS: [M-C2HF3O2 + 1] += 404.2, 1H NMR (400 MHz, DMSO) δ 14.25 (s, 2H), 8.96 (s, 1H), 8.19 (d, J = 7.6 Hz, |
| | 1H), 7.70 (d, J = 8.1 Hz, 2H), 7.59 (s, 1H), 7.24 (d, J = 8.1 Hz, 2H), 7.09 (dt, J = 10.4, 7.4 Hz, 3H), 4.76 (t, J = 7.8 Hz, |
| | 1H), 4.00-3.93 (m, 1H), 3.87 (d, J = 10.0 Hz, 2H), 3.40 (dt, J = 22.2, 9.6 Hz, 3H), 3.22 (dd, J = 13.7, 7.6 Hz, 1H), |
| | 2.21 (s, 3H), 2.13 (s, 3H), 1.72 (d, J = 12.7 Hz, 2H), 1.55 (qd, J = 12.2, 4.2 Hz, 2H). |
| 178 | [M-C2HF3O2 + 1] += 402.2. 1H NMR (400 MHz, DMSO) δ 14.30 (s, 2H), 9.86 (s, 1H), 9.07 (s, 1H), 7.56 (d, J = 8.1 |
| | Hz, 1H), 7.40 (s, 1H), 7.27 (t, J = 7.9 Hz, 1H), 7.11 (dt, J = 15.0, 7.4 Hz, 2H), 6.97 (s, 1H), 6.84 (d, J = 7.6 Hz, 1H), |
| | 6.68 (d, J = 7.5 Hz, 1H), 5.78 (s, 1H), 2.27 (s, 3H), 2.15 (d, J = 6.1 Hz, 5H), 1.78-1.56 (m, 6H), 1.17 (tt, J = 23.8, |
| | 12.0 Hz, 3H), 0.94 (q, J = 11.0 Hz, 2H). |
| 179 | LC-MS: [M + 1] += 412.2, 1H NMR (400 MHz, DMSO) δ 11.74 (s, 1H), 8.36 (d, J = 8.1 Hz, 1H), 7.54 (d, J = 8.2 Hz, |
| | 2H), 7.49 (s, 1H), 7.29 (d, J = 8.2 Hz, 2H), 7.22 (s, 1H), 6.99 (t, J = 7.5 Hz, 1H), 6.92 (d, J = 7.2 Hz, 1H), 6.74 (s, 1H), |
| | 4.53 (t, J = 7.4 Hz, 1H), 4.31 (tt, J = 21.2, 7.1 Hz, 3H), 4.09 (dd, J = 8.3, 3.5 Hz, 2H), 3.45 (dd, J = 13.3, 7.7 Hz, 1H), |
| | 3.16 (dd, J = 13.4, 8.1 Hz, 1H), 2.15 (s, 3H), 2.09 (s, 3H). |
| 180 | [M-C2HF3O2 + 1] += 389.2, 1H NMR (400 MHz, DMSO) δ 14.28 (s, 2H), 9.06 (s, 1H), 8.44 (s, 1H), 7.39 (d, J = 8.0 |
| | Hz, 1H), 7.33 (s, 1H), 7.20 (t, J = 7.9 Hz, 1H), 7.16-7.04 (m, 2H), 6.96 (s, 1H), 6.70 (dd, J = 11.2, 7.8 Hz, 2H), 5.73 |
| | (s, 1H), 3.41-3.33 (m, 4H), 2.27 (s, 3H), 2.15 (s, 3H), 1.56 (d, J = 4.6 Hz, 2H), 1.47 (d, J = 3.8 Hz, 4H). |
| 181 | [M-C2HF3O2 + 1] += 416.2. 1H NMR (400 MHz, DMSO) δ 14.21 (s, 2H), 9.75 (s, 1H), 8.96 (s, 1H), 7.60 (s, 1H), 7.42 |
| | (d, J = 8.3 Hz, 2H), 7.07 (d, J = 8.8 Hz, 5H), 4.67 (t, J = 7.8 Hz, 1H), 3.29 (dd, J = 13.7, 8.6 Hz, 1H), 3.08 (dd, J = 13.8, |
| | 7.1 Hz, 1H), 2.22 (s, 3H), 2.14 (d, J = 5.9 Hz, 5H), 1.77-1.59 (m, 6H), 1.19 (dq, J = 23.7, 12.0 Hz, 3H), 1.01-0.89 (m, 2H). |
| 182 | 1H NMR (400 MHz, DMSO) δ 14.36 (s, 2H), 11.75 (s, 1H), 9.06 (s, 1H), 7.46 (s, 2H), 7.12 (p, J = 7.4 Hz, 3H), 6.75 |
| | (d, J = 7.3 Hz, 1H), 5.88 (s, 1H), 3.67 (s, 3H), 2.28 (s, 3H), 2.18 (s, 3H)., [M-C2HF3O2 + 1] += 342.1 |
| 183 | [M + 1] += 384.1, 1H NMR (400 MHz, MeOD) δ 8.76 (s, 1H), 7.51 (s, 1H), 7.33-7.27 (m, 1H), 7.25-7.23 (m, 3H), |
| | 7.08-7.07 (m, 2H), 7.00-6.92 (m, 1H), 4.48 (t, J = 6.8 Hz, 1H), 2.97 (s, 3H), 2.94-2.86 (m, 1H), 2.83-2.73 (m, |
| | 1H), 2.50-2.35 (m, 1H), 2.31 (s, 3H), 2.23 (s, 3H), 2.21-2.12 (m, 1H). |
| 184 | [M + 1] += 322.8, 1H NMR (400 MHz, MeOD) δ 7.54 (s, 1H), 7.05-7.02 (m, 1H), 7.00-6.97 (m, 2H), 6.71 (s, 1H), |
| | 4.28 (t, J = 7.8 Hz, 1H), 3.09-2.98 (m, 2H), 2.86 (s, 3H), 2.28 (s, 3H), 2.26 (s, 3H), 2.18-2.09 (m, 1H), 1.99-1.90 |
| | (m, 1H), 1.62-1.44 (m, 2H). |
| 185 | 1H NMR (400 MHz, DMSO) δ 14.27 (s, 2H), 9.04 (s, 1H), 7.15-7.02 (m, 3H), 6.97 (s, 1H), 6.68 (d, J = 7.3 Hz, 1H), |
| | 6.39 (d, J = 7.5 Hz, 1H), 6.24 (d, J = 7.9 Hz, 1H), 6.05 (s, 1H), 5.67 (s, 1H), 4.51 (dd, J = 10.7, 5.8 Hz, 2H), 4.33 |
| | (d, J = 5.6 Hz, 2H), 2.26 (s, 3H), 2.13 (s, 3H), 1.45 (s, 3H)., LC-MS: [M-C2HF3O2 + 1] += 348.2. |
| 186 | LC-MS: [M-C2HF3O2 + 1] += 405.21, 1H NMR (400 MHz, DMSO) δ 14.21 (s, 2H), 8.97 (s, 1H), 8.53 (d, J = 8.4 Hz, |
| | 1H), 8.36 (s, 1H), 7.89 (d, J = 8.0 Hz, 1H), 7.75 (d, J = 8.0 Hz, 1H), 7.63 (s, 1H), 7.10 (dt, J = 12.2, 7.1 Hz, 3H), 4.75 |
| | (t, J = 7.8 Hz, 1H), 4.06-3.92 (m, 1H), 3.85 (d, J = 11.2 Hz, 2H), 3.50 (dd, J = 13.7, 8.0 Hz, 1H), 3.42-3.23 (m, |
| | 3H), 2.20 (s, 3H), 2.08 (s, 3H), 1.74-1.59 (m, 4H). |
| 187 | LC-MS: [M-C2HF3O2 + 1] += 422.2., 1H NMR (400 MHz, DMSO) δ 14.23 (s, 2H), 8.96 (s, 1H), 8.15 (d, J = 7.5 Hz, |
| | 1H), 7.58 (s, 1H), 7.42 (t, J = 7.7 Hz, 1H), 7.17-7.02 (m, 5H), 4.78 (t, J = 7.8 Hz, 1H), 3.97-3.89 (m, 1H), 3.84 (d, |
| | J = 11.2 Hz, 2H), 3.46-3.32 (m, 3H), 3.22 (dd, J = 13.9, 7.4 Hz, 1H), 2.22 (s, 3H), 2.18 (s, 3H), 1.73 (d, J = 11.0 Hz, |
| | 2H), 1.50 (qd, J = 12.1, 4.3 Hz, 2H). |
| 188 | LC-MS: [M-C2HF3O2 + 1] += 378.2, 1H NMR (400 MHz, DMSO) δ 14.20 (s, 2H), 8.96 (s, 1H), 7.66 (s, 1H), 7.03 (t, |
| | J = 6.2 Hz, 3H), 6.89 (s, 1H), 6.67 (s, 1H), 4.53 (dd, J = 8.5, 3.6 Hz, 1H), 2.27 (d, J = 16.9 Hz, 6H), 2.06 (dd, J = 14.4, |
| | 9.0 Hz, 1H), 1.95 (s, 2H), 1.68-1.21 (m, 13H). |
| 189 | LC-MS: [M-C2HF3O2 + 1] += 448.2, 1H NMR (400 MHz, DMSO) δ 14.10 (s, 2H), 9.92 (s, 1H), 9.03 (s, 1H), 8.83 (s, |
| | 1H), 8.64 (s, 2H), 7.30 (t, J = 7.8 Hz, 1H), 7.18-6.98 (m, 4H), 6.90 (d, J = 9.1 Hz, 2H), 6.66 (d, J = 7.1 Hz, 1H), 5.77 |
| | (s, 1H), 3.53-3.43 (m, 2H), 2.98 (t, J = 7.1 Hz, 2H), 2.23 (s, 3H), 2.10 (s, 3H). |
| 190 | LC-MS: [M-C2HF3O2 + 1] += 334.2, 1H NMR (400 MHz, DMSO) δ 14.21 (s, 2H), 9.84 (s, 1H), 8.95 (s, 1H), 7.60 (s, |
| | 1H), 7.40 (d, J = 8.2 Hz, 2H), 7.05 (d, J = 10.8 Hz, 5H), 4.66 (t, J = 7.8 Hz, 1H), 3.29 (dd, J = 13.7, 8.5 Hz, 1H), 3.07 |
| | (dd, J = 13.7, 7.1 Hz, 1H), 2.21 (s, 3H), 2.12 (s, 3H), 1.99 (s, 3H). |
| 191 | [M + 1] += 336.15, 1H NMR (400 MHz, DMSO) δ 12.13 (s, 1H), 8.15 (s, 1H), 7.50 (s, 1H), 7.06 (d, J = 7.2 Hz, 1H), |
| | 6.99-6.94 (m, 2H), 6.90 (t, J = 6.0 Hz, 1H), 6.72 (s, 1H), 4.15 (t, J = 7.6 Hz, 1H), 2.92-2.84 (m, 2H), 2.83 (s, 3H), |
| | 2.22 (d, J = 4.8 Hz, 6H), 2.05-1.95 (m, 1H), 1.85-1.70 (m, 1H), 1.50-1.39 (m, 2H), 1.29-1.15 (m, 2H). |
| 192 | LC-MS: [M-C2HF3O2 + 1] += 352.2, 1H NMR (400 MHz, DMSO) δ 14.24 (s, 2H), 8.96 (s, 1H), 8.11 (s, 1H), 7.60 (s, |
| | 1H), 7.48 (t, J = 7.8 Hz, 1H), 7.16-7.02 (m, 5H), 4.77 (t, J = 7.8 Hz, 1H), 3.41 (dd, J = 13.7, 8.5 Hz, 1H), 3.21 (dd, |
| | J = 13.8, 7.3 Hz, 1H), 2.74 (d, J = 4.3 Hz, 3H), 2.22 (s, 3H), 2.15 (s, 3H). |
| 193 | LC-MS: [M-C2HF3O2 + 1] += 352.2, 1H NMR (400 MHz, DMSO) δ 14.24 (s, 2H), 9.62 (s, 1H), 8.96 (s, 1H), 7.65 (dd, |
| | J = 21.2, 12.9 Hz, 2H), 7.06 (dd, J = 12.2, 8.4 Hz, 4H), 6.92 (d, J = 8.2 Hz, 1H), 4.71 (t, J = 7.7 Hz, 1H), 3.33 (dd, |
| | J = 13.6, 8.7 Hz, 1H), 3.12 (dd, J = 13.7, 7.2 Hz, 1H), 2.22 (s, 3H), 2.14 (s, 3H), 2.04 (s, 3H). |
| 194 | LC-MS: [M + 1] += 350.2, 1H NMR (400 MHz, DMSO) δ 11.76 (s, 1H), 9.46 (s, 1H), 7.48 (s, 1H), 7.20 (dd, J = 19.1, |
| | 7.8 Hz, 3H), 7.06-6.89 (m, 4H), 6.73 (s, 1H), 4.46 (t, J = 7.6 Hz, 1H), 3.62 (s, 3H), 3.31-3.23 (m, 1H), 2.98 (dd, J = 13.7, |
| | 7.4 Hz, 1H), 2.18 (s, 3H), 2.13 (s, 3H). |
| 195 | LC-MS: [M-C2HF3O2 + 1] += 364.3, 1H NMR (400 MHz, DMSO) δ 14.19 (s, 2H), 9.65 (s, 1H), 8.95 (s, 1H), 7.60 (s, |
| | 1H), 7.49 (d, J = 8.1 Hz, 2H), 7.08 (d, J = 6.8 Hz, 5H), 4.67 (t, J = 7.7 Hz, 1H), 3.95 (s, 2H), 3.36-3.27 (m, 4H), 3.09 |
| | (dd, J = 13.7, 7.1 Hz, 1H), 2.21 (s, 3H), 2.13 (s, 3H). |
| 196 | LC-MS: [M-C2HF3O2 + 1] += 335.2, 1H NMR (400 MHz, DMSO) δ 14.22 (s, 2H), 8.94 (s, 1H), 7.59 (s, 1H), 7.13-6.99 |
| | (m, 3H), 6.67 (dd, J = 7.5, 4.9 Hz, 2H), 6.59 (dd, J = 8.2, 1.6 Hz, 1H), 4.64 (t, J = 7.7 Hz, 1H), 4.16 (s, 4H), 3.22 |
| | (dd, J = 13.9, 8.8 Hz, 1H), 3.01 (dd, J = 13.9, 6.8 Hz, 1H), 2.23 (s, 3H), 2.15 (s, 3H). |
| 197 | LC-MS: [M-C2HF3O2 + 1] += 339.2, 1H NMR (400 MHz, DMSO) δ 14.24 (s, 2H), 13.14 (s, 1H), 8.96 (d, J = 1.2 Hz, |
| | 1H), 7.71 (t, J = 7.9 Hz, 1H), 7.60 (s, 1H), 7.16-7.00 (m, 5H), 4.77 (t, J = 7.9 Hz, 1H), 3.44 (dd, J = 13.8, 8.5 Hz, |
| | 1H), 3.23 (dd, J = 13.8, 7.4 Hz, 1H), 2.21 (s, 3H), 2.13 (d, J = 8.1 Hz, 3H). |
| 198 | LC-MS: [M-C2HF3O2 + 1] += 405.2, 1H NMR (400 MHz, DMSO) δ 14.20 (s, 2H), 8.95 (d, J = 1.0 Hz, 1H), 8.31 (s, |
| | 1H), 7.60 (s, 1H), 7.20 (d, J = 8.5 Hz, 2H), 7.10-6.87 (m, 5H), 6.44 (d, J = 6.8 Hz, 1H), 4.64 (t, J = 7.8 Hz, 1H), 4.15 |
| | (dd, J = 6.2, 2.9 Hz, 1H), 3.78-3.67 (m, 3H), 3.44 (dd, J = 8.9, 3.5 Hz, 1H), 3.26 (dd, J = 13.8, 8.5 Hz, 1H), 3.04 (dd, |
| | J = 13.8, 7.1 Hz, 1H), 2.22 (s, 3H), 2.13 (s, 3H), 2.11-2.04 (m, 1H), 1.77-1.59 (m, 1H). |
| 199 | LC-MS: [M + 1] += 357.2, 1H NMR (400 MHz, DMSO) δ 7.82 (s, 1H), 7.46 (dd, J = 12.6, 8.0 Hz, 2H), 7.23-7.13 (m, |
| | 3H), 7.06-6.90 (m, 3H), 4.58 (t, J = 7.8 Hz, 1H), 3.39 (dd, J = 13.7, 8.5 Hz, 1H), 3.10 (dd, J = 13.8, 7.0 Hz, 1H), 2.20 |
| | (s, 3H), 2.16 (s, 3H). |
| 200 | LC-MS: [M-C2HF3O2 + 1] += 376.2, 1H NMR (400 MHz, DMSO) δ 14.20 (s, 2H), 9.85 (s, 1H), 8.94 (d, J = 1.1 Hz, |
| | 1H), 7.59 (s, 1H), 7.43 (t, J = 9.2 Hz, 2H), 7.13-7.00 (m, 5H), 4.74-4.58 (m, 5H), 3.96-3.86 (m, 1H), 3.30 (dd, J = 13.8, |
| | 8.5 Hz, 1H), 3.08 (dd, J = 13.8, 7.2 Hz, 1H), 2.21 (s, 3H), 2.14 (d, J = 7.8 Hz, 3H). |
| 201 | [M + 1] += 362.17, 1H NMR (400 MHz, DMSO) δ 11.92 (s, 1H), 7.88 (s, 1H), 7.62 (s, 1H), 7.45 (s, 1H), 7.29 (t, J = 7.6 |
| | Hz, 1H), 7.21 (s, 1H), 7.06-6.98 (m, 2H), 6.83 (s, 1H), 6.61 (s, 1H), 5.86 (s, 1H), 3.93 (s, 3H), 2.24 (s, 3H), 2.12 (s, 3H). |
| 202 | LC-MS: [M-C2HF3O2 + 1] += 364.3, 1H NMR (400 MHz, DMSO) δ 14.19 (s, 2H), 9.65 (s, 1H), 8.95 (s, 1H), 7.60 (s, |
| | 1H), 7.49 (d, J = 8.1 Hz, 2H), 7.08 (d, J = 6.8 Hz, 5H), 4.67 (t, J = 7.7 Hz, 1H), 3.95 (s, 2H), 3.36-3.27 (m, 4H), 3.09 |
| | (dd, J = 13.7, 7.1 Hz, 1H), 2.21 (s, 3H), 2.13 (s, 3H). |
| 203 | LC-MS: [M-C2HF3O2 + 1] += 306.2, 1H NMR (400 MHz, DMSO) δ 14.24 (s, 2H), 8.95 (d, J = 1.2 Hz, 1H), 7.59 (s, |
| | 1H), 7.13-7.01 (m, 3H), 6.96 (d, J = 8.2 Hz, 2H), 6.59 (d, J = 6.7 Hz, 2H), 4.62 (t, J = 7.8 Hz, 1H), 3.23 (dd, J = 13.9, |
| | 8.5 Hz, 1H), 3.02 (dd, J = 13.8, 7.1 Hz, 1H), 2.67 (s, 3H), 2.21 (s, 3H), 2.13 (s, 3H). |
| 204 | LC-MS: [M-C2HF3O2 + 1] += 460.2, 1H NMR (400 MHz, DMSO) δ 14.03 (s, 2H), 8.94 (t, J = 6.0 Hz, 1H), 8.01-7.94 |
| | (m, 2H), 7.71 (dd, J = 11.1, 4.8 Hz, 3H), 7.63 (t, J = 7.6 Hz, 2H), 7.57 (s, 1H), 7.28 (d, J = 8.3 Hz, 2H), 7.11-7.02 |
| | (m, 3H), 4.74 (t, J = 7.9 Hz, 1H), 3.42 (dd, J = 13.8, 8.3 Hz, 2H), 3.21 (dd, J = 13.8, 7.5 Hz, 1H), 2.20 (s, 3H), 2.12 (d, |
| | J = 9.4 Hz, 3H). |
| 205 | LC-MS: [M-C2HF3O2 + 1] += 419.3, 1H NMR (400 MHz, DMSO) δ 14.22 (s, 2H), 8.95 (d, J = 1.2 Hz, 1H), 8.31 (s, |
| | 1H), 7.59 (s, 1H), 7.21 (d, J = 8.5 Hz, 2H), 7.13-6.92 (m, 5H), 6.22 (d, J = 7.6 Hz, 1H), 4.64 (t, J = 7.8 Hz, 1H), 3.80 |
| | (dt, J = 11.3, 3.3 Hz, 2H), 3.62 (qd, J = 10.7, 5.5 Hz, 1H), 3.36 (td, J = 11.4, 2.1 Hz, 2H), 3.26 (dd, J = 13.8, 8.5 Hz, |
| | 1H), 3.04 (dd, J = 13.8, 7.1 Hz, 1H), 2.21 (s, 3H), 2.13 (s, 3H), 1.75 (d, J = 10.3 Hz, 2H), 1.44-1.27 (m, 2H). |
| 206 | LC-MS: [M-C2HF3O2 − 1] −= 333.1, 1H NMR (400 MHz, DMSO) δ 14.19 (s, 2H), 8.95 (d, J = 1.0 Hz, 1H), 7.62 (s, |
| | 1H), 7.12-7.01 (m, 7H), 4.69 (t, J = 7.8 Hz, 1H), 3.48 (s, 2H), 3.34-3.30 (m, 1H), 3.10 (dd, J = 13.9, 7.1 Hz, 1H), |
| | 2.21 (s, 3H), 2.13 (s, 3H). |
| 207 | LC-MS: [M + 1] += 376.3., 1H NMR (400 MHz, DMSO) δ 12.02 (s, 1H), 7.57 (s, 1H), 7.13 (d, J = 7.3 Hz, 1H), 6.98 (t, |
| | J = 7.5 Hz, 1H), 6.93 (d, J = 7.0 Hz, 1H), 6.84-6.75 (m, 3H), 6.40 (d, J = 8.4 Hz, 2H), 5.19 (s, 1H), 4.41 (t, J = 7.6 Hz, |
| | 1H), 3.83 (dd, J = 8.2, 3.3 Hz, 2H), 3.36 (td, J = 11.4, 1.9 Hz, 3H), 3.17 (dd, J = 13.8, 8.0 Hz, 1H), 2.87 (dd, J = 13.8, |
| | 7.2 Hz, 1H), 2.19 (s, 3H), 2.12 (s, 3H), 1.81 (d, J = 12.7 Hz, 2H), 1.39-1.18 (m, 2H). |
| 208 | LC-MS: [M-C2HF3O2 + 1] += 374.2, 1H NMR (400 MHz, DMSO) δ 14.27 (s, 2H), 8.96 (d, J = 1.0 Hz, 1H), 7.71-7.53 |
| | (m, 4H), 7.26 (d, J = 11.2 Hz, 1H), 7.10 (tdd, J = 9.0, 7.1, 2.4 Hz, 4H), 4.79 (t, J = 7.9 Hz, 1H), 3.45 (dd, J = 13.8, 8.5 |
| | Hz, 1H), 3.25 (dd, J = 13.8, 7.4 Hz, 1H), 2.22 (s, 3H), 2.15 (s, 3H). |
| 209 | [M + 1] += 358.2, 1H NMR (400 MHz, MeOD) δ 7.72 (s, 1H), 7.57 (s, 1H), 7.37-7.30 (m, 2H), 7.26-7.23 (m, 2H), |
| | 7.18 (d, J = 7.6 Hz, 1H), 7.02 (d, J = 7.6 Hz, 1H), 6.96 (d, J = 7.6 Hz, 1H), 6.75 (s, 1H), 4.65 (t, J = 7.6 Hz, 1H), 4.03 |
| | (s, 3H), 3.51 (dd, J = 13.6, 6.8 Hz, 1H), 3.22 (dd, J = 13.6, 8.8 Hz, 1H), 2.21 (s, 3H), 2.09 (s, 3H). |
| 210 | [M + 1] += 345.17, 1 H NMR (400 MHz, MeOD) δ 8.41 (s, 1H), 7.84 (d, J = 8.0 Hz, 2H), 7.27 (s, 1H), 7.18 (d, J = 8.0 |
| | Hz, 2H), 7.10 (s, 1H), 7.10-7.06 (m, 1H), 7.05-7.02 (m, 1H), 4.74 (t, J = 8.0 Hz, 1H), 3.47 (dd, J = 13.6, 7.6 Hz, |
| | 1H), 3.25 (dd, J = 13.6, 8.0 Hz, 1H), 2.22 (s, 3H), 2.08 (s, 3H). |
| 211 | [M + 1] += 358.1, 1H NMR (400 MHz, MeOD) δ 8.78 (d, J = 1.2 Hz, 1H), 8.19 (s, 1H), 7.65-7.63 (d, J = 8.5 Hz, 2H), |
| | 7.48 (s, 1H), 7.27-7.25 (m, 2H), 7.14-7.02 (m, 3H), 4.84-4.79 (m, 2H), 3.56-3.51 (m, 1H), 2.38 (s, 3H), 2.22 (s, |
| | 3H), 2.05 (s, 3H). |
| 212 | [M + 1] += 420.2, 1H NMR (400 MHz, MeOD) δ 7.55 (s, 1H), 7.51-7.49 (m, 2H), 7.14-7.12 (m, 1H), 7.04-6.99 (m, |
| | 3H), 6.96-6.94 (m, 1H), 6.76 (s, 1H), 4.59 (t, J = 8.0 Hz, 1H), 4.02-3.98 (m, 2H), 3.52-3.46 (m, 2H), 3.44-3.39 |
| | (m, 1H), 3.15-3.10 (m, 1H), 2.98-2.87 (m, 1H), 2.21 (s, 3H), 2.14-2.05 (m, 2H), 2.04 (s, 3H), 1.70-1.67 (m, 2H). |
| 213 | LC-MS: [M-C2HF3O2 + 1] += 434.3, 1H NMR (400 MHz, DMSO) δ 14.26 (s, 2H), 8.96 (d, J = 1.1 Hz, 1H), 7.93 (d, |
| | J = 7.7 Hz, 1H), 7.74-7.50 (m, 2H), 7.21-6.98 (m, 3H), 6.98-6.73 (m, 2H), 4.76 (t, J = 7.9 Hz, 1H), 4.08-3.89 (m, |
| | 1H), 3.89-3.67 (m, 5H), 3.39 (ddd, J = 11.3, 5.5, 2.0 Hz, 3H), 3.19 (dd, J = 13.7, 7.6 Hz, 1H), 2.21 (s, 3H), 2.13 (s, |
| | 3H), 1.76 (d, J = 10.3 Hz, 2H), 1.51 (qd, J = 11.9, 4.3 Hz, 2H). |
| 214 | LC-MS: [M-C2HF3O2 + 1] += 391.2, 1H NMR (400 MHz, DMSO) δ 14.40 (s, 2H), 11.10 (d, J = 503.9 Hz, 1H), 8.96 |
| | (d, J = 1.1 Hz, 1H), 7.59 (s, 1H), 7.20 (dd, J = 27.6, 7.8 Hz, 4H), 7.15-6.96 (m, 3H), 4.85 (t, J = 8.8 Hz, 1H), 4.72 (dd, |
| | J = 15.9, 8.1 Hz, 1H), 4.66 (dd, J = 8.5, 5.3 Hz, 1H), 4.38-4.17 (m, 1H), 3.53 (ddd, J = 24.8, 11.8, 3.2 Hz, 2H), 3.39 |
| | (dd, J = 13.9, 8.3 Hz, 1H), 3.16 (dd, J = 13.9, 7.4 Hz, 1H), 2.21 (s, 3H), 2.12 (s, 3H). |
| 215 | LC-MS: [M-C2HF3O2 + 1] += 334.2, 1H NMR (400 MHz, DMSO) δ 14.21 (s, 2H), 8.95 (d, J = 1.2 Hz, 1H), 8.32 (d, |
| | J = 4.5 Hz, 1H), 7.67 (d, J = 8.3 Hz, 2H), 7.61 (s, 1H), 7.22 (d, J = 8.2 Hz, 2H), 7.12-7.01 (m, 3H), 4.74 (t, J = 7.9 Hz, |
| | 1H), 3.41 (dd, J = 13.7, 8.3 Hz, 1H), 3.19 (dd, J = 13.7, 7.5 Hz, 1H), 2.74 (d, J = 4.5 Hz, 3H), 2.20 (s, 3H), 2.11 (s, 3H). |
| 216 | LC-MS: [M-C2HF3O2 + 1] += 348.2, 1H NMR (400 MHz, DMSO) δ 14.24 (s, 2H), 8.96 (d, J = 0.9 Hz, 1H), 7.64 (s, |
| | 1H), 7.20 (dd, J = 23.5, 8.1 Hz, 4H), 7.10-7.01 (m, 3H), 4.72 (t, J = 7.8 Hz, 1H), 3.41 (dd, J = 13.7, 8.3 Hz, 1H), 3.16 |
| | (dd, J = 13.7, 7.5 Hz, 1H), 2.90 (d, J = 37.8 Hz, 6H), 2.20 (s, 3H), 2.09 (s, 3H). |
| 217 | LC-MS: [M-C2HF3O2 + 1] += 356.2, 1H NMR (400 MHz, DMSO) δ 14.24 (s, 2H), 8.95 (d, J = 0.9 Hz, 1H), 7.66 (d, |
| | J = 8.3 Hz, 2H), 7.60 (s, 1H), 7.35 (d, J = 8.3 Hz, 2H), 7.28 (s, 2H), 7.13-7.02 (m, 3H), 4.76 (t, J = 7.9 Hz, 1H), |
| | 3.47-3.41 (m, 1H), 3.23 (dd, J = 13.8, 7.4 Hz, 1H), 2.21 (s, 3H), 2.13 (s, 3H). |
| 218 | LC-MS: [M-C2HF3O2 + 1] += 337.2, 1H NMR (400 MHz, DMSO) δ 14.21 (s, 3H), 11.20 (s, 1H), 8.95 (d, J = 1.2 Hz, |
| | 1H), 7.72-7.51 (m, 2H), 7.24-7.01 (m, 3H), 6.86-6.63 (m, 2H), 4.75 (t, J = 7.9 Hz, 1H), 3.36 (dd, J = 13.8, 8.6 Hz, |
| | 1H), 3.16 (dd, J = 13.8, 7.2 Hz, 1H), 2.19 (d, J = 22.4 Hz, 6H). |
| 219 | LC-MS: [M-C2HF3O2 + 1] += 336.2, 1H NMR (400 MHz, DMSO) δ 14.16 (s, 2H), 11.12 (s, 1H), 8.94 (d, J = 23.4 Hz, |
| | 2H), 7.60 (d, J = 8.4 Hz, 3H), 7.23 (d, J = 8.2 Hz, 2H), 7.10-7.02 (m, 3H), 4.73 (t, J = 7.8 Hz, 1H), 3.44-3.40 (m, |
| | 1H), 3.18 (dd, J = 13.8, 7.3 Hz, 1H), 2.21 (s, 3H), 2.12 (s, 3H). |
| 220 | LC-MS: [M + 1] += 414.15, 1H NMR (400 MHz, MeOD) δ 7.73 (s, 1H), 7.68 (d, J = 0.8 Hz, 1H), 7.49 (t, J = 7.6 Hz, |
| | 1H), 7.35-7.32 (m, 2H), 7.11 (s, 1H), 7.07-7.05 (m, 1H), 7.02-6.98 (m, 1H), 6.72 (d, J = 7.6 Hz, 1H), 6.41 (s, |
| | 1H), 5.81 (s, 1H), 4.42-4.32 (m, 1H), 4.03-3.90 (m, 2H), 3.27-3.23 (m, 2H), 2.30 (s, 3H), 2.28-2.21 (m, 2H), |
| | 2.19 (s, 3H), 1.77-1.74 (m, 2H). |
| 221 | [M-C2HF3O2 + 1] += 357.1, 1H NMR (400 MHz, DMSO) δ 14.12 (d, J = 25.5 Hz, 2H), 8.93 (s, 1H), 7.61 (s, 1H), 7.44 |
| | (d, J = 8.0 Hz, 2H), 7.18-7.02 (m, 5H), 4.79-4.64 (m, 1H), 3.34 (dd, J = 13.8, 9.1 Hz, 1H), 3.14 (dd, J = 13.8, 6.7 |
| | Hz, 1H), 2.21 (d, J = 16.4 Hz, 6H). |
| 222 | LC-MS: [M-C2HF3O2 + 1] += 348.3, 1H NMR (400 MHz, DMSO) δ 14.25 (s, 2H), 8.97 (s, 1H), 7.66 (s, 1H), 7.31-6.81 |
| | (m, 7H), 4.70 (t, J = 7.8 Hz, 1H), 3.41 (dd, J = 13.3, 7.9 Hz, 1H), 3.24-2.96 (m, 4H), 2.18 (s, 3H), 2.04 (s, 3H), |
| | 1.75 (d, J = 51.6 Hz, 3H). |
| 223 | [M-C2HF3O2 + 1] += 309.2 1H NMR (400 MHz, DMSO) δ 11.85 (s, 1H), 7.53 (s, 1H), 7.20 (d, J = 7.6 Hz, 1H), 7.00 (t, |
| | J = 7.5 Hz, 1H), 6.93 (d, J = 7.2 Hz, 1H), 6.81-6.67 (m, 2H), 6.61 (dd, J = 8.2, 1.6 Hz, 1H), 6.56 (d, J = 1.5 Hz, 1H), |
| | 4.45 (t, J = 7.6 Hz, 1H), 3.66 (s, 3H), 3.57 (s, 3H), 3.28 (d, J = 7.7 Hz, 1H), 2.98 (dd, J = 13.6, 7.7 Hz, 1H), 2.18 (s, |
| | 3H), 2.10 (s, 3H). |
| 224 | [M + 1] += 337.2 1H NMR (400 MHz, DMSO) δ 11.85 (s, 1H), 7.53 (s, 1H), 7.20 (d, J = 7.6 Hz, 1H), 7.00 (t, J = 7.5 Hz, |
| | 1H), 6.93 (d, J = 7.2 Hz, 1H), 6.81-6.67 (m, 2H), 6.61 (dd, J = 8.2, 1.6 Hz, 1H), 6.56 (d, J = 1.5 Hz, 1H), 4.45 (t, J = 7.6 |
| | Hz, 1H), 3.66 (s, 3H), 3.57 (s, 3H), 3.28 (d, J = 7.7 Hz, 1H), 2.98 (dd, J = 13.6, 7.7 Hz, 1H), 2.18 (s, 3H), 2.10 (s, 3H). |
| 225 | LC-MS: [M-C2HF3O2 + 1] += 348.2, 1H NMR (400 MHz, DMSO) δ 14.19 (s, 2H), 10.61 (s, 1H), 8.95 (s, 1H), 7.60 (s, |
| | 1H), 7.13-6.98 (m, 3H), 6.81-6.65 (m, 3H), 4.66 (t, J = 7.8 Hz, 1H), 4.50 (s, 2H), 3.26 (dd, J = 13.9, 8.7 Hz, 1H), |
| | 3.04 (dd, J = 13.9, 7.0 Hz, 1H), 2.22 (s, 3H), 2.15 (s, 3H). |
| 226 | LC-MS: [M + 1] += 367.2, 1H NMR (400 MHz, DMSO) δ 11.96 (s, 1H), 7.58 (s, 1H), 7.21 (d, J = 7.5 Hz, 1H), 7.01 (t, |
| | J = 7.6 Hz, 1H), 6.95 (d, J = 7.2 Hz, 1H), 6.83 (s, 1H), 6.30 (s, 2H), 4.47 (t, J = 7.6 Hz, 1H), 3.61 (s, 6H), 3.57 (s, 3H), |
| | 3.38 (d, J = 33.0 Hz, 1H), 2.98 (dd, J = 13.5, 7.9 Hz, 1H), 2.18 (s, 3H), 2.07 (s, 3H). |
| 227 | LC-MS: [M-C2HF3O2 + 1] += 373.2., 1H NMR (400 MHz, DMSO) δ 14.21 (s, 2H), 8.94 (s, 1H), 7.62 (s, 1H), 7.14-7.01 |
| | (m, 7H), 4.67 (t, J = 6.3 Hz, 1H), 4.48 (q, J = 7.0 Hz, 1H), 3.33 (dd, J = 13.8, 8.5 Hz, 1H), 3.08 (dd, J = 13.8, 6.7 |
| | Hz, 1H), 2.19 (d, J = 5.2 Hz, 3H), 2.07 (d, J = 4.9 Hz, 3H), 1.61 (d, J = 7.2 Hz, 3H). |
| 228 | LC-MS: [M-C2HF3O2 + 1] += 377.3, 1H NMR (400 MHz, DMSO) δ 14.24 (s, 2H), 8.96 (s, 1H), 7.63 (s, 1H), 7.18-6.96 |
| | (m, 5H), 6.87 (d, J = 8.3 Hz, 2H), 4.66 (t, J = 7.8 Hz, 1H), 3.34 (dd, J = 13.6, 7.9 Hz, 1H), 3.08 (dd, J = 13.5, 7.9 |
| | Hz, 1H), 3.00 (s, 3H), 2.55 (s, 6H), 2.18 (s, 3H), 2.06 (s, 3H). |
| 229 | [M-C2HF3O2 − 1] −= 376.2 1H NMR (400 MHz, DMSO) δ 14.20 (s, 2H), 8.95 (s, 1H), 8.74 (t, J = 5.9 Hz, 1H), 7.72 (d, |
| | J = 8.2 Hz, 2H), 7.61 (s, 1H), 7.26 (d, J = 8.2 Hz, 2H), 7.15-7.01 (m, 3H), 4.75 (t, J = 7.9 Hz, 1H), 3.42 (dd, J = 13.7, |
| | 8.4 Hz, 1H), 3.21 (dd, J = 13.7, 7.4 Hz, 1H), 2.21 (s, 3H), 2.13 (s, 3H). |
| 230 | LC-MS: [M + 1] += 346.15, 1H NMR (400 MHz, DMSO) δ 14.13 (s, 1H), 9.99 (s, 1H), 8.95 (s, 1H), 7.62 (s, 1H), 7.05 |
| | (dd, J = 17.2, 7.2 Hz, 3H), 6.96-6.88 (m, 2H), 6.66 (d, J = 4.0 Hz, 1H), 4.64 (t, J = 6.0 Hz, 1H), 3.26-3.20 (m, 1H), |
| | 3.03 (dd, J = 12.8, 6.0 Hz, 1H), 2.79-2.73 (m, 2H), 2.41-2.36 (m, 2H), 2.22 (s, 3H), 2.14 (s, 3H). |
| 231 | LC-MS: [M + 1] += 306.15, 1H NMR (400 MHz, MeOD) δ 7.53 (s, 1H), 7.11 (d, J = 7.6 Hz, 1H), 7.00 (t, J = 7.6 Hz, |
| | 1H), 6.93 (dd, J = 14.4, 6.4 Hz, 2H), 6.74 (s, 1H), 6.43-6.38 (m, 2H), 6.29 (s, 1H), 4.57 (t, J = 7.6 Hz, 1H), |
| | 3.33-3.32(m, 1H), 3.02 (dd, J = 13.2, 8.4 Hz, 1H), 2.62 (s, 3H), 2.21 (s, 3H), 2.05 (s, 3H). |
| 232 | LC-MS: [M-C2HF3O2 + 1] += 355.2, 1H NMR (400 MHz, DMSO) δ 14.21 (s, 2H), 8.95 (s, 1H), 7.78 (d, J = 8.3 Hz, |
| | 2H), 7.61 (s, 1H), 7.43 (d, J = 8.2 Hz, 2H), 7.16-7.01 (m, 3H), 4.78 (t, J = 7.8 Hz, 1H), 3.49 (dd, J = 13.8, 8.4 Hz, |
| | 1H), 3.27 (dd, J = 13.8, 7.4 Hz, 1H), 3.16 (s, 3H), 2.21 (s, 3H), 2.12 (s, 3H). |
| 233 | LC-MS: [M-C2HF3O2 + 1] += 420.2, 1H NMR (400 MHz, DMSO) δ 14.26 (s, 2H), 12.63 (s, 1H), 8.96 (d, J = 0.9 Hz, |
| | 1H), 8.51 (d, J = 7.7 Hz, 1H), 7.70 (t, J = 14.8 Hz, 1H), 7.57 (s, 1H), 7.27-6.94 (m, 3H), 6.83-6.58 (m, 2H), 4.74 (t, |
| | J = 7.9 Hz, 1H), 4.06-3.97 (m, 1H), 3.87 (d, J = 9.7 Hz, 2H), 3.45-3.26 (m, 3H), 3.14 (dd, J = 13.8, 7.4 Hz, 1H), |
| | 2.22 (s, 3H), 2.16 (s, 3H), 1.80-1.68 (m, 2H), 1.58 (qd, J = 12.2, 4.4 Hz, 2H). |
| 234 | LCMS: [M-C2HF3O2 + 1] += 376.2, 1H NMR (400 MHz, DMSO) δ 14.21 (s, 2H), 9.78 (s, 1H), 8.95 (s, 1H), 7.63-7.52 |
| | (m, 3H), 7.07 (dt, J = 9.1, 6.6 Hz, 5H), 5.04 (dd, J = 8.9, 6.6 Hz, 1H), 4.63 (tt, J = 8.7, 6.8 Hz, 3H), 3.31 (dd, J = 13.8, |
| | 8.5 Hz, 1H), 3.10 (dd, J = 13.8, 7.2 Hz, 1H), 2.94 (tt, J = 10.9, 8.7 Hz, 1H), 2.58 (ddd, J = 15.3, 11.0, 6.7 Hz, |
| | 1H), 2.22 (s, 3H), 2.14 (s, 3H). |
| 235 | LC-MS: [M-C2HF3O2 + 1] += 334.2, 1H NMR (400 MHz, DMSO) δ 14.18 (s, 2H), 8.95 (s, 1H), 7.59 (s, 1H), 7.15-6.93 |
| | (m, 3H), 6.46 (d, J = 7.6 Hz, 2H), 6.40 (d, J = 7.8 Hz, 1H), 4.65-4.55 (m, 1H), 4.04 (dd, J = 9.3, 5.0 Hz, 2H), 3.28-3.18 |
| | (m, 2H), 3.13 (dd, J = 13.9, 9.0 Hz, 1H), 2.93 (dd, J = 14.0, 6.6 Hz, 1H), 2.24 (d, J = 7.4 Hz, 3H), 2.16 (d, J = 7.6 Hz, 3H). |
| 236 | LCMS: [M-C2HF3O2 + 1] += 390.1, 1H NMR (400 MHz, DMSO) δ 14.21 (s, 2H), 8.95 (s, 1H), 8.42 (t, J = 5.4 Hz, |
| | 1H), 7.67 (d, J = 8.2 Hz, 2H), 7.59 (s, 1H), 7.23 (d, J = 8.2 Hz, 2H), 7.11-7.03 (m, 3H), 4.74 (t, J = 7.9 Hz, 1H), 3.40 |
| | (dd, J = 12.3, 5.4 Hz, 3H), 3.20 (dd, J = 13.7, 7.4 Hz, 1H), 2.47 (d, J = 7.2 Hz, 2H), 2.21 (s, 3H), 2.12 (s, 3H). |
| 237 | LC-MS: [M-C2HF3O2 + 1] += 349.2, 1H NMR (400 MHz, DMSO) δ 14.21 (s, 2H), 8.95 (d, J = 0.9 Hz, 1H), 8.42 (s, |
| | 1H), 7.59 (s, 1H), 7.22 (d, J = 8.4 Hz, 2H), 7.11-7.02 (m, 3H), 6.98 (d, J = 8.4 Hz, 2H), 6.01 (s, 1H), 4.64 (t, J = 7.7 Hz, |
| | 1H), 3.25 (dd, J = 13.8, 8.6 Hz, 1H), 3.04 (dd, J = 13.8, 7.1 Hz, 1H), 2.63 (t, J = 13.9 Hz, 3H), 2.22 (s, 3H), 2.13 (s, 3H). |
| 238 | LC-MS: [M-C2HF3O2 + 1] += 321.2., 1H NMR (400 MHz, DMSO) δ 14.29 (s, 2H), 8.97 (d, J = 0.8 Hz, 1H), 8.35 (d, |
| | J = 1.5 Hz, 1H), 8.03 (s, 1H), 7.88 (d, J = 8.0 Hz, 1H), 7.73 (dd, J = 8.0, 2.0 Hz, 1H), 7.64 (s, 1H), 7.58 (s, 1H), 7.10 |
| | (dt, J = 11.8, 7.3 Hz, 3H), 4.76 (t, J = 7.9 Hz, 1H), 3.49 (dd, J = 13.7, 8.0 Hz, 1H), 3.26 (dd, J = 13.7, 7.9 Hz, 1H), 2.19 |
| | (s, 3H), 2.07 (s, 3H). |
| 239 | [M-HCl − 1] −= 349.1, 1H NMR (400 MHz, DMSO) δ 12.38 (s, 1H), 7.55 (s, 1H), 7.16 (d, J = 7.4 Hz, 1H), 7.02-6.91 |
| | (m, 4H), 6.78 (s, 1H), 6.69 (d, J = 8.6 Hz, 2H), 4.55 (s, 2H), 4.46 (t, J = 7.6 Hz, 1H), 3.31-3.25 (m, 1H), 2.97 (dd, J = 13.8, |
| | 7.2 Hz, 1H), 2.19 (s, 3H), 2.13 (s, 3H). |
| 240 | LC-MS: [M + 1] += 300.1, 1H NMR (400 MHz, MeOD) δ 7.55-7.51 (m, 1H), 7.04-6.95 (m, 3H), 6.67 (s, 1H), 4.25 (t, |
| | J = 7.6 Hz, 1H), 2.27 (s, 3H), 2.24 (s, 3H), 2.15 (t, J = 7.6 Hz, 2H), 2.11-2.02 (m, 1H), 1.93-1.82 (m, 1H), |
| | 1.62-1.52 (m, 2H), 1.40-1.25 (m, 4H). |
| 241 | LC-MS: [M + 1] += 359.5, 1H NMR (400 MHz, MeOD) δ 7.87 (d, J = 8.4 Hz, 2H), 7.57 (s, 1H), 7.21-7.16 (m, 3H), |
| | 7.06-7.01 (m, 1H), 6.99-6.95 (m, 1H), 6.77 (s, 1H), 4.64 (t, J = 7.6 Hz, 1H), 4.38 (s, 3H), 3.50 (dd, J = 13.6, 7.2 Hz, |
| | 1H), 3.20 (dd, J = 13.6, 8.4 Hz, 1H), 2.20 (s, 3H), 2.06 (s, 3H). |
| 242 | LC-MS: [M-C2HF3O2 + 1] += 405.2., 1H NMR (400 MHz, DMSO) δ 14.26 (s, 2H), 10.35 (s, 1H), 8.97 (d, J = 1.0 Hz, |
| | 1H), 8.02 (d, J = 1.9 Hz, 1H), 7.92 (d, J = 8.5 Hz, 1H), 7.62 (s, 1H), 7.54 (dd, J = 8.6, 2.2 Hz, 1H), 7.07 (ddd, J = 11.7, |
| | 5.5, 3.7 Hz, 3H), 4.69 (t, J = 7.9 Hz, 1H), 3.88 (dd, J = 10.3, 2.6 Hz, 2H), 3.41-3.24 (m, 3H), 3.12 (dd, J = 13.9, 7.6 |
| | Hz, 1H), 2.71 (td, J = 10.7, 4.8 Hz, 1H), 2.21 (s, 3H), 2.11 (s, 3H), 1.71-1.56 (m, 4H). |
| 243 | LC-MS: [M-C2HF3O2 + 1] += 375.2, 1H NMR (400 MHz, DMSO) δ 14.22 (s, 2H), 8.96 (d, J = 0.9 Hz, 1H), 7.61 (s, |
| | 1H), 7.40 (d, J = 8.6 Hz, 2H), 7.06 (td, J = 9.3, 5.3 Hz, 5H), 4.67 (t, J = 7.8 Hz, 1H), 3.74-3.66 (m, 2H), 3.43-3.35 |
| | (m, 2H), 3.30 (dd, J = 13.8, 8.6 Hz, 1H), 3.08 (dd, J = 13.8, 7.1 Hz, 1H), 2.73 (s, 3H), 2.21 (s, 3H), 2.14 (s, 3H). |
| 244 | LC-MS: [M-C2HF3O2 + 1] += 306.2, 1H NMR (400 MHz, DMSO) δ 14.25 (s, 2H), 8.96 (s, 1H), 7.87 (s, 1H), 7.71 (d, |
| | J = 8.1 Hz, 2H), 7.65 (s, 1H), 7.34-7.24 (m, 2H), 7.20 (d, J = 8.1 Hz, 3H), 7.16-7.06 (m, 2H), 4.64 (t, J = 7.8 Hz, |
| | 1H), 3.43 (dd, J = 13.5, 8.1 Hz, 1H), 3.21 (dd, J = 13.5, 7.8 Hz, 1H), 2.20 (s, 3H). |
| 245 | LC-MS: [M-C2HF3O2 + 1] += 419.3, 1H NMR (400 MHz, DMSO) δ 14.23 (s, 2H), 8.95 (s, 1H), 8.19 (s, 1H), 7.60 (s, |
| | 1H), 7.29 (d, J = 8.5 Hz, 2H), 7.05 (ddd, J = 19.5, 13.6, 8.1 Hz, 5H), 5.00-4.80 (m, 1H), 4.66 (t, J = 7.8 Hz, 1H), 3.91 |
| | (td, J = 8.5, 4.8 Hz, 1H), 3.70-3.52 (m, 3H), 3.27 (dd, J = 13.8, 8.6 Hz, 1H), 3.06 (dd, J = 13.8, 7.1 Hz, 1H), 2.83 (s, |
| | 3H), 2.22 (s, 3H), 2.18-2.04 (m, 4H), 1.80 (qd, J = 13.3, 8.1 Hz, 1H). |
| 246 | LC-MS: [M-C2HF3O2 + 1] += 333.2, 1H NMR (400 MHz, DMSO) δ 14.25 (s, 2H), 8.96 (s, 1H), 7.57 (s, 1H), 7.10-6.99 |
| | (m, 3H), 6.61 (s, 1H), 6.48 (d, J = 21.4 Hz, 2H), 4.62 (t, J = 7.5 Hz, 1H), 3.29 (s, 4H), 3.21-3.14 (m, 1H), 2.97 |
| | (dd, J = 13.9, 6.7 Hz, 1H), 2.23 (s, 3H), 2.18 (s, 3H). |
| 247 | LC-MS: [M + 1] += 352.05, 1H NMR (400 MHz, MeOD) δ 7.54 (d, J = 0.8 Hz, 1H), 7.40 (dd, J = 12.4, 2.0 Hz, 1H), |
| | 7.13 (d, J = 7.6 Hz, 1H), 7.00 (t, J = 7.6 Hz, 1H), 6.97-6.92 (m, 2H), 6.86 (t, J = 8.4 Hz, 1H), 6.78 (s, 1H), 4.64 (t, |
| | J = 8.0 Hz, 1H), 3.39 (dd, J = 13.6, 7.6 Hz, 1H), 3.09 (dd, J = 13.6, 8.4 Hz, 1H), 2.21 (s, 3H), 2.10 (s, 3H), 2.08 (s, 3H). |
| 248 | LC-MS: [M + 1] += 335.25, 1H NMR (400 MHz, MeOD) δ 8.80 (d, J = 1.2 Hz, 1H), 7.94-7.93 (m, 1H), 7.73-7.71 |
| | (m, 1H), 7.67-7.63 (m, 1H), 7.49 (s, 1H), 7.14-7.06 (m, 3H), 4.80-4.76 (m, 1H), 3.55-3.51 (m, 1H), 3.29-3.22 |
| | (m, 1H), 2.22 (s, 3H), 2.20 (s, 3H), 2.02 (s, 3H). |
| 249 | LC-MS: [M + 1] += 350.85, 1H NMR (400 MHz, MeOD) δ 7.53 (s, 1H), 7.05-6.95 (m, 3H), 6.68 (s, 1H), 4.26 (t, |
| | J = 7.6 Hz, 1H), 2.99 (t, J = 6.8 Hz, 2H), 2.88 (s, 3H), 2.28 (s, 3H), 2.25 (s, 3H), 2.14-2.04 (m, 1H), 1.92-1.85 (m, 1H), |
| | 1.52-1.47 (m, 2H), 1.43-1.37 (m, 2H), 1.36-1.28 (m, 2H). |
| 250 | LC-MS: [M + 1] += 406.20, 1H NMR (400 MHz, MeOD) δ 7.54 (s, 1H), 7.04-6.96 (m, 3H), 6.69 (s, 1H), 4.26 (t, J = 7.6 |
| | Hz, 1H), 4.00 (dd, J = 11.2, 4.0 Hz, 2H), 3.39 (td, J = 12.0, 1.6 Hz, 2H), 3.20-3.13 (m, 1H), 3.02 (t, J = 7.2 Hz, 2H), 2.28 |
| | (s, 3H), 2.25 (s, 3H), 2.14-2.04 (m, 1H), 1.93-1.84 (m, 3H), 1.79-1.69 (m, 2H), 1.60-1.52 (m, 2H), 1.42-1.33 (m, 2H). |
| 251 | LC-MS: [M-C2HF3O2 − 1] −= 361.2, 1H NMR (400 MHz, DMSO) δ 8.89 (s, 1H), 7.60 (s, 1H), 7.19 (d, J = 8.2 Hz, 2H), |
| | 7.14-7.04 (m, 5H), 4.70 (t, J = 7.7 Hz, 1H), 3.33 (dd, J = 13.9, 8.4 Hz, 1H), 3.09 (dd, J = 14.0, 7.1 Hz, 1H), 2.21 (s, |
| | 3H), 2.10 (s, 3H), 1.42 (s, 6H). |
| 401 | [M-C2HF3O2 + 1] += 362.1, 1H NMR (400 MHz, DMSO) δ 14.37 (s, 2H), 9.79 (s, 1H), 9.06 (d, J = 0.7 Hz, 1H), 7.34 |
| | (dd, J = 10.3, 5.4 Hz, 1H), 7.25 (d, J = 3.0 Hz, 1H), 7.17-7.11 (m, 1H), 7.06 (s, 2H), 6.99-6.92 (m, 2H), 5.58 (s, |
| | 1H), 2.97 (s, 3H), 2.46-2.37 (m, 1H), 2.35-2.23 (m, 1H), 1.08 (t, J = 7.4 Hz, 3H). |
| 402 | LC-MS = [M-C2HF3O2 + 1] += 362.1, 1H NMR (400 MHz, DMSO) δ 14.39 (s, 2H), 9.79 (s, 1H), 9.11 (s, 1H), 7.35 (t, |
| | J = 7.9 Hz, 1H), 7.15 (d, J = 8.0 Hz, 1H), 7.02 (d, J = 10.8 Hz, 2H), 6.97 (s, 1H), 6.92 (d, J = 7.7 Hz, 1H), 5.68 (s, 1H), |
| | 2.97 (s, 3H), 2.08 (s, 3H), 1.92 (s, 3H). |
| 403 | [M-C2F3O2 + 1] += 348, 1H NMR (400 MHz, DMSO) δ 14.34 (s, 2H), 9.76 (d, J = 16.2 Hz, 1H), 9.07 (s, 1H), 7.35 (t, |
| | J = 7.8 Hz, 1H), 7.24 (d, J = 2.1 Hz, 1H), 7.15 (d, J = 8.2 Hz, 1H), 7.08 (d, J = 5.7 Hz, 2H), 6.95 (dd, J = 12.3, 5.3 Hz, |
| | 2H), 5.57 (s, 1H), 2.97 (s, 3H), 2.01 (s, 3H). |
| 501 | [M-HCl + 1] += 412.1, 1H NMR (400 MHz, DMSO) δ 9.61 (s, 2H), 7.34 (dd, J = 24.5, 16.5 Hz, 3H), 7.09 (t, J = 7.8 Hz, |
| | 1H), 6.97 (d, J = 8.2 Hz, 1H), 6.89 (s, 1H), 6.72 (d, J = 7.6 Hz, 1H), 5.04 (dd, J = 11.1, 3.8 Hz, 1H), 3.86 (s, 4H), 3.61 |
| | (dd, J = 12.6, 3.8 Hz, 2H), 3.30-3.22 (m, 1H), 2.78 (s, 3H). |
| 502 | [M-C2HF3O2 + 1] += 378.0, 1H NMR (400 MHz, DMSO) δ 10.45 (s, 2H), 7.98 (s, 1H), 7.85 (s, 1H), 7.80 (d, J = 7.3 |
| | Hz, 1H), 7.55 (d, J = 8.1 Hz, 2H), 7.43 (dt, J = 14.9, 7.6 Hz, 3H), 7.26 (t, J = 8.1 Hz, 1H), 5.23 (t, J = 7.1 Hz, 1H), |
| | 3.81 (dd, J = 14.5, 6.8 Hz, 4H), 3.60 (d, J = 7.7 Hz, 1H), 3.50 (d, J = 5.7 Hz, 1H). |
| 503 | [M + 1] += 370.10, 1H NMR (400 MHz, DMSO) δ 11.69 (s, 1H), 7.66 (s, 1H), 7.53 (d, J = 6.8 Hz, 1H), 7.34 (dt, J = |
| | 18.4, 7.6 Hz, 3H), 7.21 (t, J = 8.0 Hz, 1H), 6.93 (dd, J = 16.8, 8.4 Hz, 3H), 5.06 (d, J = 6.4 Hz, 1H), 4.02 (t, J = 8.4 Hz, |
| | 2H), 3.80 (s, 3H), 3.71 (s, 3H), 3.38 (t, J = 8.4 Hz, 2H), 2.89 (dd, J = 13.6, 4.0 Hz, 1H), 2.75 (dd, J = 13.6, 10.0 Hz, 1H). |
| 504 | [M-HCl + 1] += 390.1, 1H NMR (400 MHz, MeOD) δ 7.40 (t, J = 7.9 Hz, 1H), 7.27-7.20 (m, 2H), 7.17 (t, J = 7.6 Hz, |
| | 2H), 7.00 (dd, J = 23.2, 7.5 Hz, 2H), 6.16 (s, 1H), 4.02 (t, J = 7.5 Hz, 2H), 3.65 (t, J = 7.6 Hz, 2H), 2.95 (s, 3H), 2.34 |
| | (s, 3H), 2.19 (s, 3H). |
| 505 | [M + 1] += 388.38, 1H NMR (400 MHz, DMSO) δ 9.69 (s, 1H), 8.74 (d, J = 9.2 Hz, 1H), 8.31 (s, 1H), 7.67 (s, 1H), |
| | 7.33-7.16 (m, 3H), 7.11 (s, 1H), 7.03 (dd, J = 17.2, 8.4 Hz, 3H), 6.94 (t, J = 7.6 Hz, 1H), 4.96-4.90 (m, 1H), 3.88 (s, 3H), |
| | 3.48 (s, 5H), 3.09 (dd, J = 13.6, 4.8 Hz, 1H), 2.94-2.87 (m, 4H). |
| 506 | [M + 1] += 389.70, 1H NMR (400 MHz, DMSO-D2O) δ 7.70 (s, 1H), 7.62 (dd, J = 8.6, 1.7 Hz, 1H), 7.39 (dd, J = 15.1, |
| | 8.1 Hz, 5H), 7.27 (d, J = 6.7 Hz, 1H), 5.03-4.98 (m, 1H), 4.77 (s, 0H), 3.94-3.82 (m, 1H), 3.82 (s, 4H), 3.51 (dd, |
| | J = 14.0, 7.2 Hz, 2H), 3.25 (dd, J = 14.8, 8.2 Hz, 2H) |
| 507 | [M-HCl + 1] += 438.2, 1H NMR (400 MHz, MeOD) δ 7.58 (s, 1H), 7.50 (d, J = 8.1 Hz, 1H), 7.37 (t, J = 7.9 Hz, 1H), |
| | 7.22 (d, J = 7.4 Hz, 1H), 7.16 (t, J = 7.6 Hz, 1H), 6.99 (d, J = 7.1 Hz, 2H), 6.16 (s, 1H), 4.02 (t, J = 7.5 Hz, 2H), 3.94 |
| | (dd, J = 11.2, 3.4 Hz, 2H), 3.65 (t, J = 7.4 Hz, 2H), 3.44 (td, J = 11.9, 1.8 Hz, 2H), 2.37-2.27 (m, 5H), 2.18 (s, 3H), |
| | 2.08 (dtd, J = 14.8, 7.6, 3.7 Hz, 1H), 1.67 (dd, J = 12.9, 1.6 Hz, 2H), 1.38 (qd, J = 12.2, 4.4 Hz, 2H). |
| 508 | [M-HCl + 1] += 372.1, 1H NMR (400 MHz, DMSO) δ 11.88 (s, 1H), 10.22 (t, J = 227.4 Hz, 2H), 7.79-7.66 (m, 2H), |
| | 7.55-7.42 (m, 2H), 7.42-7.32 (m, 1H), 7.21-6.88 (m, 3H), 6.21 (d, J = 162.9 Hz, 1H), 4.01-3.86 (m, 2H), 3.80 |
| | (s, 3H), 3.70 (s, 3H), 3.61-3.47 (m, 2H). |
| 509 | [M + 1] += 390.10, 1H NMR (400 MHz, DMSO) δ 9.60 (s, 1H), 8.22 (d, J = 18.8 Hz, 1H), 7.30 (d, J = 7.2 Hz, 1H), |
| | 7.25-7.19 (m, 2H), 7.13 (s, 1H), 7.06-6.99 (m, 1H), 6.96 (dd, J = 13.1, 8.0 Hz, 3H), 6.1-6.85 (m, 1H), 5.06 (dd, J = 9.2, |
| | 4.4 Hz, 1H), 4.27-4.09 (m, 2H), 3.82 (s, 3H), 3.48 (t, J = 8.8 Hz, 2H), 2.94 (s, 3H), 2.88 (s, 1H), 2.80-2.70 (m, 1H). |
| 510 | LC-MS = [M-HCl + 1] += 372.2, 1H NMR (400 MHz, DMSO) δ 9.98 (s, 2H), 7.35 (d, J = 7.1 Hz, 1H), 7.24 (t, J = 7.7 |
| | Hz, 1H), 7.15 (dd, J = 18.1, 7.2 Hz, 2H), 7.09-7.00 (m, 2H), 6.95 (d, J = 7.4 Hz, 1H), 4.44 (t, J = 7.5 Hz, 1H), 3.78 |
| | (s, 4H), 3.46-3.40 (m, 1H), 3.07 (dd, J = 13.4, 7.1 Hz, 1H), 2.90 (s, 3H), 2.22 (s, 3H), 2.11 (s, 3H). |
| 511 | [M + 1] += 383.9, 1H NMR (400 MHz, DMSO) δ 11.69 (s, 1H), 7.65 (s, 1H), 7.54 (d, J = 6.8 Hz, 1H), 7.45-7.24 (m, |
| | 4H), 7.07 (t, J = 7.6 Hz, 1H), 7.00 (d, J = 7.2 Hz, 1H), 5.17 (s, 1H), 3.73-3.61 (m, 5H), 3.10 (t, J = 7.2 Hz, 2H), 2.85 |
| | (qd, J = 14.0, 7.2 Hz, 2H), 2.21 (s, 3H), 2.16 (s, 3H). |
| 512 | [M + 1] += 423.8, 1H NMR (400 MHz, MeOD) δ 7.74 (d, J = 37.2 Hz, 1H), 7.62 (dd, J = 10.4, 8.8 Hz, 2H), 7.54-7.38 |
| | (m, 4H), 5.33 (d, J = 6.4 Hz, 1H), 5.19 (s, 1H), 3.87 (d, J = 11.2 Hz, 1H), 3.82 (s, 3H), 3.73 (dd, J = 18.8, 8.0 Hz, 1H), |
| | 3.47 (dd, J = 13.2, 5.6 Hz, 2H), 3.12 (t, J = 12.4 Hz, 1H). |
| 513 | [M + 1] += 387.05, 1H NMR (400 MHz, MeOD) δ 8.54-8.46 (m, 1H), 7.97 (d, J = 23.2 Hz, 1H), 7.44-7.31 (m, 2H), |
| | 7.26 (d, J = 7.2 Hz, 1H), 7.10-6.93 (m, 2H), 5.36-5.24 (m, 1H), 5.06-5.01 (m, 0H), 3.90 (d, J = 3.2 Hz, 2H), |
| | 3.88-3.84 (m, 2H), 3.82 (d, J = 3.2 Hz, 3H), 3.54-3.45 (m, 2H), 3.30-3.28 (m, 3H). |
| 514 | [M-HCL + 1] += 440.1, 1H NMR (400 MHz, DMSO + D2O) δ 7.59-7.47 (m, 2H), 7.40-7.29 (m, 2H), 7.17-6.90 (m, |
| | 4H), 6.07 (d, J = 139.2 Hz, 1H), 3.91 (t, J = 7.5 Hz, 2H), 3.80 (d, J = 10.3 Hz, 5H), 3.54 (t, J = 7.5 Hz, 2H), 3.27 (t, |
| | J = 11.3 Hz, 2H), 2.22 (d, J = 7.1 Hz, 2H), 1.95 (ddd, J = 11.0, 7.3, 3.9 Hz, 1H), 1.54 (d, J = 12.6 Hz, 2H), 1.22 (qd, |
| | J = 12.3, 4.2 Hz, 2H). |
| 515 | [M-HCl + 1] += 490.1, 1H NMR (400 MHz, DMSO + D2O) δ 7.29 (dt, J = 28.1, 7.6 Hz, 3H), 7.14-6.89 (m, 5H), 5.01 |
| | (d, J = 137.5 Hz, 1H), 3.77 (dd, J = 42.7, 34.7 Hz, 7H), 3.43 (s, 2H), 3.27 (t, J = 11.0 Hz, 2H), 3.20-2.86 (m, 4H), |
| | 2.06 (d, J = 4.4 Hz, 1H), 1.68 (d, J = 11.9 Hz, 2H), 1.26 (q, J = 11.6 Hz, 2H). |
| 516 | [M-HCl + 1] += 428.0, 1H NMR (400 MHz, DMSO) δ 10.52 (s, 2H), 9.82 (s, 1H), 7.55 (d, J = 8.1 Hz, 2H), 7.34-7.18 |
| | (m, 3H), 7.11 (dd, J = 18.0, 7.8 Hz, 2H), 5.16 (t, J = 7.0 Hz, 1H), 3.82 (td, J = 9.6, 3.6 Hz, 4H), 3.57-3.43 (m, 2H), |
| | 2.98 (s, 3H). |
| 517 | LC-MS: [M-HCl + 1] += 412.2., 1H NMR (400 MHz, DMSO) δ 11.58 (m, 1H), 10.71-9.43 (m, 2H), 7.79-6.87 (m, |
| | 6H), 5.06 (ddd, J = 163.0, 84.2, 66.5 Hz, 1H), 3.97-3.28 (m, 13H), 3.11-2.73 (m, 2H), 1.81 (d, J = 162.4 Hz, 2H). |
| 518 | LC-MS = [M-HCl + 1] += 372.2., 1H NMR (400 MHz, DMSO) δ 10.20 (s, 2H), 9.72 (s, 1H), 7.35 (d, J = 7.2 Hz, 1H), |
| | 7.22-7.05 (m, 6H), 4.45 (t, J = 7.8 Hz, 1H), 3.78 (s, 4H), 3.42-3.37 (m, 1H), 3.06 (dd, J = 13.7, 7.3 Hz, 1H), 2.94 |
| | (d, J = 9.4 Hz, 3H), 2.22 (s, 3H), 2.09 (s, 3H). |
| 519 | [M-C2HF3O2 + 1] += 428.0, 1H NMR (400 MHz, DMSO) δ 10.48 (s, 2H), 7.73 (d, J = 8.1 Hz, 2H), 7.53 (t, J = 7.9 Hz, |
| | 4H), 7.45 (d, J = 5.0 Hz, 1H), 7.24 (t, J = 8.1 Hz, 1H), 5.29 (t, J = 6.8 Hz, 1H), 3.83 (dd, J = 15.0, 6.7 Hz, 4H), 3.56 |
| | (ddd, J = 30.5, 14.0, 6.9 Hz, 2H), 2.38 (d, J = 4.9 Hz, 3H). |
| 520 | LC-MS: [M-C2HF3O2 + 1] += 284.1/286.1., 1H NMR (400 MHz, DMSO) δ 10.10 (s, 1H), 8.30 (s, 2H), 7.60 (dt, |
| | J = 8.9, 4.4 Hz, 1H), 7.54-7.44 (m, 2H), 4.49 (s, 2H), 3.65 (s, 4H), 3.59 (dd, J = 5.5, 3.6 Hz, 2H), 3.49 (dd, J = 5.7, |
| | 3.5 Hz, 2H), 3.27 (s, 3H). |
| 601 | [M + 1] += 294.1, 1H NMR (400 MHz, DMSO) δ 11.49 (s, 1H), 8.73 (s, 1H), 8.04 (t, J = 6.0 Hz, 3H), 7.94 (t, J = 7.4 |
| | Hz, 2H), 7.66-7.53 (m, 3H), 6.44 (d, J = 4.9 Hz, 2H), 3.49 (s, 3H). |
| 602 | [M + 1] += 455.3 1H NMR (400 MHz, DMSO) δ 13.79 (s, 1H), 10.73 (s, 1H), 8.36 (d, J = 7.0 Hz, 1H), 8.02 (ddd, |
| | J = 25.7, 14.2, 7.8 Hz, 3H), 7.77-7.63 (m, 2H), 7.56 (p, J = 6.7 Hz, 2H), 7.45 (dd, J = 6.9, 2.3 Hz, 1H), 5.96 (dd, J = 6.9, |
| | 2.2 Hz, 1H), 3.72-3.59 (m, 2H), 3.43 (tq, J = 8.6, 4.3 Hz, 16H), 3.22 (s, 3H), 3.00 (dt, J = 13.7, 6.9 Hz, 1H), 2.86 (dt, |
| | J = 13.4, 6.6 Hz, 1H). |

### Biological Assays

### Example 1. α2AAR FLIPR Assay

This experimental protocol involved cell seeding and a FLIPR assay using the α2AAR (α2A-adrenergic receptor) cell line hosted in HEK293 cells. The growth media used is DMEM (11965-092, Gibco) supplemented with 10% FBS (FSP500, Excell), 300 μg/mL G418 (10131-027, Gibco), and 2 μg/mL Blasticidin S HCl (BS) (A11139-03, Gibco). On Day 1, the cell seeding process started with the removal of the culture medium, followed by rinsing the cells with DPBS (21-031-CVC, Corning). Cells were then treated with 0.05% EDTA-Trypsin (25300-062, Gibco), incubated at 37° C. for 1-2 minutes, and monitored under an inverted microscope. The cells were detached, resuspended in growth media, and centrifuged at room temperature at 1000 rpm for 5 minutes. After discarding the supernatant, the cell pellet was resuspended in growth media to a concentration of 10× 105 cells per mL. This suspension was added to 384-well plates (19-Jul-38, Greiner) at 20 μL per well and incubated overnight at 37° C. in 5% CO₂.

On Day 2, the FLIPR assay began with the preparation of the assay buffer comprising 20 mM HEPES (15630-106, Invitrogen), 1×HBSS (14025-076, Invitrogen), and 0.5% BSA (B2064, Sigma). A 250 mM Probenecid solution was prepared in this buffer. The Fluo-4 Direct™ Loading Buffer was made by dissolving Fluo-4 Direct™ crystals (F10471, Invitrogen) in the FLIPR Assay Buffer and adding Probenecid. The buffer was then vortexed and allowed to stand for over 5 minutes, shielded from light. For the FLIPR procedure, testing compounds for agonist activite were serially diluted and transferred to a 384-well compound plate (25-Jan-39, Greiner). The cell plate was then treated with 2× Fluo-4 Direct™ loading buffer and incubated for 50 minutes at 37° C. in a 5% CO₂ atmosphere, followed by 10 minutes at room temperature. Subsequently, the FLIPR assay buffer was added to the compound plate, which is then centrifuged.

The cell plate was analyzed in the FLIPR Tetra+ System for fluorescence signals. For the agonist test, reference compounds were added to the cell plates, and fluorescence was measured. The “Max-Min” calculation began from Read 1 to the maximum allowed. The data were analyzed using Prism software to calculate activation percentage for agonists and inhibition percentage for antagonists. The results were then fitted using specific models to determine EC50 for agonists.

The experimental protocol utilized various reagents and apparatus, including Penicillin/Streptomycin (100×) (SV30010, Hyclone), Poly-L-lysine hydrobromide (P1399, Sigma), and different types of 384-well plates such as the 384-Well PP 2.0 Microplate (PP-0200, LABCYTE) and 384 well Low Dead Volume Microplate (LP-0200, LABCYTE). The use of specific reference compounds like UK14304 was also integral to the assay.

### Example 2. α2AAR Binding Assay

The α2AR Binding Assay was conducted using a stable HEK293 cell line, specifically constructed by WuXi AppTec for targeting α2AAR. This assay primarily focused on the binding activity of the radioligand [3H]—RX 821002 (PerkinElmer, NET1153250UC) to α2AAR, with the membrane concentration set at 0.5 μg/well and the radioligand concentration at 0.5 nM. Essential equipment for this assay includes Unifilter-96 GF/C filter plates (Perkin Elmer, 6005174), 96 well conical polypropylene plates (Agilent, 5042-1385), TopSeal-A sealing film (Perkin Elmer, 6050185), a MicroBeta2 reader (CNLL0153, Perkin Elemer, 1310887), and a cell harvester (UNIFILTER-96, Perkin Elemer, 1951369), all procured from Perkin Elmer. Both the assay and wash buffers consist of 50 mM Tris-HCl at a pH of 7.4 (Tris base, Sigma, T1503-1 KG).

The procedure initiated with the preparation of test compounds and a reference compound, yohimbine (Sigma, Y3125), through an 8-point 4-fold serial dilution, transferring 1 μL of each to the assay plate. The assay involved adding 100 μL of membrane stocks (0.5 μg/well) and 100 μL 0.5 nM of [3H]—RX 821002 to each well. After scaling, the plates were agitated at room temperature for one hour. Subsequently, the Unifilter-96 GF/C filter plates were pre-soaked with 0.3% PEI (Sigma, P3143) for at least half an hour. The reaction mixtures were then filtered and washed four times with cold wash buffer using a Perkin Elmer Cell harvester. Post-filtration, the plates were dried at 50° C. for one hour. The next step involved sealing the bottom of the filter plate wells with Perkin Elmer Unifilter-96 backing seal tape and adding 50 μL of MicroScint-O cocktail (PerkinElmer, 6013611) to each well. The top of the plates was then sealed with TopSeal-A sealing film. The trapped 3H was quantified using a Perkin Elmer MicroBeta2 Reader. The inhibition rate was calculated using the formula: % Inhibition=(1-(Assay well Average_LC)/(Average_HC-Average_LC))×100%. Finally, the data were analyzed with Prism 5.0 software, employing the “log (inhibitor) vs. response-Variable slope” model for data fitting. This comprehensive process ensured precise assessment of the binding affinity of compounds to the α2AAR.

The result of the α2AAR FLIPR assay and binding assay result are listed in Table 3 below.

TABLE 3
α2A AR agonist activity (EC50) and affinity (Ki)

| | | α2A | |
|---|---|---|---|
| | | agonist | α2A |
| Compound | racemate/ | activity | affinity |
| name | enantiomer | (EC50, nM) | (Ki, nM) |
| 1 | — | C | C |
| 1 | −A | E | |
| 1 | −B | B | C |
| 2 | — | B | |
| 3 | — | B | |
| 4 | — | C | |
| 5 | — | B | |
| 5 | −A | E | |
| 5 | −B | B | B |
| 6 | — | B | |
| 7 | — | D | |
| 8 | — | B | |
| 9 | — | B | |
| 10 | — | B | |
| 10 | −A | E | |
| 10 | −B | B | B |
| 11 | — | D | |
| 12 | — | E | |
| 13 | — | E | |
| 14 | — | E | |
| 15 | — | E | |
| 16 | — | B | |
| 17 | — | B | |
| 17 | −A | E | |
| 17 | −B | B | B |
| 18 | — | B | |
| 18 | −A | D | |
| 18 | −B | B | B |
| 19 | — | C | |
| 20 | — | B | C |
| 21 | — | E | |
| 22 | — | B | B |
| 23 | — | D | |
| 24 | — | D | |
| 25 | — | C | |
| 26 | — | B | C |
| 27 | — | B | A |
| 28 | — | B | B |
| 29 | — | B | A |
| 30 | — | C | C |
| 31 | — | B | C |
| 32 | — | C | C |
| 33 | — | C | |
| 34 | — | E | |
| 35 | — | C | |
| 36 | — | C | |
| 37 | — | C | |
| 38 | — | B | |
| 39 | — | C | |
| 40 | — | | E |
| 41 | — | | C |
| 42 | — | | C |
| 43 | — | B | B |
| 43 | −A | | E |
| 43 | −B | B | B |
| 44 | −A | | D |
| 44 | −B | B | A |
| 45 | −A | | E |
| 45 | −B | B | A |
| 46 | −A | | E |
| 46 | −B | A | B |
| 47 | −A | | D |
| 47 | −B | B | B |
| 48 | — | | E |
| 49 | — | | E |
| 50 | — | C | C |
| 51 | — | C | B |
| 52 | — | D | |
| 53 | — | | D |
| 54 | — | B | B |
| 55 | — | B | B |
| 56 | — | B | B |
| 57 | — | | D |
| 58 | — | | C |
| 59 | — | C | |
| 60 | — | | D |
| 61 | — | B | B |
| 62 | — | | C |
| 63 | — | B | B |
| 64 | — | B | B |
| 65 | — | | C |
| 66 | — | | D |
| 67 | — | B | B |
| 68 | — | | C |
| 69 | — | | C |
| 70 | — | B | B |
| 71 | — | | B |
| 72 | — | B | B |
| 73 | — | B | B |
| 73 | −A | B | A |
| 73 | −B | B | |
| 74 | — | C | B |
| 75 | — | | C |
| 76 | — | C | A |
| 77 | — | | D |
| 78 | — | | D |
| 79 | — | C | B |
| 80 | — | B | A |
| 81 | — | B | A |
| 82 | — | | E |
| 83 | — | | C |
| 84 | — | B | A |
| 85 | — | C | B |
| 86 | — | B | A |
| 87 | — | | C |
| 88 | — | C | B |
| 89 | — | C | B |
| 90 | — | C | C |
| 91 | — | | C |
| 92 | — | | E |
| 93 | — | | D |
| 94 | — | C | C |
| 95 | — | B | A |
| 96 | — | B | A |
| 97 | — | | C |
| 98 | −A | | D |
| 98 | −B | A | A |
| 99 | — | B | B |
| 100 | −A | | A |
| 100 | −B | | C |
| 101 | — | B | B |
| 102 | — | B | B |
| 103 | — | B | B |
| 104 | — | B | A |
| 105 | — | B | A |
| 106 | — | | C |
| 107 | — | | C |
| 108 | — | B | A |
| 109 | — | B | B |
| 110 | — | | C |
| 111 | — | B | B |
| 112 | — | B | A |
| 112 | −A | | D |
| 112 | −B | B | A |
| 113 | — | | C |
| 114 | — | | E |
| 115 | — | | D |
| 116 | — | | B |
| 117 | — | A | B |
| 118 | — | B | B |
| 119 | — | B | A |
| 120 | — | A | A |
| 121 | — | A | B |
| 121 | −A | E | |
| 121 | −B | A | A |
| 122 | −A | B | B |
| 122 | −B | | C |
| 123 | — | B | A |
| 124 | — | B | A |
| 124 | −A | | D |
| 124 | −B | B | A |
| 125 | — | | B |
| 126 | — | B | A |
| 127 | — | | B |
| 128 | — | | C |
| 129 | — | | C |
| 130 | −A | | E |
| 130 | −B | | D |
| 131 | — | | B |
| 132 | — | | C |
| 133 | — | B | B |
| 134 | — | B | A |
| 135 | — | | C |
| 136 | — | A | A |
| 136 | −A | E | |
| 136 | −B | A | A |
| 137 | −A | | C |
| 137 | −B | | E |
| 138 | — | B | A |
| 139 | — | B | B |
| 140 | — | | C |
| 141 | — | C | B |
| 142 | — | B | A |
| 143 | — | C | B |
| 144 | — | | C |
| 145 | — | B | A |
| 146 | — | B | B |
| 147 | — | B | B |
| 148 | — | B | B |
| 149 | — | B | B |
| 150 | — | C | |
| 151 | −A | B | B |
| 151 | −B | B | |
| 152 | — | B | A |
| 153 | — | B | B |
| 154 | −A | | D |
| 154 | −B | E | |
| 155 | −A | B | B |
| 155 | −B | B | |
| 156 | — | A | A |
| 156 | −A | | E |
| 156 | −B | A | A |
| 157 | — | A | A |
| 157 | −A | E | |
| 157 | −B | B | A |
| 158 | — | B | A |
| 159 | — | B | B |
| 160 | — | B | B |
| 161 | — | B | A |
| 162 | — | B | A |
| 163 | — | B | A |
| 164 | — | B | B |
| 165 | — | A | A |
| 166 | — | B | B |
| 167 | — | B | B |
| 168 | — | C | |
| 169 | — | A | A |
| 170 | — | C | |
| 171 | — | A | A |
| 172 | — | B | |
| 173 | — | B | |
| 174 | — | B | C |
| 174 | −A | E | |
| 174 | −B | B | B |
| 174 | −C | D | |
| 174 | −D | B | B |
| 175 | — | B | D |
| 175 | −A | | E |
| 175 | −B | B | C |
| 176 | — | A | A |
| 177 | — | A | B |
| 178 | — | B | |
| 179 | — | A | A |
| 180 | — | B | B |
| 181 | — | C | A |
| 182 | — | | C |
| 183 | — | | D |
| 184 | — | | D |
| 185 | — | C | |
| 186 | — | B | B |
| 187 | — | B | A |
| 188 | — | | E |
| 189 | — | | B |
| 190 | — | B | A |
| 191 | — | | C |
| 192 | — | B | A |
| 193 | — | B | A |
| 194 | — | B | A |
| 195 | — | B | A |
| 196 | — | C | A |
| 197 | — | B | C |
| 198 | — | B | A |
| 199 | — | | E |
| 200 | — | B | A |
| 201 | — | B | A |
| 202 | — | C | C |
| 203 | — | C | A |
| 204 | — | C | C |
| 205 | — | B | A |
| 206 | — | C | C |
| 207 | — | B | A |
| 208 | — | A | A |
| 209 | — | B | A |
| 210 | — | C | B |
| 211 | — | B | A |
| 212 | — | B | A |
| 213 | — | A | A |
| 214 | — | B | A |
| 215 | — | A | A |
| 216 | — | A | A |
| 217 | — | A | A |
| 218 | — | B | C |
| 219 | — | A | A |
| 220 | — | A | A |
| 221 | — | B | B |
| 222 | — | B | B |
| 223 | — | B | A |
| 224 | — | A | A |
| 225 | — | A | A |
| 226 | — | A | A |
| 227 | — | B | B |
| 228 | — | A | A |
| 229 | — | B | C |
| 230 | — | B | A |
| 231 | — | B | B |
| 232 | — | A | A |
| 233 | — | B | B |
| 234 | — | B | A |
| 235 | — | B | A |
| 236 | — | B | C |
| 237 | — | B | A |
| 238 | — | | C |
| 239 | — | B | C |
| 240 | — | | D |
| 241 | — | B | B |
| 242 | — | B | C |
| 243 | — | A | A |
| 244 | — | C | C |
| 245 | — | A | A |
| 246 | — | B | B |
| 247 | — | B | A |
| 248 | — | B | C |
| 249 | — | | C |
| 250 | — | | C |
| 251 | — | B | C |
| 401 | — | B | A |
| 402 | — | | D |
| 403 | — | C | |
| 501 | — | | D |
| 502 | — | | C |
| 503 | — | C | A |
| 504 | — | | C |
| 505 | — | C | A |
| 506 | — | C | A |
| 507 | — | | B |
| 508 | — | | D |
| 509 | — | C | A |
| 510 | — | | C |
| 511 | — | | A |
| 512 | — | | A |
| 513 | — | | A |
| 514 | — | | C |
| 515 | — | C | A |
| 516 | — | C | C |
| 517 | — | | B |
| 518 | — | | C |
| 519 | — | C | B |
| 520 | — | | D |
| 601 | — | C | |
| 602 | — | C | |

A: <10 nM
B: 10 nM-50 nM
C: 50 nM-250 nM
D: 250 nM-1000 nM
E: >1000 nM

### Example 3. MDR1-MDCK Permeability Assay

MDR1-MDCK II cells (obtained from Piet Borst at the Netherlands Cancer Institute) were seeded onto Polycarbonate membranes (PC) in 96-well insert systems at 3.33×105 cells/mL until to 4-7 days for confluent cell monolayer formation.

Selected α2AR agonist from table 3s were diluted with the transport buffer (HBSS with 10.0 mM Hepes, pH7.4) from DMSO stock solution to a concentration of 2 μM (DMSO<1%) and applied to the apical or basolateral side of the cell monolayer. Digoxin was used as a positive control for the P-glycoprotein (P-gp) substrate, while clonidine, dexmedetomidine, faldomidine and brimonidine were used as negative control. Permeation of the test compounds from A to B direction and/or B to A direction was determined in duplicate. Digoxin was tested at 10.0 μM from A to B direction and B to A direction in duplicate. The plate was incubated for 2.5 hours in CO₂ incubator at 37.0±1.0° C., with 5.0% CO₂ at saturated humidity without shaking. In addition, the efflux ratio of each compound was also determined. Test and reference compounds were quantified by LC/MS/MS analysis based on the peak area ratio of analyte/IS.

After transport assay, lucifer yellow rejection assay was applied to determine the cell monolayer integrity. Buffers were removed from both apical and basolateral chambers, followed by the addition of 75 μL of 100 μM lucifer yellow in transport buffer and 250 μL transport buffer in apical and basolateral chambers, respectively. The plate was incubated for 30 minutes at 37.0° C. with 5.0% CO₂ and 95.0% relative humidity without shaking. After 30 minutes incubation, 20 μL of lucifer yellow samples were taken from the apical sides, followed by the addition of 60 μL of transport Buffer. And then 80 μL of lucifer yellow samples were taken from the basolateral sides. The relative fluorescence unit (RFU) of lucifer yellow was measured at 425/528 nm (excitation/emission) with an Envision plate reader.

The apparent permeability coefficient Papp (cm/s) was calculated using the equation:

$$Papp = ( dCr / dt ) \times Vr / ( A \times C 0 )$$

wherein dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.143 cm2 for the area of the monolayer; and C0 is the initial concentration in the donor chamber (μM).

The efflux ratio was calculated using the equation:

$$Efflux Ratio = Papp ( BA ) / Papp ( AB )$$

The results of the MDR1-MDCK permeability assay are listed in Table 4 below.

TABLE 4
Efflux ratio of P-gp from MDCK-MDR1 assay

| compound | racemate/ | Efflux |
|---|---|---|
| name | enantiomer | ratio |
| Digoxin | | 13.74 |
| Clonidine | | 0.71 |
| Dexmedetomidine | | 0.64 |
| Fadolmidine | | 0.80 |
| Brimonidine | | 0.83 |
| 1 | −B | 51.02 |
| 5 | −B | 33.20 |
| 18 | −B | 27.47 |
| 44 | −B | 41.00 |
| 45 | −B | 14.02 |
| 46 | −B | 34.01 |
| 47 | −B | 18.17 |
| 55 | | 26.39 |
| 56 | | 11.51 |
| 64 | | 24.33 |
| 67 | | 28.97 |
| 72 | | 32.93 |
| 73 | | 96.13 |
| 74 | | 93.45 |
| 80 | | 112.12 |
| 81 | | 62.48 |
| 95 | | 102.22 |
| 98 | | 82.58 |
| 99 | | 140.52 |
| 101 | | 103.83 |
| 103 | | 149.19 |
| 105 | | 65.78 |
| 108 | | 72.22 |
| 109 | | 94.75 |
| 111 | | 172.99 |
| 112 | | 58.40 |
| 118 | | 25.26 |
| 121 | −B | 30.89 |
| 124 | −B | 10.26 |
| 136 | −B | 7.69 |
| 138 | | 7.85 |
| 141 | | 2.61 |
| 156 | −B | 2.86 |
| 157 | −B | 10.85 |
| 158 | | 23.66 |
| 163 | | 9.08 |
| 165 | | 14.79 |
| 186 | | 3.38 |
| 187 | | 2.60 |
| 190 | | 4.91 |
| 200 | | 27.73 |
| 205 | | 61.80 |
| 208 | | 8.88 |
| 213 | | 4.92 |
| 214 | | 56.11 |
| 215 | | 4.77 |
| 216 | | 3.21 |
| 217 | | 17.52 |
| 219 | | 26.89 |
| 401 | | 22.02 |

### Example 4. In Vivo Drug Distribution

The binding affinity of various compounds to plasma proteins was evaluated, including clonidine HCl, dexmedetomidine HCl, 1-B HCl, and 44-B HCl, with warfarin serving as a control. The experiment utilized a HT-Dialysis plate (HTD 96 b) and a dialysis membrane with a molecular weight cutoff of 12-14 kDa. The plasma was derived from male C57BL/6J mice, treated with EDTA-K2 as an anticoagulant. The experimental procedure commenced with plasma thawing under cold tap water, followed by centrifugation at 3220×g for 5 minutes to eliminate clots, and pH adjustment to 7.4+0.1.

Dialysis membranes were initially hydrated in ultra-pure water for about one hour and then treated in a 20:80 ethanol-water mixture for 20 minutes. These prepared membranes could be used immediately or stored at 2-8° C. for up to a month. Membranes were rinsed in ultra-pure water before use.

Test and control compounds were prepared at a 400 UM concentration by diluting stock solutions with DMSO. Working solutions were further diluted to create 2 μM loading matrix solutions, which were thoroughly mixed. In the assay, 50 μL aliquots of these solutions were dispensed in triplicate into a Sample Collection Plate, balanced with blank PBS to a final volume of 100 L per well. A stop solution containing acetonitrile, tolbutamide, and labetalol was added, and samples were mixed and cooled at 2 to 8° C.

During the dialysis, 100 μL aliquots from the loading matrix were placed in the dialysis well's donor side, matched with an equal volume of PBS on the receiver side, and incubated at 37° C. for 4 hours. Post-dialysis, samples from both sides were collected, balanced to 100 μL with corresponding blank fluids, treated with stop solution, vortexed, and centrifuged to prepare for LC-MS/MS analysis.

Data analysis involved calculating the percentages of Unbound, Bound, and Recovery of the compounds post-dialysis. % Unbound was calculated as the ratio of the compound's peak area on the receiver side to its internal standard, reflecting the fraction that crossed the membrane. % Bound was the complement of % Unbound, representing the fraction retained on the donor side. % Recovery was determined from the peak area ratios on both sides of the membrane, assessing the dialysis efficiency in retaining the compound. These metrics provided insights into the compound's free, bound, and recoverable quantities, elucidating its behavior in the dialysis system. The plasma protein binding ratio result is shown in Table 5.

TABLE 5
The plasma protein binding ratio

| | % Unbound | | | % Recovery | |
|---|---|---|---|---|---|
| Sample Name | (n = 3) | SD | % Bound | (n = 3) | SD |
| Clonidine HCl | 70.8 | 4.8 | 29.2 | 93.2 | 3.7 |
| Dexmedetomidine HCl | 6.8 | 0.2 | 93.2 | 88.9 | 1.8 |
| 1-B HCl | 5.1 | 0.3 | 94.5 | 88.8 | 3.5 |
| 44-B HCl | 6.4 | 0.8 | 93.6 | 86.7 | 7.0 |
| Warfarin | 2.3 | 0.1 | 97.7 | 82.7 | 2.2 |

The binding affinity of various compounds to brain proteins was evaluated including clonidine HCl, dexmedetomidine HCl, 1-B HCl, and 44-B HCl, with propranolol serving as a control. The initial preparation of the dialysis membrane involved thawing brain homogenate in a water bath at room temperature and subsequently heating it at 37° C. for 10 minutes. The dialysis setup utilized was from HT Dialysis LLC, featuring a HT-Dialysis plate (Model HTD 96 b) and a dialysis membrane with a molecular weight cutoff of 12-14 kDa.

The membrane underwent a comprehensive pretreatment which included hydration in ultra-pure water at room temperature for approximately one hour. This was followed by separation and immersion in a 20:80 ethanol:water solution for about 20 minutes. After this treatment, the membranes were either used immediately or stored at 2-8° C. for up to one month, with a final rinse in ultra-pure water prior to experimental use.

For compound preparation, test and control substances were first dissolved to create 400 μM working solutions by mixing 4 μL of stock solution with 96 μL of DMSO. These working solutions were then further diluted to 2 μM in a blank matrix by combining 3 μL of the prepared solution with 597 μL of matrix, ensuring thorough mixing.

During the assay, 50 μL aliquots of the 2 μM compound-matrix mixture were dispensed in triplicate into a Sample Collection Plate. Each aliquot was paired with an equal volume of blank PBS to standardize the total volume to 100 μL per well at a 1:1 matrix to PBS ratio. A stop solution comprising 500 μL of acetonitrile with tolbutamide and labetalol at 250 nM each was added to stabilize the samples at TO. The samples were then shaken at 800 rpm for 10 minutes and stored at 2-8° C.

The dialysis procedure included assembling the dialysis device according to the manufacturer's specifications, loading the matrix aliquots into the donor side of the dialysis wells, and conducting the dialysis under a humidified atmosphere with 5% CO₂ at 37° C. for 4 hours.

Post-dialysis, 50 μL samples were collected from both the receiver and donor sides into new 96-well plates. Volumes were adjusted to 100 μL by adding an equivalent amount of the opposite blank matrix or PBS. The samples were prepared for LC-MS/MS analysis after thorough vertexing and centrifugation. Blank control samples were prepared and processed similarly to mirror the test conditions.

Data analysis involved calculating the percentages of undiluted unbound and bound fractions, and recovery of the compounds. The % Undiluted Unbound was determined using the formula: % Undiluted Unbound=100×1/D/((1/(F/T)−1)+1/D), where D is the dilution factor (10). % Undiluted Bound was derived as 100-% Undiluted Unbound. % Recovery was calculated using: % Recovery=100×(F+T)/TO, with F and T representing the peak area ratios of the compound to the internal standard on the receiver and donor sides respectively, after 4 hours of incubation. The brain protein binding result is shown in Table 6

TABLE 6
The brain protein binding result

| | % Undiluted | | % Undiluted | | |
|---|---|---|---|---|---|
| | Unbound | | Bound | % Recovery | |
| Sample Name | (n = 3) | SD | (n = 3) | (n = 3) | SD |
| Clonidine HCl | 23.0 | 5.4 | 77.0 | 105.2 | 1.3 |
| Dexmedetomidine | 6.3 | 0.9 | 93.7 | 93.8 | 2.7 |
| HCl | | | | | |
| 1-B HCl | 5.1 | 0.3 | 94.9 | 88.8 | 3.5 |
| 44-B HCl | 2.4 | 0.3 | 97.6 | 88.6 | 3.7 |
| Propranolol | 2.6 | 0.2 | 97.4 | 108.6 | 1.8 |

Male C57BL/6J mouse was use in the in vivo distribution assay. The sample of brain, spinal cord and serum were collected for the drug distribution calculation.

Before commencing the study, the mice were acclimated to the test facility for at least 3 days. During this period, their general health was assessed by veterinary staff or other authorized personnel. The mice were housed in groups of up to four per cage in polysulfone cages, using either certified aspen shaving bedding or corncob bedding. This bedding was regularly tested for environmental contaminants by the manufacturer. The facility's environment was carefully controlled to maintain a temperature range of 20-26° C., relative humidity between 40 to 70%, and a 12-hour light/12-hour dark cycle, although this cycle can be interrupted for study-related activities. Temperature and humidity were continuously monitored by the Vawasala ViewLinc Monitoring system.

For dosing, an appropriate amount of the compounds was accurately weighed and mixed with a suitable volume of vehicle to achieve a clear solution. This process may require vertexing or sonication in a water bath. The animals were dosed within four hours of formulation preparation. Samples from each formulation were then collected for dose validation using either LC/UV or LC-MS/MS analysis.

Oral gavage was employed for dosing following the facility's SOPs, with the dose volume based on the animal's body weight measured on the morning of the dosing day. Compounds such as 5 mg/kg Clonidine HCl, 5 mg/kg dexmedetomidine HCl, 5 mg/kg and 80 mg/kg compound 1-B, and 5 mg/kg and 80 mg/kg compound 44-B were administered in a 20% HP-β-CD solution in water, with sample collections scheduled at 0.5, 1, 2, and 8 hours post-dosing.

Blood collections were performed from the saphenous vein or another suitable site, with approximately 0.1 mL collected per time point into pre-chilled commercial EDTA-K2 tubes. The samples were kept on wet ice until centrifugation at 4° C. and 3,200 g for 10 minutes. The plasma was then transferred into pre-labeled 96-well plates or polypropylene tubes, quick-frozen over dry ice, and stored at −60° C. or lower until LC-MS/MS analysis.

The brain and spinal cord tissues were harvested immediately, washed with cold saline, dried, and weighed. These samples were homogenized in a cold 15 mM PBS (pH 7.4):MeOH=2:1 solution at a 1:9 tissue-to-buffer ratio. The homogenates were split into two aliquots: one for immediate LC-MS/MS analysis and one stored at −70+10° C. as a backup. This comprehensive method ensures the detailed and standardized collection and analysis of pharmacokinetic data in a controlled and scientifically rigorous manner.

We calculated AUC ratio from the equation AUC ratio=Tissue AUC₀₋ₗₐₛₜ/plasma AUC₀₋ₗₐₛₜ. Log BB=log₁₀ (brain AUC₀₋ₗₐₛₜ/plasma AUC₀₋ₗₐₛₜ), Log SB=log₁₀ (brain AUC₀₋ₗₐₛₜ/spinal cord AUC₀₋ₗₐₛₜ), Kp=brain AUC₀₋ₗₐₛₜ/plasma AUC₀₋ₗₐₛₜ and Kp,uu,brain=AUCb,u/AUCp,u=AUCbrain/AUCplasma×(fu,brain/fu,plasma). The in vivo drug distribution result is shown in Table 7

TABLE 7
the in vivo drug distribution

| | | Brain/ | Spinal cord/ | | | Kp, uu, |
|---|---|---|---|---|---|---|
| Compounds | dosage | plasma, Kp | plasma | logBB | logSB | brain |
| Clonidine HCl | 5 mg/kg | 2.27 | 1.78 | 0.356 | 0.252 | 0.739 |
| Dexmedetomidine HCl | 5 mg/kg | 1.26 | 1.14 | 0.101 | 0.133 | 1.17 |
| 1-B HCl | 5 mg/kg | | below detection limit* | | | |
| | 80 mg/kg | 0.0598 | 0.0153 | −1.22 | −0.732 | 0.032 |
| 44-B HCl | 5 mg/kg | | below detection limit* | | | |
| | 80 mg/kg | 0.0334 | 0.0511 | −1.48 | −1.29 | 0.013 |

*drug in brain and spinal cord is below the detection limit (DL). DL = 10 ng/mL. The distribution parameters are not available

### Example 5. Efficacy Study on Spared Nerve Injury in Mice

50 male C57BL/6 mice weighing between 20-30 g were subjected to spared nerve injury (SNI) surgery, of which 6 mice were as sham surgery and the others were of SNI surgery. A few days after SNI surgery, all animals were subjected to mechanical allodynia test to obtain baseline paw withdrawal threshold (PWT). The qualified mice baseline PWT<0.6 g were randomly assigned to different groups (Vehicle group and test articles groups) based on baseline PWT and 6 sham mice as Sham group for evaluating efficacy of the test compounds, 8 mice in each group.

The animals were acclimated to the environment for 3-7 days after arriving at the animal facility. Three days before 1st mechanical allodynia test, the animals were habituated to the test environment for 15 minutes per day.

Aseptic techniques were employed by all surgeons, and all surgical instruments, including scissors, sharp forceps, scalpels, sterile cotton pads, needles, and metal clips, were sterilized prior to surgery. The animals were anesthetized with Zoletil 50 (50 mg/kg, 2.5 mL/kg, i.p.) and Xylazine Hydrochloride (8 mg/kg, 2.5 ml/kg, i.p.), with a toe pinch used to ensure full anesthesia before incision, and ophthalmic ointment applied to the rodents' eyes to prevent drying of the corneas. The fur on the posterior thigh was closely shaved, and the surgical area's skin was swabbed with three rounds of alternating Betadine and 70% ethanol, then allowed to dry. An incision was made on the lateral surface of the thigh, cutting through the biceps femoris muscle to expose the sciatic nerve and its terminal branches: the sural, common peroneal, and tibial nerves, with the common peroneal and tibial nerves being cut, leaving the sural nerve intact. The wound was closed in layers, with the skin sutured. Surgical instruments were cleaned and sterilized using a glass bead sterilizer post-operation. The animals recovered from anesthesia on a warm pad, were injected with 1 mL sterile saline subcutaneously to prevent dehydration, and returned to their home cage once fully awake and mobile.

On day 11, the animals were individually placed in plastic enclosures with mesh bottoms, allowing full paw access. For three consecutive days, mice were acclimated for 15 minutes each day. Mechanical allodynia baseline measurements were performed on day 14. Animals not exhibiting allodynia (PWT>0.6 g) were excluded, leaving 24 qualified animals (PWT<0.6 g) who were then randomly divided into three groups based on their baseline PWT, in addition to 6 sham mice forming a Sham group, totaling four groups with 6-8 mice each.

The administration route for the therapeutic intervention for compounds 1-B with a dosage from 1 mg/mL to 20 mg/mL, and 10-B, 44-B, 45-B, 46-B, 47-B, 121, 136, 118, 156 and 175 was oral (p.o.) with a dosage of 1 mg/mL, while the ones for 1 mg/kg morphine via s.c. and 3 mg/kg pregabalin via p.o. as positive control, which were prepared in a 20% HP-β-CD solution. 1-B, 10-B, 44-B 45-B, 46-B and 47-B are the active enantiomer of 1, 10, 44, 45, 46, 47, respectively, while 121, 136, 118, and 156 are racemate. The solution was vortexed to ensure thorough mixing until homogeneous. The dosage administered to the mice was 10 ml/kg.

Mechanical allodynia tests were conducted on the left hind paw of mice, which were individually placed in plastic enclosures with mesh bottoms for full paw access and acclimated for 15 minutes prior to testing. Following acclimation, the mid-plantar hind paw was probed using a series of eight Von Frey filaments with logarithmically incremental stiffness: 0.02 g (2.36), 0.04 g (2.44), 0.07 g (2.83), 0.16 g (3.22), 0.4 g (3.61), 0.6 g (3.84), 1 g (4.08), and 1.4 g (4.17). The filaments were applied perpendicularly to the paw's plantar surface with enough force to slightly buckle against it, maintaining contact for 6-8 seconds. Tests were spaced by 5-second intervals to ensure clear resolution of any response to the prior stimulus, with a sharp withdrawal or flinching upon filament removal indicating a positive response. Ambulatory reactions were deemed ambiguous, prompting a repeat of the stimulus. Testing began with the 0.16 g (3.22) filament, adjusting the force of subsequent filaments up or down depending on the mouse's response, following the Dixon up-down method. The maximum force used was the 1.4 g (4.17) filament, with the criteria for a positive response being a distinct withdrawal of the paw or flinching immediately after the filament's removal.

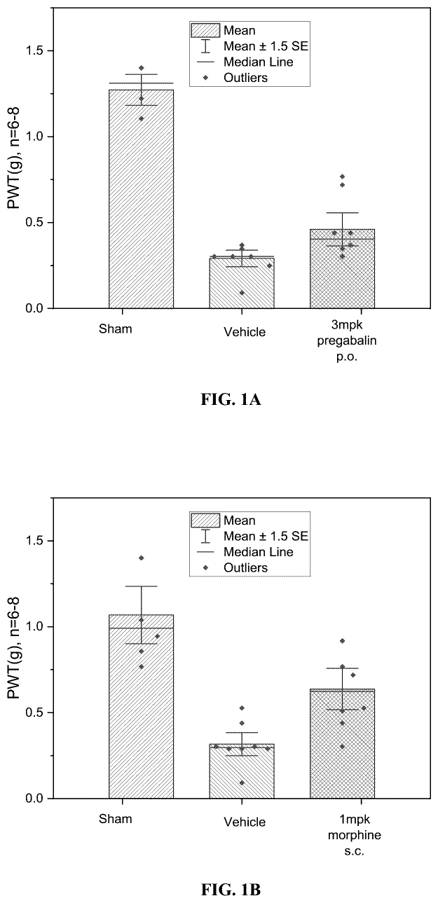

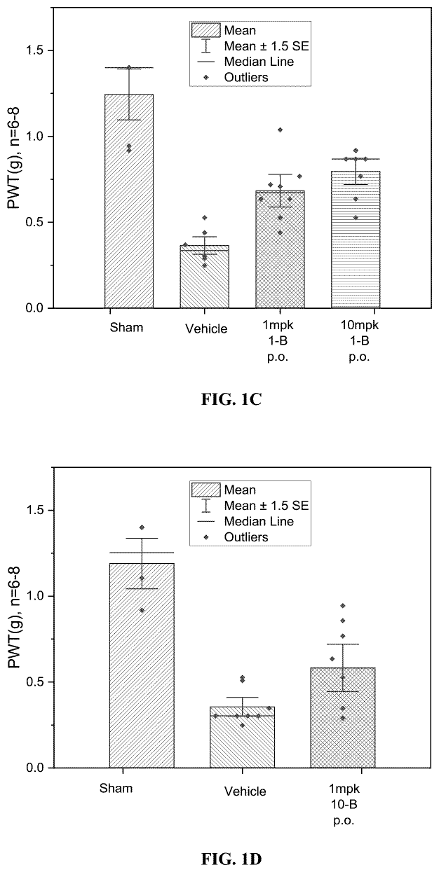

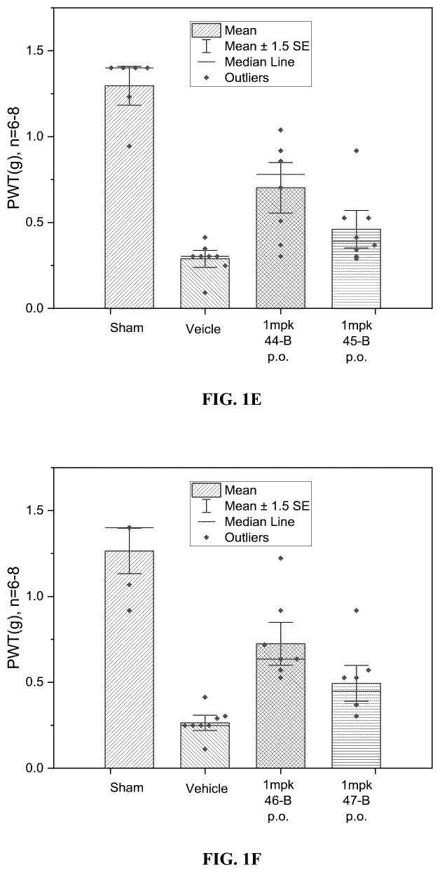

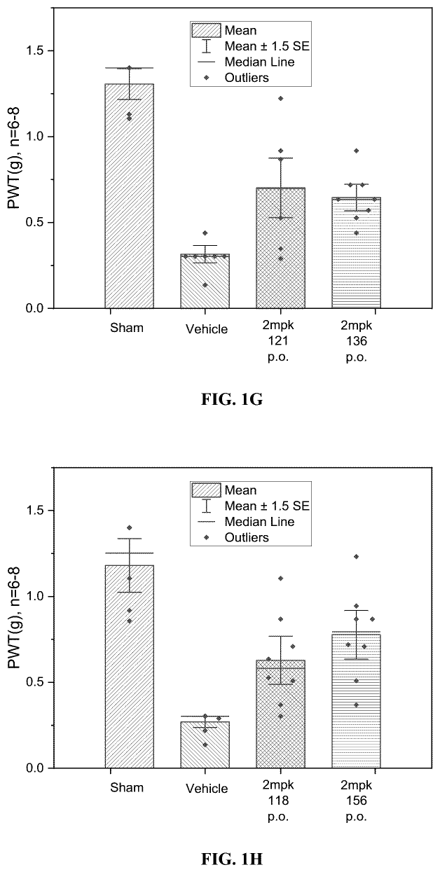

Data were presented in Prism 8.0 (Graph Pad Software, Inc.) by one-way ANOVA or two-way ANOVA followed by Dunnett's or Tukey's multiple comparison or by t test followed by two-tailed comparison test. The results are demonstrated by FIGS. 1A-1H.

### Example 7. Efficacy on Bone Cancer Pain Model in Mice

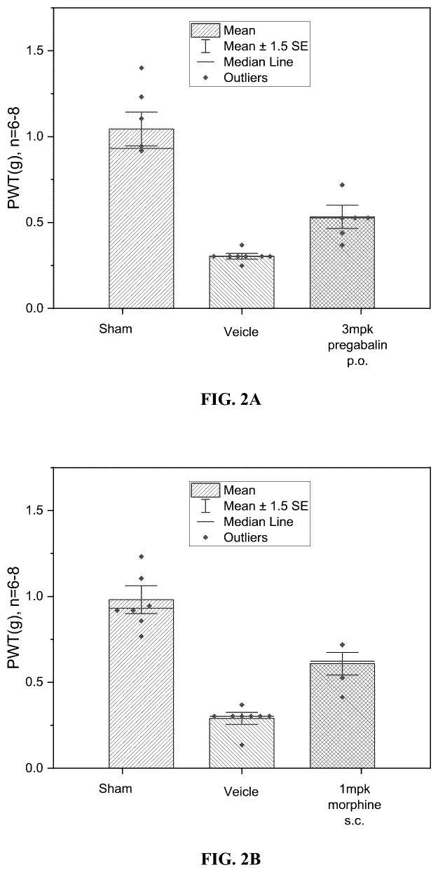

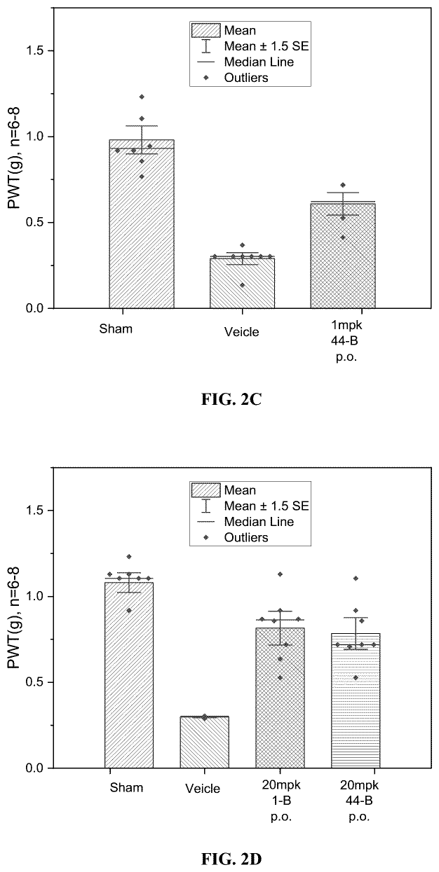

Animals were acclimatized to the environment for 3-7 days upon arrival at the facility. Male C3H/He mice were anesthetized with a combination of Zoletil 50 (50 mg/kg) and Xylazine Hydrochloride (8 mg/kg) administered via intraperitoneal injection, and positioned supinely. The right hind limb was shaved and sterilized. A minimal incision was made on the right hind leg to sever the patellar ligaments and expose the condyles of the distal femur. The proximal femur was perforated using a 0.3 mL syringe needle. A 10 μL suspension containing 2×10⁴ NCTC-2472 cells (suspended in a pellet formed from 2 mL of cell stock by centrifugation at 1000 rpm for 4 minutes, washed twice with 2 mL PBS, and resuspended in PBS at a concentration of 2×10⁶ cells/mL) was slowly injected into the intramedullary cavity of the femur. Control group animals received a 10 μL PBS injection (day 0). Animals were subsequently acclimatized to the testing environment for an additional three days before baseline PWT measurements were initiated. On day 14, baseline measurements for mechanical allodynia were conducted. Animals not exhibiting allodynia (PWT>0.6 g) were excluded. The remaining qualified animals were then randomly assigned into four groups based on their baseline PWT values as outlined in section 5.1. The animals received a single injection of test compounds, including pregabalin 3 mg/kg p.o., morphine 1 mg/kg s.c., 44-B 1 mg/kg p.o., as well as a group for 1-B 20 mg/kg p.o. and 44-B 20 mg/kg p.o. at a dose of 10 mL/kg based on body weight, and mechanical allodynia tests were performed at various time points post-administration as dictated by different experimental requirements with sham group and vehicle group. Each mouse was placed in a separate plastic enclosure with a mesh floor to freely access the paws and allowed to acclimate for 15 minutes prior to testing. Mechanical allodynia tests were conducted and analyzed as described in SNI model in example 6. The results are demonstrated by FIGS. 2A-2D.

### Example 8. Evaluation of the Efficacy in Post-Surgery Pain Model in Mice

Upon arrival at the facility, the animals were adaptively fed for 3 to 7 days. Additionally, for three days preceding the surgical procedures, all animals were placed in the test environment and acclimated daily for at least 15 minutes.

Aseptic techniques were rigorously applied by all surgeons. All surgical tools-including scissors, sharp forceps, scalpels, sterile cotton pads, needles, and metal clips-were sterilized prior to use. Animals were anesthetized using Zoletil 50 (50 mg/kg, 2.5 ml/kg, intraperitoneal) and Xylazine Hydrochloride (8 mg/kg, 2.5 ml/kg, intraperitoneal). A toe pinch confirmed deep anesthesia before any incisions were made. Ophthalmic ointment was applied to the animals' eyes to prevent corneal drying. The plantar aspect of the left hind paw was cleansed with three rounds of alternating Betadine and 70% ethanol applications, allowing the surface to air-dry. A 0.5-mm longitudinal incision was then made through the skin and fascia from 2 mm proximal to the heel towards the toes. The plantar muscle was longitudinally incised while preserving the origin and insertion points. Hemostasis was achieved with gentle pressure, and the skin was closed with two mattress sutures. Post-surgery, all surgical instruments were cleaned and re-sterilized using a glass bead sterilizer. Animals were allowed to recover from anesthesia on a heated recovery pad and were hydrated with 1 mL of sterile saline orally to prevent dehydration. Once fully awake and mobile, the animals were returned to their home cages.

On the first day post-surgery, all animals, including those in the Naive group, were assessed for mechanical allodynia using a Touch-Test Sensory Evaluator. Surgical animals not displaying allodynia (PWT>0.6 g) were excluded, leaving only 24 qualified surgical animals who were randomly assigned into three groups based on their baseline PWT, forming a total of four groups including the Naive group.

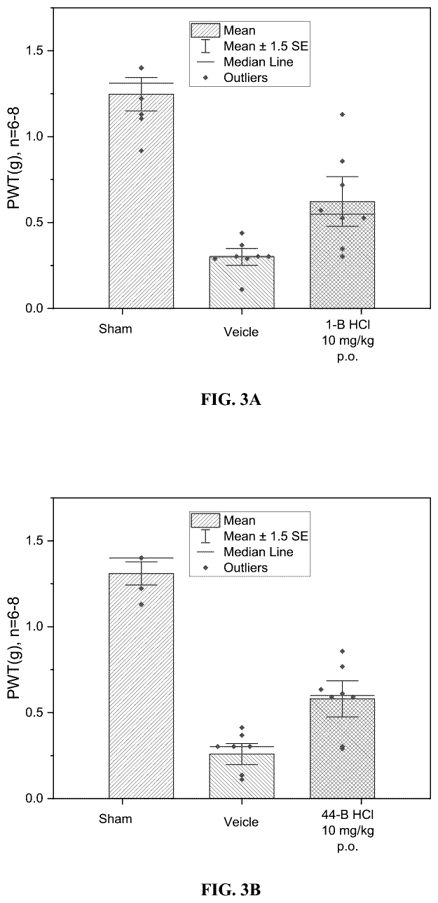

Animals were administered test compounds: morphine at 3 mg/mL s.c., 1-B HCl at 10 mg/mL p.o. and 44-B HCl at 10 mg/mL p.o., all at a dosage of 10 mL/kg based on body weight. Animals in the Naive group were also assessed but received no treatment. Mechanical allodynia tests were conducted and analyzed as described in SNI model in example 6. The results are demonstrated by FIGS. 3A-3C.

### Example 9. In Vivo Efficacy Study in the Treatment of Subcutaneous Colorectal Cancer Syngeneic Model MC38 in Female C57BL6/J Mice

The objective of this study is to evaluate the in vivo efficacy study of test articles in the Treatment of Subcutaneous Colorectal Cancer Syngeneic Model MC38 in Female C57BL6/J mice. The mice are Mus musculus C57BL6/J, female, supplied by Beijing HFK Bioscience Co. LTD, with an average age of 6-8 weeks. The cage is polysulfone IVC cage, with a temperature 20-26° C. and humidity 40-70%. The light cycle is 12 hours light and 12 hours dark. The mice is feed by a diet of standard rodent chow, irradiated, ad libitum. The water is autoclaved filtered RO (reverse osmosis) softened, filtered water, ad libitum.

Clonidine and compound 1-B HCl were used as control and test articles, respectively, and the study is designed according to the following table. Due to the poor state of mice caused by high dose, the dose of clonidine in G2 group and 1-B HCl in G4 group was adjusted from 5 mg/mL to 2 mg/mL and 10 mg/kg to 5 mg/kg, respectively, starting from Day 4. The detailed design and formulation is in Table 8.

TABLE 8
Study design and formulation

| | | | Dose | Dosing | Dosing | | |
|---|---|---|---|---|---|---|---|
| | No. of | | Level | Solution | Volume | | Dosing Frequency & |
| Group | mice | Treatment | (mg/kg) | (mg/mL) | (μL/g) | ROA | Duration |
| 1 | 6 | Vehicle | — | — | 10 | p.o. | QD × Day 0~Day 17 |
| 2 | 6 | Clonidine | 5 | 0.5 | 10 | p.o. | QD × Day 0~Day 3 |
| | | | 2* | 0.2 | 10 | p.o. | QD × Day 4~Day 17 |
| 3 | 6 | 1-B HCl | 5 | 0.5 | 10 | p.o. | BID × Day 0~Day 17 |
| 4 | 6 | 1-B HCl | 10 | 1 | 10 | p.o. | BID × Day 0~Day 3 |
| | | | 5* | 0.5 | 10 | p.o. | QD × Day 4~Day 17 |

*dosage adjusted on day 4

The MC38 cancer cells were maintained in vitro with DMEM medium supplemented with 10% fetal bovine serum and 50 μg/mL Hygromycin B at 37° C. in an atmosphere of 5% CO₂ in air. The cells in exponential growth phase were harvested and quantitated by cell counter before tumor inoculation. Each mouse was inoculated subcutaneously at the right rear flank region with MC38 tumor cells (1×10⁶) in 0.1 mL of PBS mixed with PBS for tumor development. The randomization started when the mean tumor size reached approximately 121.36 mm³. 30 mice were enrolled in the study. All animals were randomly allocated to 5 study groups, 6 mice in each group. Randomization was performed based on “Matched distribution” method. The date of randomization was denoted as day 0.

The treatment was initiated on the same day of randomization (day 0) per study design. After tumor cells inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail. Tumor volumes were measured twice per week after randomization in two dimensions using a caliper, and the volume was expressed in mm3 using the formula: “V=(L×W×W)/2, where V was tumor volume, L was tumor length (the longest tumor dimension) and W was tumor width (the longest tumor dimension perpendicular to L). Dosing as well as tumor and body weight measurements were conducted in a Laminar Flow Cabinet. The body weights and tumor volumes were measured by using StudyDirector™ software (version 3.1.399.19).

The body weights of all animals were monitored throughout the study and animals were euthanized if they lose over 20% of their body weight relative to the weight on the day of randomization. Meanwhile, the individual mouse was euthanized if its tumor volume exceeds 3000 mm3. To deter cannibalization, any animal exhibiting an ulcerated or necrotic tumor were separated immediately and singly housed and monitored daily before the animal was euthanized or until tumor regression was completed. The mouse was euthanized rapidly if a) tumor ulcerates, and the ulceration diameter was greater than 5 mm, or pus or necrosis observed, and b) tumor burden, including metastasis, compromises animal's normal physiologic performances, e.g., orientation, access to food or water, etc.

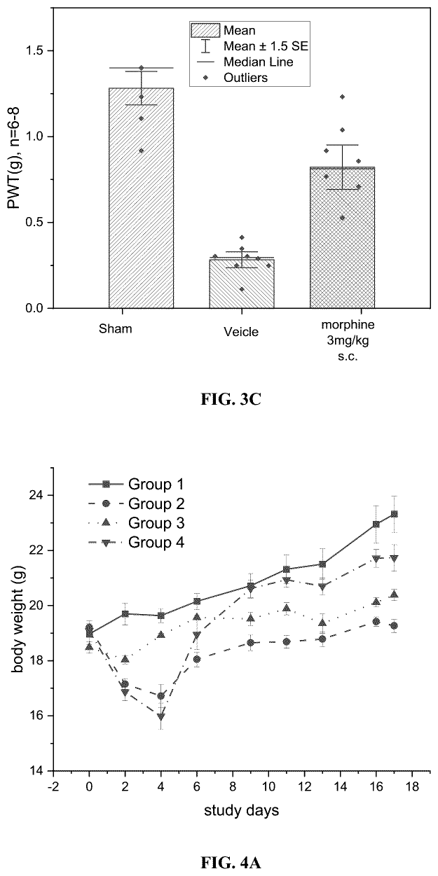

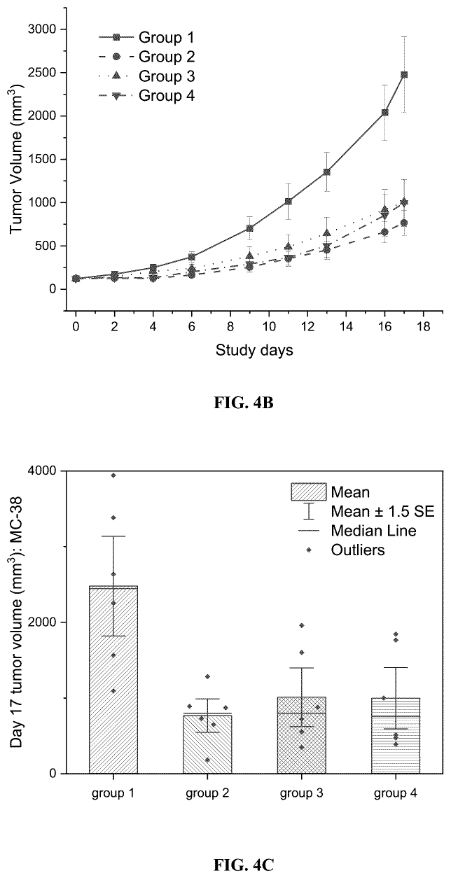

The body weight between randomization grouping is shown in FIG. 4A. The tumor growth of each treatment group and control group is shown in FIG. 4B. In day 17, the mice were sacrificed and the tumors were removed and measured. The data are shown in FIG. 4C. The tumor growth inhibition (TGI) and T/C were calculated based on the tumor size data of day 17, which is the last dosing day of the treatment. The Tumor volume were shown as the mean±SEM, while the T/C %=tumor volume of treatment group/tumor volume of control group×100%. The TGI %=(1-T/C)×100%. The pharmacodynamic analysis result is shown in Table 9.

TABLE 9
Pharmacodynamic analysis of each group in Subcutaneous
Colorectal Cancer Syngeneic Model MC38

| | D17 | | | |
|---|---|---|---|---|
| | Tumor volume | | | P Value |
| | (mm³) | TGI | T/C | (Compared with |
| Group | (mean ± SEM) | (%) | (%) | control group) |
| G1 | 2478.07 ± 439.10 | — | — | — |
| G2 | 766.52 ± 147.17 | 72.62% | 27.38 | <0.05 |
| G3 | 1009.16 ± 258.31 | 62.32% | 37.68 | <0.05 |
| G4 | 996.54 ± 270.05 | 62.86% | 37.14 | <0.05 |

### Example 10. Spontaneous Locomotor Activity in Mice

The study evaluated the effects of clonidine, brimonidine tartrate, compound 1-B HCl, compound 44-B HCl on spontaneous locomotor activity in male C57BL/6 mice. Initially, mice were acclimatized to the testing environment for 8 hours the day before the experiment, followed by at least 2 hours of habituation on the day of the test. The mice were then grouped randomly based on their body weight into six per group, ensuring a balanced distribution for the administration of the drug, which was dissolved in 20% HP-β-CD in water. On the test day, in one test, clonidine at a dose of 1 mg/kg and compound 1-B HCl at concentrations of 1 mg/kg, 10 mg/kg, and 20 mg/kg were freshly prepared and administered orally at a volume of 10 mL/kg. In another test, clonidine at a dose of 1 mg/kg, brimonidine tartrate at a dose of 1 mg/kg and compound 44-B HCl at a dose of 1 mg/kg were freshly prepared and administered orally at a volume of 10 ml/kg.

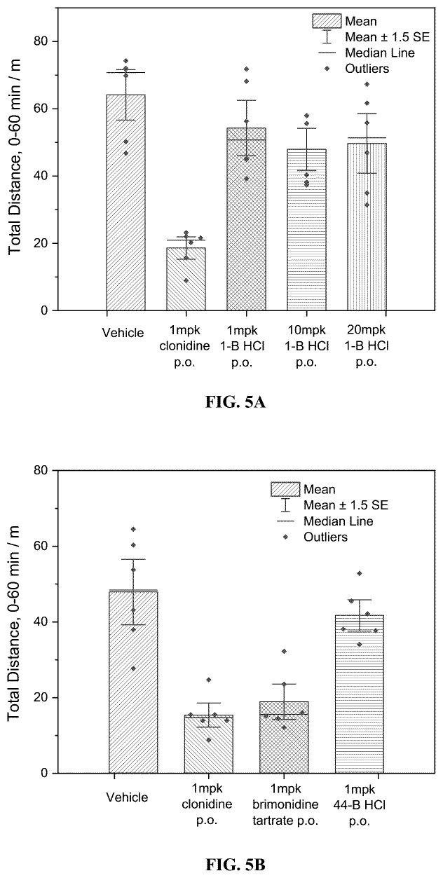

The locomotor activity was monitored by placing the mice in the center of a test box, with a video tracking system measuring the distance traveled every 5 minutes for 60 minutes. The testing began at T=0 minutes, immediately after administering the vehicle or compounds, and concluded at T=60 minutes. For data analysis, Prism 8.3.0 software was utilized, employing Two-way ANOVA followed by Bonferroni's multiple comparison test to analyze distance variations across different time points and One-way ANOVA followed by Dunnett's multiple comparisons test for assessing the total distance covered by the groups. A significance level of p<0.05 was established for determining significant differences. As demonstrated in FIG. 5, no significant sedation was observed after the treatment of compound 1-B HCl at 1 mg/kg, 10 mg/kg and 20 mg/kg in the first assay and compound 44-B HCl at 1 mg/kg in the second assay, while 1 mg/kg of clonidine in the first assay and 1 mg/kg brimonidine tartrate in the second assay leads to a significant sedation. The total distance between 0 to 60 minutes of the two tests is shown in FIGS. 5A and 5B.

### Example 11. The Effects on Motor Function in Mice

Upon arrival at the facility, the animals were acclimated for one week. The day before the rotarod training commenced, mice were randomly assigned to groups based on their body weight to ensure homogeneity across the groups in terms of weight before any treatment was administered.

Rotarod training occurred two days prior to the testing phase. On the first training day, the mice underwent three trials on the rotarod at a speed of 6 rpm, each lasting 120 seconds, with 30-minute intervals between trials. If a mouse fell off before completing 120 seconds, it was immediately placed back on the rotarod to complete the training duration. The following day, the training consisted of a single trial at the same speed of 6 rpm but extended to 300 seconds. Mice that fell before the 300-second mark were similarly returned to the rotarod to ensure they reached the full training time.

On the test day, treatments were administered orally to the mice at a dosage volume of 10 mL/kg based on their body weight. The treatments included a vehicle, clonidine (1 mg/kg), and 44-B HCl at three dosages (1 mg/kg, 10 mg/kg, and 20 mg/kg). The time of compound administration was designated as time zero.

The rotarod test was conducted at 30, 60, and 120 minutes post-administration, with each session lasting 300 seconds at a speed of 6 rpm. The primary measure was the latency time until a mouse fell from the rotarod, which served as an indicator of the compounds' effects on motor function. Data were recorded in Microsoft Excel and subsequently analyzed using GraphPad Prism. Statistical significance was assessed with a threshold P-value of less than 0.05, indicating meaningful differences between treatment groups.

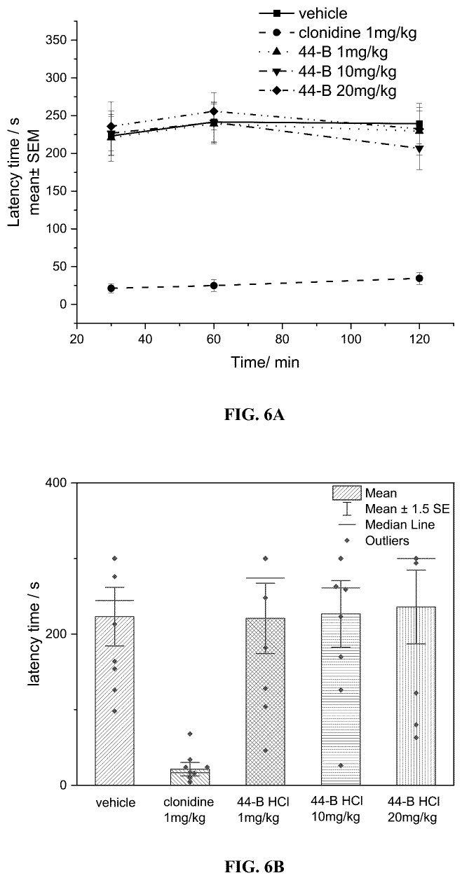

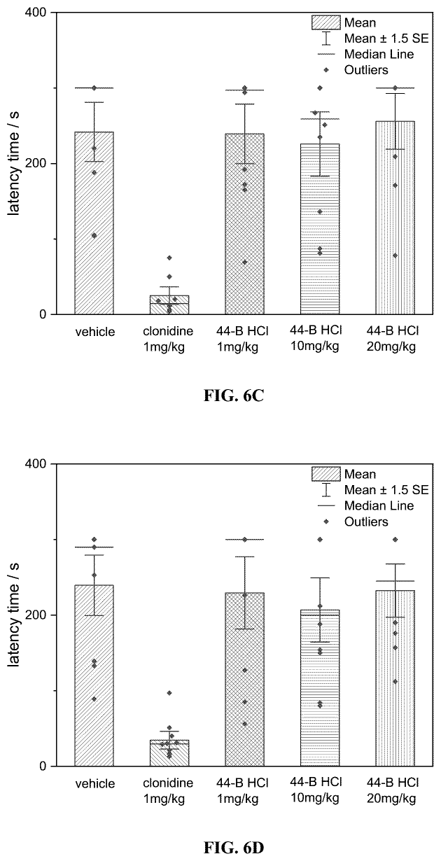

The detailed analysis method is described as follows. Initially, the data are assessed for normal distribution and homogeneity of variance. If the data adhere to both normal distribution and homogeneity of variance, a T-test is applied for comparisons involving two data sets, and a one-way ANOVA is utilized for analyses involving multiple data sets. In cases where the data exhibit normal distribution but heterogeneity of variance, Welch's T-test is used for two data sets, and a nonparametric test is employed for multiple data sets. If the data do not fit a normal distribution, the Mann-Whitney test is applied for two data sets, and the Kruskal-Wallis test is used for multiple data sets. The results are displayed in FIG. 6A to 6D.

1. A pharmaceutical composition comprising:
(a) a means for increasing the activation of α2 adrenergic receptor (α2AR) of the peripheral nervous system of a subject, and
(b) a pharmaceutically acceptable carrier.

2. A pharmaceutical composition comprising:
(a) a molecule comprising a moiety having formula 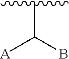
covalently linked to a means for increasing the distribution of said molecule to the peripheral nervous system of a subject, and
(b) a pharmaceutically acceptable carrier;
wherein,
A is chosen from: 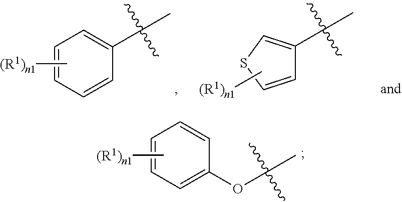
n1 is 1 or 2;
each R¹ is independently chosen from hydrogen, halogen, haloalkyl, hydroxyl, hydroxyalkyl, alkoxy, alkyl, and —COOH;
B is chosen from: 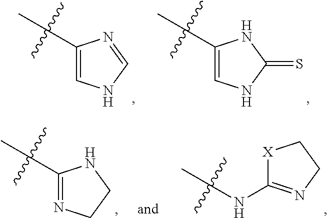
wherein X is S, O, or NH.

3. A pharmaceutical composition comprising:
(a) a molecule comprising a moiety having formula 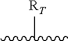
covalently linked to a means for activating α2 adrenergic receptor (α2AR), and
(b) a pharmaceutically acceptable carrier;
wherein,
R_{T} is 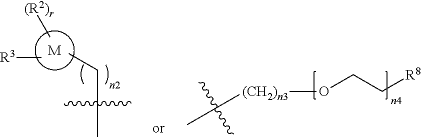
ring M is C₃₋₁₂ cycloalkyl, C₂₋₁₂ heterocyclyl, C₆₋₁₂ aryl, or C₁₋₁₂ heteroaryl, wherein the C₃₋₁₂ cycloalkyl or C₂₋₁₂ heterocyclyl is optionally fused with an aryl;
r is 1 or 2;
n2 is 0, 1, or 2;
each R₂ is independently chosen from hydrogen, halogen, hydroxyl, and alkoxy;
R³ is chosen from CN, hydroxy, alkoxy, —C(O)—C₀₋₁₂ alkylene-CN, —C₀₋₁₂ alkylene-C₂₋₁₂ heterocyclyl, —SO₂-alkyl, —C(O)—NR⁴R^{4′}, —SO₂—NR⁴R⁴, —C₀₋₁₂ alkylene-R^{3′}, —O—C₀₋₁₂ alkylene-COOH, —C₀₋₁₂ alkylene-N(R⁴)—C(O)—R⁵, —C₀₋₁₂ alkylene-N(R⁴)—SO₂—R⁵, —C₀₋₁₂ alkylene-C₁₋₁₂ heteroaryl, —C₀₋₁₂ alkylene-O—C₀₋₁₂ alkylene-N(R⁴)—SO₂—R⁵, —C₀₋₁₂ alkylene-P(═O)(R⁴)(R^{4′}), 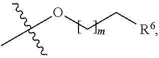
—NH—R⁷, or 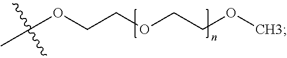
wherein one-CH₂— group in the —C₀₋₁₂ alkylene-R^{3′} is optionally replaced by oxygen atom or 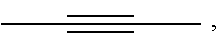
the —C₀₋₁₂ alkylene-COOH is optionally substituted with one or more substitutes chosen from amino and alkylamino, and the C₂₋₁₂ heterocyclyl and C₁₋₁₂ heteroaryl are each optionally substituted with one or more R^{4a};
R^{3′} is chosen from —C(O)—NR⁴R^{4′}, —SO₂—NR⁴R^{4′}, —C₀₋₁₂ alkylene-COOH, —C₀₋₁₂ alkylene-N(R⁴)—C(O)—R⁵, —C₀₋₁₂ alkylene-N(R⁴)—SO₂—R⁵, C₀₋₁₂ alkylene-C₁₋₁₂ heteroaryl;
each R^{4a} is independently chosen from hydroxy, alkyl, oxo, ketone, and —C₂₋₁₂ heterocyclyl;
each of R⁴ and R^{4′} is independently hydrogen, alkyl, alkoxy, —SO₂—N(R^{6a})ₜ, —C₀₋₁₂ alkylene-COOH, —C₀₋₁₂ alkylene-N(R^{6a})ₜ, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₀₋₁₂ alkylene-OR^{6a}, or hydroxyalkyl, wherein the hydroxyalkyl is optionally substituted with alkoxy; wherein each of the alkyl, C₃₋₁₂ cycloalkyl, C₂₋₁₂ heterocyclyl, and C₁₋₁₂ heteroaryl is optionally substituted with one or more R^{4a};
or R⁴ and R^{4′}, together with the nitrogen atom that they are attached to, form a heterocycle comprising one or more heteroatoms chosen from O, N, and S;
alternatively, when one R² is adjacent to R³, the R² and R³, together with the atoms that they are attached to, form a ring optionally substituted with one or more R^{4a};
R⁵ is amino, alkylamino, C₁₋₁₂ haloalkyl, —C₀₋₁₂ alkylene-OR^{6a}, —C₀₋₁₂ alkylene-N(R^{6a})ₜ, —C₀₋₁₂ alkylene-SR^{6a}, —C₀₋₁₂ alkylene-CN, —C₀₋₁₂ alkylene-C₃₋₁₂ cycloalkyl, —C₀₋₁₂ alkylene-C₂₋₁₂ heterocyclyl, —C₀₋₁₂ alkylene-C₁₋₁₂ heteroaryl, —C₂₋₁₂ alkenyl, or alkyl optionally substituted with cyano, amido, trialkylammonium, or thiolate; wherein each of the C₃₋₁₂ cycloalkyl, C₂₋₁₂ heterocyclyl, and C₁₋₁₂ heteroaryl is optionally substituted with one or more R^{4a},
each R^{6a} is independently chosen from hydrogen, C₁₋₁₂ alkyl, C₁₋₁₂ alkoxy, —C₀₋₁₂ alkylene-C₃₋₁₂ cycloalkyl, —C₀₋₁₂ alkylene-C₂₋₁₂ heterocyclyl, —C₀₋₁₂ alkylene-C₆₋₁₂ aryl, and —C₀₋₁₂ alkylene-C₁₋₁₂ heteroaryl; wherein each of the alkyl, C₃₋₁₂ cycloalkyl, C₂₋₁₂ heterocyclyl, and C₁₋₁₂ heteroaryl, is optionally substituted with one or more R⁴ª,
R⁶ is alkoxy, amino, sulfonamide, carbamide, or alkyl optionally substituted with cyano;
R⁷ is hydrogen, alkyl, —C₀₋₁₂ alkylene-COOH, optionally substituted C₃₋₁₂ cycloalkyl, C₂₋₁₂ aryl, C₁₋₁₂ heteroaryl, —C₀₋₁₂ alkylene-N(R+)—SO₂—R⁵, —C₀₋₁₂ alkylene-P(═O)(R⁴) (R^{4′}), —C₀₋₁₂ alkylene-N(R⁴)—C(═S)—R⁵, —C(═S)—R⁵, or alkyl optionally substituted with cyano;
R⁸ is alkoxy, amino, alkylamino, amide, sulfonamide, or carbamide;
n3 is 0, 1, 2, 3, or 4;
n4 is 1, 2, 3, 4, 5, or 6;
tis 2 or 3;
m is 0, 1, 2, 3, 4, or 5; and
n is 0, 1, 2, 3, or 4.

4. A pharmaceutical composition comprising:
(a) a molecule comprising (i) a first means for activating «2 adrenergic receptor (α2AR), and (ii) a second means for increasing distribution of the molecule to the peripheral nervous system of a subject, wherein the first means is covalently linked to the second means, and
(b) a pharmaceutically acceptable carrier.

5. A method for treating or preventing a disease in a subject in need thereof, the method comprising administering to the subject an effective amount of the pharmaceutical composition of claim 1.

6. The method of claim 5, wherein the disease is pain.

7. The method of claim 5, wherein the disease is neuropathic pain or post surgery pain.

8. The method of claim 5, wherein the disease is glaucoma, rosacea, or cancer.

9. A process for making a peripheral acting α2 agonist, the process comprising covalently linking a systemic α2 agonist to a means for increasing distribution to the peripheral nervous system of a subject.

10. A method for treating or preventing a disease in a subject in need thereof, the method comprising administering to the subject an effective amount of the pharmaceutical composition of claim 2.

11. The method of claim 10, wherein the disease is pain.

12. The method of claim 10, wherein the disease is neuropathic pain or post surgery pain.

13. The method of claim 10, wherein the disease is glaucoma, rosacea, or cancer.

14. A method for treating or preventing a disease in a subject in need thereof, the method comprising administering to the subject an effective amount of the pharmaceutical composition of claim 3.

15. The method of claim 14, wherein the disease is pain.

16. The method of claim 14, wherein the disease is neuropathic pain or post surgery pain.

17. The method of claim 14, wherein the disease is glaucoma, rosacea, or cancer.

18. A method for treating or preventing a disease in a subject in need thereof, the method comprising administering to the subject an effective amount of the pharmaceutical composition of claim 3.

19. The method of claim 18, wherein the disease is pain.

20. The method of claim 18, wherein the disease is neuropathic pain or post surgery pain.

21. The method of claim 18, wherein the disease is glaucoma, rosacea, or cancer.
